(19) United States
(12) Patent Application Publication
(10) Pub. No.: US 2024/0327864 A1
(43) Pub. Date: Oct. 3, 2024

(54) DIAPHRAGM-SPECIFIC NUCLEIC ACID REGULATORY ELEMENTS AND METHODS AND USE THEREOF

(71) Applicant: VRIJE UNIVESITEIT BRUSSEL, Brussel (BE)

(72) Inventors: Lay Khim CHUAH, Bierbeek (BE); Thierry Vandendriessche, Bierbeek (BE); Warut Tulalamba, Brussel (BE)

(73) Assignee: VRIJE UNIVESITEIT BRUSSEL, Brussel (BE)

(21) Appl. No.: 18/417,465

(22) Filed: Jan. 19, 2024

Related U.S. Application Data
(62) Division of application No. 16/498,690, filed on Sep. 27, 2019, now Pat. No. 11,920,149.

(30) Foreign Application Priority Data
Mar. 27, 2017 (EP) 17163080.9

(51) Int. Cl. C12N 15/86; A61K 48/00; A61P 21/00; C12N 9/26

(52) U.S. Cl. CPC C12N 15/86; A61K 48/00; A61P 21/00; C12N 9/2408; C12Y 302/0102; C12N 2750/14143; C12N 2800/22; C12N 2830/008

(57) ABSTRACT

The present invention relates to nucleic acid regulatory elements that are able to enhance diaphragm-specific expression of genes, in particular expression in diaphragm as such, or in combination with expression in cardiac muscle and/or skeletal muscle, methods employing these regulatory elements and uses of these elements. Expression cassettes and vectors containing these nucleic acid regulatory elements are also disclosed. The present invention is particularly useful for applications using gene therapy, more particularly diaphragm-directed gene therapy, and for vaccination purposes.

### FIELD

The present invention relates to nucleic acid regulatory elements that are able to enhance diaphragm-specific expression of genes, methods employing these regulatory elements and use thereof. The invention further encompasses expression cassettes, vectors and pharmaceutical compositions comprising these regulatory elements. The present invention is particularly useful for applications using gene therapy, more particularly diaphragm-directed gene therapy, or gene editing, and for vaccination purposes.

### BACKGROUND

The diaphragm is a sheet of internal skeletal muscle that extends across the bottom of the thoracic cavity. The diaphragm separates the thoracic cavity containing the heart and lungs, from the abdominal cavity and performs an important function in respiration: as the diaphragm contracts, the volume of the thoracic cavity increases, and air is drawn into the lungs. As with any organ or muscle, the diaphragm is subject to disorders and abnormalities, which come in many different forms and can stem from injury or illness. Consequently, diaphragm dysfunction can result in severe respiratory problems with potential fatal consequences. Diaphragmatic weakness and paralysis can be classified according to the anatomic region of abnormality. Diaphragmatic paralysis and weakness may be unilateral or bilateral, temporary or permanent, depending on the cause.

There are currently no effective cures available to treat the potential life-threatening diaphragm dysfunction in many of these and other muscle disorders. Hence, there is a need to establish effective cures by gene therapy to enable robust expression of the cognate therapeutic genes in the diaphragm. This requires the development of potent expression cassettes containing the genes of interest.

Consequently, there is a need to identify robust nucleic acid regulatory elements capable of substantially increasing transcription in the diaphragm. These nucleic acid regulatory elements are critically important for the regulation of gene expression in a tissue-specific manner. They are typically composed of clusters of transcription factor binding site (TFBS) motifs. The types and arrangement of TFBS and epigenetic modification patterns influence gene expression levels and specificity. Conventional methods of vector design relied on haphazard trial-and-error approaches whereby transcriptional enhancers were combined with promoters to boost expression levels. Though this could sometimes be effective, it often resulted in non-productive combinations that resulted in either modest or no increased expression levels of the gene of interest and/or loss of tissue specificity. Moreover, these conventional approaches did not take account of the importance of including evolutionary conserved regulatory motifs into the expression modules, which is particularly relevant for clinical translation.

There remains a need in the art for safe and efficient gene delivery to diaphragm, skeletal muscles and/or heart.

### SUMMARY OF THE INVENTION

The present inventors have relied on a computational approach (cf. FIG. 1) to identify robust nucleic acid regulatory elements that boost gene expression at the transcriptional level in the diaphragm (i.e. diaphragm: Dph). This requires the following computational steps: (1) diaphragm-specific genes were identified that are highly and specifically expressed based on expression data from diaphragm; (2) publicly available databases (ENSEMBL) were used for extracting the sequences upstream of the Transcriptional Start Site (TSS) of the selected genes. 3) These sequences were then submitted into UCSC Genome Browser Database for locating the transcription start site in human genome. To extract the corresponding diaphragm nucleic acid regulatory elements, defined herein as nucleic acid regulatory elements, the sequences were selected based on the following criteria: a) rich TFBS content, b) epigenetic signatures associated with high DNase hypersensitivity or chromatin accessibility (i.e. histone modifications), and c) evolutionary conserved clusters of TFBS associated with highly expressed diaphragm-specific genes.

As shown in the experimental section, the inventors identified nucleic acid regulatory elements that will specifically enhance gene expression in diaphragm. In addition, the inventors identified nucleic acid regulatory elements with combined diaphragm-specific and other tissue-specific expression such as for skeletal muscle and cardiac and skeletal muscle expression. Exemplary, but not limiting, muscle- or heart-muscle-specific regulatory elements are those that identified previously in European Patent Applications WO2015/110449A1 and WO2011/051450A1.

This combination approach will hence allow robust expression in diaphragm as well as other tissues that are affected by particular diseases that affect both diaphragm and skeletal muscles such as e.g. MTM. However, for diseases such as GSD-II and DMD which affects different tissues such as the diaphragm, skeletal muscles and heart, a combination of diaphragm nucleic acid regulatory element with the skeletal muscle nucleic acid regulatory element and/or the cardiac nucleic acid element will be developed. The diaphragm regulatory elements and the combination of diaphragm and other tissues elements (skeletal muscle and/or cardiac elements) will subsequently be validated in vivo yielding efficient and multiple tissue-specific gene expression. This approach hence, allows for the use of lower and thus safer vector doses, while maximizing therapeutic efficacy.

The invention therefore provides the following aspects:

In the screening, 89 regulatory elements were identified that can increase the expression of a transgene in a gene therapy delivery system such as in a vector system such as in an AAV vector system, increasing expression in diaphragm tissue. These elements are depicted in Tables 3 and 4 below. Table 3 depicts the Diaphragm cis regulatory elements (denominations “Dph-CREs”, “CREs” or “CRMs” or “SH” used herein are the same and interchangeable) (Dph-CREs) which are particularly relevant for increasing expression in diaphragm and skeletal muscle, while Table 4 depicts the Diaphragm CREs (Dph-CREs) which are particularly relevant for increasing expression in diaphragm, skeletal muscle and heart tissue. Seven of these CREs are present in both groups.

In particular, Dph-CRE02, Dph-CRE04, Dph-CRE58, Dph-CRE59, Dph-CRE60, Dph-CRE64, Dph-CRE06, Dph-CRE21, Dph-CRE41 and Dph-CRE18 (Table 3 & FIGS. 5A to 5I) have been validated in vivo for their high expression in diaphragm and skeletal muscle tissue. More particularly, Dph-CRE-02 Dph-CRE-64 and Dph-CRE-21 are preferred. Even more preferably, Dph-CRE64 is used for treating diseases which require upregulation of transgene expression in Diaphragm and skeletal muscle, and less in heart. In a specific embodiment, these Dph-CREs, more preferably the Dph-CRE64 (or Dph-CRE02 or CRE-21) are combined with muscle specific CRE Sk-SH4 (SEQ ID NO; 121). In a more preferred embodiment, said combination of CREs is coupled to the human desmin promoter, more preferably the 1,4kb variant thereof (SEQ ID NO; 92). For treatment of MTM disease, said CRE-promoter combination is preferably driving the hMTM1 (SEQ ID NO: 95) or more preferably the codon optimized hMTM1 transgene (SEQ ID NO; 96).

Myotubular myopathy (MTM) affects primarily the skeletal muscles and diaphragm, whereas the heart is rarely affected. Hence, ideally expression of the therapeutic MTM1 transgene should be targeted to skeletal muscles and diaphragm but not the heart. Therefore the SK-SH4-hDes1.4kb expression cassette is referred since in this cassette expression in skeletal muscle is high, while it is much lower in heart when combined with the (Dph-CRE-64) compared to e.g. the CSk-SH5-SPc which result in widespread cardiac gene expression when combined with the top Dph-CREs such as the Dph-CRE-02, -04 & 06.

As an example, the most preferred AAV vector combination for treating MTM is defined by SEQ ID NO: 131, pAAVss-CRE64-Sk-SH4-hDES1.4kb-MVM-hMTM1co-SynthpA.

Alternatively, Dph-CRE69, 70, 71, 66, 68, 77, 02, 04, 06, 07 (Table 4 and FIGS. 5A to 5I) have been validated in vivo for their high expression in diaphragm, skeletal muscle and heart tissue. More particularly, Dph-CRE-02, Dph-CRE-04 and CRE-06 are preferred. In a specific embodiment, said Dph-CREs, more preferably Dph-CRE-02,Dph-CRE04 (or Dph-CRE06) are combined with skeletal muscle and heart specific CRE CSk-SH5 (SEQ ID NO: 122). In a more preferred embodiment, said combination of CREs is coupled to the SPc5-12 promoter (SEQ ID NO: 124). SPc5-12 (sometimes called SPC-5-12-GTRM, which term is interchangeable). For treatment of e.g. Pompe disease, said CRE-promoter combination is preferably driving the hGAA (SEQ ID NO: 93) or more preferably the codon optimized hGAAco transgene (SEQ ID NO; 94).

The rationale for this design is based on the fact that in Pompe disease the GAA transgene is defective, which affects mainly skeletal muscles, diaphragm and the heart. Hence, ideally expression of the therapeutic GAA gene should be targeted to these affected tissues. The CSk-SH5-SPc combination leads to robust and specific expression in diaphragm, the skeletal muscles and heart and is therefore well suited to target those tissues that are affected in Pompe. As an example, the most preferred AAV vector combination for treating Pompe disease is defined by SEQ ID NO: 130). pAAVss-CRE04-CSk-SH5-SPc5-12GTRM-MVM-hGAAco-SynthpA (SEQ ID NO: 130).

Based on the above arguments, the present invention provides 10 different AAV constructs which were tested for expressing therapeutic genes:
1) pAAVss-hDes1.4kb-MVM-hMTM1-SynthpA (no diaphragm CRE, no muscle CRE Sk-SH4, only Desmin1.4kb promoter driving the MTM1 gene expression+MTM1) (SEQ ID NO; 135)
2) pAAVss-hDes1.4kb-MVM-hMTM1co-SynthpA (no diaphragm CRE, no muscle CRE Sk-SH4, +Des1.4kb+codon opt MTM1) (SEQ ID NO; 134)
3) pAAVss-Sk-SH4-hDes1.4kb-MVM-hMTM1-SynthpA (no diaphragm CRE, +muscle CRE Sk-SH4+Des1.4kb+MTM1) (SEQ ID NO; 137)
4) pAAVss-Sk-SH4-hDes1.4kb-MVM-hMTM1co-SynthpA (no diaphragm CRE, +muscle CRE Sk-SH4+Des1.4kb+codon opt MTM1) (SEQ ID NO; 136)
5) pAAVss-CRE64-Sk-SH4-hDes1.4kb-MVM-hMTM1co-SynthpA (contain best selected Diaphragm CRE64 combined with muscle CRE Sk-SH4) (SEQ ID NO; 131)
6) pAAVss-SPc5-12GTRM-MVM-hGAA-SynthpA (no diaphragm CRE, no muscle CRE, only SPc5-12-GTRM promoter driving the GAA gene expression)) (SEQ ID NO; 139)
7) pAAVss-SPc5-12GTRM-MVM-hGAAco-SynthpA (no diaphragm, no muscle CRE CSk-SH5, +SPc5-12-GTRM+codon opt GAA) (SEQ ID NO; 138)
8) pAAVss-CSK-SH5-SPc5-12GTRM-MVM-hGAA-SynthpA (no diaphragm CRE, +muscle CRE CSk-SH5+SPc5-12-GTRM+GAA) (SEQ ID NO; 133)
9) pAAVss-CSk-SH5-SPc-5-12GTRM-MVM-hGAAco-SynthpA ((no diaphragm CRE, +muscle CRE CSK-SH5+SPc5-12-GTRM+codon opt GAA) (SEQ ID NO; 132)
10) pAAVss-CRE04-CSk-SH5-SPc-5-12GTRM-MVM-hGAAco-SynthpA ((contain best selected Diaphragm CRE04 combined with muscle CRE CSk-SH5) (SEQ ID NO; 130)

The invention further provides the following aspects:

Aspect 1: a nucleic acid regulatory element for enhancing diaphragm-specific gene expression comprising, consisting essentially of, or consisting of the sequence selected from the group consisting of: SEQ ID NO:1 to 89, a sequence having at least 80%, preferably at least 85%, more preferably at least 90%, even more preferably at least 95%, such as 95%, 96%, 97%, 98%, or 99%, identity to any of these sequences. This aspect hence preferably provides for a nucleic acid regulatory element for enhancing gene expression in diaphragm, having a maximal length of 1000 nucleotides, preferably 800 nucleotides, more preferably 700 nucleotides, 600 nucleotides or 550 nucleotides, comprising the sequence defined by SEQ ID NO: 4, a sequence having at least 80%, preferably at least 85%, more preferably at least 90%, even more preferably at least 95%, such as 95%, 96%, 97%, 98%, or 99%, identity to any of these sequences, the complement of said sequence, or a sequence hybridizing under stringent conditions to said nucleic acid regulatory element, or the use thereof for enhancing diaphragm-specific gene expression. In either one of these embodiments, SEQ ID NO:4 can be replaced by either one of SEQ ID NO: 1-89.

Aspect 2: the nucleic acid regulatory element according to aspect 1 for enhancing gene expression in the diaphragm and skeletal muscle, comprising consisting essentially of, or consisting of the sequence selected from the group consisting of: SEQ ID NO:1 to 7 and 10 to 65, 82, and 83 (cf. Dph-CRE-1 to 7, 10 to 65, 82, and 83 in Table 3), or a sequence having at least 80%, preferably at least 85%, more preferably at least 90%, even more preferably at least 95%, such as 95%, 96%, 97%, 98%, or 99%, identity to any of these sequences, or the use thereof for enhancing gene expression in the diaphragm and skeletal muscle. In a preferred embodiment of said aspect, said regulatory element is Dph-CRE64 (SEQ ID NO: 64) or Dph-CRE02 (SEQ ID NO: 2) or Dph-CRE21 (SEQ ID NO: 21).

Aspect 3: the nucleic acid regulatory element according to aspect 1 or 2, having a maximal length of 1000 nucleotides, preferably 800 nucleotides, more preferably 700 nucleotides, still comprising the regulatory element defined by any one of SEQ ID NO: 1 to 7, 10 to 65, 82, and 83 (cf. Dph-CRE1 to 7, 10 to 65, 82, and 83 in Table 3). In a preferred embodiment of said aspect, said regulatory element is Dph-CRE64 (SEQ ID NO: 64) or Dph-CRE02 (SEQ ID NO: 2) or Dph-CRE21 (SEQ ID NO: 21).

Aspect 4: the nucleic acid regulatory element according to aspect 1 for enhancing gene expression in diaphragm, skeletal muscle and heart tissue comprising a functional fragment of a sequence selected from the group consisting of: SEQ ID NO: 1 to 9, 66 to 81, and 84 to 89, or a sequence having at least 80%, preferably at least 85%, more preferably at least 90%, even more preferably at least 95%, such as 95%, 96%, 97%, 98%, or 99%, identity to any of these sequences. (cf. Dph-CRE1 to 9, 66 to 81, and 84 to 89 in Table 4), or the use thereof for enhancing gene expression in diaphragm, skeletal muscle and heart tissue. In a preferred embodiment of said aspect, said regulatory element is Dph-CRE04 (SEQ ID NO: 4) or Dph-CRE06 (SEQ ID NO: 6) or Dph-CRE02 (SEQ ID NO: 2).

Aspect 5: the nucleic acid regulatory element according to aspect 1 or 4, having a maximal length of 1000 nucleotides, preferably 800 nucleotides, more preferably 700 nucleotides even more preferably 600 nucleotides, such as 500 nucleotides, still comprising the regulatory element defined by any one of SEQ ID No: 1 to 9, 66 to 81, and 84 to 89. (cf. Dph-CRE1 to 9, 66 to 81, and 84 to 89 in Table 4). In a preferred embodiment of said aspect, said regulatory element is Dph-CRE04 (SEQ ID NO: 4) or Dph-CRE06 (SEQ ID NO: 6) or Dph-CRE02 (SEQ ID NO: 2).

Aspect 6: a nucleic acid regulatory element for enhancing gene expression in diaphragm, skeletal muscle and heart tissue comprising, consisting essentially of, or consisting of the complement of a sequence as defined in any one of aspects 1 to 3, or hybridizing under stringent conditions to the nucleic acid regulatory element according to any one of aspects 1 to 3.

Aspect 7: a nucleic acid regulatory element for enhancing gene expression in diaphragm, skeletal muscle and heart tissue comprising, consisting essentially of, or consisting of the complement of a sequence as defined in any one of aspects 1, 4 or 5, or hybridizing under stringent conditions to the nucleic acid regulatory element according to any one of aspects 1, 4 or 5.

Aspect 8: use of the nucleic acid regulatory element according to any one aspects 1 to 3, and 6 in a nucleic acid expression cassette, or a vector, more particularly for enhancing gene expression in diaphragm and skeletal muscle of said nucleic acid expression cassette or vector.

Aspect 9: use of the nucleic acid regulatory element according to any one aspects 1, 4, 5, and 7 in a nucleic acid expression cassette, or a vector, more particularly for enhancing gene expression in diaphragm, skeletal muscle and heart tissue expression of said nucleic acid expression cassette or vector.

Aspect 10: a nucleic acid expression cassette comprising at least one, such as one, two, three, four, five or more, nucleic acid regulatory elements according to any one of aspects 1 to 7, operably linked to a promoter.

Aspect 11: the nucleic acid expression cassette according to aspect 10, wherein the nucleic acid regulatory element is operably linked to a promoter and a transgene.

Aspect 12: the nucleic acid expression cassette according any one of aspects 10 or 11, wherein the promoter is a diaphragm and skeletal muscle-specific promoter, such as the promotor of any one of the genes defined in Table 3, namely the ACTA1, CKM, TPM2, MYL1, TNNC2, FHL1, TNNT1, TNNI2, MYLPF, TNNT3, MYH2, SLN, MYBPC1, ENO3, CA3, ATP2A1, and MYH1 genes; or wherein the promoter is a diaphragm, skeletal muscle and heart-specific promoter, such as the promotor of any one of the genes defined in Table 4, namely ACTA1, CKM, MYL2, MB, DES, TNNC1, TCAP, MYH7, ALDOA, and TPM1 genes. In preferred embodiments of said aspect, and particularly of aspects 2 and 3, the promoter is the mouse or human Desmin promoter, more preferably the human 1.4kb Desmin promoter according to SEQ ID NO: 92. In preferred embodiments of said aspect and particularly of aspects 4 and 5, the promoter is the SPc5-12 promoter according to SEQ ID NO: 124.

Aspect 13: the nucleic acid expression cassette according to any one of aspects 10 to 12, wherein the transgene encodes a therapeutic protein or an immunogenic protein.

Aspect 14: the nucleic acid expression cassette according to any one of aspects 9 to 13, wherein the transgene encodes a secretable protein or a structural protein, such as myotubularin (MTM, SEQ ID NO: 95), acid glucosidase (GAA, SEQ ID NO: 93), follistatin, dystrophin, sarcoglycan, or dysferlin. In a preferred embodiment of said aspect, said transgenes are codon-optimised such as MTMco (SEQ ID NO: 96) or GAAco (SEQ ID NO: 94).

Additionally, the Dph-CRE can be supplemented by a further regulatory element (CRE) e.g. resulting in further expression in skeletal muscle or skeletal muscle and heart tissue such as the ones disclosed in WO2015/110449. Said additional CRE can be placed before or after the Dph-CRE (Exemplary vector backbones are depicted in FIG. 8). In a preferred embodiment, the skeletal muscle CRE Sk-SH4 (SEQ ID NO: 121), or the skeletal muscle and cardiac CREs CSk-SH5 (SEQ ID NO: 122) and CSk-SH1 (SEQ ID NO: 123) regulatory elements are used in combination with the Dph-CREs.

Specifically preferred is the combination of Dph-CRE64, 02 or 21 with CRE Sk-SH4, in particular the combination of Dph-CRE64 with CRE Sk-SH4. This embodiment is preferably coupled to the mouse or human Desmin promoter, more preferably the human 1.4kb Desmin promoter according to SEQ ID NO: 92. Even more preferred, said CRE-promoter combination is driving the MTM1 transgene (SEQ ID NO: 94) or its codon-optimised variant MTM1co (SEQ ID NO: 96). Said combination is particularly interesting for use in treating MTM disease.

Further specifically preferred is the combination of Dph-CRE04, 06 or 02 with CRE CSk-SH5, in particular the combination of Dph-CRE04 with CRE CSk-SH5. This embodiment is preferably coupled to the SPc5-12 promoter according to SEQ ID NO: 124. Even more preferred, said CRE-promoter combination is driving the hGAA (SEQ ID NO:93 transgene or its codon optimised variant hGAAco (SEQ ID NO: 94). Said combination is particularly interesting for use in treating Pompe disease.

Aspect 15: the nucleic acid expression cassette according to any one of aspects 9 to 14, further comprising an intron, preferably the Minute Virus of Mouse (MVM) intron (SEQ ID NO: 125).

Aspect 16: the nucleic acid expression cassette according to any one of aspects 9 to 15, further comprising a polyadenylation signal, preferably the synthetic poly-A site (SEQ ID NO: 127) or the Simian Virus 40 (SV40) polyadenylation signal.

Aspect 17: a vector comprising the nucleic acid regulatory element according to any one of aspects 1 to 7, or the nucleic acid expression cassette according to any one of aspects 10 to 16.

Aspect 18: the vector according to aspect 17, which is a viral vector, preferably an adeno-associated viral (AAV) vector, more preferably an AAV9 vector.

Aspect 19: the vector according to aspect 17, which is a non-viral vector, preferably a plasmid, a minicircle, an episomal vector, or a transposon-based vector, such as a PiggyBac-based vector or a Sleeping Beauty-based vector.

Particularly interesting embodiments of said aspect are the vectors defined by SEQ ID NO: 131 (especially for use in treating MTM disease) or 130 (especially for use in treating Pompe disease).

Aspect 20: a pharmaceutical composition comprising the nucleic acid expression cassette according to any one of aspects 10 to 16, or the vector according to any one of aspects 17 to 19, and a pharmaceutically acceptable carrier.

Aspect 21: the nucleic acid regulatory element according to any one of aspects 1 to 7, the nucleic acid expression cassette according to any one of aspects 10 to 16, the vector according to any one of aspects 17 to 19, or the pharmaceutical composition according to aspect 20 for use in medicine, more preferably for use in gene therapy.

Aspect 22: the nucleic acid regulatory element according to any one of aspects 1 to 3, and 6, the nucleic acid expression cassette according to any one of aspects 10 to 16, the vector according to any one of aspects 17 to 19, or the pharmaceutical composition according to aspect 20, for use in treating MTM.

Aspect 23: the nucleic acid regulatory element according to any one of aspects 1, 4, 5, and 7, the nucleic acid expression cassette according to any one of aspects 10 to 16, the vector according to any one of aspects 17 to 19, or the pharmaceutical composition according to aspect 20, for use in treating Pompe disease.

Alternatively, either one of the following diseases could be treated using the nucleic acid regulatory element according to any one of aspects 1 to 7, the nucleic acid expression cassette according to any one of aspects 10 to 16, the vector according to any one of aspects 17 to 19, or the pharmaceutical composition according to aspect 20: neuromuscular disorders and heart diseases, such as Abetalipoproteinemia (Bassen Kornzwieg), Acetylcholine Receptor Deficiency (Congenital Myasthenic Syndrome), Charlevoix-Saguenay Syndrome/Disease, Benign Congenital Myopathy, Brody Disesase, Centronuclear Myopathy (Myotubular Myopathy), Chondrodystrophic Myotonia (Schwartz-Jampel Syndrome), Chudley Sydrome, Fingerprint Myopathy, Hereditary Neuralgic Amyotrophy (Parsonage-Turner Syndrome), Inclusion Body Myopathy (e.g. Type 2 or Type 3), Inclusion Body Myositis, Isaac's Syndrome (Neuromyotonia), Kennedy's Disease (Spinal Bulbar (Muscular) Atrophy), Macrophagic Myofascitis, McAdle's Disease (Myophosphorylase Deficiency/Glycogen Storage Type V), Mononeuritis Multiplex, Muscle-Eye-Brain Disease, Nemaline Myopathy, Nonaka Myopathy, Rippling Muscle Disease, Tibial Muscular Dystrophy (Udd Distal Myopathy), Welander's Distal Myopathy, Acid Maltase Deficiency (Pompe's Disease/Glycogen Storage Disease Type II), Danon Disease (Gylcogen Storage Disease Type IIb/Vacuolar Myopathies), Debranching Enzyme Deficiency (Glycogen Storage Disease Type III/Forbe's Disease), Andersen Disease/Syndrome (Glycogen Storage Disease Type IV/Branching Enzyme Deficiency), Tauri's Disease (Glycogen Storage Disease Type VII/Phosphofructokinase Deficiency), Desmin Storage Myopathy (Myofibrillar Myopathy), Myodenylate Deaminase Deficiency, Adrenoleukodystrophy, Arthrogryposis Multiplex Congenita, Ataxia with Congenital Glaucoma, Ataxia with Vitamin E Deficiency, Barth Syndrome, Bethlem Myopathy, Carnitine Palmityl Transferase Deficiency, Carnitine Deficiency, Central Core Disease, Hereditary Motor and Sensory Neuropathy (e.g. Charcot-Marie-Tooth Diseases (CMT) such as CMT Type I, CMT Type II, CMT Type III (Dejerine-Sottas Disease), CMT Type IV (Refsum's Disease), CMT Type V; Peroneal Muscular Atrophy; Neuronal Type of Peroneal Muscular Atrophy), Hereditary Sensory and Autonomic Neuropathy (e.g. Type I, Type III (Familial Dysautonomia/Riley-Day Syndrome), Type IV (Congenital insensitivity to pain and anhidrosis), Congenital Fibre Type Disproportion Myopathy, Distal Spinal Muscular Atrophy, Familial Amyloid Neuropathy, Familial Dilated Cardiomyopathy with Muscular Dystrophy, Friedreich's Ataxia, Hyperkalemic Periodic Paralysis (Gamstorp Disease), Giant Axonal Neuropathy, Guillain-Barre Syndrome (Acute inflammatory Demyelinating/Polyradiculoneuropathy), Hyperthermia (Malignant Hyperthermia), Hypokalemic Periodic Paralysis, Iatrogenic Myopathy, Kearns-Sayre Syndrome, Kugelberg Welander Disease (Spinal Muscular Atrophy Type III), Laing Distal Myopathy, Lambert-Eaton (Myasthenic) Syndrome, Leigh's Syndrome, Minicore Myopathy/Multicore Myopathy, Mitochondrial Myopathy and/or Neuropathy, Mixed Connective Tissue Overlap Disease, Miyoshi Myopathy, Multifocal Motor Neuropathy with Conduction Block, Myasthenia Gravis, Myotonia Congenita (Thomsen's Disease), Myotonic Muscular Dystrophy (e.g. Type I (Steinert's Disease), Type II (Proximal Myotonic Myopathy)), Oculopharyngeal Muscular Dystrophy, Olivopontocerebellar Atrophy, Paramyotonia Congenita, Paraneoplastic neuropathy, Polymyopsitis, Reducing Body Myopathy, Scapuloperoneal Muscular Atrophy, Tubular Aggregate Myopathy, Walker-Warburg Syndrome, Werdnig-Hoffman Disease (Spinal Muscular Atrophy Type I), Zebra Body Myopathy, Nuclear Envelop Disease, muscular dystrophy (e.g. Duchenne muscular dystrophy (DMD)/Becker muscular dystrophy (BMD)), motor neuron diseases (MND), such as e.g. Charcot-Marie-Tooth Diseases (CMT) such as CMT Type I, CMT Type II, CMT Type III (Dejerine-Sottas Disease), CMT Type IV (Refsum's Disease), CMT Type V, spinal muscular atrophy (SMA), and amyotrophic lateral sclerosis (ALS), Emery-Dreifuss muscular dystrophy, facioscapulohumeral muscular dystrophy (FSHD), congenital muscular dystrophies, congenital myopathies, limb girdle muscular dystrophy, metabolic myopathies, muscle inflammatory diseases, myasthenia, mitochondrial myopathies, anomalies of ionic channels, nuclear envelop diseases, cardiomyopathies, cardiac hypertrophy, heart failure, distal myopathies, cardiovascular diseases, hemophilia, including hemophilia A and B, and diabetes.

In one embodiment, the diseases and disorders that may benefit from gene therapy using the Dph-CRE Sk-SH4 CRE in combination with, the Desmin promoter (e.g. the human DES 1.4kb promoter (SEQ ID NO:92) are diseases that affect the skeletal muscle, and include Abetalipoproteinemia (Bassen Kornzwieg), Acetylcholine Receptor Deficiency (Congenital Myasthenic Syndrome), Charlevoix-Saguenay Syndrome/Disease, Benign Congenital Myopathy, Brody Disesase, Centronuclear Myopathy (Myotubular Myopathy), Chondrodystrophic Myotonia (Schwartz-Jampel Syndrome), Chudley Sydrome, Fingerprint Myopathy, Hereditary Neuralgic Amyotrophy (Parsonage-Turner Syndrome), Inclusion Body Myopathy (e.g. Type 2 or Type 3), Inclusion Body Myositis, Isaac's Syndrome (Neuromyotonia), Kennedy's Disease (Spinal Bulbar (Muscular) Atrophy), Macrophagic Myofascitis, McAdle's Disease (Myophosphorylase Deficiency/Glycogen Storage Type V), Mononeuritis Multiplex, Muscle-Eye-Brain Disease, Nemaline Myopathy, Nonaka Myopathy, Rippling Muscle Disease, Tibial Muscular Dystrophy (Udd Distal Myopathy) and Welander's Distal Myopathy. In another embodiment, the diseases and disorders that may benefit from gene therapy using the Dph-CRE CSk-SH5 CRE combination with e.g. the SPc5-12 promoter are diseases that affect both the skeletal muscle and heart, and include Acid Maltase Deficiency (Pompe's Disease/Glycogen Storage Disease Type II), Danon Disease (Gylcogen Storage Disease Type IIb/Vacuolar Myopathies), Debranching Enzyme Deficiency (Glycogen Storage Disease Type III/Forbe's Disease), Andersen Disease/Syndrome (Glycogen Storage Disease Type IV/Branching Enzyme Deficiency), Tauri's Disease (Glycogen Storage Disease Type VII/Phosphofructokinase Deficiency), Desmin Storage Myopathy (Myofibrillar Myopathy), Myodenylate Deaminase Deficiency, Adrenoleukodystrophy, Arthrogryposis Multiplex Congenita, Ataxia with Congenital Glaucoma, Ataxia with Vitamin E Deficiency, Barth Syndrome, Bethlem Myopathy, Carnitine Palmityl Transferase Deficiency, Carnitine Deficiency, Central Core Disease, Hereditary Motor and Sensory Neuropathy (e.g. Charcot-Marie-Tooth Diseases (CMT) such as CMT Type I, CMT Type II, CMT Type III (Dejerine-Sottas Disease), CMT Type IV (Refsum's Disease), CMT Type V; Peroneal Muscular Atrophy; Neuronal Type of Peroneal Muscular Atrophy), Hereditary Sensory and Autonomic Neuropathy (e.g. Type I, Type III (Familial Dysautonomia/Riley-Day Syndrome), Type IV (Congenital insensitivity to pain and anhidrosis), Congenital Fibre Type Disproportion Myopathy, Distal Spinal Muscular Atrophy, Familial Amyloid Neuropathy, Familial Dilated Cardiomyopathy with Muscular Dystrophy, Friedreich's Ataxia, Hyperkalemic Periodic Paralysis (Gamstorp Disease), Giant Axonal Neuropathy, Guillain-Barre Syndrome (Acute inflammatory Demyelinating/Polyradiculoneuropathy), Hyperthermia (Malignant Hyperthermia), Hypokalemic Periodic Paralysis, Iatrogenic Myopathy, Kearns-Sayre Syndrome, Kugelberg Welander Disease (Spinal Muscular Atrophy Type III), Laing Distal Myopathy, Lambert-Eaton (Myasthenic) Syndrome, Leigh's Syndrome, Minicore Myopathy/Multicore Myopathy, Mitochondrial Myopathy and/or Neuropathy, Mixed Connective Tissue Overlap Disease, Miyoshi Myopathy, Multifocal Motor Neuropathy with Conduction Block, Myasthenia Gravis, Myotonia Congenita (Thomsen's Disease), Myotonic Muscular Dystrophy (e.g. Type I (Steinert's Disease), Type II (Proximal Myotonic Myopathy)), Oculopharyngeal Muscular Dystrophy, Olivopontocerebellar Atrophy, Paramyotonia Congenita, Paraneoplastic neuropathy, Polymyopsitis, Reducing Body Myopathy, Scapuloperoneal Muscular Atrophy, Tubular Aggregate Myopathy, Walker-Warburg Syndrome, Werdnig-Hoffman Disease (Spinal Muscular Atrophy Type I), Zebra Body Myopathy and Nuclear Envelop Disease.

Aspect 24: the nucleic acid regulatory element according to any one of aspects 1 to 7, the nucleic acid expression cassette according to any one of aspects 10 to 16, the vector according to any one of aspects 17 to 19, or the pharmaceutical composition according to aspect 20 for use as a vaccine, preferably a prophylactic vaccine, or for use in vaccination therapy, preferably prophylactic vaccination.

Aspect 25: A method, preferably an in vitro or ex vivo method, for expressing a transgene product in diaphragm and skeletal muscle cells, comprising:
introducing the nucleic acid expression cassette according to any one of aspects 10 to 16, or the vector according to any one of aspects 17 to 19, comprising the nucleic acid regulatory element according to any one of aspects 1 to 3 and 6, into the cells; and
expressing the transgene product in the cells.

Aspect 26: A method, preferably an in vitro or ex vivo method, for expressing a transgene product in diaphragm, skeletal muscle, and heart cells, comprising:
introducing the nucleic acid expression cassette according to any one of aspects 10 to 16, or the vector according to any one of aspects 17 to 19, comprising the nucleic acid regulatory element according to any one of aspects 1, 4, 5, and 7, into the cells; and
expressing the transgene product in the cells.

Aspect 27: a method for treating MTM comprising the administration of a therapeutically effective amount of the nucleic acid expression cassette according to any one of aspects 10 to 16, the vector according to any one of aspects 17 to 19, or the pharmaceutical composition according to aspect 20, each comprising the nucleic acid regulatory element according to any one of aspects 1 to 3, and 6, to a subject in need thereof.

Aspect 28: a method for treating Pompe disease comprising the administration of a therapeutically effective amount of the nucleic acid expression cassette according to any one of aspects 10 to 16, the vector according to any one of aspects 17 to 19, or the pharmaceutical composition according to aspect 20, each comprising the nucleic acid regulatory element according to any one of aspects 1, 4, 5, and 7, to a subject in need thereof.

Aspect 29: a method for treating any one of the diseases or disorders selected from the group comprising: muscular dystrophy (e.g. Duchenne muscular dystrophy (DMD)/Becker muscular dystrophy (BMD)), myotonic dystrophy, myotonic muscular dystrophy (DM), Duchenne dystrophinopathy, sarcoglycanopathies, Miyoshi myopathy, Fukuyama type congenital muscular dystrophy, dysferlinopathies, neuromuscular disease, motor neuron diseases (MND), such as e.g. Charcot-Marie-Tooth disease (CMT), spinal muscular atrophy (SMA), and amyotrophic lateral sclerosis (ALS), Emery-Dreifuss muscular dystrophy, facioscapulohumeral muscular dystrophy (FSHD), congenital muscular dystrophies, congenital myopathies, limb girdle muscular dystrophy, metabolic myopathies, muscle inflammatory diseases, myasthenia, mitochondrial myopathies, anomalies of ionic channels, nuclear envelop diseases, cardiomyopathies, cardiac hypertrophy, heart failure, distal myopathies, cardiovascular diseases, hemophilia, including hemophilia A and B, and diabetes, comprising the administration of a therapeutically effective amount of the nucleic acid expression cassette according to any one of aspects 10 to 16, the vector according to any one of aspects 17 to 19, or the pharmaceutical composition according to aspect 20, each comprising the nucleic acid regulatory element according to any one of aspects 1 to 7.

Aspect 30. A codon-optimised human GAA gene as defined in SEQ ID NO: 94 Aspect 31. The codon-optimised human GAA gene for use in gene therapy, more particularly for use in treatment of diseases requiring restoration of GAA expression or increase of GAA expression. More preferably, said disease is Pompe disease or Duchenne muscular dystrophy.

Aspect 32. A codon-optimised human MTM1 gene as defined in SEQ ID NO: 96 Aspect 33. The codon-optimised human MTM1 gene for use in gene therapy, more particularly for use in treatment of diseases requiring restoration of MTM1 expression or increase of MTM expression. More preferably, said disease is MTM disease or Duchenne muscular dystrophy.

Aspect 34. A vector comprising the codon-optimised human GAA gene of aspect 30.

Aspect 35. A vector comprising the codon-optimised human MTM1 gene of aspect 32.

Aspect 36. A pharmaceutical composition comprising the vector according to aspect 34 or 35.

Aspect 37. A vector according to any one of aspects 1, and 4 to 5, comprising the codon-optimised human GAA gene of aspect 30.

Aspect 38. A vector according to any one of aspects 1-3, and 5, comprising the codon-optimised human MTM1 gene of aspect 32.

Aspect 39. A method, preferably an in vitro or ex vivo method, for expressing a transgene product in diaphragm, skeletal muscle, and heart cells, comprising:
introducing the nucleic acid expression cassette according to aspects 10 to 16, or the vector according to any one of aspects 17 to 19, comprising a nucleic acid regulatory element having a maximal length of 1000 nucleotides, preferably 800 nucleotides, more preferably 700 nucleotides, 600 nucleotides or 550 nucleotides, comprising the sequence defined by SEQ ID NO: 4 or SEQ ID NO: 64, a sequence having at least 80%, preferably at least 85%, more preferably at least 90%, even more preferably at least 95%, such as 95%, 96%, 97%, 98%, or 99%, identity to said sequence, the complement of said sequence, or a sequence hybridizing under stringent conditions to said nucleic acid regulatory element, into the cells; and
expressing the transgene product in the cells.

### BRIEF DESCRIPTION OF DRAWINGS

FIG. 1: Flow diagram of the identification of potential nucleic acid regulatory elements specific for diaphragm and skeletal muscle, or specific for diaphragm, skeletal muscle and heart. A computational approach was used to identify the nucleic acid regulatory elements involving the following steps: (1) gene expression profiling of total RNA samples from different human tissues, including human diaphragm tissue, by RNA (Quantification) Next-generation sequencing, (2) comparison of the gene expression profiles to identify genes that are highly and specifically expressed in i) diaphragm and skeletal muscle (17 genes), and ii) diaphragm, skeletal muscle and heart (10 genes), (3) locating the transcription start site of the identified genes using ENSEMBL, and (4) selecting candidate nucleic acid regulatory elements using UCSC Genome Browser database based on i) high DNase hypersensitivity sites; ii) high content of epigenetic markers associated with open chromatin (acetylation, methylation); iii) high content of transcription factor binding sites; iv) strong evolutionary conservation among vertebrates; and v) conserved transcription factor binding sites in 3 species (human, rat, mouse).

FIG. 2: Expression of the genes ACTA1 (A), CKM (B), TPM2 (C), MYL1 (D), TNNC2 (E), FHL1 (F), TNNT1 (G), TNNI2 (H), MYLPF (I), TNNT3 (J), MYH2 (K), SLN (L), MYBPC1 (M), ENO3 (N), CA3 (0), ATP2A1 (P), MYH1 (Q) in total RNA samples from the recited human tissues. Expression is indicated as Fragments Per Kilobase of transcript per Million mapped reads (FPKM).

FIG. 3: Expression of the genes MYL2 (A), MB (B), DES (C), TNNC1 (D), TCAP (E), MYH7 (F), ALDOA (G), TPM1 (H) in total RNA samples from the recited human tissues. Expression is indicated as Fragments Per Kilobase of transcript per Million mapped reads (FPKM).

FIG. 4: In vivo comparison of desmin promoters. SCID mice were injected with 1×10¹⁰ vg/mouse of AAV9-luciferase vector containing the most robust skeletal muscle specific CRE (designated as Sk-SH4) in combination with different desmin promoters: Sk-SH4-hDES1.4kb, (SEQ ID NO: 92) Sk-SH4-hDES1.0kb & (SEQ ID NO: 91) Sk-SH4-mDES (SEQ ID NO: 90), quantified as Photons signal, in murine Biceps (A), diaphragm (B), gastrocnemius (C), heart (D), quadriceps (E), tibialis (F) and triceps (G).

FIGS. 5A to 5I: Comparison of tissue expression levels in absence or presence of Diaphragm CREs (Dph-CREs) according to the invention. FIG. 5A Expression in Diaphragm FIG. 5B Expression in Quadricep FIG. 5C Expression in Gastrocnemius FIG. 5D Expression in Tibialis FIG. 5E Expression in Bicep FIG. 5F Expression in Tricep FIG. 5G Expression in total skeletal muscle tissues FIG. 5H Expression in heart. In the left-hand graphs, the fold difference in expression of the different new Diaphragm CREs (Dph-CREs) in a vector backbone comprising the hDES1.4kb promotor and a muscle-specific CRE Sk-SH4 (SEQ ID NO: 121) is indicated versus the same vector backbone with hDES1.4kb promotor only (bottom numbers) and versus the same vector backbone without the new CREs (upper numbers). In the right-hand graphs, the fold difference in expression of the different new Diaphragm CREs in a vector backbone comprising the SPc5-12 promotor and a muscle-specific CRE CSk-SH5 (SEQ ID NO: 122) is indicated versus the same vector backbone with SPc5-12 promotor only (bottom numbers) and versus the same vector backbone without the new CRE's (upper numbers) FIG. 5I Specificity of Diaphragm CREs (mainly expressed in diaphragm, skeletal muscle and heart but not in other tissues. The numbers of the Diaphragm CREs correspond to the numbering of Tables 3 and 4.

FIGS. 6A and 6B: FIG. 6A Effect of the muscle-specific CRE (CSk-SH5—SEQ ID NO: 122) on the mRNA expression of human codon optimized GAA gene. FIG. 5B The increasing of GAA protein expression. The total proteins from the mice organs were extracted according to the manufacturer's protocol. The total proteins were diluted with the samples buffer and loaded into the hGAA ELISA plate. The background was subtracted with non-injected mice. The results show that codon-optimized hGAA sequence can improve the translation efficiency resulting in 2-3 folds higher expression.

FIGS. 7A and 7B: FIG. 7A Effect of the skeletal muscle specific CRE (Sk-SH4—SEQ ID NO: 121) on the mRNA expression of human codon optimized MTM1 gene. FIG. 7B Codon optimization led to an increase of 2.2 to 3.8 folds of hMTM1 protein expression in gastrocnemius and diaphragm respectively.

FIG. 8: Schematic view of AAV vector backbone. A) reference backbone with Dph-CREs coupled to a promotor, MVM intron, a transgene or reporter gene, and a polyA site. B-C) Same as A), but with an additional muscle CRE (Sk-CRE) or heart-muscle CRE (CSk-CRE) coupled after B) the Dph-CRE or before C) the Dph-CRE.

FIG. 9: Schematic view of AAV vectors for expression of A) hMTMco or B) hGAAco transgene. Vector A) further comprises one of the Dph-CREs (called Dia-CRM), the Sk-SH4-CRE (SEQ ID NO: 121), the hDes1,4kb promoter, the MVM intron, the hMTM1co transgene, and a synthetic poly-A site, cloned between the ITRs of an AAV vector. Vector B) further comprises one of the Dph-CREs (called Dia-CRM), the CSk-SH5-CRE (SEQ ID NO: 122), the SPc5-12 promoter, the MVM intron, the hGAAco transgene, and a synthetic poly-A site, cloned between the ITRs of an AAV vector.

FIG. 10: Increase of GAA therapeutic gene transcription using diaphragm-specific cis-regulatory element (Dph-CRE) CRE04 (SEQ ID NO: 4) in combination with CSk-SH5 (SEQ ID NO: 122). The fold-difference indicated on the top of each bar is calculated from relative expression between Dph-CRE-containing AAV (dark gray) and its control without Dph-CRE04 and CSk-SH5 (light gray).

FIG. 11: Increase of MTM1 therapeutic gene transcription using diaphragm-specific cis-regulatory element (Dph-CRE) CRE64 (SEQ ID NO:64) in combination with Sk-SH4 (SEQ ID NO: 121—called SKCRM4 in the figure). The fold-difference indicated on the top of each bar is calculated from relative expression between Dph-CRE-containing AAV (light gray) and its control without Dph-CRE64 and Sk-SH4 (black).

### DESCRIPTION

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms also encompass “consisting of” and “consisting essentially of”, which enjoy well-established meanings in patent terminology.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” refers is itself also specifically, and preferably, disclosed.

Whereas the terms “one or more” or “at least one”, such as one or more members or at least one member of a group of members, is clear per se, by means of further exemplification, the term encompasses inter alia a reference to any one of said members, or to any two or more of said members, such as, e.g., any 3, 4, 5, 6 or 7 etc. of said members, and up to all said members. In another example, “one or more” or “at least one” may refer to 1, 2, 3, 4, 5, 6, 7 or more.

The discussion of the background to the invention herein is included to explain the context of the invention. This is not to be taken as an admission that any of the material referred to was published, known, or part of the common general knowledge in any country as of the priority date of any of the claims.

Throughout this disclosure, various publications, patents and published patent specifications are referenced by an identifying citation. All documents cited in the present specification are hereby incorporated by reference in their entirety. In particular, the teachings or sections of such documents herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the invention. When specific terms are defined in connection with a particular aspect of the invention or a particular embodiment of the invention, such connotation is meant to apply throughout this specification, i.e., also in the context of other aspects or embodiments of the invention, unless otherwise defined.

In the following passages, different aspects or embodiments of the invention are defined in more detail. Each aspect or embodiment so defined may be combined with any other aspect(s) or embodiment(s) unless clearly indicated to the contrary. In particular, any feature indicated as being preferred or advantageous may be combined with any other feature or features indicated as being preferred or advantageous.

Reference throughout this specification to “one embodiment”, “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the appended claims, any of the claimed embodiments can be used in any combination.

For general methods relating to the invention, reference is made inter alia to well-known textbooks, including, e.g., “Molecular Cloning: A Laboratory Manual, 2nd Ed.” (Sambrook et al., 1989), “Current Protocols in Molecular Biology” (Ausubel et al., 1987).

In one aspect, the invention relates to a nucleic acid regulatory element for enhancing gene expression in diaphragm and skeletal muscle cells or tissue or in diaphragm, heart, and skeletal muscle cells or tissue comprising, consisting essentially of (i.e., the regulatory element may for instance additionally comprise sequences used for cloning purposes, but the indicated sequences make up the essential part of the regulatory element, e.g. they do not form part of a larger regulatory region such as a promoter), or consisting of a sequence selected from the group consisting of: SEQ ID NO:1 to 89, a sequence having at least 80%, preferably at least 85%, more preferably at least 90%, even more preferably at least 95%, such as 95%, 96%, 97%, 98%, or 99%, identity to any of these sequences, or a functional fragment of a sequence selected from the group consisting of: SEQ ID NO:1 to 89.

Table 3 below depicts the core nucleotide sequence of the different nucleic acid regulatory elements for enhancing gene expression in diaphragm and skeletal muscle cells or tissue and their corresponding genes and lengths.

TABLE 3
The CREs sequence for high expression in diaphragm and skeletal muscle.

| SEQ ID | | Dph-CRE | Size | |
|---|---|---|---|---|
| NO. | Gene | (CRE) | (bp) | Sequence |
| 1 | ACTA1 | Dph-CRE01 | 328 | GGAGACACTCCATATACGGCCCGGCCCGCGTTACCTGGG |
| | | | | ACCGGGCCAACCCGCTCCTTCTTTGGTCAACGCAGGGGA |
| | | | | CCCGGGCGGGGGCCCAGGCCGCGAACCGGCCGAGGGAGG |
| | | | | GGGCTCTAGTGCCCAACACCCAAATATGGCTCGAGAAGG |
| | | | | GCAGCGACATTCCTGCGGGGTGGCGCGGAGGGAATGCCC |
| | | | | GCGGGCTATATAAAACCTGAGCAGAGGGACAAGCGGCCA |
| | | | | CCGCAGCGGACAGCGCCAAGTGAAGCCTCGCTTCCCCTC |
| | | | | CGCGGCGACCAGGGCCCGAGCCGAGAGTAGCAGTTGTAG |
| | | | | CTACCCGCCCAGGTAG |
| 2 | ACTA1 | Dph-CRE02 | 452 | GACAGGTGCGGTTCCCGGAGCGCAGGCGCACACATGCAC |
| | | | | CCACCGGCGAACGCGGTGACCCTCGCCCCACCCCATCCC |
| | | | | CTCCGGCGGGCAACTGGGTCGGGTCAGGAGGGGCAAACC |
| | | | | CGCTAGGGAGACACTCCATATACGGCCCGGCCCGCGTTA |
| | | | | CCTGGGACCGGGCCAACCCGCTCCTTCTTTGGTCAACGC |
| | | | | AGGGGACCCGGGCGGGGGCCCAGGCCGCGAACCGGCCGA |
| | | | | GGGAGGGGGCTCTAGTGCCCAACACCCAAATATGGCTCG |
| | | | | AGAAGGGCAGCGACATTCCTGCGGGGTGGCGCGGAGGGA |
| | | | | ATGCCCGCGGGCTATATAAAACCTGAGCAGAGGGACAAG |
| | | | | CGGCCACCGCAGCGGACAGCGCCAAGTGAAGCCTCGCTT |
| | | | | CCCCTCCGCGGCGACCAGGGCCCGAGCCGAGAGTAGCAG |
| | | | | TTGTAGCTACCCGCCCAGGTAGG |
| 3 | ACTA1 | Dph-CRE03 | 239 | AGAGAGAGGGACAGGCACCAACTGGGTAACCTCTGCTGA |
| | | | | CCCCCACTCTACTTTACCATAAGTAGCTCCAAATCCTTC |
| | | | | TAGAAAATCTGAAAGGCATAGCCCCATATATCAGTGATA |
| | | | | TAAATAGAACCTGCAGCAGGCTCTGGTAAATGATGACTA |
| | | | | CAAGGTGGACTGGGAGGCAGCCCGGCCTTGGCAGGCATC |
| | | | | ATCCTCTAAATATAAAGATGAGTTTGTTCAGCCTTTGCA |
| | | | | GAAGG |
| 4 | ACTA1 | Dph-CRE04 | 509 | CCTTTTAGAGAATCCACACCTGTCCCAGTTGCTGGGTTC |
| | | | | CACTACCAAAAGTGAATTGCAACTATTTTAGGAGCACTT |
| | | | | AAGCACATCCGAAAAATGAGTGATTCTGTTCTGGCCCAC |
| | | | | ACCACATCACTGATGTACCCCCTTAAAGCATGTCCCTGA |
| | | | | GTTCATCACAGAAGACTGCTCCTCCTGTGCCCTCCACAA |
| | | | | GGTTAGAACTGTCCTTGTCTTAGGGAAAAAGGAGAGAGA |
| | | | | GAGAGAGAGAGAGAGAGAGAGAGAGAGAGAGAGAGAGAG |
| | | | | AGAGGGACAGGCACCAACTGGGTAACCTCTGCTGACCCC |
| | | | | CACTCTACTTTACCATAAGTAGCTCCAAATCCTTCTAGA |
| | | | | AAATCTGAAAGGCATAGCCCCATATATCAGTGATATAAA |
| | | | | TAGAACCTGCAGCAGGCTCTGGTAAATGATGACTACAAG |
| | | | | GTGGACTGGGAGGCAGCCCGGCCTTGGCAGGCATCATCC |
| | | | | TCTAAATATAAAGATGAGTTTGTTCAGCCTTTGCAGAAG |
| | | | | GA |
| 5 | ACTA1 | Dph-CRE05 | 169 | CCTTTTAGAGAATCCACACCTGTCCCAGTTGCTGGGTTC |
| | | | | CACTACCAAAAGTGAATTGCAACTATTTTAGGAGCACTT |
| | | | | AAGCACATCCGAAAAATGAGTGATTCTGTTCTGGCCCAC |
| | | | | ACCACATCACTGATGTACCCCCTTAAAGCATGTCCCTGA |
| | | | | GTTCATCACAGAA |
| 6 | CKM | Dph-CRE06 | 400 | GGGCCAGGGGACGGTGGCTTCTACGTGCTTGGGACGTTC |
| | | | | CCAGCCACCGTCCCATGTTCCCGGCGGGGGGCCAGCTGT |
| | | | | CCCCACCGCCAGCCCAACTCAGCACTTGGTCAGGGTATC |
| | | | | AGCTTGGTGGGGGGGCGTGAGCCCAGCCCCTGGGGCGGC |
| | | | | TCAGCCCATACAAGGCCATGGGGCTGGGCGCAAAGCATG |
| | | | | CCTGGGTTCAGGGTGGGTATGGTGCGGGAGCAGGGAGGT |
| | | | | GAGAGGCTCAGCTGCCCTCCAGAACTCCTCCCTGGGGAC |
| | | | | AACCCCTCCCAGCCAATAGCACAGCCTAGGTCCCCCTAT |
| | | | | ATAAGGCCACGGCTGCTGGCCCTTCCTTTGGGTCAGTGT |
| | | | | CACCTCCAGGATACAGACAGCCCCCCTTCAGCCCAGCCC |
| | | | | AGCCAGGTAC |
| 7 | CKM | Dph-CRE07 | 255 | CCCAGCCCCCTTCCCCGGGAGGTGGGAGCGGCCACCCAG |
| | | | | GGCCCCGTGGCTGCCCTTGTAAGGAGGCGAGGCCCGAGG |
| | | | | ACACCCGAGACGCCCGGTTATAATTAACCAGGACACGTG |
| | | | | GCGAACCCCCCTCCAACACCTGCCCCCGAACCCCCCCAT |
| | | | | ACCCAGCGCCTCGGGTCTCGGCCTTTGCGGCAGAGGAGA |
| | | | | CAGCAAAGCGCCCTCTAAAAATAACTCCTTTCCCGGCGA |
| | | | | CCGAGACCCTCCCTGTCCCCC |
| 10 | TPM2 | Dph-CRE10 | 608 | ACTCAGGGTAAACTGAGGCACTCAAACTGCCGAGGAGCT |
| | | | | CCGCCTCCCGAGAGACATTTAATCCGGGGGGATTTGCAG |
| | | | | GAAACTTCTAAATTAAGGGTAGCGGCTGCTGCAGCTGAG |
| | | | | GGGGGGCACGCCGGTCCCTGCGCCCGGGCAGCTGCCGTG |
| | | | | AGCTCACGCCCCGAAATAGCCCCAGGGGCCCCAGCCGCA |
| | | | | GCTGCCACTGGGTCCGGCTGTCACTCAGAGGAAGCACGG |
| | | | | AGCCCCCAGCCCAAGGGTCCCCTCCCCTTCGCATCGCCG |
| | | | | GGGTTTTTCCAGCCGACCGTCGGCCACTTTTTCCTCCGA |
| | | | | CGGCTGGCAGGGAAGAGGGGGATGGGGGCGGGACCCCAA |
| | | | | GGGAGGCGGTCCCCAGTGGGTGGGCGAAGGGGGCGGCCG |
| | | | | CACCCCCCGGCCGGGCCGTGCTTCTGCCCCTACAAGGTT |
| | | | | TGGGCCGAGGTGGGGGAGGGTCCTGGTTGCCGGCCCCGC |
| | | | | CCGGTCCCTCCCCGCCTTTTAGGCGCCCGCGTGGCCGGG |
| | | | | ACGTCCCAGTCCCGCTCCGTCCTCCTCGCCTGCCACCGG |
| | | | | TGCACCCAGTCCGCTCACCCAGCCCAGTCCGTCCGGTCC |
| | | | | TCACCGCCTGCCGGCCGGCCCAC |
| 11 | TPM2 | Dph-CRE11 | 308 | TTTTTCCTCCGACGGCTGGCAGGGAAGAGGGGGATGGGG |
| | | | | GCGGGACCCCAAGGGAGGCGGTCCCCAGTGGGTGGGCGA |
| | | | | AGGGGGCGGCCGCACCCCCCGGCCGGGCCGTGCTTCTGC |
| | | | | CCCTACAAGGTTTGGGCCGAGGTGGGGGAGGGTCCTGGT |
| | | | | TGCCGGCCCCGCCCGGTCCCTCCCCGCCTTTTAGGCGCC |
| | | | | CGCGTGGCCGGGACGTCCCAGTCCCGCTCCGTCCTCCTC |
| | | | | GCCTGCCACCGGTGCACCCAGTCCGCTCACCCAGCCCAG |
| | | | | TCCGTCCGGTCCTCACCGCCTGCCGGCCGGCCCAC |
| 12 | TPM2 | Dph-CRE12 | 263 | GACTCAGGGTAAACTGAGGCACTCAAACTGCCGAGGAGC |
| | | | | TCCGCCTCCCGAGAGACATTTAATCCGGGGGGATTTGCA |
| | | | | GGAAACTTCTAAATTAAGGGTAGCGGCTGCTGCAGCTGA |
| | | | | GGGGGGGCACGCCGGTCCCTGCGCCCGGGCAGCTGCCGT |
| | | | | GAGCTCACGCCCCGAAATAGCCCCAGGGGCCCCAGCCGC |
| | | | | AGCTGCCACTGGGTCCGGCTGTCACTCAGAGGAAGCACG |
| | | | | GAGCCCCCAGCCCAAGGGTCCCCTCCCCT |
| 13 | MYL1 | Dph-CRE13 | 324 | CTCAGGGAATGAGCTGGATACAACTAAAAATCAGCACAT |
| | | | | TTTTGTTTGGTTAAAACATTCTTTGTGGTCAATTTCTTT |
| | | | | CTAACAGATCGAGTTCTCTAAGGAACAGCAGGATGGTAA |
| | | | | GTTTAAAAGCTATGGTTCTTAAATGTGCACACTCATAAA |
| | | | | GATGGCATGTGTGAAAACCAACTCCCTTGGGATGAGTTT |
| | | | | AGCTCTTCCTAATTATCCCCACTGGTGTTGCCATTCTGA |
| | | | | ATATAAATTGCTCTCGATCACTCTAAATATAAATTAGCT |
| | | | | CAATCTGAAACCCTTGCTGATACTCAGTAATCAAAGGTT |
| | | | | TAACAAACAAAA |
| 14 | MYL1 | Dph-CRE14 | 326 | CATTTAGTTAAATCATTAAGATGCAGAAAGATATTCCCT |
| | | | | AATTATCTTTTGCAATAGCTTTTTATGTTCCTAGATTCA |
| | | | | AAAGTATCCTAAGGCAACCTTCTAATGCCAAGACACCTA |
| | | | | CATAGCTTCCTAAAAATCTCAGAGCAACCAGCACTCATT |
| | | | | CTTCTGTCAATGCATTTGAAAGAGTACTAGTTTTTTTTC |
| | | | | TTTCTTTCTCTTCTTCGCCACTGCAGTCCTTCAGTGCTG |
| | | | | ACCAGATTGCTGGTAAGTGAATTGAGTTTGTCTGCTACA |
| | | | | GATGTAGGCACAGCACTGCCTAGTTTCTGCAATATTACT |
| | | | | TATCTTCAAGGCAT |
| 15 | MYL1 | Dph-CRE15 | 596 | TTTGTTTGTTAAACCTTTGATTACTGAGTATCAGCAAGG |
| | | | | GTTTCAGATTGAGCTAATTTATATTTAGAGTGATCGAGA |
| | | | | GCAATTTATATTCAGAATGGCAACACCAGTGGGGATAAT |
| | | | | TAGGAAGAGCTAAACTCATCCCAAGGGAGTTGGTTTTCA |
| | | | | CACATGCCATCTTTATGAGTGTGCACATTTAAGAACCAT |
| | | | | AGCTTTTAAACTTACCATCCTGCTGTTCCTTAGAGAACT |
| | | | | CGATCTGTTAGAAAGAAATTGACCACAAAGAATGTTTTA |
| | | | | ACCAAACAAAAATGTGCTGATTTTTAGTTGTATCCAGCT |
| | | | | CATTCCCTGAGTAATCTTCAAAGTAAGCTCCAAAAGTCA |
| | | | | GCCTACTTTTCAGAGTTCAGGCGTAGCTTGGACACTAAC |
| | | | | AGGTTAAGCCATAGTGTAACATGCCTTGAAGATAAGTAA |
| | | | | TATTGCAGAAACTAGGCAGTGCTGTGCCTACATCTGTAG |
| | | | | CAGACAAACTCAATTCACTTACCAGCAATCTGGTCAGCA |
| | | | | CTGAAGGACTGCAGTGGCGAAGAAGAGAAAGAAAGAAAA |
| | | | | AAAACTAGTACTCTTTCAAATGCATTGACAGAAGAATGA |
| | | | | GTGCTGGTTGC |
| 16 | MYL1 | Dph-CRE16 | 290 | AGACCATAAAAGTACCGGCAGGCTTCCATCTCACTATGG |
| | | | | CTGTCTACTCCAAGGTTCTGGTCATCTAAAAATAGCTCT |
| | | | | CAGGGTACAGATCCATCTTTCCCTTTGCCCTAAGAAAGC |
| | | | | TAAAGAACTCTCCAAGGGGTGTGGCAACTTATCTCTGAA |
| | | | | ACCTGATGCTAGCTGTGAGGTCAAAGCTTGCCCAGAAAT |
| | | | | AAAAGGAAGCCTCAGCCAGGGATGACCCCACTCAGGGAC |
| | | | | CGGAGCAGCCCTCAACTCACTCTTCAGCTTCCCTGCTGT |
| | | | | GTTGCAGCCCAGCCGCT |
| 17 | MYL1 | Dph-CRE17 | 632 | GTGCTCCCTAGAAGGGAAGCCACTTTAAGACATGATGGG |
| | | | | CTTTCCTTATACGTCTGCTTTTTTATTTTTAACCACCAC |
| | | | | CACCTCTTTGCTGACCAACATTTCCTTGCAAATTATGGC |
| | | | | AAGGGGGGAGGAGCATGAGCAAAGGAGCTGTGTGAAAGT |
| | | | | TGGTGATACTCCCTGCCAAAATTCCAAGTTGAAATGGAA |
| | | | | TCCTTCAGTAAAGTCTCTTAGGCAAGGTGGGAAAAAAAG |
| | | | | ATTTACAACTTCAGCAGAACTGTAATTCTACCAAGAAAA |
| | | | | AGTACAGTCAATCTACCTCACTGTCACCAAAGGATCTGA |
| | | | | AGCTGGCTTTGTCATATATCTTTCTCCTTTATATATGGA |
| | | | | GGTGGGGGGGGGAGAAAAAAAGGAAAGAGAGTTAAAAAA |
| | | | | AATTGATGAAGCCCTGACCCTTTAGATTCCATTTATAGT |
| | | | | CTGAGCCGGAATGCCATCCCCCTTGACTAGAGAACTGTC |
| | | | | CAATCCAGCCGCATGTGTCAAGATTCTATTAGGCACTAA |
| | | | | GTGAAATATATATGCATGCCCTTATGCCGTTTAACACTC |
| | | | | TGGGTCCATCTTCAAGACACTGGGCTGTGGATCAACCCA |
| | | | | ACCACCACTCCTCTTCCAAGAATCATTTTGACAGGTTCT |
| | | | | TTTGGAGG |
| 18 | TNNC2 | Dph-CRE18 | 297 | CCTTTGACTTTTCTTGAAAGGGGAGGGCTGGGATATTCC |
| | | | | AGAGATTGATCCTTAAGGCTTGCTGACTGCCTACTCACT |
| | | | | TCTGGAAACTTCCAGCAGTGTCATTCATAGACCTGTGAA |
| | | | | GAGCTCTTAGCTTGTTTCCTTCACACAGTGGGGACTCTG |
| | | | | AGGGGTCAGAGTGAGTCACCCAGCAGGCCAGTGGCAGGG |
| | | | | GTGAGCCAGAAGCCTGGCCAAACCCTCCTATCATCATGG |
| | | | | AGAGAAGAAAGCCTCCTCCAGAAGACGGGAGGCCGGCAG |
| | | | | GGCGTGGGGCCTGCTCAGATGCAG |
| 19 | TNNC2 | Dph-CRE19 | 309 | GGAACAGGGCAGGCCTTCCTCACAAGAATCTAGGACGTC |
| | | | | AAGGCCTGCCACCTGCTTGGAGGCTTAAATTTCTCTGCA |
| | | | | AGGGCCCTTGGCTAAATTAGGTAATGGGTTCAGACTGTG |
| | | | | GGAGGGGTGGGACTCGCTGACCCCAGGATCTGATTGGGC |
| | | | | AGGGTCTCCAGTGCTGGGGAGCAGGGAGGTGGGAGGGGA |
| | | | | GGGTGCCCCTACAAATCCCGGGGGCTAGAGCAGGCCAGG |
| | | | | TCATCTTTGGGTGGTGGAGTGCAAAGGAGGCGACCTGCA |
| | | | | ACAGAGGAGTCCCGGTCACCAGCAACCATGGTAAGG |
| 20 | FHL1 | Dph-CRE20 | 267 | AATTGTGACATTTAAGCAGTGACTTTGCAAAATTGGACG |
| | | | | TGTCAGTAGGAAAAAGTACTCTTCCCACCTTAAAGATTG |
| | | | | ACAGGAACATGAGATGGCTGAAGCTGTTCTGAGCTTTTT |
| | | | | TCTGGCAAGAGTACATAGGACAAGAAATACTCTGGAGAG |
| | | | | CTCAACATTGAATCACATCATATCATAGTGCATGACTAG |
| | | | | TTGCCTTAATCTTGCAGTTGGACTCACTCAGGAGAAATT |
| | | | | GTTGGCAGAGGGATTAAGTAGCCTGACAAAATT |
| 21 | FHL1 | Dph-CRE21 | 493 | CTTTTCTTTTCTAATTAGGTCAGTTACTTCATGTCTGTT |
| | | | | TTTCTTTTTTGACATGGTTTCTTCTGGCTTTGGCACCAC |
| | | | | ATATTTTCCCCATATGTCATTGCCTCTGGAGCTTCATGT |
| | | | | TGCAATAGTTTTTCAAGGGGAACGGAGAGCACATTGCTA |
| | | | | AGGGTGGGGGATGGCTTTTGCCTCTTTTGCCTGCCCTTT |
| | | | | GCTTCAGTGAGTGTTCGTATTTCTGTTGGGCCTAGTTCT |
| | | | | GTTTGGTTTTGTAGTCTTCAGAGTCAGTTATGTTTGGAC |
| | | | | TGAAAGATACTTAAGTAAAAATAATGGCAAGTCAGAGAT |
| | | | | ATGTCTGGCAAAGGTGCAGCACATTTGTTAAGGTTACTG |
| | | | | GTTTATTTATCTCCCTTGTCTCTATGGTGACTAAATCTG |
| | | | | GGTCTGGGATTTAATGGACTTAGTTTTGACCTCTTGTAA |
| | | | | CATCTCCTAACTTTTCCCAGCCTCTGATTTAGAAAGAAT |
| | | | | TCATTTTCACTTGAGGAGAGAAACT |
| 22 | FHL1 | Dph-CRE22 | 197 | TCATTTGTTTTGATTACCCGGGTGATTGCCAAATGTTAA |
| | | | | TTTAAAACTGAAATAGGCCATGAGCTCACTCCCCTAACA |
| | | | | ATGCTGAGTGTTTGAGCTAATACGGCTTGATAATGAGGG |
| | | | | AGCCAGATCCACTGCCAGTGCTCATGTTATATGATAAAC |
| | | | | AGACATTTGGAGAAGTATGCAGAACATCTTGGCAGTTTT |
| | | | | CC |
| 23 | FHL1 | Dph-CRE23 | 637 | AAAAATTGGCTTTAAGGTCATGAGCCTCCTTACAGCAGT |
| | | | | CCCTCTCACCTTCAGCTGTATTCCCTATTTCAGATATAC |
| | | | | TTGTTTCTCTGCTGTAAGTACACTTTTCTTTTCTAATTA |
| | | | | GGTCAGTTACTTCATGTCTGTTTTTCTTTTTTGACATGG |
| | | | | TTTCTTCTGGCTTTGGCACCACATATTTTCCCCATATGT |
| | | | | CATTGCCTCTGGAGCTTCATGTTGCAATAGTTTTTCAAG |
| | | | | GGGAACGGAGAGCACATTGCTAAGGGTGGGGGATGGCTT |
| | | | | TTGCCTCTTTTGCCTGCCCTTTGCTTCAGTGAGTGTTCG |
| | | | | TATTTCTGTTGGGCCTAGTTCTGTTTGGTTTTGTAGTCT |
| | | | | TCAGAGTCAGTTATGTTTGGACTGAAAGATACTTAAGTA |
| | | | | AAAATAATGGCAAGTCAGAGATATGTCTGGCAAAGGTGC |
| | | | | AGCACATTTGTTAAGGTTACTGGTTTATTTATCTCCCTT |
| | | | | GTCTCTATGGTGACTAAATCTGGGTCTGGGATTTAATGG |
| | | | | ACTTAGTTTTGACCTCTTGTAACATCTCCTAACTTTTCC |
| | | | | CAGCCTCTGATTTAGAAAGAATTCATTTTCACTTGAGGA |
| | | | | GAGAAACTGTCTCACTTAGAAAAGGGGTCCTAACTGGAC |
| | | | | TCTCGAAAAGTGG |
| 24 | FHL1 | Dph-CRE24 | 561 | GTCTGCATCCTAATCCTGCTGTTGATTGGAGATAACTTC |
| | | | | AGCGGTCACAGGGCACCTCGTCTCCATGGCAACCCCTCT |
| | | | | AAATATTCCTCTAGTGACTTGTGCTCTACATTCTGGCTG |
| | | | | GATCAGAGCCCTCCTGTGGCAAAATATTCTAAAGATGTT |
| | | | | AAAAGAGAGAGAGAGAAAAAAAAAGGCTCAACTAATAAA |
| | | | | TCTATTCTAAACGACTGAGTCATTTGTTTTGATTACCCG |
| | | | | GGTGATTGCCAAATGTTAATTTAAAACTGAAATAGGCCA |
| | | | | TGAGCTCACTCCCCTAACAATGCTGAGTGTTTGAGCTAA |
| | | | | TACGGCTTGATAATGAGGGAGCCAGATCCACTGCCAGTG |
| | | | | CTCATGTTATATGATAAACAGACATTTGGAGAAGTATGC |
| | | | | AGAACATCTTGGCAGTTTTCCTTTAACTCTAAGGGTTGT |
| | | | | AAATACAAAGTAGAATGTGTTACCATGAGCCATTTCATA |
| | | | | GGAAGTTGACTTTATTTTTTTAGAATTCAGAACTTTGCT |
| | | | | TCTAAAAAGGTAACTTTGCCTCTTGATTCTCATTTTTTT |
| | | | | CCCTAACTTAGGCAC |
| 25 | FHL1 | Dph-CRE25 | 511 | AAAGACAGGGAGGTGCAATCTAAGTTAGGAAATTGGTGA |
| | | | | CAAATAGTCATAGAACATGGGTCACCTGGCTTCAATTAG |
| | | | | TTATGTGACCTTTAAGAAAACAAGTGCCCGTTTCAGAAA |
| | | | | AGACTGACCTTTGAGGCTAGAGACATCAAGCTACTCCTC |
| | | | | TACTTTTAGGAAAAAACAATGACAGGGTTAATTTAAAAC |
| | | | | TCTGATGCAGACAGAGTTGAAAAATCCATGACAGATTTA |
| | | | | TATAACGAACGTGGATGGTGTTTTGTTTTCCCAGAATGC |
| | | | | AGTTTTAAGGTCAGACCTATGACTCAGAGCTGTGCACTT |
| | | | | CCCTGGTGGTATGCATCAGTGACATGTTGCCAGTCTAAT |
| | | | | CGTCCAGAAGGGCTTGTGCCCAGCTCGGCTTGCTCCATT |
| | | | | ATAATCCCCAGCTGAGAAGCCACCTTTCATTTTTTTTAA |
| | | | | ATAGATAAGGAAGACAGCATGAACATTTGAAACTCAAAT |
| | | | | ACTATATGTAGTTTATAACTTTTCAGCCTGAAGACTTGA |
| | | | | TGCA |
| 26 | FHL1 | Dph-CRE26 | 399 | AATGCAGCATACCAGTGAAGTTGCTAATTACATTGAATC |
| | | | | ATTCTGTACACGTTCTATTTTGAAGGGAAAAAACACTTT |
| | | | | CTGATTTTTGTATGAAATTGTATGAAAGACTACTTTTCA |
| | | | | GCCGTGACTCATGAGCTCCGTCTCATTGCAGCTACTTAG |
| | | | | GTATTTATTCCCAAATGTCTGACGACACCTCCAGGTGCA |
| | | | | TTAGCATGTCTATTACACAAGTGTCCTATTCATTGGCTA |
| | | | | AGGCTTTGCTCCATCTGTTGAATGTTCTGAGGAAAGCAT |
| | | | | TATACCCTTTCTTTAGAAGCCAGTGTTTATATGTGGAGT |
| | | | | CACTAATGTACTGGTAGGTTGGATTTAGTGAAGAACAGA |
| | | | | TCTGAAATAACCTGATTTTTTTCCATTTGGGTTGTTGTT |
| | | | | CATTGTTTA |
| 27 | TNNT1 | Dph-CRE27 | 360 | CCCAACCTTTGAATGTATATGGGGGAGAAAATAACAGAC |
| | | | | AAGGTTGGAGGCGAAGATAAGGGGGGATGGGGTGGTGCT |
| | | | | TCTCCGCAAACATCCAACCCTTCTGTAAAGATTCCTCCA |
| | | | | GACTGGGGAAGAAGGCTTGGGTGTCGGTCCCTTTAAGAA |
| | | | | GGAAGGGAAAGGGTTAAAGCCACTGCGGGGCCATTTCCC |
| | | | | TTCCCGGCCCCGAGAGGGCGCAGGAGCTCTCAGGGGCTT |
| | | | | AAAGGACCGGGCCTGGGGGCGGGTTATGGGGGCGGAGGG |
| | | | | AGGGAAGGGTGGTCTTGGAGGTTGGGGCCCGAGGATATC |
| | | | | GGGGGTCCCCCCGGGCCCCCGACATCGGTCTCGGGAAGC |
| | | | | GAAGCAGCC |
| 28 | TNNT1 | Dph-CRE28 | 471 | CTCACCTTCCTCCTCCTCCTCCGCAGCCTCCTCTGGAGA |
| | | | | TGGGGGCACAGAAGAGAAGGCGTTAGGAGCTGGGGGAGG |
| | | | | GATGGGGGCGGTGGCCAGAGACCAGGGTTCCAGTCTCTG |
| | | | | CTGGACGGGGGTCCCTCTGGCCTCGGCTGCGATGGGCAC |
| | | | | GTTCCCCCTGGCGGTGCAGGGATGGCGCGAGGAGACGCT |
| | | | | CCAGACCCGGAGAGGGCGGGCGAGGGCCCGAGGGCTGAG |
| | | | | CCGCCCCTTCCCCGCCAAAGTGCCACACTCCTCGCCTCC |
| | | | | CCTCCCAAGAGCTCCTGGGCGTGTCCCCATCCCATAGGG |
| | | | | CCGGCCCACTCCCTACTCACCTTCCGGCTGCTCCCTGCG |
| | | | | GACGGGTGTGGGGAGAGAGGAGGGAGGGGAGAGTTAGAC |
| | | | | CTGGGGTGGGAGAGCCTCTCCACCACTGCACGCCCCAAC |
| | | | | CCCTCCCAGTGCAGCACTCACTCCTCATATTCCTGCTCC |
| | | | | TCG |
| 29 | TNNI2 | Dph-CRE29 | 393 | GGCCGCCTGGGAAGACCGGGGAGGTGGGGGCCAGAGAGT |
| | | | | CGGGACCACATGGGATGGGGCGGGCTAGAGCCAGCCCTG |
| | | | | TAGGGACAGAGTGGGCAATGGTGTCTGGGGGCTGGTGAG |
| | | | | CATGGAGGGAGGACCCCCAACACCTCAGAGACCTGTGCT |
| | | | | GCAGGGCCTTCCCTGCATGAGCAGCAGGGGGCAGCAGAT |
| | | | | GCCTTCGGAAGCCGCTGGGGCCTTTAGGGGCTCAGTCCT |
| | | | | GGCCGGATGCCCCTCCCAGTCCCCACACATCTGGGCTGC |
| | | | | CTTAGCGGCTGGGGCCTCCACGCTGCTGTCTCCTCTGAT |
| | | | | CCTCCCCACGGGGCTGCGAAGCCGGCAGGGCTGGGGCCA |
| | | | | AGAGCCCCCACTCCGAGAGGGTGCCAAGTCACGGTCCAG |
| | | | | GGG |
| 30 | TNNI2 | Dph-CRE30 | 331 | CTGTCCTCAGGCTCCTTACGAGAACGACAGAGGCATCTC |
| | | | | CAGCGCGTCACCGAGCCCTAAATAGAGTAGCCCAGCCAC |
| | | | | GGCACCCCCCACCAAGACTTCTTGGACTGGGCGGCAGCA |
| | | | | CGCGGCCAGGCCAGGCGGCCGGACAGGTGGGGAGGTCTC |
| | | | | TGTGGCTCTCCACGCCCCCATTGGTCTGAGGAGGACTCT |
| | | | | ATGCCCTTTCTGAGCAGGGGCCCAGCCTGGGGGAGGCCA |
| | | | | TTTATACCCCTCCCCCTGGGCCCACCAGCCCAACTCGCC |
| | | | | GCTGCCGGCCTGACCTCGCTCCCAGCCCTGCTGCCCAGA |
| | | | | TTCTAGGTGAGGCCCAGCC |
| 31 | TNNI2 | Dph-CRE31 | 306 | GCTCCCCCAACTCAGCATAGGTCATAGGTCACAGCCTCA |
| | | | | AGGCCCTAAGGCCCAGAGAAGATGGCCTGGCCCTCCCCG |
| | | | | CTGGCAAAAGTGCCCCACCCACCCACCAAGACGCTGCTG |
| | | | | AAAGAGAGGAAGTGGCTGCCCCAAGCCTTCTGGGGCAGG |
| | | | | GGAAGTGTGGCTGTGGCCTGGTCACAGGGGAATCACTTC |
| | | | | TTCTTTCTGATTCCTGCACTTCCCGCCCCCACCTCACCG |
| | | | | GCCGACCTGCCCCCAACTGGCCCTCCTCTCCCCCAGCCT |
| | | | | CTGGCCTCCCAGCACCATGTGCCACCTGGCAAA |
| 32 | MYLPF | Dph-CRE32 | 268 | AACTCTGGCACATTCCAGCCCCTCTTTGGGAGAAAGAAC |
| | | | | ATCTCTTCTTGCCCAGGCCCTAGTGAGACATGCATCGCA |
| | | | | AGACATGCTCCCCCCTTCCTCCCCGCTGGGCTCCAGCTG |
| | | | | CTGCCAGCCCTCCAAGAAAGGAGAGGCCCTGAGTTGGGC |
| | | | | TATTTTGGTATCTGGGGTGGGCACCCGCAGGGCTAAGGT |
| | | | | TACCTTGGTATGTTAAGGGCTCCCTGGGGCAGGACTATA |
| | | | | TAACCCCAGAGGGACTGCCCCATGCTGACTCCTT |
| 33 | TNNT3 | Dph-CRE33 | 214 | TACCTGGTGCAGGCCCTCGAGGCCGCTGGTGCCTGCTCT |
| | | | | GTGCAGTCCCTGCTACGCCTCAGGCCCAGCGCCCGAGGG |
| | | | | GGAGGTCGCCGATACCCAAAATAAAACCTGCCCTATCCC |
| | | | | TCTGACCTCAGCTGGCTGGGCGGAGAGCGCCTGCCAAAG |
| | | | | CTCCAGGAGCTGGGCGGGCAGCACCTTCCCCTGCTGGCC |
| | | | | ACACGGGGCCAGTGCAGGG |
| 34 | TNNT3 | Dph-CRE34 | 129 | TCTTGGGTTTCTGGGCAGAAACGTCTTGCCTATTTCTGG |
| | | | | ACT+CCTCAGCTGCCACTGGCTCCTTATAAATAACCCACC |
| | | | | CCAGGCGAGGGCCACGCTGCCCCCATCTTGTGGCAGCCG |
| | | | | ACAGGTGTCTGC |
| 35 | TNNT3 | Dph-CRE35 | 403 | TTACCTGGTGCAGGCCCTCGAGGCCGCTGGTGCCTGCTC |
| | | | | TGTGCAGTCCCTGCTACGCCTCAGGCCCAGCGCCCGAGG |
| | | | | GGGAGGTCGCCGATACCCAAAATAAAACCTGCCCTATCC |
| | | | | CTCTGACCTCAGCTGGCTGGGCGGAGAGCGCCTGCCAAA |
| | | | | GCTCCAGGAGCTGGGCGGGCAGCACCTTCCCCTGCTGGC |
| | | | | CACACGGGGCCAGTGCAGGGTTATGGCACACCCACCTTC |
| | | | | AGGTGCGCCCCTCAGGACCCCTGGCCTGGGTGAGGGCTT |
| | | | | GGGTGGGCACAGGCTTCCTAGGCCTGCAGACTGAACCAC |
| | | | | GTGTAACCAGAATGGTGGGCTCAGGGCTCCGTTAGAGAA |
| | | | | GCCGGCAGAGCGAGCCTGGAAAAGGAGGTTGGCCCCACC |
| | | | | ATGGGAGAGGATG |
| 36 | TNNT3 | Dph-CRE36 | 415 | AGAGTGAATGCATGGGAATGAATAGATGGGTGGATGAAT |
| | | | | GGACAACTGTGTGAAATAGCAGACAGCCTGGGGACACCC |
| | | | | TCCCTCCTGTCCCTCCTCTCCCAGGTCTTGCACTGGGCC |
| | | | | CTGACACTCAGCCCTGCAGCCTTCAGCGTCTGGGAAGTC |
| | | | | CCTTCGAGGAGCAGCACCCCAGACTCTGATGTTCTGGAC |
| | | | | TTCACTTTGCCATTCCCTTGGGTCCTCACAGCCACTATT |
| | | | | TTCTGAAGGCTTCATGTCAAACCCTGCTTGCCTCATGAA |
| | | | | TACACACAGTTCAGAAAAGCAAAACCTCGACAGAGCGAC |
| | | | | CACCCACACAAGGGAGGTGACGTGCCCCCATCCTTTCTC |
| | | | | AGATGCCCCAGCCACTGAGAGAGGATCCAGGCCACCCTC |
| | | | | CCCAGCTGGGGAACCACCCAGTGAC |
| 37 | TNNT3 | Dph-CRE37 | 352 | ACCAATCTGTGCCTGAACCGAAGAGTTCCGCAGACACCA |
| | | | | CCGCCCACGCTCAGCCTGTGGGGTCTGAGGGGAGTGAGG |
| | | | | GGGTGGGTCAGGGTGCCAGACTGCTCTCCCCCACCTGGT |
| | | | | GCCTCAGTTTCCCTGATCCAAGAGGCAGGGTTATCCAAT |
| | | | | CTGGTGGCCTCACTGGCCAGCATACAGGCCATCAGAGGC |
| | | | | CAGGAAGGATGTCTGCTAGTCGGACGGTGCCACCTGGAG |
| | | | | TCCTGAGGAAAGAAAACCAGTCTCCACACGCCACAATGA |
| | | | | GCCTGTGGGAGATGCTGGCACAGATGGTAACGGCCACTG |
| | | | | CGAGTGCCCAGGGTGGGGCCCCAGGGTTAGAAAGGGCCT |
| | | | | A |
| 38 | TNNT3 | Dph-CRE38 | 349 | CTCCTCAGCCCATCCAGAATGTACCCGCTGCTGGGAGTA |
| | | | | AAAATAGCAGCTGACACCTCCTGGAGGCGGAGGGAGGAC |
| | | | | CTTGCCTCCTTCTCCAAGCACGTCCTCTGCATCCTGGCC |
| | | | | TCCTTCAGCCTCCTCCTCTGGCCATTCCTATACTTGGTA |
| | | | | AGGGGCCTGCACGGGCATAGCCCCCCCCAGCAAGACTCC |
| | | | | GCACACACCCCGGCCACCCAGTCACTGGCCAATGGGCTC |
| | | | | CTAGGAAGATCAAATGTCACTATAACACGAGGGTGTGAG |
| | | | | CCGGGCGCCAGTGCCTGCAGCCGGTGCTGTCCACAGGGA |
| | | | | GCTCCAGCCCTTCTCACACTCGACCCGCAGGTGGGTA |
| 82 | MYH2 | Dph-CRE82 | 390 | GAAGCCAGGTATCCTGGCTGCCCAGCCACTGCCTTTATC |
| | | | | CACCAGCTTGACTCTTTCAGATCATCTTCTCAAATTATC |
| | | | | CATAGGAGATTTATCCACATACTCAATAAGAAAAATATT |
| | | | | TCTAATTATATCCAACCATTCTTAATATGAATGAGAATT |
| | | | | ATGCGGGGACGCTAGATTGCCAAGAGGTATTTTGCCAAA |
| | | | | CAATTCCTTTTGACTTAAGAAAGAAGAGGCAGCTGCATT |
| | | | | GTTTCCATAGCTATCCATATAAAAGAGCCCTTGGAATGA |
| | | | | GGCTGACTCGTCCTGCTTTAAAAAGCTCCAAGGTAAGTG |
| | | | | GGAGCAGGACGGGCCTTTCAAGAGGGACACTGGTCACAC |
| | | | | CGCCCAGTGTCAGCAGCAGCTGCTAGTTCTGGTACTGCT |
| 83 | MYH2 | Dph-CRE83 | 294 | CAGCTGCTAGTTCTGGTACTGCTCCTTCTGGAACTGCAC |
| | | | | GTTTTACCTCTATGAATTTTGTTACTTCTGTTTACAATA |
| | | | | GCAAGCCATGCCATAAATGGGATTTTTTCCCCCTCTTTT |
| | | | | TGGTCAACTAATCAAGTGCTTTTCCCTTTTCTTTTATAG |
| | | | | AACTGTCTCACTCCCAGGCTACATCTTCTCACTTGCTAA |
| | | | | CAAGGTAAGATTTGGACTAACCAGTTCCTGGAGGAGAAT |
| | | | | GCAAGAGGCTCTGGGATGGATTTCTGTCACTTAGCAACC |
| | | | | TTTCAGAAAGTGCTTGTCTCA |
| 39 | SLN | Dph-CRE39 | 380 | TAACTATGTTTAATGTGCTGAGAGAAAGCCATTTGTTTA |
| | | | | CAGAATCTTATTATAGAACAGAAATATACTTGTTCCAGA |
| | | | | GAAGAGGAGTTTAATTGGTTCCAAGTCAAATGATGCTAA |
| | | | | CAGGCGTCACCTCACCAATCCAGGACTTAAGATGGGTTA |
| | | | | TTATAAATAGGAGTAACACATATTTTTTTTGGCTCTTTT |
| | | | | TCCCACCTGTTATGCAAGACTGCATACCACTATGATGTT |
| | | | | CCTCCTGATTATTTCCAGGAACTTTGGGGGTGGCTCACG |
| | | | | TGTGCTCATGAACAAGTACTGTCAGGTATCACATACGGT |
| | | | | CTGTCATTTTCTTGCTCTTGAGCTCTGGCACTTCTCTGC |
| | | | | TGCTGTCTGCTGCTGAGGCCTGCAGAGAT |
| 40 | SLN | Dph-CRE40 | 575 | TTCTCTCTCACTGTAAAAGTGTAAAAGGTTAGGTTACTC |
| | | | | CCCAGCCTGTCGAACATGTGCCTGATTGCCTTCTGAGAT |
| | | | | AGCACATCCCTAATCCAAAAGAAAACCTAGCCTGGAGCT |
| | | | | TGCATCCAGCTAATCTTCTTCCACATTGCTCCAAACAGG |
| | | | | GAGTTCAGTTTTCTCATTACTCATACAGAGAGCAGAGAT |
| | | | | AGATTTTCATGATTCTTCAGGACTCTGTTCTCTCTGCTT |
| | | | | TAATAGCCAAAAGCTAAAGCCAAATGAACTTCTTCACAG |
| | | | | AGAACAACAAGCAATTTTATCTAATCCTTATTATTCTTG |
| | | | | CTGTAACTGCAATTGTGTTATTCTGACTGACAGATTCCC |
| | | | | AGAAGAGATTCAGTGAAAATACCCTTGATCAGATTCACA |
| | | | | AAGGAACAGCATATTTCAAGTGGGCGTGTCTGGCTAGAA |
| | | | | TCTCCCTGGACAGTTCAGTAACAGCACATCAACAGACTG |
| | | | | CTGAATTCTTATTTCCCCGTCCAAAAAATAACAAAGCCA |
| | | | | ATCTGAAATCTGGAAGTGTTCTGAGGGCAGATGGTATTA |
| | | | | GCCCAGTAGTAAACATATTTTTGGCTGCT |
| 41 | SLN | Dph-CRE41 | 463 | CTACAATAAATATATAACTTTTTTAATTTGACAAAAAAT |
| | | | | AATTTTGAGTGGTTACAAAAAGGGACAGGTGTGGCTTGG |
| | | | | TAACTAGAAAAACATTCCCAGCCTGGGAAACAATCTAGT |
| | | | | GAGAGCTTGGAGGCAGAAGACAGAACTCTGATTACATTC |
| | | | | AAGGAGTTTGCCAGATGGCAACAGATAACTGCCAATGTT |
| | | | | CGGTTGCACTATAATTATTATACACTCTCTGTCACTTCA |
| | | | | GCAAGCGCTCTTTTCACAAGACAAGTGGTGACAGAATGT |
| | | | | TGTATTAAGATTACCCGTTGCTAAGCTTATGTTAAAATG |
| | | | | AGGAAATGAAATGGAAAGTCTTGTTTTGGTAATGTCTCT |
| | | | | GGGGTATGAGGAATGGAGGGAAAGGTTTGACTATGAGCA |
| | | | | TAACTGCATAGAGAATTTTGTTTGTTTATGCCTTTTGGG |
| | | | | AATGCATTATTTTGATTGACCCTAATTGGGAATT |
| 42 | SLN | Dph-CRE42 | 379 | ATTCATGAAGATGTAAAAGACACATTTAAAACCCACATC |
| | | | | AATTGCACTAAAAGTCAACTGGAAACAGAAGGTTTTTAA |
| | | | | ATCCTGGTGGTGGCAAATTGGCAGCCAAAGGAACAAAGT |
| | | | | CTTAGTCAACATATTTCCATGTCCCCCGAACACAAATAG |
| | | | | CCTCGAACCTTCGGAGGGTGTTCCTTTGAGATGTTTCAT |
| | | | | CAGTGACATCACAGTGATTGCCAGCTTCCACGGACTACT |
| | | | | TTAGGCGCTGCCCAGAGTCCAGGAGTCCAGACAGCCTGG |
| | | | | GAGGGGAGAAGGAGTTGGAGCTCAAGTTGGAGACAGCGA |
| | | | | GGAGAAACCTGCCATAGCCAGGGTGTGTCTTTGATCCTC |
| | | | | TTCAGGTAACTGCAGGATTTTTATGCTT |
| 43 | MYBPC1 | Dph-CRE43 | 250 | AAGAAGGAATACCCTAGGGCAAATGCGTGCTAGAGGGGC |
| | | | | TTAATACTTGGCAGAATCCTTCTTTTTCAGTAGCCTGCT |
| | | | | TTTATGACTGCTCCGACTGCACCTGTTAGGAAGATAAAT |
| | | | | AAAGGACTGGCCAATTCTTTCCTCTGAAGCGCCCTAAAT |
| | | | | ATATCACTTTAAAAAGACTTTGGAAAAACAAGTTGTATC |
| | | | | CTTACTTCTCACCCAGTAAATCTAAAGAAATTCCCTCTT |
| | | | | GTTCCCAGGCCTTTCA |
| 44 | MYBPC1 | Dph-CRE44 | 194 | CCACATAACTAAAGCATGATGCTATTGAGTTCTAAGTCT |
| | | | | AGTTTCCACCCTTATATGAAAAGTCAGCTTGGCACAGAG |
| | | | | TTTGGTAGAGCTAAGTCTGACCAGCTGCACTCTCAGCCA |
| | | | | CAAGGGAAATTAGACGAGACAGTGTGGCTGCTTCAGTAC |
| | | | | CAATCAATCAAAGTTTTTGAAAATTATCTTGAAAACAC |
| 45 | MYBPC1 | Dph-CRE45 | 264 | ACTTTCTCTGTGGATTGCAATATTTCCTGGCATATGTTC |
| | | | | ACTTCAGTAAGACGATCAGTGTATTAAAAACCCAATGGG |
| | | | | GTTTCTGCAGAAGCCAATGATGGCATGGCACCTCTTTTG |
| | | | | TGGCATGTGTGAAATCTGCCCTTCGCTGTAAACATTCTT |
| | | | | ATGCAACATGACACTCTGCCCTATGTCCATTTCATATGT |
| | | | | GGTGTTCAGTGTGCTGGGAATGCTGAAAAAAACAATTCT |
| | | | | CTCTTAGAGATTTCCCAAGTAGAATCACAG |
| 46 | MYBPC1 | Dph-CRE46 | 427 | AGAAGGAATACCCTAGGGCAAATGCGTGCTAGAGGGGCT |
| | | | | TAATACTTGGCAGAATCCTTCTTTTTCAGTAGCCTGCTT |
| | | | | TTATGACTGCTCCGACTGCACCTGTTAGGAAGATAAATA |
| | | | | AAGGACTGGCCAATTCTTTCCTCTGAAGCGCCCTAAATA |
| | | | | TATCACTTTAAAAAGACTTTGGAAAAACAAGTTGTATCC |
| | | | | TTACTTCTCACCCAGTAAATCTAAAGAAATTCCCTCTTG |
| | | | | TTCCCAGGCCTTTCAAACAGTGAAGTCAGGAGTAAGGGA |
| | | | | GACTGTGGACTGGTCAAAGGTGGAAGTTAGACTCACTAG |
| | | | | CTGGTCTAAGGAATCCAGGAGGGTGGAAAACATGAAGAA |
| | | | | CTTACAATCCTTTATGCTCTCAATGACTCACTTTATTTT |
| | | | | GGCCATGAGACACAGCCTTGAAACTTTTTTTTTTTTT |
| 47 | MYBPC1 | Dph-CRE47 | 436 | GGTCATAAAATACTTTCTCTGTGGATTGCAATATTTCCT |
| | | | | GGCATATGTTCACTTCAGTAAGACGATCAGTGTATTAAA |
| | | | | AACCCAATGGGGTTTCTGCAGAAGCCAATGATGGCATGG |
| | | | | CACCTCTTTTGTGGCATGTGTGAAATCTGCCCTTCGCTG |
| | | | | TAAACATTCTTATGCAACATGACACTCTGCCCTATGTCC |
| | | | | ATTTCATATGTGGTGTTCAGTGTGCTGGGAATGCTGAAA |
| | | | | AAAACAATTCTCTCTTAGAGATTTCCCAAGTAGAATCAC |
| | | | | AGTGTTAAACCTATCAGCAGCCTCCAAGATCATTTTTTT |
| | | | | TAAGCCCCTCATTTTACACAAAAAGAAACTGAGGCCCAG |
| | | | | AAAGCCCCCTGACTTTCTCAGAGTAACTAAGCTGGTTAA |
| | | | | TGATAGATCCAGAAATAGAACCCAAGGGACCAACTTCAG |
| | | | | ATTCTAG |
| 48 | MYBPC1 | Dph-CRE48 | 359 | CTGAGTCAGCACATGCATCTGTCAGAGAGAGAAGAGCAA |
| | | | | GACGGAGCACGAGGCTGCAGCTGAATTTAGAAAGTTAGA |
| | | | | GGTGTTCTCATCAGCCACATGAGATGGGCAGGAGATTCC |
| | | | | TTCCAGTAGGCGAGAGAGCAGAGTGCAGACAACAGATGT |
| | | | | GAGAAAGGTCCCCATAGCTGTCAAGTTCCTGCATCCTTG |
| | | | | TGCTGAAGAAAGGGAGGTGCCATCATGATTGATGTTCAG |
| | | | | AGGCAGTATAAAAGCACTGGGTTTCTCCCCAACTGCTTG |
| | | | | TCACACCGACCTGCACCATCTCTCGCCTGCCTGTGGGGT |
| | | | | TTCTGTCAACTAGTCGTGGAGGGAAGGAGACTCTTTAAA |
| | | | | GAATAACA |
| 49 | MYBPC1 | Dph-CRE49 | 353 | GAGATTTCATCACATTTTAAAGTTTTGCCAAGCCAAAAA |
| | | | | GCTACCTGAATGTAGAGCAGCATTCATCCCCTCTTGCCT |
| | | | | ATTTCTAAGCATTTCTCAGGGCTTAGTCATCCATCAGAA |
| | | | | AAGGACCTCCCTTGATGGGAAAACTCCTTCTCATATCTC |
| | | | | TTCTTTGACCTTCAGTTTCTACCTGCAAGAGAGTGAATG |
| | | | | GCCTATTTCAAACACCGAAATCAATTTGTCTTTGGAAAG |
| | | | | TCATCTTATATACAAATATTATCCTTACCTCAAATTGGG |
| | | | | GAAAGGTTAATAATTTATGCAGAAAACTAAGAGAAAGGA |
| | | | | ATAGGGGAGGAGGAAAGATAGTTTATCAAGGGCAATTGT |
| | | | | TT |
| 50 | MYBPC1 | Dph-CRE50 | 354 | TCAAAAGTCTTTTAAACATATTTTCTTTATATAATTAAG |
| | | | | TAACCAGTGTTGACAGGCTGTGGTGATCAGTTTACAGGT |
| | | | | TGGCCTTACTTAGTGGCAGAGGGCATTGATGGATGAAGT |
| | | | | CTTTCACCCAAAATAACCACACCAAATGCATGTATTAAT |
| | | | | ATGGGAACTTAATCCTGACTGTCAGGCAGGGAGTCCAAC |
| | | | | CCCTGCTAAAGTATCTTATTTGTAATGCAAATGATTTAC |
| | | | | GTGTGTACTAAGTGTCCTTTTTATCTGCTCTGTCACTTT |
| | | | | AACCTCTCCGAAGTGTACCCAGAAGCAATACATTCTACC |
| | | | | ATGTTTCCAAACTGGAAACACAATATTGTTTTAATTATT |
| | | | | CTG |
| 51 | ENO3 | Dph-CRE51 | 351 | GCTGTCCCAGCGTTATCAGTCGGGCGCCTTGCCAGCCGA |
| | | | | AAGGGCCTGTCTAAATTCGTTTCCTGTCCCCTAACTCAT |
| | | | | CCCGGCGCTGGCTGGCCTGGAGAGGGTAGGATGGGGCGG |
| | | | | CGCCGAGAATGGCCGTTATGAGGACCCTAAGAGGTGAGA |
| | | | | CCCTCTCGCCTTCTGGGGTGGGGGGTCCCGTCCTTTCCC |
| | | | | CCACTGAGGACAGAGGCCCGCCCAGCGATCTGAGCATGT |
| | | | | GTGGACGTCAATCTTGCAGCCCCTCTTCCAGGCCCCCTC |
| | | | | CCCAGCCTTGCAGGGCTCAGGTTACCCCTGGCCTTTCCT |
| | | | | AAAGGTCACTCATTCCTCTTGACGTTTGCAAAAGGGGAA |
| 52 | ENO3 | Dph-CRE52 | 607 | TGTCCCAGCGTTATCAGTCGGGCGCCTTGCCAGCCGAAA |
| | | | | GGGCCTGTCTAAATTCGTTTCCTGTCCCCTAACTCATCC |
| | | | | CGGCGCTGGCTGGCCTGGAGAGGGTAGGATGGGGCGGCG |
| | | | | CCGAGAATGGCCGTTATGAGGACCCTAAGAGGTGAGACC |
| | | | | CTCTCGCCTTCTGGGGTGGGGGGTCCCGTCCTTTCCCCC |
| | | | | ACTGAGGACAGAGGCCCGCCCAGCGATCTGAGCATGTGT |
| | | | | GGACGTCAATCTTGCAGCCCCTCTTCCAGGCCCCCTCCC |
| | | | | CAGCCTTGCAGGGCTCAGGTTACCCCTGGCCTTTCCTAA |
| | | | | AGGTCACTCATTCCTCTTGACGTTTGCAAAAGGGGAATG |
| | | | | TAATCCTGGGGTGGGGGGAGACCCCTCATCTGTAGCCCC |
| | | | | TCCCTTGCTCCTCCCAAAGGGTGGAATTAGAACAGGGAC |
| | | | | TGTTATTGGGAGACAGAAAGTGGGGGATAGTAGTTGACC |
| | | | | TTTGGTAAGGGGGCAGGTGCCCAGGGCCAGAGGCTTCTG |
| | | | | CTTCAGGCTGTAGTGGGCACTTGGCTGCCAGCCCAGTGT |
| | | | | GAAGGGGGGAGGATGGAGAGAAAGAGAGGCGGGGCTGGC |
| | | | | TGGGGACCGAGTGGCTCAGGGA |
| 53 | ENO3 | Dph-CRE53 | 469 | AGGGCTGGGCGGCTTCCGGAGCCTGGGCTAGGGGCAAGG |
| | | | | GGTCACCGAGCGCGGCCCGGGCGGAAAGGGGGTTGGTTT |
| | | | | TCTCTCTCCTCCCGAGCGCGGCGCGCATCCCTGGCGTCC |
| | | | | ACGCCGAATCCCACAGTCCCCGACGCCCCTCGAGTCCGT |
| | | | | GTTCCTCGACAGCCGCGCGGCTGAGTCACTGGCGGGCTC |
| | | | | GGGCGGGGCCGCACCCGGGCACGTGGCGGCGCTCCCCGC |
| | | | | CCGCCATCTCCTGACCCCTGGCCCACCGCACCCCTACCC |
| | | | | CAGGGTGGAAAAATCCCGGGAGGAGCGGCCTGAGATGAG |
| | | | | GGGGCGGGGCGAGAGGGGAGACTGGACGGGTGGCGGGGC |
| | | | | AGGTGGCCTGGGGTGGGGGCTGGGAGGCCGCGCGGGCCG |
| | | | | GCGGGGCGGGGAGCAGGGGTGGGGAGAGGGCGGCGGGGG |
| | | | | TGAGTCACCGGGCGCGCGCTGCCCCGGCGCCGACGGGAA |
| | | | | G |
| 54 | ENO3 | Dph-CRE54 | 332 | TGGGTTCACCAGGGCCCATTCTCAGTCTAGAGGATTGTC |
| | | | | CCTTCTCTGGATCGGCTCTAGCCCAGGGCCTCACCCGAC |
| | | | | ATCCCAGCCCCGCCCGCCCCAGGCAGGCAATGTCTGGAT |
| | | | | CACCGGCCGTGCCCCTTGGCCTGTGTCTGCAGAGGTCAC |
| | | | | AGAGCGCGGACACCCGCGGGGCTTAACAGGCGGGGCTAT |
| | | | | TTTTCGAGGCGGAAGAATACTCAAGTTGCAAGAGTGGCA |
| | | | | ACTTTTTCAAGAAGGGGAAAGTCCCACTTCGGGCCCTTT |
| | | | | GGGTTTCTTCGCAGTCAGATGTGCGCTCACAGCCACCTG |
| | | | | GGGAGGGCGGGACGGGCGGG |
| 55 | CA3 | Dph-CRE55 | 206 | TACATTCCAGGGAGGGAGACCAGTTGGGACAAGGCCTAG |
| | | | | GCTGCTCTCCTACAAATTTGCCATGAACAGGAGCCAAAC |
| | | | | TCTAAGACCTGATTCAAGTGGCCCAGCTGTCACTGGTGA |
| | | | | CACACTCAGGGGCTGGGCCTATCATGTTCTTATACAACC |
| | | | | TAGTGAGTCAAGCTTTGGAAAATAACATTGTCAGGGAAG |
| | | | | AACGTAAAAGT |
| 56 | CA3 | Dph-CRE56 | 289 | GGAGCTGAGAGTTTAAGGGCATTTGTTTACTATGTTTGC |
| | | | | TTGGATTTTCCGAACTGCCTTAGTTCCTGATTCCACACT |
| | | | | GCTATTGTGTGGATCAAATAATCCCTTGTCACAAAAATA |
| | | | | CCCTGGGGAATGACTCCCCTGGCGTCCAGGTTATGAGTG |
| | | | | TGGTCAGTCATTCCACATGCCTTAGGGATGAGCTATCTG |
| | | | | TGCCATGACGAGCGATTTCCTTTTCGAGTTTTGTAATCC |
| | | | | CCAAATACTAAATACTATTATCAATAAAAGTTAATTAAG |
| | | | | GAAATGTATATCGAAA |
| 57 | CA3 | Dph-CRE57 | 195 | GACAGCTGTCCCGCTCTTGGAATTCATTGGCTTCCTCTA |
| | | | | CCCGGCCTCCCAAACACCACCCCAATCTAGTTTAGCCCC |
| | | | | CCGCCCCACCCTCGCTGACCTAATAAGGCCATGCAGTGT |
| | | | | GCGGGGGAGCTACATAAAAGCGCGGGCTCGCGGCGACTC |
| | | | | TGCACCACGCAGGGGAAGAGAAAGCAGGAGCCGTCCAGC |
| 58 | ATP2A1 | Dph-CRE58 | 345 | CCCCTCAGCTTCAGCCCCCACCTCCAGGAGGCCCTACCC |
| | | | | ACGCTCATGACCTTGCTATTCTGGGCCTTGTGTCCTGTA |
| | | | | GGGAGATGGACAGGAGACAGCTGGGCTTCCAGGCCACCC |
| | | | | AGGCGGGGGGCTAGCCGAGGGAAGCCTGCTGGCTCTCCT |
| | | | | GCTTGCTCTAATTTCTGGGGCTCCCCAAACCTTGGCCTC |
| | | | | AGGAGACTGGGGATAGGACCGGCCTTGAAAGTGGGGGAA |
| | | | | GCTTTGGAGAGCCGGGTGCTGGGTTCTTAGTGAGATGGC |
| | | | | CAGTGAAGGCTGTGGTGCCCCGAGGTAAGCAGGGCCTGA |
| | | | | TCCCCTCCTAATCTTCCAGCAGCAACTGGTGCT |
| 59 | ATP2A1 | Dph-CRE59 | 220 | CTCCTCCAGCAGCTGCCCTGGTGGTAACCAAGAGACGCC |
| | | | | CCCATCCTGGAGCAGGGGTGGGGAGGGGCAGCTCAGAGC |
| | | | | AGCTGCTTCTCTGAGGAAGCTGACACCAAGGCCAGCATT |
| | | | | CAGCAACAACTTGTGGCTTTGCACCCAGCGCCGGGGTCC |
| | | | | CCGCCCACCTGGCTCCCTGCTGTCCCTCTTCCCCACTGC |
| | | | | TGCTCGGACTTCCCTCTGACCCTGG |
| 60 | ATP2A1 | Dph-CRE60 | 663 | CTTTGCTCCCTGGGAGGGCCTGGCCCTGTGGGCATTTGA |
| | | | | GTTTATAACACCACCCCCATTGTGGCACACCCCTCCACC |
| | | | | CCGTAAAACACAGGCTCTGCTCTTGGAATCAGTCTTCCT |
| | | | | GATCTGTGGCTGTGCCCTCCAACAGAGGGCACCCCTGGG |
| | | | | CTTCCCAGCTCTGGGGGTAGTGGGTGCCAACAAGGAGGG |
| | | | | GCCTGGGGCTGAAGAATCCCACCCGCTGAGCTCGGCCTT |
| | | | | CTCCCTTCCCCACTGTCCAGCTCCGCCTTTCAGCATCCT |
| | | | | GCCTCACTCCCCGCCCAGGCAGCAAGGAGCCCACACCCT |
| | | | | CATGCCCCTCAGCTTCAGCCCCCACCTCCAGGAGGCCCT |
| | | | | ACCCACGCTCATGACCTTGCTATTCTGGGCCTTGTGTCC |
| | | | | TGTAGGGAGATGGACAGGAGACAGCTGGGCTTCCAGGCC |
| | | | | ACCCAGGCGGGGGGCTAGCCGAGGGAAGCCTGCTGGCTC |
| | | | | TCCTGCTTGCTCTAATTTCTGGGGCTCCCCAAACCTTGG |
| | | | | CCTCAGGAGACTGGGGATAGGACCGGCCTTGAAAGTGGG |
| | | | | GGAAGCTTTGGAGAGCCGGGTGCTGGGTTCTTAGTGAGA |
| | | | | TGGCCAGTGAAGGCTGTGGTGCCCCGAGGTAAGCAGGGC |
| | | | | CTGATCCCCTCCTAATCTTCCAGCAGCAACTGGTGCTCT |
| 61 | ATP2A1 | Dph-CRE61 | 594 | CAGCAGCTGCCCTGGTGGTAACCAAGAGACGCCCCCATC |
| | | | | CTGGAGCAGGGGTGGGGAGGGGCAGCTCAGAGCAGCTGC |
| | | | | TTCTCTGAGGAAGCTGACACCAAGGCCAGCATTCAGCAA |
| | | | | CAACTTGTGGCTTTGCACCCAGCGCCGGGGTCCCCGCCC |
| | | | | ACCTGGCTCCCTGCTGTCCCTCTTCCCCACTGCTGCTCG |
| | | | | GACTTCCCTCTGACCCTGGTGGCTCTGTGTCTCTGCTCC |
| | | | | CTTTCCCCCTAGGTCTAGACATCTGTCCTTATTTCCCCC |
| | | | | AGACCTGTCCCCAGAAGTCCACCCTTCCCCATTCCTTTG |
| | | | | GTCTGGAGCCCCTGCTTGGTCCAGCTTCCCCAGGCCCCG |
| | | | | ACACCTTTCTGTGGGGTCTGCCTAGCTCCTGCACGGACA |
| | | | | CAGCATGGGCCTGATCCTGTTCCCCTCGTGGACAGATGC |
| | | | | AGCAGGGCAGAGTGCAGCGCAGACCACAGGCCTCTGGGG |
| | | | | CTGGCCACAGAAACCCCGTTGGTTAGAGCACAGTGTGGG |
| | | | | ATGAGGTGACCCTCAGTGCACGACTTGGGGTGACCCCTG |
| | | | | CCCCCATCCTGAGACAGTTACCCCTCCCCCTCTGCCATC |
| | | | | AGCACATTC |
| 62 | ATP2A1 | Dph-CRE62 | 634 | CCTTCCCCATTCCTTTGGTCTGGAGCCCCTGCTTGGTCC |
| | | | | AGCTTCCCCAGGCCCCGACACCTTTCTGTGGGGTCTGCC |
| | | | | TAGCTCCTGCACGCACACAGCATGGGCCTGATCCTGTTC |
| | | | | CCCTCGTGGACAGATGCAGCAGGGCAGAGTGCAGCGCAG |
| | | | | ACCACAGGCCTCTGGGGCTGGCCACAGAAACCCCGTTGG |
| | | | | TTAGAGCACAGTGTGGGATGAGGTGACCCTCAGTGCACG |
| | | | | ACTTGGGGTGACCCCTGCCCCCATCCTGAGACAGTTACC |
| | | | | CCTCCCCCTCTGCCATCAGCACATTCTGTAGCCTCTTGG |
| | | | | GTTACTTGGCTGCCTTGGTGTCCCATTTTCTTGGGGGTG |
| | | | | GGGTGGGGATTCCCTATCCAGGATGGGGGGGCCCTCAGG |
| | | | | GCTCTGTTCCCAGAGGCTGAGTTAGAGCGATGGGGAAGG |
| | | | | GGGGGGGCAGTTTTGGGGAGAGACAGGCAGTGCTGGCTT |
| | | | | TGCTCACCAGGGCCTGGACACTAAATCCCTTGTTGATGG |
| | | | | CTGTGGCAACCCCTCCCTAGGGTAGGGTTACCATCTTCG |
| | | | | GCCCTGTCCCCTTGACTCTCTCCCCTCACTTCCCCTTGT |
| | | | | CCCTCTAGGAGCCACTCACTTCCTCTAGCCCCCAAAAGA |
| | | | | TGTTCTCCCT |
| 63 | ATP2A1 | Dph-CRE63 | 445 | CCTCCCCTGCACCCCAGAGGCAGGTTTTATTTTAAGCTT |
| | | | | TAAGGGTGTTCTCAGCCAAAACACCGAAGCTAAGCCACC |
| | | | | CTCGCGGCTTCAAGAGCTTGGAGAGCTCGGGTTACCCAC |
| | | | | CCGAACTCCGGGCTCCGGGTCCCGCCGCGATGCCGGCTG |
| | | | | CGGCGCGGGGGGCCACTGCCACTCCCGGCATGCGCCGGG |
| | | | | CGGACGGCCGCTCCACCAATCCCCGCGCCCGTCGGCGCC |
| | | | | CCTGCCCCGCCCTCCCCAGCCTCCTGACGCTGATTGGTC |
| | | | | GAGGGGAGGACTCGCTCCTAGTGGCGGGAAAGCGCGGCG |
| | | | | GTGTGATGATGACTCCAAGGAGCCCGGCGCCCGGTCAGG |
| | | | | GAGGGCACTGGCATCCCTCATTACCCGCCCAGCCTGGCC |
| | | | | TTAGCCCTTCCCCGCGCTCCCTAGGCACCCCCACCCCCG |
| | | | | CAGGGCATCTCCAGGG |
| 64 | ATP2A1 | Dph-CRE64 | 408 | GTCTCCGAACGCAGGCCCCGTCGCGTTAAGCACAAGCTG |
| | | | | GCAGGGCCTCTCCTCTCCCTTCTCAGATTTGCTCCTTGA |
| | | | | CATTTGCCTGCTGCCTGGCGGTGGCAACAGCTGGGGCGG |
| | | | | GGCGCGCGCAGGAGGCCCCGTAACCCTATCCCCGCTCCG |
| | | | | GCTCCCTCGTGAAACCGGAGCTTCCCTGCCTTGGCCGAG |
| | | | | GGGGAGGGCTGCGGGGGCCAGACCGCCTGCGAAGACCAC |
| | | | | AGGGTTTTTCCTCTCGGGTTTTGGCTCCCGTGGGATGGA |
| | | | | TGTGGCTGTGCGGGGGGTTGGCCTGAGCTTCGCTTCTAA |
| | | | | GCCAGCAGCTTGGTCAGGGAAACCTGAAAGCATTCCCAG |
| | | | | CTAATCCCCCAAGTGGTGCAAGTCTGTGCGCGCCCATCC |
| | | | | CGCTGAGTAAGGCGGTGG |
| 65 | MYH1 | Dph-CRE65 | 496 | GGCAACAAAGTCCAGGGATTCCAGAACTTAGCTGTCTTT |
| | | | | GAAACAACACCTTACATTCCAAAGGCCAACAACTTTAAG |
| | | | | ATTGCCAACGACTCAAATTATTTATAGGAGATTGATTCA |
| | | | | TATACTTAACCCAATATGAGAGGAAATATTTCTAATTAT |
| | | | | ATCCAAACATCTTTAATATGAGGGGAATTAGGCTTGGAG |
| | | | | ACCTTCAATGCCAAAAACATGTTTTGCCAAATATGTTTT |
| | | | | AAGAAAGAGGAGGAGGTTACGTGTTTTCCATATTTGAGC |
| | | | | CTATAAAAGTACCCTTGGAATGAGTATGACTTGTCTCTC |
| | | | | CTCATAAAGCTTCAAGGTAAGTGTGTGTGAGGACAGGGC |
| | | | | TCTGTCTGGAGGACGGTAAGGGATGTCTGGCTGCCCGCG |
| | | | | TGCATGCCACACATCCTGGCTCCCACTGTCCCTGGCAGC |
| | | | | CCTGCCAGTGAAGCACCAGCTTACTCCGTAGCAATTTTA |
| | | | | ATTAGATGTTGTAGTCTTCCCAGTCATT |

Table 4 below depicts the core nucleotide sequence of the different nucleic acid regulatory elements for enhancing gene expression in diaphragm, heart and skeletal muscle cells or tissue and their corresponding genes and lengths.

TABLE 4
The CREs sequence for highly expression in diaphragm,
skeletal muscle, and heart.

| SEQ ID | | Dph-CRE | Size | |
|---|---|---|---|---|
| NO. | Gene | (CRE) | (bp) | Sequence |
| 1 | ACTA1 | Dph-CRE01 | 328 | GGAGACACTCCATATACGGCCCGGCCCGCGTTACCTGG |
| | | | | GACCGGGCCAACCCGCTCCTTCTTTGGTCAACGCAGGG |
| | | | | GACCCGGGCGGGGGCCCAGGCCGCGAACCGGCCGAGGG |
| | | | | AGGGGGCTCTAGTGCCCAACACCCAAATATGGCTCGAG |
| | | | | AAGGGCAGCGACATTCCTGCGGGGTGGCGCGGAGGGAA |
| | | | | TGCCCGCGGGCTATATAAAACCTGAGCAGAGGGACAAG |
| | | | | CGGCCACCGCAGCGGACAGCGCCAAGTGAAGCCTCGCT |
| | | | | TCCCCTCCGCGGCGACCAGGGCCCGAGCCGAGAGTAGC |
| | | | | AGTTGTAGCTACCCGCCCAGGTAG |
| 2 | ACTA1 | Dph-CRE02 | 452 | GACAGGTGCGGTTCCCGGAGCGCAGGCGCACACATGCA |
| | | | | CCCACCGGCGAACGCGGTGACCCTCGCCCCACCCCATC |
| | | | | CCCTCCGGCGGGCAACTGGGTCGGGTCAGGAGGGGCAA |
| | | | | ACCCGCTAGGGAGACACTCCATATACGGCCCGGCCCGC |
| | | | | GTTACCTGGGACCGGGCCAACCCGCTCCTTCTTTGGTC |
| | | | | AACGCAGGGGACCCGGGCGGGGGCCCAGGCCGCGAACC |
| | | | | GGCCGAGGGAGGGGGCTCTAGTGCCCAACACCCAAATA |
| | | | | TGGCTCGAGAAGGGCAGCGACATTCCTGCGGGGTGGCG |
| | | | | CGGAGGGAATGCCCGCGGGCTATATAAAACCTGAGCAG |
| | | | | AGGGACAAGCGGCCACCGCAGCGGACAGCGCCAAGTGA |
| | | | | AGCCTCGCTTCCCCTCCGCGGCGACCAGGGCCCGAGCC |
| | | | | GAGAGTAGCAGTTGTAGCTACCCGCCCAGGTAGG |
| 3 | ACTA1 | Dph-CRE03 | 239 | AGAGAGAGGGACAGGCACCAACTGGGTAACCTCTGCTG |
| | | | | ACCCCCACTCTACTTTACCATAAGTAGCTCCAAATCCT |
| | | | | TCTAGAAAATCTGAAAGGCATAGCCCCATATATCAGTG |
| | | | | ATATAAATAGAACCTGCAGCAGGCTCTGGTAAATGATG |
| | | | | ACTACAAGGTGGACTGGGAGGCAGCCCGGCCTTGGCAG |
| | | | | GCATCATCCTCTAAATATAAAGATGAGTTTGTTCAGCC |
| | | | | TTTGCAGAAGG |
| 4 | ACTA1 | Dph-CRE04 | 509 | CCTTTTAGAGAATCCACACCTGTCCCAGTTGCTGGGTT |
| | | | | CCACTACCAAAAGTGAATTGCAACTATTTTAGGAGCAC |
| | | | | TTAAGCACATCCGAAAAATGAGTGATTCTGTTCTGGCC |
| | | | | CACACCACATCACTGATGTACCCCCTTAAAGCATGTCC |
| | | | | CTGAGTTCATCACAGAAGACTGCTCCTCCTGTGCCCTC |
| | | | | CACAAGGTTAGAACTGTCCTTGTCTTAGGGAAAAAGGA |
| | | | | GAGAGAGAGAGAGAGAGAGAGAGAGAGAGAGAGAGAGA |
| | | | | GAGAGAGAGAGGGACAGGCACCAACTGGGTAACCTCTG |
| | | | | CTGACCCCCACTCTACTTTACCATAAGTAGCTCCAAAT |
| | | | | CCTTCTAGAAAATCTGAAAGGCATAGCCCCATATATCA |
| | | | | GTGATATAAATAGAACCTGCAGCAGGCTCTGGTAAATG |
| | | | | ATGACTACAAGGTGGACTGGGAGGCAGCCCGGCCTTGG |
| | | | | CAGGCATCATCCTCTAAATATAAAGATGAGTTTGTTCA |
| | | | | GCCTTTGCAGAAGGA |
| 5 | ACTA1 | Dph-CRE05 | 169 | CCTTTTAGAGAATCCACACCTGTCCCAGTTGCTGGGTT |
| | | | | CCACTACCAAAAGTlAATTGCAACTATTTTAGGAGCAC |
| | | | | TTAAGCACATCCGAAAAATGAGTGATTCTGTTCTGGCC |
| | | | | CACACCACATCACTGATGTACCCCCTTAAAGCATGTCC |
| | | | | CTGAGTTCATCACAGAA |
| 6 | CKM | Dph-CRE06 | 400 | GGGCCAGGGGACGGTGGCTTCTACGTGCTTGGGACGTT |
| | | | | CCCAGCCACCGTCCCATGTTCCCGGCGGGGGGCCAGCT |
| | | | | GTCCCCACCGCCAGCCCAACTCAGCACTTGGTCAGGGT |
| | | | | ATCAGCTTGGTGGGGGGGCGTGAGCCCAGCCCCTGGGG |
| | | | | CGGCTCAGCCCATACAAGGCCATGGGGCTGGGCGCAAA |
| | | | | GCATGCCTGGGTTCAGGGTGGGTATGGTGCGGGAGCAG |
| | | | | GGAGGTGAGAGGCTCAGCTGCCCTCCAGAACTCCTCCC |
| | | | | TGGGGACAACCCCTCCCAGCCAATAGCACAGCCTAGGT |
| | | | | CCCCCTATATAAGGCCACGGCTGCTGGCCCTTCCTTTG |
| | | | | GGTCAGTGTCACCTCCAGGATACAGACAGCCCCCCTTC |
| | | | | AGCCCAGCCCAGCCAGGTAC |
| 7 | CKM | Dph-CRE07 | 255 | CCCAGCCCCCTTCCCCGGGAGGTGGGAGCGGCCACCCA |
| | | | | GGGCCCCGTGGCTGCCCTTGTAAGGAGGCGAGGCCCGA |
| | | | | GGACACCCGAGACGCCCGGTTATAATTAACCAGGACAC |
| | | | | GTGGCGAACCCCCCTCCAACACCTGCCCCCGAACCCCC |
| | | | | CCATACCCAGCGCCTCGGGTCTCGGCCTTTGCGGCAGA |
| | | | | GGAGACAGCAAAGCGCCCTCTAAAAATAACTCCTTTCC |
| | | | | CGGCGACCGAGACCCTCCCTGTCCCCC |
| 8 | MYL2 | Dph-CRE08 | 354 | CTCCTGGGCTCGAGCAATTCTCCTGCCTTGACCTCCCA |
| | | | | AAGTGCTGGGATTACAGGCATGAGCCACTACACCCTGC |
| | | | | CCTAGTGTGCTTTATATCAAAGGGGA/vACCATTTGGGG |
| | | | | CTTCCAAACAGGAAATGAACAGTCGTACCTTTTGTCCT |
| | | | | CACTAACATTCAGATTGCAGGAATGCCGTGTCCCATAG |
| | | | | GGAAAGAGAAATTCCTGAGGCAAGTGTOYTATCTGAAC |
| | | | | TTGAGATAGTCACCTTCCAGGCAGAAAGCTACACCCGC |
| | | | | CTCTTTTCCCAGCCTCTGGCATCAGCTGCCGGCGGTGT |
| | | | | GGGTAAGGGATGCAAAGAACTCAAAACATGTAGCCAGG |
| | | | | ATTCCCCATTTG |
| 9 | MYL2 | Dph-CRE09 | 297 | TGACAAGCACAAGTGTCCCCGGCCCAAGCACCGCAGAG |
| | | | | AGCGCGCAGCATCTCTCCCCGTGACCATGACCCAGCTA |
| | | | | CTGCCTCTTTAACCTTGAATGCCTTTTTGGGGGCTCAC |
| | | | | GTGTCACCCAGTGGCGAGTGAGCCACCCTTACTTCAGA |
| | | | | AGAACGGCATGGGGTGGGGGGGCCTTAGGTGGTGCCCG |
| | | | | CCTCACCTATGACTGCCAAAAGCGGTCATGGGGTTATT |
| | | | | TTTAAACATGGGGAGGAAGTATTTATTGTTCCTGGGCT |
| | | | | GCAGAGAGCTGGGCGGAGTGTGGAATTCTTC |
| 66 | MB | Dph-CRE66 | 319 | CCAGGTGACAGGTGGATGGTGGAGCTGGAGGAGCCACT |
| | | | | CGGAGCCTCCAAGGCTGGGGAGGGTGGAGGGGGGAGGC |
| | | | | AGGTCGGTGTCCCAGGCCACACCTGTTGACTAAGGGAT |
| | | | | TAGGATGTTGTGTCCCTGCCAGCCCTCCTCCAATAAGC |
| | | | | CCCTCTGGGCCTGCAGGGAGAGGAGGAGCCTTGCCATG |
| | | | | TAAACTGTATTTTTAGTTCCCTGTGCCTCTCCCCGGCT |
| | | | | GCTATAAGACACCTCTCCCCACCCCCAGCCCTGGCCGC |
| | | | | TTGGCTGGAGGCTCTGCGAGGACAGCTGGGGAGAAGGG |
| | | | | GAGCTGTGGTCAGTA |
| 67 | MB | Uph-CRE67 | 372 | TCATCTTCTAATAATTTGGCAAGCCATCCATGGTCTTC |
| | | | | TAAGCAAGGCGCCATGAATATAACGAATATAGAGACAG |
| | | | | AAAAAGACCCCTCCCCACTGGCTGGGTTGAGGGACTGG |
| | | | | AGAGCCCAGATGGAAGGGCAGAGGTGCAGGCTTTTTTC |
| | | | | CTTGTTGCCACTAGATGGCAGTAGGGCACCCGTTGTCA |
| | | | | GCCCTGGGGCAAGGTCACCGACTGTCTTTGCCTTTGCC |
| | | | | TCCAGCAAGCCAAAACCCTGGGCAGACTCAATCCAAAA |
| | | | | ATAAACAATCAAAGAGCATGTTGGCCTGGTCCTTTGCT |
| | | | | AGGTACTGTAGAGCAGGTGAGAGAGTGAGGGGGAAGGA |
| | | | | CTCCAAATTAGACCAGTTCTTAGCCATGAA |
| 68 | MB | Dph-CRE68 | 293 | AGAAAGGAGCTGCCCTGGGGATGGCTGTGTATCCCTAC |
| | | | | AACTGCCAGGCACATGCCCCTTGAACACCCGATGCTAC |
| | | | | GTGTCCCAAAGGAAACTGGTCTCCACCCACCCCCGGCC |
| | | | | CGTCCTCGTCCTGGGTACCCCACCTTAGTAAATGGCGC |
| | | | | CACCATCTGCCCGGTCACTCAGCGAGAAACTCAACTCC |
| | | | | TGGCAGCAGATGACGGGCACTCTGGTTAAATGACTCTC |
| | | | | TCCAGCCTCCAAGTTCAACCTGCAGGGAAGCCCTGGAA |
| | | | | ATCCTGTCTCCCTCTGCCCTGCCTCTC |
| 69 | DES | Dph-CRE69 | 482 | GAAACATGGCTTCCACAGGTTCCAGTTGAAGAATCCCA |
| | | | | GTTCCGTCTATAAATTCCAGGGAAGGTCTCTGATTGGC |
| | | | | CCTGCTCATTCCCAGGCCCATTCCTTGACCCAGTCACT |
| | | | | GAAGTCAGGGAGATGCAGTAATAAGACTGGCTGGAATC |
| | | | | AGGGTCTTTAGGGGTGGAGGGATGGGGAGGAGGCACAG |
| | | | | CATGTCATCAAAATAAGGAAATTGCAAAAGAAAGCTTG |
| | | | | CAGGCTACTTTGAATGACAATGAGAAAGACGGTGCTGC |
| | | | | CTGAGTGTGTTAAGGATCCACATGGTCTCCAAAATCCT |
| | | | | CCAGGAGCATACAGTCTAGTCTGGGAGATGAGACACAA |
| | | | | AAATAACCAGAACACAACAGCTTGCACTGACTCGAGGG |
| | | | | CTGGATAAGAATATCTGGAACTCCCCCATCTATTTCAG |
| | | | | AAGCTTGTCTCTTGGATGAAAATTAGACACTTAATGGG |
| | | | | AAAGGGCTTTGAAAAGAGTGCAGTAA |
| 70 | DES | Dph-CRE70 | 398 | CCTCAGGTACCCCCTGCCCCCCACAGCTCCTCTCCTGT |
| | | | | GCCTTGTTTCCCAGCGATGCGTTCTCCTCTATAAATAC |
| | | | | CCGCTCTGGTATTTGGGGTTGGCAGCTGTTGCTGCCAG |
| | | | | GGAGATGGTTGGGTTGACATGCGGCTCCTGACAAAACA |
| | | | | CAAACCCCTGGTGTGTGTGGGCGTGGGTGGTGTGAGTA |
| | | | | GGGGGATGAATCAGGGAGGGGGCGGGGGACCCAGGGGG |
| | | | | CAGGAGCCACACAAAGTCTGTGCGGGGGTGGGAGCGCA |
| | | | | CATAGCAATTGGAAACTGAAAGCTTATCAGACCCTTTC |
| | | | | TGGAAATCAGCCCACTGTTTATAAACTTGAGGCCCCAC |
| | | | | CCTCGACAGTACCGGGGAGGAAGAGGGCCTGCACTAGT |
| | | | | CCAGAGGGAAACTGAGGC |
| 71 | DES | Dph-CRE71 | 241 | CAGGGGCGCAGGCCTCTTGCGGGGGAGCTGGCCTCCCC |
| | | | | GCCCCCACGGCCACGGGCCGCCCTTTCCTGGCAGGACA |
| | | | | GCGGGATCTTGCAGCTGTCAGGGGAGGGGAGGCGGGGG |
| | | | | CTGATGTCAGGAGGGATACAAATAGTGCCGACGGCTGG |
| | | | | GGGCCCTGTCTCCCCTCGCCGCATCCACTCTCCGGCCG |
| | | | | GCCGCCTGCCCGCCGCCTCCTCCGTGCGCCCGCCAGCC |
| | | | | TCGCCCGCGCCGT |
| 72 | TNNC1 | Dph-CRE72 | 238 | CCCCCAGGCAGCGGCACGGTGGACTTTGATGAGTTCCT |
| | | | | GGTCATGATGGTTCGGTGCATGAAGGACGACAGCAAAG |
| | | | | GGAAATCTGAGGAGGAGCTGTCTGACCTCTTCCGCATG |
| | | | | TTTGACAAGTGAGCACGTGACCCTTGACCTCTGACCCT |
| | | | | GACCCACACTCAAGCCGAGCTGTACAGGAGGGCAGTCT |
| | | | | CAGATTCCAGGCCTAGGGACCCTGTGGCCTCTGCCTGA |
| | | | | TAGGGGAGAG |
| 73 | TNNC1 | Dph-CRE73 | 465 | AGCCAGCTCTGGGGGCACGCCTGCTTATCCTGTGGGAG |
| | | | | TCCATGGAGCCGGGGTTGGGACAGCCCTCCACCCAGTG |
| | | | | CCCATACAAGGCCTGGCGGAGTTGGGGACTAATTTTGG |
| | | | | CTTCTGAGGCGGCACTAGCAGGCCAGGGGGCCAGATAA |
| | | | | CGCTGCCCCACCCCCTGCATGCCAAAGTCCCCAGAACA |
| | | | | ATCACCAGGTTTAACTTTGTTCCTCGTTAAAAATAGCC |
| | | | | CAGTGGCCACCCTGGTCAGGTTACCGTGGGTGGCTTGC |
| | | | | CTGCCTCCACACTGGTTTTATTATCCCAACTTGAGGGA |
| | | | | CAGCTGTCCTTCGGGCCACCCAGCTTGAGTTTCATCAG |
| | | | | GGGCCGAAAGGGCATTGAGTGGTCACTGACTATTGTTA |
| | | | | CTGAGGGTCACCTTGGTCCTGAAGGGGGTGCCCACCTG |
| | | | | TCACCCTGGCCCTGAGCCCAGTCGCAGTGAGGCCAGCT |
| | | | | GGGTCACGT |
| 74 | TNNC1 | Dph-CRE74 | 285 | TTTTCATTCATTCCAGAAACCTTTTCAGAGAGTCCCTT |
| | | | | TGGGGAGTGTGGGGGACAGGAGGGAAAGAAACCTGGTC |
| | | | | CTTGTAGCCGTTCGTCTGCTCCCTGCCCTGGGCAGAGG |
| | | | | ACGTGGGGACTCAGGCCAGCCTGAGATCACTGGGACCA |
| | | | | GAGGAGGGGCTGGAGGATACTACACGCAGGGGTGGGCT |
| | | | | GGGCTGGGCTGGGCTGGGCCAGGAATGCAGCGGGGCAG |
| | | | | GGCTATTTAAGTCAAGGGCCGGCTGGCAACCCCAGCAA |
| | | | | GCTGTCCTGTGAGCCGCCA |
| 75 | TCAP | Dph-CRE75 | 332 | TGCTCTGTTTACAGACAAGCTGCTGTCCTCCCTGCAAA |
| | | | | GGGGAGTGGGTGGGGCAGAGGGCAAGTGCCAGGGGGGC |
| | | | | ACAAGGCTGGGCATGTGGCTGGCATGAGACGGTGTCTG |
| | | | | AGTAATGTCAGGCACCTGGAGGCATTGACCCCAGGACC |
| | | | | TTGGACCCCAGACCTCTGACCGGGGGGCAGCCAGCGTC |
| | | | | CAGGTACCCCAACCCCTGCCCTGGGTCCGGCGTCCCCC |
| | | | | CATTAGTGAGTCTTGGCTCTACTTATAGCATCTGACAC |
| | | | | CAGAGGGGCCGAAAATAGCCCCTGGAGAAGGGGGAGGA |
| | | | | GGGGGCTATTTAAAGGGCCTGGGAGGGG |
| 76 | MYH7 | Dph-CRE76 | 381 | GCCTGTGGTCTTGGTGGTCGTGGTCAGTTCCCTCTCCT |
| | | | | GCCAGCTGTGGAATGTGAGGCCTGGCCTGGGAGATATT |
| | | | | TTTGCTGCACTTTGAGCCACCCCGCCCCCTGGAACTCA |
| | | | | GACCCTGCACAGTCCATGCCATAACAATGACGACCACT |
| | | | | TCCAATTGTTTCCTAGCTGGAGAGGCGGGGAGGGGAGC |
| | | | | ACTGTTTGGGAAGGGGGGGAGCCTGGGGGAAATGCTTC |
| | | | | TAGTGACAACAGCCCTTTCTAAATCCGGCTAGGGACTG |
| | | | | GGTGCCGTTGGGGGTGGGGGTGCCCTGCTGCCCCATAT |
| | | | | ATACAGCCCCTGAGACCAGGTCTGGCTCCACAGCTCTG |
| | | | | TCCTGCTCTGTGTCTTTCCCTGCTGCTCTCAGGTAGGA |
| | | | | G |
| 77 | MYH7 | Dph-CRE77 | 316 | GGAATACCCTACCTTAAAAACAAAACAAACCGTGCTAC |
| | | | | TTGGCCCTCTTCCCTTGAGTTGTTGTCTAATCTCATTC |
| | | | | ATTTCACTGCCAAACTCCTCAAATGCATGGTGTACACC |
| | | | | CACTGCTTCCACTTCCTGTCCACTCATCTTTTTCCTCA |
| | | | | TTAACTCCCTGTGGCCTGGCTTTGCCTGTGACACTACA |
| | | | | CAAGCTGCTTGCTCCAAGATAATCCAAGTTCTTTTCTC |
| | | | | TGTCTTCCTGATACACAGGCTTTCTTTGGCATCTGACA |
| | | | | CTGCTGGCCACCTGCTCTTCTTGAAATGCTTCTTTTGG |
| | | | | CTTCTAGGAAAC |
| 78 | ALDOA | Dph-CRE78 | 401 | CCAAGTGGTGGGTGTAGCCTGGGGGTTCAATCCTCCTG |
| | | | | TGGCGCCCCAGAACCCGGTGCCTCCTCCAACGTCCGGC |
| | | | | ATCTGATGAGGATCCGACCCAGGCGGGCGGCGGCGGGA |
| | | | | TTCGCTCTTCCCCTTCGCTCCCCGCGAGAAAGCCCCGA |
| | | | | GGGCCGCGGCGGCGCAGAGCCGGTGACAGTTGAAGCTT |
| | | | | AGGCGGGAAGAGGGAGGCGCGAGGCGGGAAGAGGGAGT |
| | | | | TTGGGCCTCGGCAGCCGCCGTACAAACACCGCTCTGGT |
| | | | | CACCATGGCAACAGCGGGATGCCGCGAACGGCTTCTGG |
| | | | | GCGGGGCCGGTCCCTCGGACGATTGGACCTAGCTTGGC |
| | | | | GCGGAATCCGTGAATTGCCCGCGGCCCGAGGGTGCAGG |
| | | | | TGATGGGTGCTGACCGACTGG |
| 79 | ALDOA | Dph-CRE79 | 281 | GACTACAACCTGCCAGCTCAGGACGAGAGCTGTCAGGA |
| | | | | AGAGTCCAGGAATGGACTTCCCACGGGAGGGCACATTT |
| | | | | CTGGTATTCCTGGCAAGATAAGGAGTTGACTAAGTAAT |
| | | | | CCACGAGAAAAGGCATTTCCGGCAGAGGAAACAGTCTG |
| | | | | GGGGTGAGAGGGAGGCTGCAGCATTTGGGGAACTGCTA |
| | | | | GGGCTATCGTGTGTTTGGAAGAGGGGGAGGGAGAGAGG |
| | | | | TAGGCAGGGCTAAATTGGGAATTTTGTCACTGACATAA |
| | | | | ATTTTAAGTGCCAGG |
| 80 | ALDOA | Dph-CRE80 | 393 | CGTTTTGTGGTACCAGGGGGTCCCTCCTCTCCTGTCCC |
| | | | | CAGCCAAACCTTTTCCTTTCCCCTCGGGAAAGCTGCCT |
| | | | | TGGCTGTCACTACCTGCTGCCTATTCCACATCCTGAAC |
| | | | | CCTGTGACCTAGGCCCAGGGCTGCTGCGCGGACGGTAG |
| | | | | CTCCCCCTGCAGGAAGCAAGGTTCCTCCGGGCCCCCAG |
| | | | | ACTGCTGCTGGACCTGTGCAGAAGCCTGCAACTTTCCT |
| | | | | CTGCCTAGCCCGGCCCACTTCCTGGATGCTTGCTGCCC |
| | | | | CCAGCCCACCAGAGCTGTGAGTTCCATTCCTACCCCCT |
| | | | | GCCCCACTGAGCCCTGATCTAGGTATGATCGGTGCATT |
| | | | | CATTTTTTTGCTCAACAACATTTATTACTGAGCACCTT |
| | | | | CTCAAGGCCAGGC |
| 81 | ALDOA | Dph-CRE81 | 169 | CCTAGGCCCAGGGCTGCTGCGCGGACGGTAGCTCCCCC |
| | | | | TGCAGGAAGCAAGGTTCCTCCGGGCCCCCAGACTGCTG |
| | | | | CTGGACCTGTGCAGAAGCCTGCAACTTTCCTCTGCCTA |
| | | | | GCCCGGCCCACTTCCTGGATGCTTGCTGCCCCCAGCCC |
| | | | | ACCAGAGCTGTGAGTTC |
| 84 | TPM1 | Dph-CRE84 | 460 | TAAAGACCTTTCTCAGAGATCCAAACCAGCCCCACCCC |
| | | | | CCGCCCCCAGGAAGCATAGCATATTTTCTGTGAGGCCT |
| | | | | TGATGGTAGCATCACAAACCCTTGGGAAACACAACTCC |
| | | | | CAGTGTTTGAGGAGAGCATGCCGTATCTTGTTCCAGGG |
| | | | | ACACTGAGTCGTGAGCATAAAACGCTGCATTCCAAAAG |
| | | | | AGACGAAGAAGCAGTCGTCTCTCCATTTAATTATAGAT |
| | | | | TCTTCACTTTCCCTTAATTGCTTCAGTCAGCACTGTTG |
| | | | | ACTCTGGGGGAGTCACAGTACACCGGCAGGGCTATTGC |
| | | | | TGTTAAACAAGGGGTGACTATCAGGTAATGAGGTTTTC |
| | | | | ATTTTGTTTTTTCAAACAAACAAACCCTGATGTACATA |
| | | | | TTCAAGTGGGCATTCCTGTTAAAGGTGTCACATTGGGA |
| | | | | AATGATGCTCATGTTGACTCTCCTTTGTAACCAAATAT |
| | | | | TGAT |
| 85 | TPM1 | Dph-CRE85 | 292 | TAGGAAGGAACAAGAAGAGGTAGAAATGAAATGGCTCC |
| | | | | CAAAAGAAAAACCTCCCTTGGGTGGTATTTTAGGACAT |
| | | | | TAGCTCAGGCTGCCTTGTCCTCAGTCATCTCAGTGGCA |
| | | | | TTTAAGTAGCCCCTTGGGTTACAGATCACGGCAGGTGC |
| | | | | TGGGAAGTGAAGAAGGCCATGCTAAAAATACTGGCCTC |
| | | | | TTCTGGAACTCTGCCGGCAGCCCTGATGGAGCTCCCTC |
| | | | | CTCAAGCAGCAGTACCTCAGCAAAAGAACAGTTTCCTC |
| | | | | CGCCTTAAGCAGTAAGAAAAGTCTGG |
| 86 | TPM1 | Dph-CRE86 | 472 | AAGAAAGGGTCTTTACAAAACCAAAAGAAAGCCCATTG |
| | | | | CTTACTTTTAATAACCTAGGCCCACCCATAGGCCTCTA |
| | | | | AATCAAATAACCTCCACTGAGAATTAAGCTGTTAGCAC |
| | | | | TAGGTCTGGCATAGGTGCAGAAACAAAAGTTTAGAAAT |
| | | | | CCTAATCTTGAATTTACCTAACACGGCTGCCTCCTAGA |
| | | | | GCCTGAGTGGGTTGTAGCGAGACCTCAAGTAGAGGAGA |
| | | | | GGCACCTGGGCCATCCGCCTCCATGGGCTTTTTTTTTT |
| | | | | TTGCATCGAATTTCATGGTCTGCAAAGTAAGGGTGGGG |
| | | | | CTTTACTTGCCATTCAGGAGGTTGCAAAGGTCACCTCC |
| | | | | ACATATGTTCCTGTTAAGGACATCAGCAGAAACTTGAG |
| | | | | AAGCAAGTATAAAAATATAAAAATGAGCAGGTGGCATT |
| | | | | CAATAGGAAAAGAATGCAGGGTTTGCTGGGAAGTCATG |
| | | | | ATTGAAACCAGTCCAA |
| 87 | TFM1 | Dph-CRE87 | 476 | AAGGAAGTGACCAGGCCAGGTCTGGATCCCTGGAACTG |
| | | | | ACTCCTAACTCCCTATTCTTGCCTCCTCTGCTGAGCTC |
| | | | | CAACCTGGGGAGTCAGCTCCCTGGAGTCCACGCATCTG |
| | | | | GATATCGTCCACATGCCTGGAACTTTGTCACTGTTCCG |
| | | | | GTGGCTGGGTTGACACACCGTGATTAAAGGGCTCTGGT |
| | | | | CAATTTCTGTGCCACGCAGCCCCTATGCTAATGGTCAG |
| | | | | ATCTAATGTTCTGACCATTAGGTCAGTGTATCTTTTCC |
| | | | | CTCCTGGGCAGTTAGGTTAGTGGAAGAAACCCGATAAA |
| | | | | ATCTTGGAGAGGAATTTGATCATTCTCTGAAGGACTTA |
| | | | | CAAGTTGTTTGGCCTGCCTAATCTGCTCAGATACCGTC |
| | | | | CGGGGAATATTTGGTAACCATGCAGTACAGACTGTGAA |
| | | | | TTATTCTGTGGGGACTATTAACAAGACCCTCACCAACC |
| | | | | CTGCCTCAGCTGATCTCAGG |
| 88 | TPM1 | Dph-CRE88 | 459 | ACAGACCAAATTGCCTGAGTCTAAACTCAATGTTCTTT |
| | | | | CCCCTTAGACTGCCACCCGCACACACACGCACACATGC |
| | | | | ACACCTTTTTGGAGGCACTGCACAGCAAGCACAGAAGT |
| | | | | ACGCCACTGAGTCCTGTGTCCCTAAAGTATTTGACTGT |
| | | | | CCCTCCGCTGGGGGAAAGGGCAGGCAGAAAACAAGACT |
| | | | | CCGTGTGCTGTCGCTGTGCCGCCCCCTGCCTCTCTGAC |
| | | | | CCGCGCCCGCAGAGAAAGTCTCAAGAGCCGCCCCAGGC |
| | | | | TTTCTCCCACGTTCTCCCTTTCTCTGCTGCAGTTGAGT |
| | | | | TTCCAGAGCGTGAGCGCGCAGGATGACACCTGGCTGGC |
| | | | | TGAGAGCTGCCGGGGAGGCGCTGGCGGGTGCCGAGAGC |
| | | | | GCACTGACCCTGACGCGGGGTGCAGCACGGCTGGGAAG |
| | | | | CCCCCGGGCCTTTGGCTAAGCGCGCCGGGGGACGGCAC |
| | | | | AGG |
| 89 | TPM1 | Dph-CRE89 | 378 | GCCCCAACGCCCTCTCCCTGGCGCGCAGGTTTAGAAAC |
| | | | | AGGGCGGCCTCTCCGGCCGCCGCCTCGGCGGCTCGGGT |
| | | | | CCCCATATATAGTCATATCCACCGTCAACTGGGAGGCC |
| | | | | GGCGGCCGGCAGCGAATGGGCGAGCGGCCCCCGCGGGA |
| | | | | GGAGCGGGGAGGGGGCACGGGGCGGAGGGAGGAGAGGA |
| | | | | GGAAGGGGGGCAGGAGAAAAAAGCTTTTCCAAAAAAGT |
| | | | | ATTGGCTGTCTTGAGGAATGCGGTCGCCCCCTTGGGAA |
| | | | | AGTACATATCTGGGAGAAGCAGGCGGCTCCGCGCTCGC |
| | | | | ACTCCCGCTCCTCCGCCCGACCGCGCGCTCGCCCCGCC |
| | | | | GCTCCTGCTGCAGCCCCAGGGCCCCTCGCCGCCGCC |

A ‘nucleic acid regulatory element’ or ‘regulatory element’, also called “CRE” (cis-regulatory element), “CRM” (cis-regulatory module), or “SH” as used herein refers to a transcriptional control element, in particular a non-coding cis-acting transcriptional control element, capable of regulating and/or controlling transcription of a gene, in particular tissue-specific transcription of a gene. Regulatory elements comprise at least one transcription factor binding site (TFBS), more in particular at least one binding site for a tissue-specific transcription factor, most particularly at least one binding site for a muscle-specific transcription factor. Typically, regulatory elements as used herein increase or enhance promoter-driven gene expression when compared to the transcription of the gene from the promoter alone, without the regulatory elements. Thus, regulatory elements particularly comprise enhancer sequences, although it is to be understood that the regulatory elements enhancing transcription are not limited to typical far upstream enhancer sequences, but may occur at any distance of the gene they regulate. Indeed, it is known in the art that sequences regulating transcription may be situated either upstream (e.g. in the promoter region) or downstream (e.g. in the 3′UTR) of the gene they regulate in vivo, and may be located in the immediate vicinity of the gene or further away. Of note, although regulatory elements as disclosed herein typically comprise naturally occurring sequences, combinations of (parts of) such regulatory elements or several copies of a regulatory element, i.e. regulatory elements comprising non-naturally occurring sequences, are themselves also envisaged as regulatory element. Regulatory elements as used herein may comprise part of a larger sequence involved in transcriptional control, e.g. part of a promoter sequence. However, regulatory elements alone are typically not sufficient to initiate transcription, but require a promoter to this end. The regulatory elements disclosed herein are provided as nucleic acid molecules, i.e. isolated nucleic acids, or isolated nucleic acid molecules. Said nucleic acid regulatory elements hence have a sequence which is only a small part of the naturally occurring genomic sequence and hence is not naturally occurring as such, but is isolated therefrom.

The term “nucleic acid” as used herein typically refers to an oligomer or polymer (preferably a linear polymer) of any length composed essentially of nucleotides. A nucleotide unit commonly includes a heterocyclic base, a sugar group, and at least one, e.g. one, two, or three, phosphate groups, including modified or substituted phosphate groups. Heterocyclic bases may include inter alia purine and pyrimidine bases such as adenine (A), guanine (G), cytosine (C), thymine (T) and uracil (U) which are widespread in naturally-occurring nucleic acids, other naturally-occurring bases (e.g., xanthine, inosine, hypoxanthine) as well as chemically or biochemically modified (e.g., methylated), non-natural or derivatised bases. Sugar groups may include inter alia pentose (pentofuranose) groups such as preferably ribose and/or 2-deoxyribose common in naturally-occurring nucleic acids, or arabinose, 2-deoxyarabinose, threose or hexose sugar groups, as well as modified or substituted sugar groups. Nucleic acids as intended herein may include naturally occurring nucleotides, modified nucleotides or mixtures thereof. A modified nucleotide may include a modified heterocyclic base, a modified sugar moiety, a modified phosphate group or a combination thereof. Modifications of phosphate groups or sugars may be introduced to improve stability, resistance to enzymatic degradation, or some other useful property. The term “nucleic acid” further preferably encompasses DNA, RNA and DNA/RNA hybrid molecules, specifically including hnRNA, pre-mRNA, mRNA, cDNA, genomic DNA, amplification products, oligonucleotides, and synthetic (e.g., chemically synthesised) DNA, RNA or DNA/RNA hybrids. A nucleic acid can be naturally occurring, e.g., present in or isolated from nature; or can be non-naturally occurring, e.g., recombinant, i.e., produced by recombinant DNA technology, and/or partly or entirely, chemically or biochemically synthesised. A “nucleic acid” can be double-stranded, partly double stranded, or single-stranded. Where single-stranded, the nucleic acid can be the sense strand or the antisense strand. In addition, nucleic acid can be circular or linear.

As used herein “transcription factor binding site”, “transcription factor binding sequence” or “TFBS” refers to a sequence of a nucleic acid region to which transcription factors bind. Non-limiting examples of TFBS include binding sites for transcription factor 3, also known as TCF3 or E2A; binding sites for nuclear factor I, also known as NF1; binding sites for CCAAT-enhancer-binding protein, also known as C/EBP; binding sites for myogenic differentiation, also known as MyoD; binding sites for sterol regulatory element-binding protein, also known as SREBP; binding sites for leukemia/lymphoma-related factor, also known as LRF; binding sites for protein 53, also known as p53; binding sites for hepatocyte nuclear factor 3-alpha, also known as HNF3a; binding sites for hepatocyte nuclear factor 3-beta, also known as HNF3b; binding sites for hepatocyte nuclear factor 4, also known as HNF4; binding sites for myocyte-specific enhancer factor 2A, also known as MEF2A or RSRFC4; binding sites for peroxisome proliferator-activated receptor, also known as PPAR; binding sites for serum response factor, also known as SRF; binding sites for transcription activator-like protein 1b, also known as Tal1_b. Transcription factor binding sites may be found in databases such as Transfac®.

Sequences disclosed herein may be part of sequences of regulatory elements capable of controlling transcription of diaphragm-, and skeletal muscles-specific genes in vivo. Particular examples for diaphragm and skeletal muscle specific regulatory elements may in particular be controlling the following genes (cf. Table 2): ACTA1, CKM, TPM2, MYL1, TNNC2, FHL1, TNNT1, TNNI2, MYLPF, TNNT3, MYH2, SLN, MYBPC1, ENO3, CA3, ATP2A1, or MYH1.

Accordingly, in embodiments, the nucleic acid regulatory elements disclosed herein comprise a sequence from ACTA1 regulatory elements, i.e. regulatory elements that control expression of the ACTA1 gene (Alpha-actin-1 gene) in vivo, e.g. regulatory elements comprising any one or more of SEQ ID NOs: 1 to 5, or functional fragments thereof. In other embodiments, the nucleic acid regulatory elements disclosed herein comprise a sequence from CKM regulatory elements, i.e. regulatory elements that control expression of the CKM gene (Muscle Creatine Kinase gene) in vivo, e.g. regulatory elements comprising any one or more of SEQ ID NOs: 6 or 7, or functional fragments thereof. In other embodiments, the nucleic acid regulatory elements disclosed herein comprise a sequence from MYL2 regulatory elements, i.e. regulatory elements that control expression of the MYL2 (Myosin, Light Chain 2 gene) gene in vivo, e.g. regulatory elements comprising any one or more of SEQ ID NOs: 8 or 9, or functional fragments thereof. In other embodiments, the nucleic acid regulatory elements disclosed herein comprise a sequence from TPM2 regulatory elements, i.e. regulatory elements that control expression of the TPM2 gene (Tropomyosin 2 gene) in vivo, e.g. regulatory elements comprising any one or more of SEQ ID NOs: 10 to 12, or functional fragments thereof. In other embodiments, the nucleic acid regulatory elements disclosed herein comprise a sequence from MYL1 regulatory elements, i.e. regulatory elements that control expression of the MYL1 gene (gene) in vivo, e.g. regulatory elements comprising any one or more of SEQ ID NOs: 13 to 17, or functional fragments thereof. In other embodiments, the nucleic acid regulatory elements disclosed herein comprise a sequence from TNNC2 regulatory elements, i.e. regulatory elements that control expression of the TNNC2 gene (Troponin T2, Cardiac Type gene) in vivo, e.g. regulatory elements comprising any one or more of SEQ ID NOs: 18 or 19, or functional fragments thereof. In other embodiments, the nucleic acid regulatory elements disclosed herein comprise a sequence from FHL1 regulatory elements, i.e. regulatory elements that control expression of the FHL1 gene (Four And A Half LIM Domains 1 gene) in vivo, e.g. regulatory elements comprising any one or more of SEQ ID NOs: 20 to 26, or functional fragments thereof. In other embodiments, the nucleic acid regulatory elements disclosed herein comprise a sequence from TNNT1 regulatory elements, i.e. regulatory elements that control expression of the TNNT1 gene (Troponin T Type 1 gene) in vivo, e.g. regulatory elements comprising any one or more of SEQ ID NOs: 27 or 28, or functional fragments thereof. In other embodiments, the nucleic acid regulatory elements disclosed herein comprise a sequence from TNNI2 regulatory elements, i.e. regulatory elements that control expression of the TNNI2 gene (Troponin I Type 2 gene) in vivo, e.g. regulatory elements comprising any one or more of SEQ ID NOs: 29 to 31, or functional fragments thereof. In other embodiments, the nucleic acid regulatory elements disclosed herein comprise a sequence from MYLPF regulatory elements, i.e. regulatory elements that control expression of the MYLPF gene (Myosin Light Chain, Phosphorylatable, Fast Skeletal Muscle gene) in vivo, e.g. regulatory elements comprising SEQ ID NO: 32, or functional fragments thereof. In other embodiments, the nucleic acid regulatory elements disclosed herein comprise a sequence from TNNT3 regulatory elements, i.e. regulatory elements that control expression of the TNNT3 gene (Troponin T Type 3 gene) in vivo, e.g. regulatory elements comprising any one or more of SEQ ID NOs: 33 to 38, or functional fragments thereof. In other embodiments, the nucleic acid regulatory elements disclosed herein comprise a sequence from MYH2 regulatory elements, i.e. regulatory elements that control expression of the MYH2 gene (Myosin, Heavy Chain 2 gene) in vivo, e.g. regulatory elements comprising any one or more of SEQ ID NOs: 82 or 83, or functional fragments thereof. In other embodiments, the nucleic acid regulatory elements disclosed herein comprise a sequence from SLN regulatory elements, i.e. regulatory elements that control expression of the SLN gene (Sarcolipin gene) in vivo, e.g. regulatory elements comprising any one or more of SEQ ID NOs: 39 to 42, or functional fragments thereof. In other embodiments, the nucleic acid regulatory elements disclosed herein comprise a sequence from MYBPC1 regulatory elements, i.e. regulatory elements that control expression of the MYBPC1 gene (Myosin Binding Protein C, Slow Type gene) in vivo, e.g. regulatory elements comprising any one or more of SEQ ID NOs: 43 to 50, or functional fragments thereof. In other embodiments, the nucleic acid regulatory elements disclosed herein comprise a sequence from ENO3 regulatory elements, i.e. regulatory elements that control expression of the ENO3 gene (Enolase 3 gene) in vivo, e.g. regulatory elements comprising any one or more of SEQ ID NOs: 51 to 54, or functional fragments thereof. In other embodiments, the nucleic acid regulatory elements disclosed herein comprise a sequence from CA3 regulatory elements, i.e. regulatory elements that control expression of the CA3 gene (Carbonic Anhydrase III gene) in vivo, e.g. regulatory elements comprising any one or more of SEQ ID NOs: 55 to 57, or functional fragments thereof. In other embodiments, the nucleic acid regulatory elements disclosed herein comprise a sequence from ATP2A1 regulatory elements, i.e. regulatory elements that control expression of the ATP2A1 gene (ATPase, Ca++Transporting, Cardiac Muscle, Fast Twitch 1 gene or Sarcoplasmic/endoplasmic reticulum calcium ATPase 1 gene) in vivo, e.g. regulatory elements comprising any one or more of SEQ ID NOs: 58 to 64, or functional fragments thereof. In other embodiments, the nucleic acid regulatory elements disclosed herein comprise a sequence from MYH1 regulatory elements, i.e. regulatory elements that control expression of the MYH1 gene (Myosin, Heavy Chain 1 gene) in vivo, e.g. regulatory elements comprising SEQ ID NO: 65, or functional fragments thereof.

Sequences disclosed herein may be part of sequences of regulatory elements capable of controlling transcription of diaphragm-, skeletal muscle and heart-specific genes in vivo. Particular examples for diaphragm-, skeletal muscle and heart specific regulatory elements may in particular be controlling the following genes: ACTA1, CKM, MYL2, MB, DES, TNNC1, TCAP, MYH7, ALDOA, or TPM1. Accordingly, in embodiments, the nucleic acid regulatory elements disclosed herein comprise a sequence from ACTA1 regulatory elements, i.e. regulatory elements that control expression of the ACTA1 gene (Alpha-actin-1 gene) in vivo, e.g. regulatory elements comprising any one or more of SEQ ID NOs: 1 to 5, or functional fragments thereof. In other embodiments, the nucleic acid regulatory elements disclosed herein comprise a sequence from CKM regulatory elements, i.e. regulatory elements that control expression of the CKM gene (Muscle Creatine Kinase gene) in vivo, e.g. regulatory elements comprising any one or more of SEQ ID NOs: 6 or 7, or functional fragments thereof. In other embodiments, the nucleic acid regulatory elements disclosed herein comprise a sequence from MYL2 regulatory elements, i.e. regulatory elements that control expression of the MYL2 (Myosin, Light Chain 2 gene) gene in vivo, e.g. regulatory elements comprising any one or more of SEQ ID NOs: 8 or 9, or functional fragments thereof. In other embodiments, the nucleic acid regulatory elements disclosed herein comprise a sequence from MB regulatory elements, i.e. regulatory elements that control expression of the MB gene (Myoglobin gene) in vivo, e.g. regulatory elements comprising any one or more of SEQ ID NOs: 66 to 68, or functional fragments thereof. In other embodiments, the nucleic acid regulatory elements disclosed herein comprise a sequence from DES regulatory elements, i.e. regulatory elements that control expression of the DES gene (Desmin gene) in vivo, e.g. regulatory elements comprising any one or more of SEQ ID NOs: 69 to 71, or functional fragments thereof. In other embodiments, the nucleic acid regulatory elements disclosed herein comprise a sequence from TNNC1 regulatory elements, i.e. regulatory elements that control expression of the TNNC1 gene (Troponin C Type 1 gene) in vivo, e.g. regulatory elements comprising any one or more of SEQ ID NOs: 72 to 74, or functional fragments thereof. In other embodiments, the nucleic acid regulatory elements disclosed herein comprise a sequence from TCAP regulatory elements, i.e. regulatory elements that control expression of the TCAP gene (Titin-Cap gene) in vivo, e.g. regulatory elements comprising SEQ ID NOs: 75, or functional fragments thereof. In other embodiments, the nucleic acid regulatory elements disclosed herein comprise a sequence from MYH7 regulatory elements, i.e. regulatory elements that control expression of the MYH7 gene (Myosin, Heavy Chain 7 gene) in vivo, e.g. regulatory elements comprising any one or more of SEQ ID NOs: 76 or 77, or functional fragments thereof. In other embodiments, the nucleic acid regulatory elements disclosed herein comprise a sequence from ALDOA regulatory elements, i.e. regulatory elements that control expression of the ALDOA gene (Aldolase A gene) in vivo, e.g. regulatory elements comprising any one or more of SEQ ID NOs: 78 to 81, or functional fragments thereof. In other embodiments, the nucleic acid regulatory elements disclosed herein comprise a sequence from TPM1 regulatory elements, i.e. regulatory elements that control expression of the TPM1 gene (Tropomyosin 1 gene) in vivo, e.g. regulatory elements comprising any one or more of SEQ ID NOs: 84 to 89, or functional fragments thereof.

As used herein, the terms “identity” and “identical” and the like refer to the sequence similarity between two polymeric molecules, e.g., between two nucleic acid molecules, e.g., two DNA molecules. Sequence alignments and determination of sequence identity can be done, e.g., using the Basic Local Alignment Search Tool (BLAST) originally described by Altschul et al. 1990 (J Mol Biol 215: 403-10), such as the “Blast 2 sequences” algorithm described by Tatusova and Madden 1999 (FEMS Microbiol Lett 174: 247-250). Typically, the percentage sequence identity is calculated over the entire length of the sequence. As used herein, the term “substantially identical” denotes at least 90%, preferably at least 95%, such as 95%, 96%, 97%, 98% or 99%, sequence identity.

The term ‘functional fragment’ as used in the application refers to fragments of the regulatory element sequences disclosed herein that retain the capability of regulating muscle-specific expression, i.e. they can still confer tissue specificity and they are capable of regulating expression of a (trans)gene in the same way (although possibly not to the same extent) as the sequence from which they are derived. Functional fragments may preferably comprise at least 20, at least 25, at least 30, at least 35, at least 40, at least 45, at least 50, at least 60, at least 70, at least 80, at least 90, at least 100, at least 120, at least 150, at least 200, at least 250, at least 300, at least 350, or at least 400 contiguous nucleotides from the sequence from which they are derived. Also preferably, functional fragments may comprise at least 1, more preferably at least 2, at least 3, or at least 4, even more preferably at least 5, at least 10, or at least 15, of the transcription factor binding sites (TFBS) that are present in the sequence from which they are derived.

“Diaphragm and skeletal muscle-specific expression” as used in the application, refers to the preferential or predominant expression of a (trans)gene (as RNA and/or polypeptide) in diaphragm and skeletal muscle cells or diaphragm or skeletal muscle tissue, as compared to other (i.e. non-diaphragm or skeletal muscle) tissues. According to particular embodiments, at least 50%, more particularly at least 60%, at least 70%, at least 75%, at least 80%, at least 85%, at least 90%, at least 95%, at least 98%, at least 99% or 100% of the (trans)gene expression occurs within diaphragm and/or skeletal muscle cells or tissue. According to a particular embodiment, diaphragm and skeletal muscle specific expression entails that there is less than 10%, less than 5%, less than 2% or even less than 1% ‘leakage’ of expressed gene product to other organs or tissue than muscle, such as lung, liver, brain, kidney and/or spleen.

As used herein “diaphragm, skeletal muscle and cardiac-specific expression” refers to the preferential or predominant expression of a (trans)gene in diaphragm, heart, skeletal muscle cells or tissue and in particular heart muscle. According to particular embodiments, at least 50%, more particularly at least 60%, at least 70%, at least 75%, at least 80%, at least 85%, at least 90%, at least 95%, at least 98%, at least 99% or 100% of the (trans)gene expression occurs within diaphragm, skeletal muscle cells and heart tissue. Thus, according to particular embodiments, less than 10%, less than 5%, less than 2% or even less than 1% of the (trans)gene expression occurs in an organ or tissue other than diaphragm, heart and skeletal muscle.

The same applies mutatis mutandis for myocyte-specific and myoblast-specific expression, which may be considered as a particular form of muscle-specific expression. Throughout the application, where muscle-specific is mentioned in the context of expression, myocyte-specific and myoblast-specific expression are also explicitly envisaged. Similarly, where cardiac and skeletal muscle-specific expression is used in the application, cardiomyocyte and skeletal myocyte-specific expression and cardiac myoblast and skeletal myoblast-specific expression is also explicitly envisaged. Similarly, where skeletal muscle-specific expression is used in the application, skeletal myocyte-specific and skeletal myoblast-specific expression is also explicitly envisaged.

As used herein, the terms “heart muscle” or “cardiac muscle” refer to the autonomically regulated, striated muscle type found in the heart.

As used herein, the term “skeletal muscle” refers to the voluntarily controlled, striated muscle type that is attached to the skeleton. Non-limiting examples of skeletal muscle include the biceps, the triceps, the quadriceps, the tibialis interior, and the gastrocnemius muscle.

The term “myocyte,” as used herein, refers to a cell that has been differentiated from a progenitor myoblast such that it is capable of expressing muscle-specific phenotype under appropriate conditions. Terminally differentiated myocytes fuse with one another to form myotubes, a major constituent of muscle fibers. The term “myocyte” also refers to myocytes that are de-differentiated. The term includes cells in vivo and cells cultured ex vivo regardless of whether such cells are primary or passaged.

The term “myoblast” as used herein, refers to an embryonic cell in the mesoderm that differentiates to give rise to a muscle cell or myocyte. The term includes cells in vivo and cells cultured ex vivo regardless of whether such cells are primary or passaged.

In embodiments, the invention relates to a nucleic acid regulatory element for enhancing gene expression in diaphragm and skeletal muscle comprising, consisting essentially of, or consisting of a sequence selected from the group consisting of: SEQ ID NO:1 to 7, 10 to 65, 82, and 83 (cf. Dph-CRE1 to 7, 10 to 65, 82, and 83 in Table 3); a sequence having at least 80%, preferably at least 85%, more preferably at least 90%, even more preferably at least 95%, such as 95%, 96%, 97%, 98%, or 99%, identity to any of the sequences selected from the group consisting of SEQ ID NO: 1 to 7, 10 to 65, 82, and 83 (cf. Dph-CRE1 to 7, 10 to 65, 82, and 83 in Table 3); or a functional fragment thereof, wherein said functional fragment comprises at least 20, preferably at least 25, more preferably at least 50, at least 100, at least 200 or at least 250, contiguous nucleotides from the sequence from which it is derived, and wherein said functional fragment comprises at least 1, preferably at least 5, more preferably at least 10 or at least 15, of the transcription factor binding sites (TFBS) that are present in the sequence from which it is derived.

In further embodiments, the invention relates to a nucleic acid regulatory element for enhancing gene expression in diaphragm, skeletal muscle and cardiac comprising, consisting essentially of, or consisting of a sequence selected from the group consisting of: SEQ ID No: 1 to 9, 66 to 81, and 84 to 89. (cf. Dph-CRE1 to 9, 66 to 81, and 84 to 89 in Table 4); or a sequence having at least 80%, preferably at least 85%, more preferably at least 90%, even more preferably at least 95%, such as 95%, 96%, 97%, 98%, or 99%, identity to any one of the sequences selected from the group consisting of SEQ ID No: 1 to 9, 66 to 81, and 84 to 89. (cf. Dph-CRE1 to 9, 66 to 81, and 84 to 89 in Table 4); or a functional fragment thereof, wherein said functional fragment comprise at least 20, preferably at least 25, more preferably at least 50, at least 100, at least 200 or at least 250, contiguous nucleotides from the sequence from which it is derived, and wherein said functional fragment comprises at least 1, preferably at least 5, more preferably at least 10 or at least 15, of the transcription factor binding sites (TFBS) that are present in the sequence from which it is derived.

It is also possible to make nucleic acid regulatory elements that comprise an artificial sequence by combining two or more identical or different sequences disclosed herein or functional fragments thereof. Accordingly, in certain embodiments a nucleic acid regulatory element for enhancing gene expression in diaphragm, and skeletal muscle cells or tissue is provided comprising at least two sequences selected from the group consisting of: SEQ ID NO:1 to 7, 10 to 65, 82, and 83 (cf. Dph-CRE1 to 7, 10 to 65, 82, and 83 in Table 3).

Alternatively, in certain embodiments a nucleic acid regulatory element for enhancing gene expression in diaphragm, skeletal muscle and heart cells or tissue is provided comprising at least two sequences selected from the group consisting of: SEQ ID No: 1 to 9, 66 to 81, and 84 to 89. (cf. Dph-CRE1 to 9, 66 to 81, and 84 to 89 in Table 4).

For example, disclosed herein is a nucleic acid regulatory element comprising, consisting essentially of, or consisting of 2, 3, 4, or 5 repeats, e.g. tandem repeats, of any one of SEQ ID NOs:1 to 89, or combinations thereof.

Particular examples of nucleic acid regulatory elements that comprise an artificial sequence include the regulatory elements that are obtained by rearranging the transcription factor binding sites (TFBS) that are present in the sequences disclosed herein. Said rearrangement may encompass changing the order of the TFBSs and/or changing the position of one or more TFBSs relative to the other TFBSs and/or changing the copy number of one or more of the TFBSs. For example, also disclosed herein is a nucleic acid regulatory element for enhancing muscle-specific gene expression, in particular cardiac and skeletal muscle-specific gene expression, comprising binding sites for E2A, HNH1, NF1, C/EBP, LRF, MyoD, and SREBP; or for E2A, NF1, p53, C/EBP, LRF, and SREBP; or for E2A, HNH1, HNF3a, HNF3b, NF1, C/EBP, LRF, MyoD, and SREBP; or E2A, HNF3a, NF1, C/EBP, LRF, MyoD, and SREBP; or for E2A, HNF3a, NF1, CEBP, LRF, MyoD, and SREBP; or for HNF4, NF1, RSRFC4, C/EBP, LRF, and MyoD, or NF1, PPAR, p53, C/EBP, LRF, and MyoD. Further for example, also disclosed herein is a nucleic acid regulatory element for enhancing diaphragm and skeletal muscle-specific gene expression, in particular comprising binding sites for one or more of: NFYA, SIN3A, TCF12, PHF8, IRF1 and combinations thereof, such as NFYA, SIN3A, TCF12, PHF8, and IRF1. Further for example, also disclosed herein is a nucleic acid regulatory element for enhancing diaphragm, heart, and skeletal muscle-specific gene expression, in particular comprising binding sites for one or more of: MAFF, FOXA2, TAL1, CEBPB, RFX5, HSF1, SRF and combinations thereof, such as MAFF, FOXA2, TAL1, CEBPB, RFX5, HSF1, and SRF. In some embodiments, these nucleic acid regulatory elements comprise at least two, such as 2, 3, 4, or more copies of any one or more of the recited TFBSs.

In case the regulatory element is provided as a single stranded nucleic acid, e.g. when using a single-stranded AAV vector, the complement strand is considered equivalent to the disclosed sequences. Hence, also disclosed herein is a nucleic acid regulatory element for enhancing muscle-specific gene expression comprising, consisting essentially of, or consisting of the complement of a sequence described herein, in particular a sequence selected from the group consisting of: SEQ ID NOs:1 to 89; a sequence having at least 80%, preferably at least 85%, more preferably at least 90%, even more preferably at least 95%, such as 95%, 96%, 97%, 98%, or 99%, identity to any of these sequences; or a functional fragment thereof.

Also disclosed herein is a nucleic acid regulatory element for enhancing muscle-specific gene expression hybridizing under stringent conditions to a nucleic acid regulatory element described herein, in particular to the nucleic acid regulatory element comprising, consisting essentially of, or consisting of a sequence selected from the group consisting of: SEQ ID NOs:1 to 89; a sequence having at least 90%, preferably at least 95%, such as 95%, 96%, 97%, 98%, or 99%, identity to any of these sequences; a functional fragment thereof; or to its complement. Said nucleic acid regulatory elements do not need to be of equal length as the sequence they hybridize to. In preferred embodiments, the size of said hybridizing nucleic acid regulatory element does not differ more than 25% in length, in particular 20% in length, more in particular 15% in length, most in particular 10% in length from the sequence it hybridizes to.

The expression ‘hybridize under stringent conditions’ refers to the ability of a nucleic acid molecule to hybridize to a target nucleic acid molecule under defined conditions of temperature and salt concentration. Typically, stringent hybridization conditions are no more than 25° C. to 30° C. (for example, 20° C., 15° C., 10° C. or 5° C.) below the melting temperature (Tm) of the native duplex. Methods of calculating Tm are well known in the art. By way of non-limiting example, representative salt and temperature conditions for achieving stringent hybridization are: 1×SSC, 0.5% SDS at 65° C. The abbreviation SSC refers to a buffer used in nucleic acid hybridization solutions. One liter of the 20× (twenty times concentrate) stock SSC buffer solution (pH 7.0) contains 175.3 g sodium chloride and 88.2 g sodium citrate. A representative time period for achieving hybridization is 12 hours.

Preferably the regulatory elements as described herein are fully functional while being only of limited length. This allows their use in vectors or nucleic acid expression cassettes without unduly restricting their payload capacity. Accordingly, in embodiments, the regulatory element disclosed herein is a nucleic acid of 1500 nucleotides or less, 1000 nucleotides or less, 900 nucleotides or less, 800 nucleotides or less, 700 nucleotides or less, more preferably 600 nucleotides or less, such as 550 nucleotides or less, 500 nucleotides or less, 450 nucleotides or less, 400 nucleotides or less, 350 nucleotides or less, or 300 nucleotides or less (i.e. the nucleic acid regulatory element has a maximal length of 1500 nucleotides, 1000 nucleotides, 900 nucleotides, 800 nucleotides, 700 nucleotides, preferably 600 nucleotides, such as 550 nucleotides, 500 nucleotides, 450 nucleotides, 400 nucleotides, 350 nucleotides, or 300 nucleotides).

However, it is to be understood that the disclosed nucleic acid regulatory elements retain regulatory activity (i.e. with regard to specificity and/or activity of transcription) and thus they particularly have a minimum length of 20 nucleotides, 25 nucleotides, 30 nucleotides, 35 nucleotides, 40 nucleotides, 45 nucleotides, 50 nucleotides, 100 nucleotides, 150 nucleotides, 200 nucleotides, 250 nucleotides, 300 nucleotides, 350 nucleotides or 400 nucleotides.

In certain embodiments, the invention provides for a nucleic acid regulatory element of 1000 nucleotides or less, preferably 900 nucleotides or less, preferably 800 nucleotides or less, preferably 700 nucleotides or less of a sequence selected from the group consisting of: SEQ ID NOs:1 to 89; a sequence having at least 80%, preferably at least 85%, more preferably at least 90%, even more preferably at least 95%, such as 95%, 96%, 97%, 98%, or 99%, identity to any of said sequences; or a functional fragment thereof.

The nucleic acid regulatory elements disclosed herein may be used in a nucleic acid expression cassette. Accordingly, in an aspect the invention provides for the use of the nucleic acid regulatory elements as described herein in a nucleic acid expression cassette.

In an aspect the invention provides a nucleic acid expression cassette comprising a nucleic acid regulatory element as described herein, operably linked to a promoter. In embodiments, the nucleic acid expression cassette does not contain a transgene. Such nucleic acid expression cassette may be used to drive expression of an endogenous gene. In preferred embodiments, the nucleic acid expression cassette comprises a nucleic acid regulatory element as described herein, operably linked to a promoter and a transgene.

As used herein, the term ‘nucleic acid expression cassette’ refers to nucleic acid molecules that include one or more transcriptional control elements (such as, but not limited to promoters, enhancers and/or regulatory elements, polyadenylation sequences, and introns) that direct (trans)gene expression in one or more desired cell types, tissues or organs. Typically, they will also contain a transgene, although it is also envisaged that a nucleic acid expression cassette directs expression of an endogenous gene in a cell into which the nucleic acid cassette is inserted.

The term ‘operably linked’ as used herein refers to the arrangement of various nucleic acid molecule elements relative to each such that the elements are functionally connected and are able to interact with each other. Such elements may include, without limitation, a promoter, an enhancer and/or a regulatory element, a polyadenylation sequence, one or more introns and/or exons, and a coding sequence of a gene of interest to be expressed (i.e., the transgene). The nucleic acid sequence elements, when properly oriented or operably linked, act together to modulate the activity of one another, and ultimately may affect the level of expression of the transgene. By modulate is meant increasing, decreasing, or maintaining the level of activity of a particular element. The position of each element relative to other elements may be expressed in terms of the 5′ terminus and the 3′ terminus of each element, and the distance between any particular elements may be referenced by the number of intervening nucleotides, or base pairs, between the elements. As understood by the skilled person, operably linked implies functional activity, and is not necessarily related to a natural positional link. Indeed, when used in nucleic acid expression cassettes, the regulatory elements will typically be located immediately upstream of the promoter (although this is generally the case, it should definitely not be interpreted as a limitation or exclusion of positions within the nucleic acid expression cassette), but this needs not be the case in vivo. E.g., a regulatory element sequence naturally occurring downstream of a gene whose transcription it affects is able to function in the same way when located upstream of the promoter. Hence, according to a specific embodiment, the regulatory or enhancing effect of the regulatory element is position-independent.

In particular embodiments, the nucleic acid expression cassette comprises one nucleic acid regulatory element as described herein. In alternative embodiments, the nucleic acid expression cassette comprises two or more, such as, e.g., 2, 3, 4, 5, 6, 7, 8, 9, or 10, nucleic acid regulatory elements as described herein, i.e. they are combined modularly to enhance their regulatory (and/or enhancing) effect. In further embodiments, at least two of the two or more nucleic acid regulatory elements are identical or substantially identical. In yet further embodiments, all of the two or more regulatory elements are identical or substantially identical. The copies of the identical or substantially identical nucleic acid regulatory elements may be provided as tandem repeats in the nucleic acid expression cassette. In alternative further embodiments, at least two of the two or more nucleic acid regulatory elements are different from each other, that is to say, are defined by a different SEQ ID NO. The nucleic acid expression cassette may also comprise a combination of identical and substantially identical nucleic acid regulatory elements and non-identical nucleic acid regulatory elements.

For example, the nucleic acid expression cassette may comprise a nucleic acid regulatory element comprising SEQ ID NO:1, and a nucleic acid regulatory element comprising any one or more of SEQ ID Nos: 2 to 689; or the nucleic acid expression cassette may comprise a nucleic acid regulatory element comprising any one or more of SEQ ID NO: 1-89 and a nucleic acid regulatory element (cis-regulatory element, CRE, CRM, or SH) specific for another tissue type such as e.g. the ones disclosed in previous applications from the same authors: WO2015110449; WO2009130208; WO2009071679; WO2011051450; WO2016146757, WO2014063753; or WO2014064277. Alternatively, this can be done for remaining regulatory elements defined by SEQ ID NOs: 2 to 89 respectively. In a specific embodiment, the muscle-specific regulatory element designated Sk-SH4 (SEQ ID NO:121) or CSk-SH5 (SEQ ID NO:122) can be used in combination with any one of the Diaphragm cis-regulatory elements defined by SEQ ID NOs: 1 to 89.

As used in the application, the term ‘promoter’ refers to nucleic acid sequences that regulate, either directly or indirectly, the transcription of corresponding nucleic acid coding sequences to which they are operably linked (e.g. a transgene or endogenous gene). A promoter may function alone to regulate transcription or may act in concert with one or more other regulatory sequences (e.g. enhancers or silencers, or regulatory elements). In the context of the present application, a promoter is typically operably linked to a regulatory element as disclosed herein to regulate transcription of a (trans)gene. When a regulatory element as described herein is operably linked to both a promoter and a transgene, the regulatory element can (1) confer a significant degree of diaphragm-specific, in particular diaphragm and skeletal muscle-specific or diaphragm, cardiac and skeletal muscle-specific, expression in vivo (and/or in vitro in cell lines derived from cardiac, diaphragm, and skeletal muscle cells or tissue) of the transgene, and/or (2) can increase the level of expression of the transgene in diaphragm and skeletal muscle or in diaphragm, cardiac and skeletal muscle (and/or in vitro in cell lines derived from cardiac, diaphragm, and skeletal muscle cells or tissue).

The promoter may be homologous (i.e. from the same species as the animal, in particular mammal, to be transfected with the nucleic acid expression cassette) or heterologous (i.e. from a source other than the species of the animal, in particular mammal, to be transfected with the expression cassette). As such, the source of the promoter may be any virus, any unicellular prokaryotic or eukaryotic organism, any vertebrate or invertebrate organism, or any plant, or may even be a synthetic promoter (i.e. having a non-naturally occurring sequence), provided that the promoter is functional in combination with the regulatory elements described herein. In preferred embodiments, the promoter is a mammalian promoter, in particular a murine or human promoter.

The promoter may be an inducible or constitutive promoter.

Non-limiting exemplary skeletal muscle and/or diaphragm-specific promoter are the Desmin promotor, other muscle specific promoters such as muscle creatine kinase promoter (MCK) (Wang B et al, Gene Ther, 2008), alpha-myosin heavy chain (a-MHC), myosin light chain (MLC-2), cardiac troponin C (cTnC), myogenin MYF4 promoters (Pacak C. A. et al., Genet Vaccines Ther, 2008), viral promoters such as murine stem cell virus (MSCV) promoter (Suga T et al., Plos One, 2011), and all potential promoters that can be used to cloned downstream of the diaphragm nucleic acid regulatory elements. The regulatory elements disclosed herein can be used in nucleic acid expression cassettes in conjunction with their natural promoter, as well as with another promoter. Preferably, the nucleic acid expression cassettes disclosed herein comprise a diaphragm, heart, and/or skeletal muscle-specific promoter, in order to increase diaphragm, heart, and/or skeletal muscle-specificity and/or avoid leakage of expression in other tissues. Examples of such promotors are diaphragm and/or skeletal muscle-specific promoters, such as the promotor of one of the genes defined in Table 3, namely the ACTA1, CKM, TPM2, MYL1, TNNC2, FHL1, TNNT1, TNNI2, MYLPF, TNNT3, MYH2, SLN, MYBPC1, ENO3, CA3, ATP2A1, and MYH1 genes; or diaphragm-, skeletal muscle- and heart-specific promoters, such as the promotor of any one of the genes defined in Table 4, namely ACTA1, CKM, MYL2, MB, DES, TNNC1, TCAP, MYH7, ALDOA, and TPM1 genes. Non-limiting examples of muscle-specific promoters include the desmin (DES) promoter, the synthetic SPc-5-12 promoter (SPc5-12-GTRM), the alpha-actin1 promoter (ACTA1), the Creatine kinase, muscle (CKM) promoter, the Four and a half LIM domains protein 1 (FHL1) promoter, the alpha 2 actinin (ACTN2) promoter, the filamin-C(FLNC) promoter, the sarcoplasmic/endoplasmic reticulum calcium ATPase 1 (ATP2A1) promoter, the troponin I type 1 (TNNI1) promoter, the myosin-1 (MYH1) promoter, the phosphorylatable, fast skeletal muscle myosin light chain (MYLPF) promoter, the alpha-3 chain tropomyosin (TPM3) promoter, the ankyrin repeat domain-containing protein 2 (ANKRD2) promoter the myosin heavy-chain (MHC) promoter, the myosin light-chain (MLC) promoter, the muscle creatine kinase (MCK) promoter, synthetic muscle promoters as described in Li et al. (1999, Nat Biotechnol. 17:241-245), such as the SPc5-12 promoter, the muscle creatine kinase (MCK) promoter, the dMCK promoter and the tMCK promoter consisting of respectively, a double or triple tandem of the MCK enhancer to the MCK basal promoter as described in Wang et al. (2008, Gene Ther, 15:1489-1499).

In particularly preferred embodiments, the promoter is a mammalian promoter, in particular a murine or human promoter.

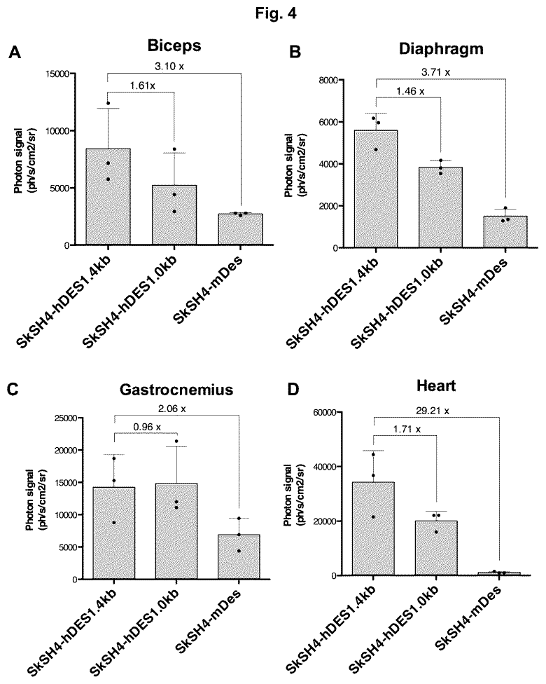

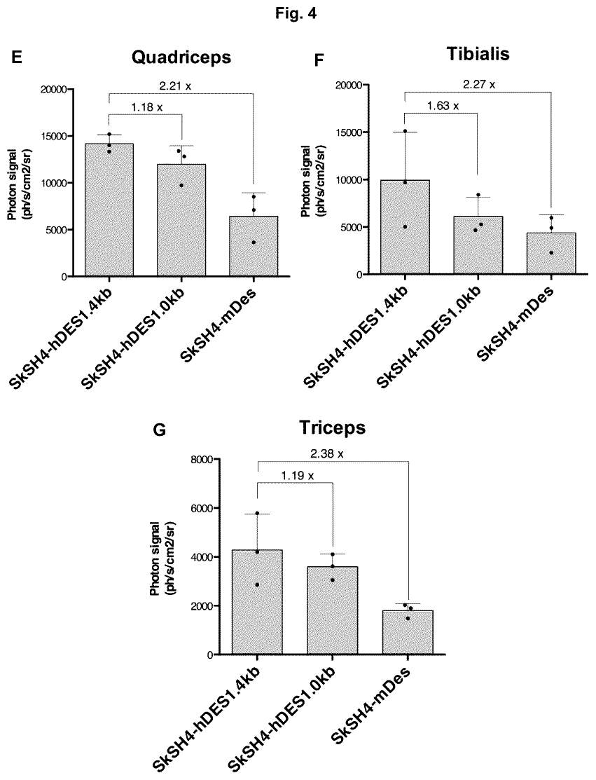

In preferred embodiments, the promoter is from the desmin gene, in particular the murine desmin gene, such as the promoter as defined in SEQ ID NO: 90 (cf. FIG. 4), or the human desmin gene, such as the promoter as defined in SEQ ID NO: 91 (1,0kb) or SEQ ID NO: 92 (1,4kb). In preferred embodiments, the desmin promotor is the 1,4kb promotor of SEQ ID NO: 92 which has been shown to further increase expression levels (cf. examples) For example, the murine desmin promoter is commercially available as pDRIVE-mDesmin (Invivogen). The desmin promoter is expressed in both cardiac muscle and skeletal muscle. In embodiments, the promoter is a skeletal muscle-specific promoter, in particular a muscle creatine kinase (MCK) promoter, more particularly the double MCK promoter or triple MCK promoter consisting of a double or triple tandem of MCK enhancer and MCK basal promoter as described in Wang et al. (2008, Gene Ther, 15:1489-1499). Furthermore, the promoter does not need to be the promoter of the transgene in the nucleic acid expression cassette, although it is possible that the transgene is transcribed from its own promoter.

To minimize the length of the nucleic acid expression cassette, the regulatory elements may be linked to minimal promoters, or shortened versions of the promoters described herein. A ‘minimal promoter’ (also referred to as basal promoter or core promoter) as used herein is part of a full-size promoter still capable of driving expression, but lacking at least part of the sequence that contributes to regulating (e.g. tissue-specific) expression. This definition covers both promoters from which (tissue-specific) regulatory elements have been deleted—that are capable of driving expression of a gene but have lost their ability to express that gene in a tissue-specific fashion and promoters from which (tissue-specific) regulatory elements have been deleted that are capable of driving (possibly decreased) expression of a gene but have not necessarily lost their ability to express that gene in a tissue-specific fashion. Preferably, the promoter contained in the nucleic acid expression cassette disclosed herein is 1000 nucleotides or less in length, 900 nucleotides or less, 800 nucleotides or less, 700 nucleotides or less, 600 nucleotides or less, 500 nucleotides or less, 400 nucleotides or less, 300 nucleotides or less, or 250 nucleotides or less.

The term ‘transgene’ as used herein refers to particular nucleic acid sequences encoding a polypeptide or a portion of a polypeptide to be expressed in a cell into which the nucleic acid sequence is introduced. However, it is also possible that transgenes are expressed as RNA, typically to control (e.g. lower) the amount of a particular polypeptide in a cell into which the nucleic acid sequence is inserted. These RNA molecules include but are not limited to molecules that exert their function through RNA interference (shRNA, RNAi), micro-RNA regulation (miR) (which can be used to control expression of specific genes), catalytic RNA, antisense RNA, RNA aptamers, etc. How the nucleic acid sequence is introduced into a cell is not essential to the invention, it may for instance be through integration in the genome or as an episomal plasmid. Of note, expression of the transgene may be restricted to a subset of the cells into which the nucleic acid sequence is introduced. The term ‘transgene’ is meant to include (1) a nucleic acid sequence that is not naturally found in the cell (i.e., a heterologous nucleic acid sequence); (2) a nucleic acid sequence that is a mutant form of a nucleic acid sequence naturally found in the cell into which it has been introduced; (3) a nucleic acid sequence that serves to add additional copies of the same (i.e., homologous) or a similar nucleic acid sequence naturally occurring in the cell into which it has been introduced; or (4) a silent naturally occurring or homologous nucleic acid sequence whose expression is induced in the cell into which it has been introduced.

The transgene may be homologous or heterologous to the promoter (and/or to the animal, in particular a mammal or human, in which it is introduced, e.g. in cases where the nucleic acid expression cassette is used for gene therapy).

The transgene may be a full-length cDNA or genomic DNA sequence, or any fragment, subunit or mutant thereof that has at least some biological activity. In particular, the transgene may be a minigene, i.e. a gene sequence lacking part, most or all of its intronic sequences. The transgene thus optionally may contain intron sequences. Optionally, the transgene may be a hybrid nucleic acid sequence, i.e., one constructed from homologous and/or heterologous cDNA and/or genomic DNA fragments. By ‘mutant form’ is meant a nucleic acid sequence that contains one or more nucleotides that are different from the wild-type or naturally occurring sequence, i.e., the mutant nucleic acid sequence contains one or more nucleotide substitutions, deletions, and/or insertions. The nucleotide substitution, deletion, and/or insertion can give rise to a gene product (i.e. e., protein or nucleic acid) that is different in its amino acid/nucleic acid sequence from the wild type amino acid/nucleic acid sequence. Preparation of such mutants is well known in the art. In some cases, the transgene may also include a sequence encoding a leader peptide or signal sequence such that the transgene product will be secreted from the cell.

The transgene that may be contained in the nucleic acid expression cassettes described herein typically encodes a gene product such as RNA or a polypeptide (protein).

In embodiments, the transgene encodes a therapeutic protein. The therapeutic protein may be a secretable protein. Non-limiting examples of secretable proteins, in particular secretable therapeutic proteins, include glucosidase, follistatin, clotting factors, such as factor VIII or factor IX, insulin, erythropoietin, lipoprotein lipase, antibodies or nanobodies, growth factors, cytokines, chemokines, plasma factors etc. The therapeutic protein may also be a structural protein. Non-limiting examples of structural proteins, in particular structural therapeutic proteins, include myotubularin, dysferlin, follistatin, microdystrophin 1, dystrophin and sarcoglycans. In preferred embodiments, the transgene comprises the microdystrophin 1 (MD1 & MD2) gene or the follistatin (F57) gene, preferably the exon-skipping construct of follistatin. A non-exhaustive and non-limiting list of transgenes envisaged in the application includes angiogenic factors for therapeutic angiogenesis such as VEGF, PIGF, or guidance molecules such as ephrins, semaphorins, Slits and netrins or their cognate receptors; cytokines and/or growth factors such as erythropoietin (EPO), interferon-α, interferon-β, interferon-γ, interleukin 1 (IL-1), interleukin 2 (IL-2), interleukin 3 (IL-3), interleukin 4 (IL-4), interleukin 5 (IL-5), interleukin 6 (IL-6), interleukin 7 (IL-7), interleukin 8 (IL-8), interleukin 9 (IL-9), interleukin 10 (IL-10), interleukin 11 (IL-11), interleukin 12 (IL-12), chemokine (C-X-C motif) ligand 5 (CXCL5), granulocyte-colony stimulating factor (G-CSF), granulocyte-macrophage colony stimulating factor (GM-CSF), macrophage colony stimulating factor (M-CSF), stem cell factor (SCF), keratinocyte growth factor (KGF), monocyte chemoattractant protein-1 (MCP-1), tumor necrosis factor (TNF), proteins involved in calcium handling such as SERCA (Sarco/Endoplasmic Reticulum Ca2+-ATPase), calcineurin, microdystrophin 1 (MD1), follistatin (F57), alpha-glucosidase (GAA), myotubularin 1 (MTM1), transgenes encoding antibodies, nanobodies, anti-viral dominant-negative proteins, and fragments, subunits or mutants thereof.

In embodiments, the transgene encodes an immunogenic protein. Non-limiting examples of immunogenic proteins include epitopes and antigens derived from a pathogen.

As used herein, the term “immunogenic” refers to a substance or composition capable of eliciting an immune response.

Other sequences may be incorporated in the nucleic acid expression cassette disclosed herein as well, typically to further increase or stabilize the expression of the transgene product (e.g. introns and/or polyadenylation sequences).

Any intron can be utilized in the expression cassettes described herein. The term “intron” encompasses any portion of a whole intron that is large enough to be recognized and spliced by the nuclear splicing apparatus. Typically, short, functional, intron sequences are preferred in order to keep the size of the expression cassette as small as possible which facilitates the construction and manipulation of the expression cassette. In some embodiments, the intron is obtained from a gene that encodes the protein that is encoded by the coding sequence within the expression cassette. The intron can be located 5′ to the coding sequence, 3′ to the coding sequence, or within the coding sequence. An advantage of locating the intron 5′ to the coding sequence is to minimize the chance of the intron interfering with the function of the polyadenylation signal. In embodiments, the nucleic acid expression cassette disclosed herein further comprises an intron. Non-limiting examples of suitable introns are Minute Virus of Mice (MVM) intron, beta-globin intron (betaIVS-II), factor IX (FIX) intron A, Simian virus 40 (SV40) small-t intron, and beta-actin intron.

Preferably, the intron is MVM intron.

Any polyadenylation signal that directs the synthesis of a polyA tail is useful in the expression cassettes described herein, examples of those are well known to one of skill in the art. Exemplary polyadenylation signals include, but are not limited to, polyA sequences derived from the Simian virus 40 (SV40) late gene, the bovine growth hormone (BGH) polyadenylation signal, the minimal rabbit L-globin (mRBG) gene, and the synthetic polyA s(SPA) site as described in Levitt et al. (1989, Genes Dev 3:1019-1025).

Preferably, the polyadenylation signal is derived from SV40 (i.e. SV40 pA).

In particular embodiments, the invention provides a nucleic acid expression cassette comprising, consisting essentially of, or consisting of a nucleic acid regulatory element selected from the group consisting of SEQ ID NO: 1 to 89 or a sequence having 95% identity to said sequence, operably linked to a promoter, preferably a promoter selected from the group consisting of the promoter from the desmin gene or the SPc5-12 promoter, and a transgene, preferably a transgene encoding a luciferase. In further embodiments, the nucleic acid expression cassette further comprises an MVM intron. In yet further embodiments the nucleic acid expression cassette further comprises a polyadenylation signal, preferably a polyadenylation signal derived from synthetic polyA (SynthpA: SEQ ID: 127 & FIGS. 9 A) and B)).

In particular embodiments, the invention provides a nucleic acid expression cassette comprising, consisting essentially of, or consisting of a nucleic acid regulatory element selected from the group consisting of SEQ ID NO: 1 to 89, or a sequence having 95% identity to said sequence, operably linked to a promoter, preferably the promoter from the desmin gene, and a transgene, preferably a transgene encoding microdystrophin, exon-skipping construct or follistatin. In further embodiments, the nucleic acid expression cassette further comprises an MVM intron. In yet further embodiments, the nucleic acid expression cassette further comprises a polyadenylation signal. Alternatively, any one of the following transgenes can introduced: secretable proteins, in particular secretable therapeutic proteins, including glucosidase, follistatin, clotting factors, such as factor VIII or factor IX, insulin, erythropoietin, lipoprotein lipase, antibodies or nanobodies, growth factors, cytokines, chemokines, plasma factors etc.; or structural proteins such as structural therapeutic proteins, including myotubularin, dysferlin, follistatin, microdystrophin 1, dystrophin and sarcoglycans. In embodiments, the transgene comprises the microdystrophin 1 (MD1) gene or the follistatin (F57) gene, preferably the exon-skipping construct of follistatin.

The nucleic acid regulatory element and the nucleic acid expression cassette disclosed herein may be used as such, or typically, they may be part of a nucleic acid vector. Accordingly, a further aspect relates to the use of a nucleic acid regulatory element as described herein or a nucleic acid expression cassette as described herein in a vector, in particular a nucleic acid vector.

In an aspect, the invention also provides a vector comprising a nucleic acid regulatory element as disclosed herein. In further embodiments, the vector comprises a nucleic acid expression cassette as disclosed herein.

The term ‘vector’ as used in the application refers to nucleic acid molecules, e.g. double-stranded DNA, which may have inserted into it another nucleic acid molecule (the insert nucleic acid molecule) such as, but not limited to, a cDNA molecule. The vector is used to transport the insert nucleic acid molecule into a suitable host cell. A vector may contain the necessary elements that permit transcribing the insert nucleic acid molecule, and, optionally, translating the transcript into a polypeptide. The insert nucleic acid molecule may be derived from the host cell, or may be derived from a different cell or organism. Once in the host cell, the vector can replicate independently of, or coincidental with, the host chromosomal DNA, and several copies of the vector and its inserted nucleic acid molecule may be generated. The vectors can be episomal vectors (i.e., that do not integrate into the genome of a host cell), or can be vectors that integrate into the host cell genome. The term ‘vector’ may thus also be defined as a gene delivery vehicle that facilitates gene transfer into a target cell. This definition includes both non-viral and viral vectors. Non-viral vectors include but are not limited to cationic lipids, liposomes, nanoparticles, PEG, PEI, plasmid vectors (e.g. pUC vectors, bluescript vectors (pBS) and pBR322 or derivatives thereof that are devoid of bacterial sequences (minicircles)) transposons-based vectors (e.g. PiggyBac (PB) vectors or Sleeping Beauty (SB) vectors), etc. Viral vectors are derived from viruses and include but are not limited to retroviral, lentiviral, adeno-associated viral, adenoviral, herpes viral, hepatitis viral vectors or the like. Typically, but not necessarily, viral vectors are replication-deficient as they have lost the ability to propagate in a given cell since viral genes essential for replication have been eliminated from the viral vector. However, some viral vectors can also be adapted to replicate specifically in a given cell, such as e.g. a cancer cell, and are typically used to trigger the (cancer) cell-specific (onco)lysis. Virosomes are a non-limiting example of a vector that comprises both viral and non-viral elements, in particular they combine liposomes with an inactivated HIV or influenza virus (Yamada et al., 2003). Another example encompasses viral vectors mixed with cationic lipids.

In preferred embodiments, the vector is a viral vector, such as a retroviral, lentiviral, adenoviral, or adeno-associated viral (AAV) vector, more preferably an AAV vector. AAV vectors are preferably used as self-complementary, double-stranded AAV vectors (scAAV) in order to overcome one of the limiting steps in AAV transduction (i.e. single-stranded to double-stranded AAV conversion) (McCarty, 2001, 2003; Nathwani et al, 2002, 2006, 2011; Wu et al., 2008), and also the use of single-stranded AAV vectors (ssAAV) are also encompassed herein (FIGS. 9 A) and B)).

AAV serotype 9 (AAV9) is ideally suited to achieve efficient transduction in heart and skeletal muscle. Accordingly, in particularly preferred embodiments, the vector is an AAV9 vector.

Production of AAV vector particles can e.g. be achieved by transient co-transfection of AAV-reporter and AAV helper constructs, encoding AAV serotype 9 capsids into HEK293 cells, followed by a purification step based on cesium chloride (CsCl) density gradient ultracentrifugation, as described (Vanden Driessche et al., 2007).

Since the nucleic acid regulatory elements are de facto modular, also combinations of the best diaphragm-specific nucleic acid regulatory elements with any other muscle-specific and/or cardiac specific nucleic acid regulatory elements to maximize expression in the desired target tissue are tested. Consequently, this can lead to the generation of a versatile muscle-specific nucleic acid regulatory element platform tailor-made for diseases that affect skeletal muscle, diaphragm and in some cases also heart (e.g. MTM or GSD II). Furthermore, the diaphragm-specific nucleic acid regulatory elements can also be combined with other promoters or nucleic acid regulatory elements active in other target tissues.

In other embodiments, the vector is a non-viral vector, preferably a plasmid, a minicircle, or a transposon-based vector, such as a Sleeping Beauty(SB)-based vector or piggyBac(PB)-based vector.

In yet other embodiments, the vector comprises viral and non-viral elements.

In particular embodiments, the invention provides a vector comprising a nucleic acid expression cassette comprising a nucleic acid regulatory element comprising, consisting essentially of, or consisting of a nucleic acid regulatory element selected from the group consisting of SEQ ID NO:1 to 89 a promoter, preferably the promoter from the desmin gene, an MVM intron, a transgene, preferably a transgene encoding microdystrophin 1/2 or an exon-skipping construct thereof, and a polyadenylation signal. Alternatively, any one of the following transgenes can introduced: secretable proteins, in particular secretable therapeutic proteins, including glucosidase, follistatin, clotting factors, such as factor VIII or factor IX, insulin, erythropoietin, lipoprotein lipase, antibodies or nanobodies, growth factors, cytokines, chemokines, plasma factors etc.; or structural proteins such as structural therapeutic proteins, including myotubularin, dysferlin, follistatin, microdystrophin 1, dystrophin and sarcoglycans. In embodiments, the transgene comprises the microdystrophin 1 (MD1 or 2) gene or the follistatin (FS7) gene, preferably the exon-skipping construct of follistatin.

In particular embodiments, the invention provides a vector comprising a nucleic acid expression cassette comprising a nucleic acid regulatory element comprising, consisting essentially of, or consisting of a nucleic acid regulatory element selected from the group consisting of SEQ ID NO:1 to 89, a promoter, preferably the promoter from the desmin gene, an MVM intron, a transgene, preferably a transgene encoding follistatin, and a polyadenylation signal. Alternatively, any one of the following transgenes can introduced: secretable proteins, in particular secretable therapeutic proteins, including glucosidase, follistatin, clotting factors, such as factor VIII or factor IX, insulin, erythropoietin, lipoprotein lipase, antibodies or nanobodies, growth factors, cytokines, chemokines, plasma factors etc.; or structural proteins such as structural therapeutic proteins, including myotubularin, dysferlin, follistatin, microdystrophin 1, dystrophin and sarcoglycans. In embodiments, the transgene comprises the microdystrophin 1 (MD1 or 2) gene or the follistatin (FS7) gene, preferably the exon-skipping construct of follistatin.

The nucleic acid expression cassettes and vectors disclosed herein may be used, for example, to express proteins that are normally expressed and utilized in muscle (i.e. structural proteins), or to express proteins that are expressed in muscle and that are then exported to the blood stream for transport to other portions of the body (i.e. secretable proteins). For example, the expression cassettes and vectors disclosed herein may be used to express a therapeutic amount of a gene product (such as a polypeptide, in particular a therapeutic protein, or RNA) for therapeutic purposes, in particular for gene therapy. Typically, the gene product is encoded by the transgene within the expression cassette or vector, although in principle it is also possible to increase expression of an endogenous gene for therapeutic purposes. In an alternative example, the expression cassettes and vectors disclosed herein may be used to express an immunological amount of a gene product (such as a polypeptide, in particular an immunogenic protein, or RNA) for vaccination purposes.

The nucleic acid expression cassettes and vectors as taught herein may be formulated in a pharmaceutical composition with a pharmaceutically acceptable excipient, i.e., one or more pharmaceutically acceptable carrier substances and/or additives, e.g., buffers, carriers, excipients, stabilisers, etc. The pharmaceutical composition may be provided in the form of a kit.

The term “pharmaceutically acceptable” as used herein is consistent with the art and means compatible with the other ingredients of the pharmaceutical composition and not deleterious to the recipient thereof.

Accordingly, a further aspect of the invention relates to a pharmaceutical composition comprising a nucleic acid expression cassette or a vector described herein.

The use of nucleic acid regulatory elements described herein for the manufacture of these pharmaceutical compositions is also disclosed herein.

In embodiments, the pharmaceutical composition may be a vaccine. The vaccine may further comprise one or more adjuvants for enhancing the immune response. Suitable adjuvants include, for example, but without limitation, saponin, mineral gels such as aluminium hydroxide, surface active substances such as lysolecithin, pluronic polyols, polyanions, peptides, oil or hydrocarbon emulsions, bacilli Calmette-Guerin (BCG), Corynebacterium parvum, and the synthetic adjuvant QS-21. Optionally, the vaccine may further comprise one or more immunostimulatory molecules. Non-limiting examples of immunostimulatory molecules include various cytokines, lymphokines and chemokines with immunostimulatory, immunopotentiating, and pro-inflammatory activities, such as interleukins (e.g., IL-1, IL-2, IL-3, IL-4, IL-12, IL-13); growth factors (e.g., granulocyte-macrophage (GM)-colony stimulating factor (CSF)); and other immunostimulatory molecules, such as macrophage inflammatory factor, Flt3 ligand, B7.1; B7.2, etc.

In a further aspect, the invention relates to the nucleic acid regulatory elements, the nucleic acid expression cassettes, the vectors, or the pharmaceutical compositions described herein for use in medicine.

As used herein, the terms “treat” or “treatment” refer to both therapeutic treatment and prophylactic or preventative measures. Beneficial or desired clinical results include, but are not limited to, prevention of an undesired clinical state or disorder, reducing the incidence of a disorder, alleviation of symptoms associated with a disorder, diminishment of extent of a disorder, stabilized (i.e., not worsening) state of a disorder, delay or slowing of progression of a disorder, amelioration or palliation of the state of a disorder, remission (whether partial or total), whether detectable or undetectable, or combinations thereof. “Treatment” can also mean prolonging survival as compared to expected survival if not receiving treatment.

As used herein, the terms “therapeutic treatment” or “therapy” and the like, refer to treatments wherein the object is to bring a subjects body or an element thereof from an undesired physiological change or disorder to a desired state, such as a less severe or unpleasant state (e.g., amelioration or palliation), or back to its normal, healthy state (e.g., restoring the health, the physical integrity and the physical well-being of a subject), to keep it at said undesired physiological change or disorder (e.g., stabilization, or not worsening), or to prevent or slow down progression to a more severe or worse state compared to said undesired physiological change or disorder.

As used herein the terms “prevention”, “preventive treatment” or “prophylactic treatment” and the like encompass preventing the onset of a disease or disorder, including reducing the severity of a disease or disorder or symptoms associated therewith prior to affliction with said disease or disorder. Such prevention or reduction prior to affliction refers to administration of the nucleic acid regulatory elements, the nucleic acid expression cassettes, the vectors, or the pharmaceutical compositions described herein to a patient that is not at the time of administration afflicted with clear symptoms of the disease or disorder. “Preventing” also encompasses preventing the recurrence or relapse-prevention of a disease or disorder for instance after a period of improvement. In embodiments, the nucleic acid regulatory elements according to any one of SEQ ID NO:1 to 7, 10 to 65, 82, and 83 (cf. Dph-CRE1 to 7, 10 to 65, 82, and 83 in Table 3), the nucleic acid expression cassettes, the vectors, or the pharmaceutical compositions described herein may be for use in gene therapy, in particular diaphragm and skeletal muscle-directed gene therapy. Alternatively, the nucleic acid regulatory elements according to any one of SEQ ID No: 1 to 9, 66 to 81, and 84 to 89. (cf. Dph-CRE1 to 9, 66 to 81, and 84 to 89 in Table 4). the nucleic acid expression cassettes, the vectors, or the pharmaceutical compositions described herein may be for use in gene therapy, in particular diaphragm-, skeletal muscle- and heart-directed gene therapy.

Also disclosed herein is the use of the nucleic acid regulatory elements according to any one of SEQ ID NO: 1 to 7, 10 to 65, 82, and 83 (cf. Dph-CRE1 to 7, 10 to 65, 82, and 83 in Table 3), the nucleic acid expression cassettes, the vectors, or the pharmaceutical compositions described herein for the manufacture of a medicament for gene therapy, in particular diaphragm and skeletal muscle-directed gene therapy.

Also disclosed herein is the use of the nucleic acid regulatory elements according to any one of SEQ ID No: 1 to 9, 66 to 81, and 84 to 89. (cf. Dph-CRE1 to 9, 66 to 81, and 84 to 89 in Table 4), the nucleic acid expression cassettes, the vectors, or the pharmaceutical compositions described herein for the manufacture of a medicament for gene therapy, in particular diaphragm-, skeletal muscle- and heart-directed gene therapy.

Also disclosed herein is a method for gene therapy, in particular diaphragm and skeletal muscle-directed gene therapy in a subject in need of said gene therapy comprising:
introducing in the subject, in particular in diaphragm and/or skeletal muscle tissue or cells of the subject, a nucleic acid expression cassette, a vector or a pharmaceutical composition described herein, wherein the nucleic acid expression cassette, the vector or the pharmaceutical composition comprises a nucleic acid regulatory element according to any one of SEQ ID NO: 1 to 7, 10 to 65, 82, and 83 (cf. Dph-CRE1 to 7, 10 to 65, 82, and 83 in Table 3), operably linked to a promoter and a transgene; and
expressing a therapeutically effective amount of the transgene product in the subject, in particular in diaphragm and/or skeletal muscle tissue or cells of the subject.

Also disclosed herein is a method for gene therapy, in particular diaphragm-, skeletal muscle- and heart-directed gene therapy in a subject in need of said gene therapy comprising:
introducing in the subject, in particular in diaphragm-, skeletal muscle- and heart-tissue or cells of the subject a nucleic acid expression cassette, a vector or a pharmaceutical composition described herein, wherein the nucleic acid expression cassette, the vector or the pharmaceutical composition comprises a nucleic acid regulatory element according to any one of SEQ ID No: 1 to 9, 66 to 81, and 84 to 89. (cf. Dph-CRE1 to 9, 66 to 81, and 84 to 89 in Table 4), operably linked to a promoter and a transgene; and
expressing a therapeutically effective amount of the transgene product in the subject, in particular in diaphragm-, skeletal muscle-, and heart-tissue or cells of the subject.

The transgene product may be any one of the following transgenes can introduced: secretable proteins, in particular secretable therapeutic proteins, including glucosidase, follistatin, clotting factors, such as factor VIII or factor IX, insulin, erythropoietin, lipoprotein lipase, antibodies or nanobodies, growth factors, cytokines, chemokines, plasma factors etc.; or structural proteins such as structural therapeutic proteins, including myotubularin, dysferlin, follistatin, microdystrophin 1, dystrophin and sarcoglycans. In embodiments, the transgene comprises the microdystrophin 1 (MD1 or 2) gene or the follistatin (FS7) gene, preferably the exon-skipping construct of follistatin. In particular embodiments, the transgene product is follistatin or microdystrophin, in particular microdystrophin 1 (MTM1). Alternatively, the transgene product may be RNA, such as siRNA.

Exemplary diseases and disorders that may benefit from gene therapy using the nucleic acid regulatory elements, the nucleic acid expression cassettes, the vectors, or the pharmaceutical compositions described herein include myotubular myopathy (MTM), Pompe disease, muscular dystrophy (e.g. Duchenne muscular dystrophy (DMD)/Becker muscular dystrophy (BMD)), myotonic dystrophy, Myotonic Muscular Dystrophy (DM), Miyoshi myopathy, Fukuyama type congenital, muscular dystrophy, dysferlinopathies neuromuscular disease, motor neuron diseases (MND), such as e.g. Charcot-Marie-Tooth disease (CMT), spinal muscular atrophy (SMA), and amyotrophic lateral sclerosis (ALS), Emery-Dreifuss muscular dystrophy, facioscapulohumeral muscular dystrophy (FSHD), congenital muscular dystrophies, congenital myopathies, limb girdle muscular dystrophy, metabolic myopathies, muscle inflammatory diseases, myasthenia, mitochondrial myopathies, anomalies of ionic channels, nuclear envelop diseases, cardiomyopathies, cardiac hypertrophy, heart failure, distal myopathies, cardiovascular diseases, hemophilia, including hemophilia A and B, and diabetes. In addition, many neuromuscular disorders affect respiratory function due to weakening of the diaphragm and respiratory muscles (www.medscape.com/viewarticle/805299_3) Semin Respir Crit Care Med. 2002 June; 23(3):191-200). Causes of diseases of the diaphragm vary, but they can be due to gene defects that influence diaphragm function directly. In particular, there are multiple genetic disorders that are due to mutations in genes that affect the function of the diaphragm, often in combination with abnormalities at the level of skeletal muscles and/or heart. For example, myotubular myopathy (MTM) is due to mutations in the myotubularin gene and affects the skeletal muscle and diaphragm. Patients suffering from MTM typically present with hypotonia, generalized muscle weakness and respiratory failure at birth. Survival beyond the postnatal period requires intensive support, often including gastrostomy feeding and mechanical ventilation. Because of their severe breathing problems, patients suffering from MTM typically do not live past age 2. For MTM, muscle-directed gene therapy is currently the only clinically relevant option. Alternatively, Pompe's disease (also referred to as glycogen storage disorder type II or GSD II) mainly affects skeletal muscle, diaphragm and heart. GSD II results in deficiency of the lysosomal enzyme acid α-glucosidase (GAA) that leads to a lysosomal storage defect. In GSD II patients, glycogen cannot be broken down effectively into glucose. The accumulation of glycogen in GSD II patients causes myopathy with progressive muscle weakness. Without medical intervention, patients suffering from the most severe form of GSD II die because of respiratory failure within the first year of life. Other muscle diseases such as Duchenne muscular dystrophy (DMD) afflicts approximately one in 3500 live male births. The disease leads to a progressive destruction of skeletal muscles, including the diaphragm, the most affected individuals die of ventilatory failure in the third decade of life. Many other myopathies also affect pulmonary function, including—but not limited to—polymyositis/dermatomyositis, hereditary channel disorders, mitochondrial encephalomyopathies, acid maltase deficiency, and congenital myopathy, disuse atrophy. Other diseases affecting diaphragm include Congenital Muscular Dystrophy (CMD), Becker Muscular Dystrophy (BMD), Facioscapulohumeral Muscular Dystrophy (FSHD), Limb Girdle Muscular Dystrophy (LGMD), Myotonic Muscular Dystrophy (DM), Miyoshi myopathy, Fukuyama type congenital muscular dystrophy, dysferlinopathies. Also many neuropathic disorders weaken the diaphragm and respiratory muscles. This includes amyotrophic lateral sclerosis, poliomyelitis, postpolio syndrome, Kennedy syndrome, stroke, multiple sclerosis, spinal muscular atrophy, syringomyelia, neuralgic neuropathy, and motor neuron diseases. Brachial plexitis and isolated unilateral or bilateral phrenic neuropathies can also weaken the diaphragm significantly. Peripheral neuropathies affecting respiration are primarily acute disorders such as Guillain-Barre syndrome, porphyria, and critical illness neuropathy, but chronic diseases such as chronic inflammatory demyelinating polyneuropathy (CIDP) and Charcot-Marie-Tooth disease (CMT) can also cause respiratory insufficiency. Disorders of neuromuscular transmission such as Lambert-Eaton syndrome, and myasthenia gravis often affect respiration. Alternatively, diaphragm dysfunction can be the result of congenital defects resulting in anatomical abnormalities (e.g. Arnold-Chiari malformation) or acquired defects, which occur as the result of an injury, trauma, infection (e.g. West Nile virus, botulism), exposure to, organophosphates, radiation therapy, malnutrition, tumour compression or surgery. Cold cardioplegia used in cardiac surgery is another common cause of phrenic nerve injury. In addition, radiation therapy can affect the phrenic nerve resulting in diaphragmatic dysfunction. Obstructive airway diseases that affect the lungs, such as chronic obstructive pulmonary disease (COPD) and asthma, can result in significant hyperinflation resulting in diaphragmatic disadvantage and weakness. Finally, it is known that lupus and thyroid disorders can also contribute to diaphragm dysfunction.

Gene therapy protocols have been extensively described in the art. These include, but are not limited to, intramuscular injection of plasmid (naked or in liposomes), hydrodynamic gene delivery in various tissues, including muscle, interstitial injection, instillation in airways, application to endothelium, intra-hepatic parenchyme, and intravenous or intra-arterial administration. Various devices have been developed for enhancing the availability of DNA to the target cell. A simple approach is to contact the target cell physically with catheters or implantable materials containing DNA. Another approach is to utilize needle-free, jet injection devices which project a column of liquid directly into the target tissue under high pressure. These delivery paradigms can also be used to deliver vectors. Another approach to targeted gene delivery is the use of molecular conjugates, which consist of protein or synthetic ligands to which a nucleic acid- or DNA-binding agent has been attached for the specific targeting of nucleic acids to cells (Cristiano et al., 1993). In embodiments, the nucleic acid regulatory elements, the nucleic acid expression cassettes, the vectors, or the pharmaceutical compositions described herein may be for use as a vaccine, more particularly for use as a prophylactic vaccine.

Also disclosed herein is the use of the nucleic acid regulatory elements, the nucleic acid expression cassettes, the vectors, or the pharmaceutical compositions described herein for the manufacture of medicament or a vaccine, in particular for the manufacture of a prophylactic vaccine.

Also disclosed herein is a method of vaccination, in particular prophylactic vaccination, of a subject in need of said vaccination comprising:
introducing in the subject, in particular in diaphragm-, and skeletal muscle-tissue or cells of the subject, a nucleic acid expression cassette, a vector or a pharmaceutical composition described herein, wherein the nucleic acid expression cassette, the vector or the pharmaceutical composition comprises a nucleic acid regulatory element according to any one of SEQ ID NO:1 to 7, 10 to 65, 82, and 83 (cf. Dph-CRE1 to 7, 10 to 65, 82, and 83 in Table 3), operably linked to a promoter and a transgene; and
expressing an immunologically effective amount of the transgene product in the subject, in particular in diaphragm- and skeletal muscle-cells or tissue of the subject.

Also disclosed herein is a method of vaccination, in particular prophylactic vaccination, of a subject in need of said vaccination comprising:
introducing in the subject, in particular in diaphragm, skeletal muscle- and heart-tissue or cells of the subject, a nucleic acid expression cassette, a vector or a pharmaceutical composition described herein, wherein the nucleic acid expression cassette, the vector or the pharmaceutical composition comprises a nucleic acid regulatory element according to any one of SEQ ID No: 1 to 9, 66 to 81, and 84 to 89. (cf. Dph-CRE1 to 9, 66 to 81, and 84 to 89 in Table 4), operably linked to a promoter and a transgene; and
expressing an immunologically effective amount of the transgene product in the subject, in particular in diaphragm-, skeletal muscle- and heart-cells or tissue of the subject.

As used herein, a phrase such as “a subject in need of treatment” includes subjects that would benefit from treatment of a recited disease or disorder. Such subjects may include, without limitation, those that have been diagnosed with said disease or disorder, those prone to contract or develop said disease or disorder and/or those in whom said disease or disorder is to be prevented.

The terms “subject” and “patient” are used interchangeably herein and refer to animals, preferably vertebrates, more preferably mammals, and specifically include human patients and non-human mammals. “Mammalian” subjects include, but are not limited to, humans, domestic animals, commercial animals, farm animals, zoo animals, sport animals, pet and experimental animals such as dogs, cats, guinea pigs, rabbits, rats, mice, horses, cattle, cows; primates such as apes, monkeys, orang-utans, and chimpanzees; canids such as dogs and wolves; felids such as cats, lions, and tigers; equids such as horses, donkeys, and zebras; food animals such as cows, pigs, and sheep; ungulates such as deer and giraffes; rodents such as mice, rats, hamsters and guinea pigs; and so on. Preferred patients or subjects are human subjects.

A ‘therapeutic amount’ or ‘therapeutically effective amount’ as used herein refers to the amount of gene product effective to treat a disease or disorder in a subject, i.e., to obtain a desired local or systemic effect. The term thus refers to the quantity of gene product that elicits the biological or medicinal response in a tissue, system, animal, or human that is being sought by a researcher, veterinarian, medical doctor or other clinician. Such amount will typically depend on the gene product and the severity of the disease, but can be decided by the skilled person, possibly through routine experimentation.

An “immunologically effective amount” as used herein refers to the amount of (trans)gene product effective to enhance the immune response of a subject against a subsequent exposure to the immunogen encoded by the (trans)gene. Levels of induced immunity can be determined, e.g. by measuring amounts of neutralizing secretory and/or serum antibodies, e.g., by plaque neutralization, complement fixation, enzyme-linked immunosorbent, or microneutralization assay.

Typically, the amount of (trans)gene product expressed when using an expression cassette or vector as described herein (i.e., with at least one nucleic acid regulatory element) are higher than when an identical expression cassette or vector is used but without a nucleic acid regulatory element therein. More particularly, the expression is at least double as high, at least five times as high, at least ten times as high, at least 20 times as high, at least 30 times as high, at least 40 times as high, at least 50 times as high, or even at least 60 times as high as when compared to the same nucleic acid expression cassette or vector without nucleic acid regulatory element. Preferably, the higher expression remains specific to diaphragm, heart and skeletal muscle tissues or cells. Furthermore, the expression cassettes and vectors described herein direct the expression of a therapeutic amount of the gene product for an extended period. Typically, therapeutic expression is envisaged to last at least 20 days, at least 50 days, at least 100 days, at least 200 days, and in some instances 300 days or more. Expression of the gene product (e.g. polypeptide) can be measured by any art-recognized means, such as by antibody-based assays, e.g. a Western Blot or an ELISA assay, for instance to evaluate whether therapeutic expression of the gene product is achieved. Expression of the gene product may also be measured in a bioassay that detects an enzymatic or biological activity of the gene product.

Also disclosed herein is the use of the nucleic acid regulatory elements according to SEQ ID NO:1 to 7, 10 to 65, 82, and 83 (cf. Dph-CRE1 to 7, 10 to 65, 82, and 83 in Table 3), or the nucleic acid expression cassettes, or the vectors disclosed herein comprising said nucleic acid regulatory elements, for transfecting or transducing diaphragm, and/or skeletal muscle cells.

Also disclosed herein is the use of the nucleic acid regulatory elements according to SEQ ID No: 1 to 9, 66 to 81, and 84 to 89. (cf. Dph-CRE1 to 9, 66 to 81, and 84 to 89 in Table 4), or the nucleic acid expression cassettes, or the vectors disclosed herein comprising said nucleic acid regulatory elements, for transfecting or transducing diaphragm, skeletal muscle and/or heart, cells.

Further disclosed herein is the use of the nucleic acid expression cassettes or the vectors disclosed herein comprising the nucleic acid regulatory elements according to SEQ ID NO:1 to 7, 10 to 65, 82, and 83 (cf. Dph-CRE1 to 7, 10 to 65, 82, and 83 in Table 3), for expressing a transgene product in diaphragm and/or skeletal muscle cells, wherein the nucleic acid expression cassette or the vector comprises a nucleic acid regulatory element disclosed herein operably linked to a promoter and a transgene.

Further disclosed herein is the use of the nucleic acid expression cassettes or the vectors disclosed herein comprising the nucleic acid regulatory elements according to SEQ ID No: 1 to 9, 66 to 81, and 84 to 89. (cf. Dph-CRE1 to 9, 66 to 81, and 84 to 89 in Table 4), for expressing a transgene product in diaphragm, skeletal muscle, and heart cells, wherein the nucleic acid expression cassette or the vector comprises a nucleic acid regulatory element disclosed herein operably linked to a promoter and a transgene.

Further disclosed herein is a method for expressing a transgene product in diaphragm, and/or skeletal muscle cells, comprising:
transfecting or transducing said cells with a nucleic acid expression cassette or a vector disclosed herein, wherein the nucleic acid expression cassette or the vector comprises a nucleic acid regulatory element according to any one of SEQ ID NO:1 to 7, 10 to 65, 82, and 83 (cf. Dph-CRE1 to 7, 10 to 65, 82, and 83 in Table 3), operably linked to a promoter and a transgene; and
expressing the transgene product in said cells.

Further disclosed herein is a method for expressing a transgene product in diaphragm, skeletal muscle and heart cells, comprising:
transfecting or transducing said cells with a nucleic acid expression cassette or a vector disclosed herein, wherein the nucleic acid expression cassette or the vector comprises a nucleic acid regulatory element according to any one of SEQ ID No: 1 to 9, 66 to 81, and 84 to 89. (cf. Dph-CRE1 to 9, 66 to 81, and 84 to 89 in Table 4) operably linked to a promoter and a transgene; and
expressing the transgene product in said cells.

Non-viral transfection or viral vector-mediated transduction of diaphragm, heart, and/or skeletal muscle cells may be performed by in vitro, ex vivo or in vivo procedures. The in vitro approach requires the in vitro transfection or transduction of diaphragm, heart, and/or skeletal muscle cells, e.g. cells previously harvested from a subject, cell lines or cells differentiated from e.g. induced pluripotent stem cells or embryonic cells. The ex vivo approach requires harvesting of the diaphragm, skeletal muscle and heart, cells from a subject, in vitro transfection or transduction, and optionally re-introduction of the transfected cells into the subject. The in vivo approach requires the administration of the nucleic acid expression cassette or the vector disclosed herein into a subject. In preferred embodiments, the transfection of the diaphragm, skeletal muscle, and heart cells is performed in vitro or ex vivo.

It is understood by the skilled person that the use of the nucleic acid regulatory elements, the nucleic acid expression cassettes and vectors disclosed herein has implications beyond gene therapy, e.g. coaxed differentiation of stem cells into diaphragm, skeletal muscle, and heart cells, transgenic models for over-expression of proteins in diaphragm, skeletal muscle, and heart etc.

The invention is further explained by the following non-limiting examples

### EXAMPLES

### Example 1: Identification of Diaphragm-Specific Nucleic Acid Regulatory Elements

To identify the diaphragm genes that are highly expressed, the gene expression profiling of the human diaphragm tissue has been investigated compared to that of other tissues such as skeletal muscle, heart, liver, spleen, and kidneys etc. The total RNA samples extracted from each tissue were purchased commercially (Table 1).

TABLE 1
overview of RNA samples
Diaphragm RNA samples (from Biochain, #R1234169-10)

| | |
|---|---|
| Diaphragm | Male 26 years |
| sample 1 | |
| Diaphragm | Male 24 years |
| sample 2 | |

Other organs RNA samples (from Clontech, #636643)

| | |
|---|---|
| Fetal liver | pooled from 3 males/females, age 20-38 |
| | weeks |
| Bone marrow | pooled from 4 males/female, age 58-76 |
| Brain (whole) | pooled from 4 males, age 21-29 |
| Kidney | pooled from 1 female, age 40 |
| Liver | pooled from 3 males, age 24-64 |
| Lung | pooled from 3 males/females, age 32-61 |
| Skeletal | pooled from 1 male, age 20 |
| muscle | |
| Heart | pooled from 3 males, age 30, 30, 39 |
| Spleen | pooled from 15 males/females, age 22-69 |
| Testis | pooled from 7 males, age 24-87 |
| Colon | pooled from 3 males, age 24-29 |
| Spinal cord | pooled from 7 males, age 20-59 |

Gene expression profiling was performed by RNA next-generation sequencing (RNA-seq). This method provides deep coverage and base pair-level resolution. RNA sequencing quantification is proven to be an efficient alternative to microarray technique in gene expression studies, and it is a critical technology in differential expression analysis.

In this study, RNA-sequencing was achieved using Illumina™ Hiseq 4000 sequencing 50SE (20 clean reads per sample) by BGI (Hong Kong). All samples showed good to excellent quality as high RNA integrity number (RIN) index.

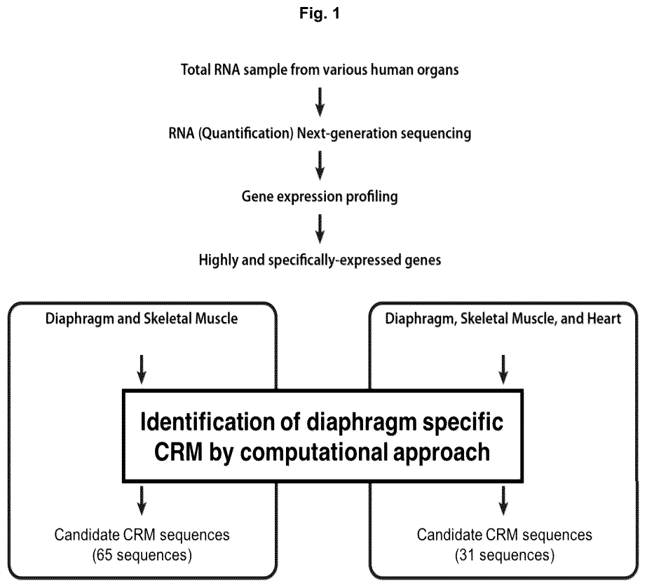

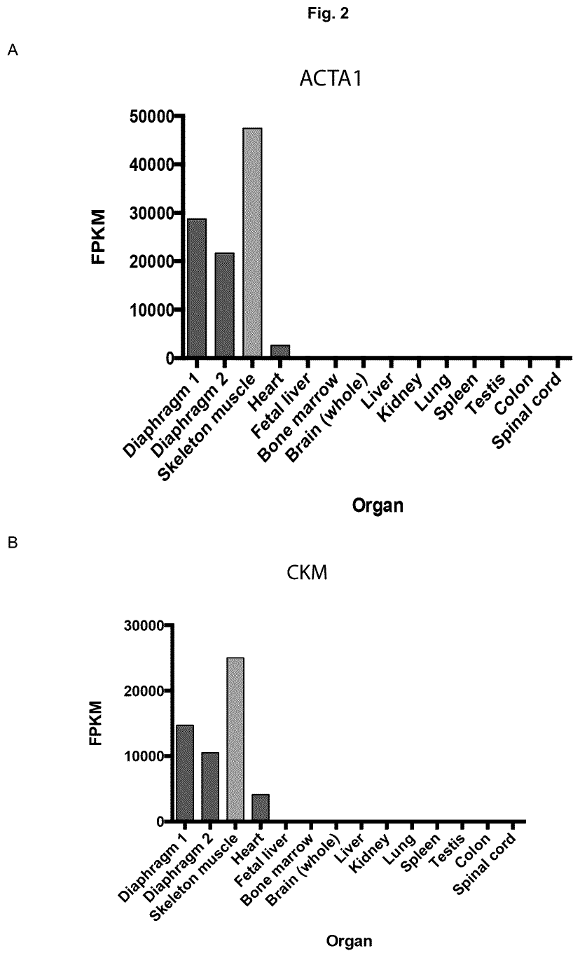

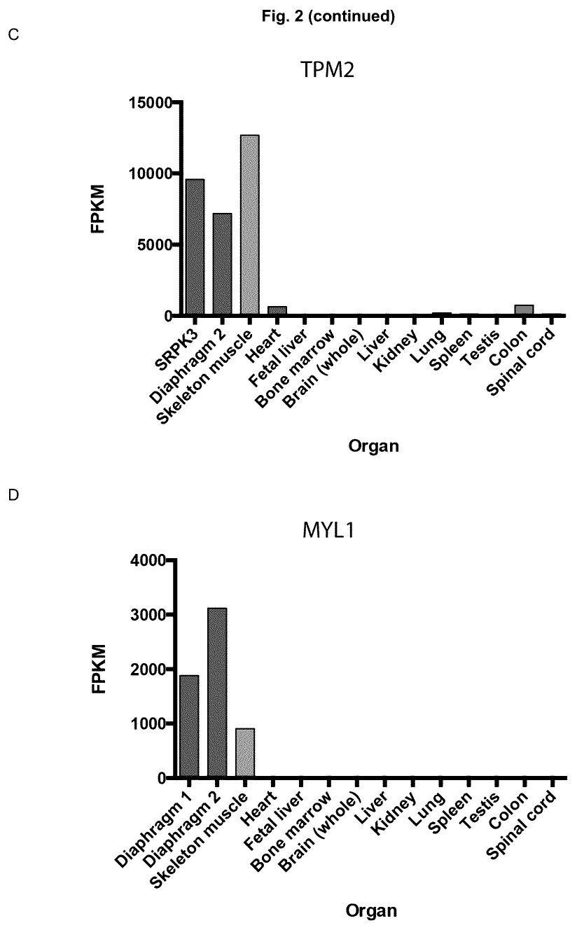

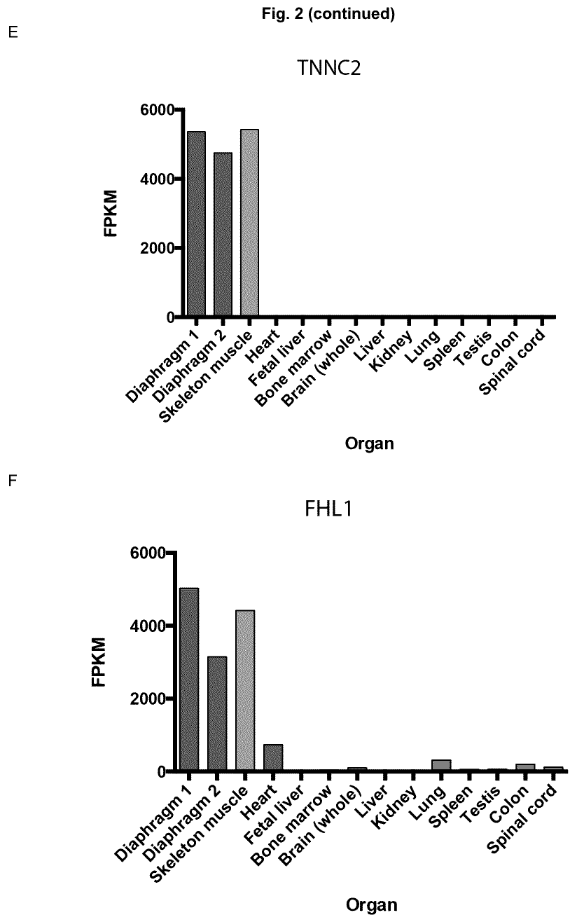

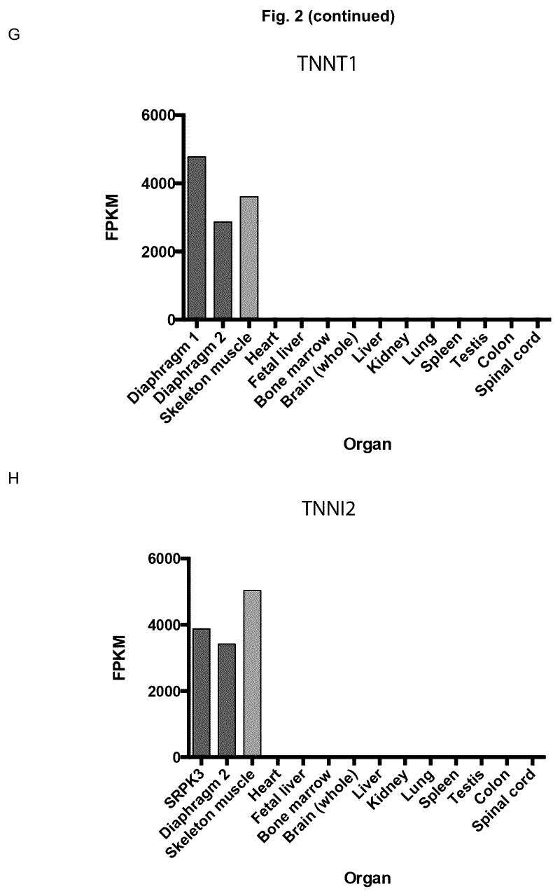

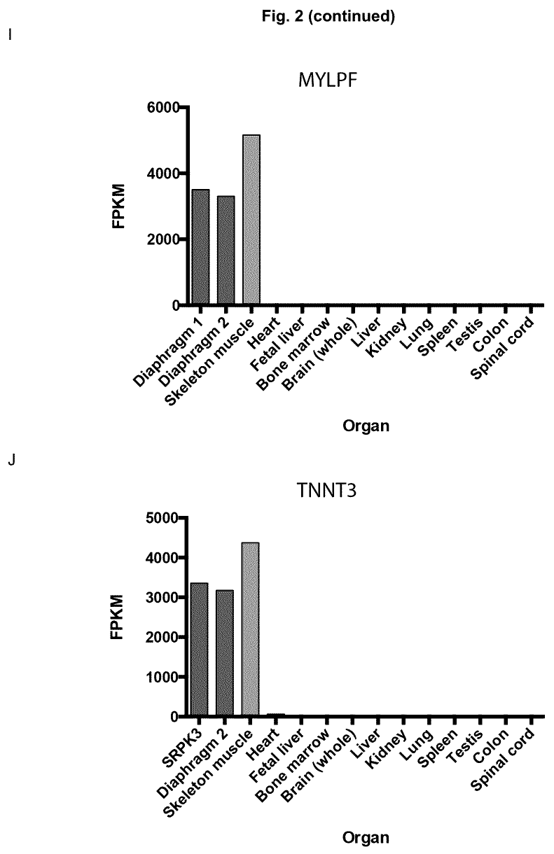

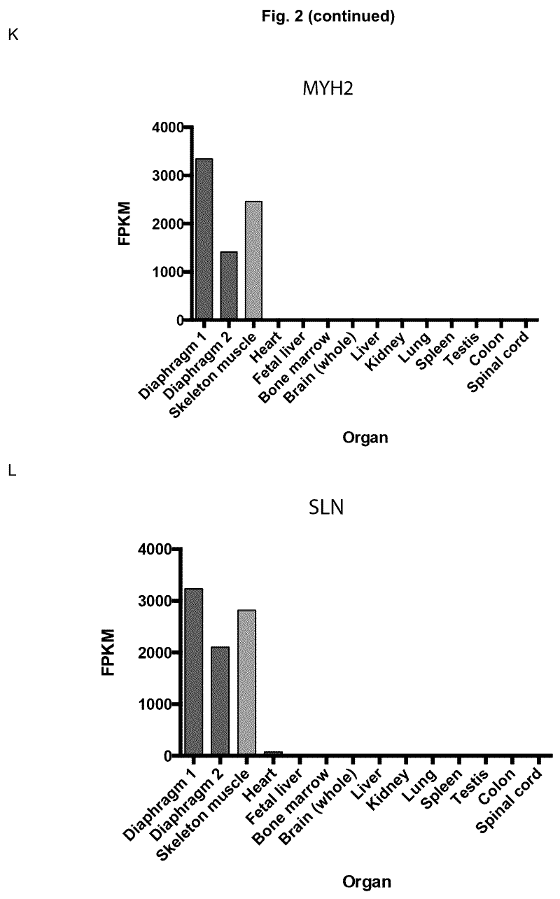

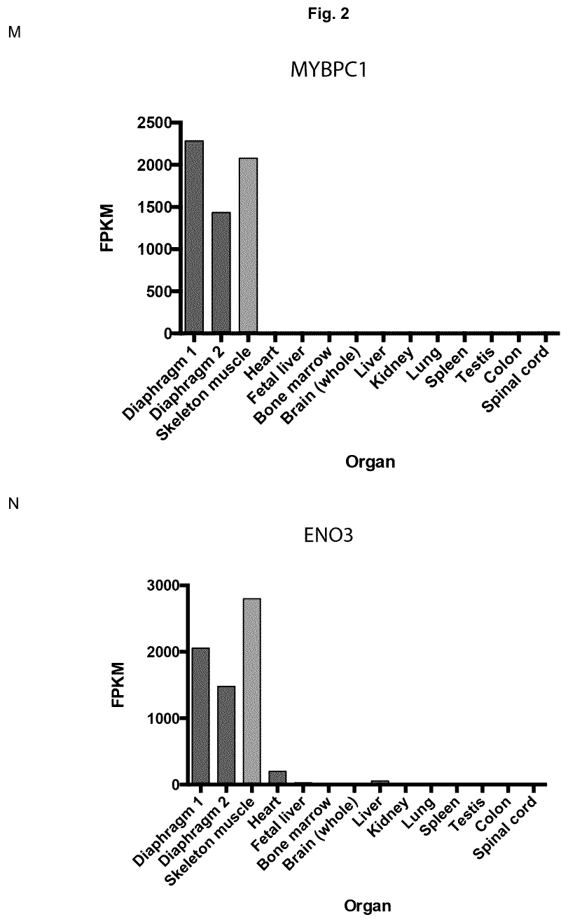

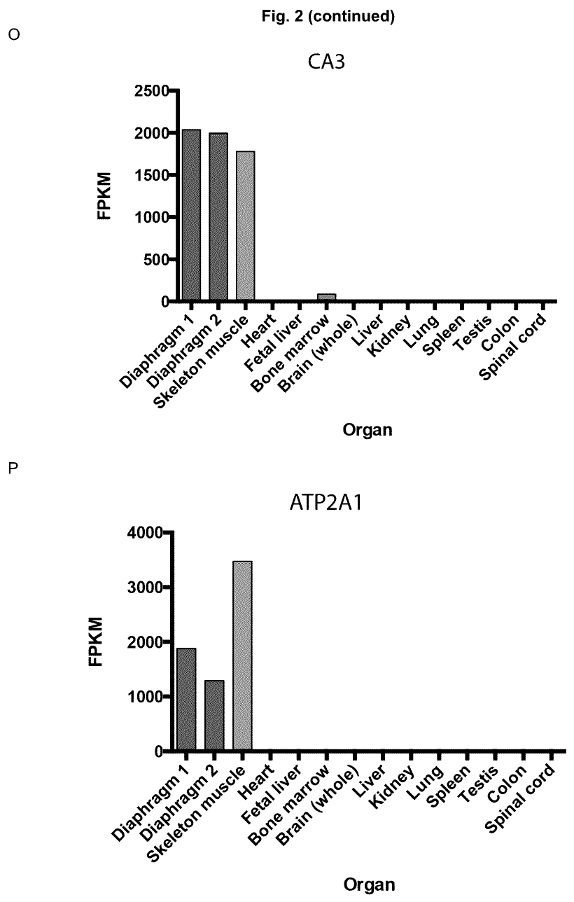

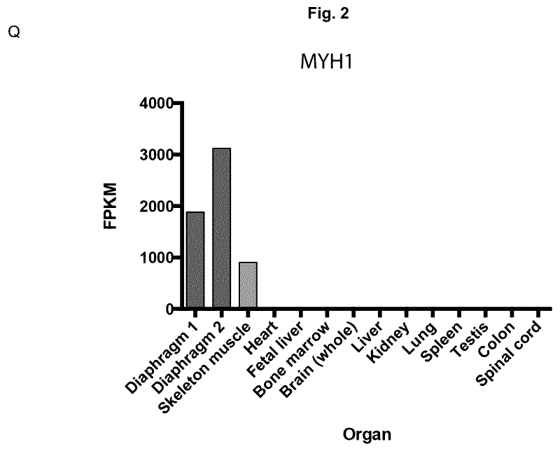

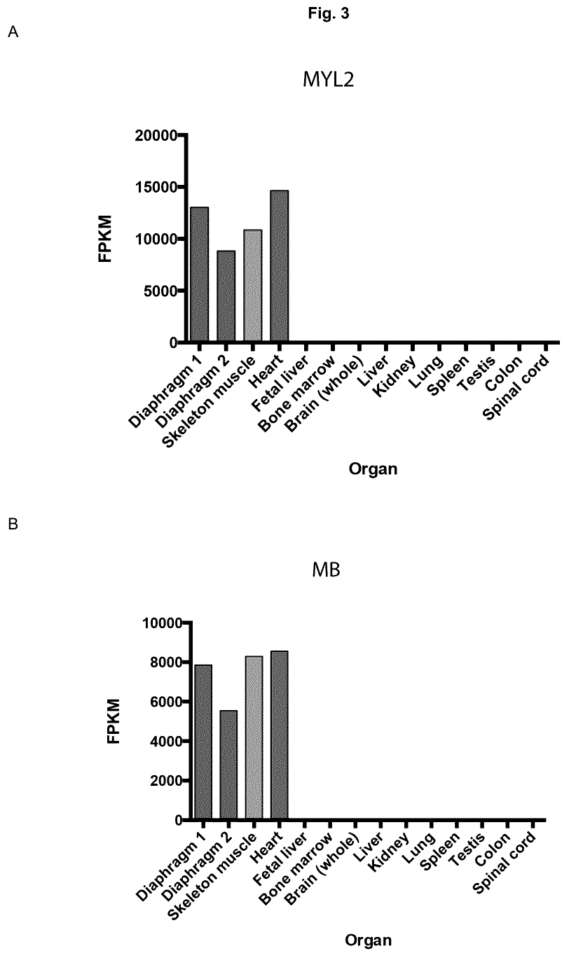

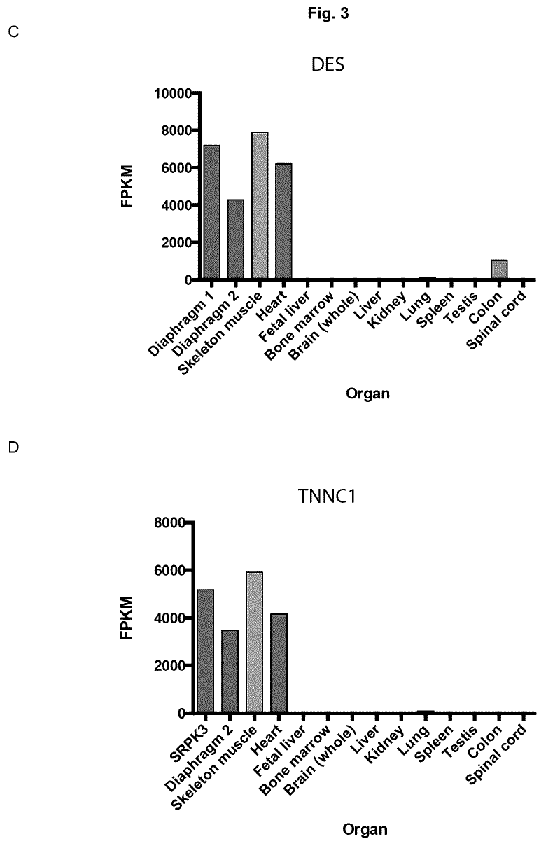

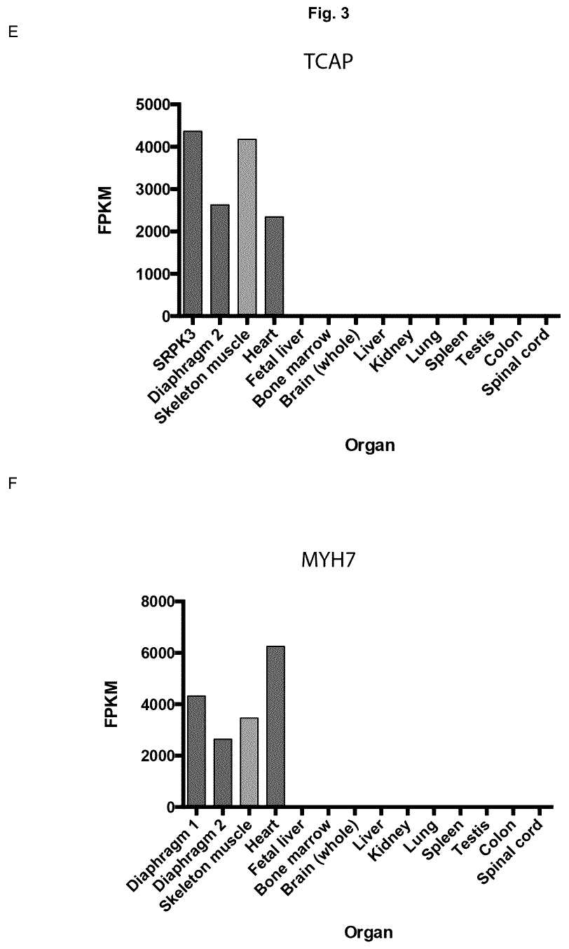

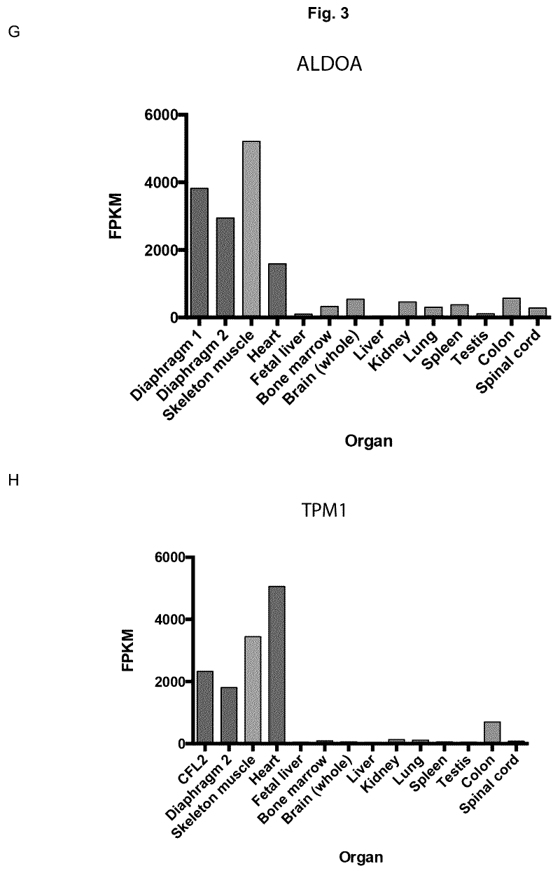

Initially, the gene expression level in diaphragm samples was ranked from highest to lowest values based on the RNA-seq data. The 25 highest expressed genes in the diaphragm were selected. The gene expression profiles were then compared between diaphragm, skeletal muscle, with or without heart versus other tissues to identify only highly and specifically expressed genes in i) diaphragm and skeletal muscle (Dph+SkM) or ii) diaphragm, skeletal muscle, and heart (Dph+SkM+Hrt). Based on this comparison, 18 genes were identified (Table 3), which are highly and specifically expressed in diaphragm and skeletal muscle and 8 genes (Table 4) that are highly expressed in diaphragm, skeletal muscle, and heart. All 25 genes (17 genes for Dph+SkM and 10 genes for Dph+SkM+Hrt: 2 genes ACTA1 and CKM are overlapping with the Dph+SkM group) were analyzed using ENSEMBL for locating the transcription start site (TSS). Subsequently, these TSS were mapped into UCSC Genome Browser Database for nucleic acid regulatory element identification. The nucleic acid regulatory elements were selected based on i) high DNase hypersentitivity sites ii) high content of epigenetic markers associated with open chromatin (i.e. acetylation, methylation) iii) high content of transcription factor binding sites iv) strong evolutionary conservation among vertebrates and v) conserved transcription factor binding sites in 3 species (human, rat, mouse) (FIG. 1). Finally, based on these 5 criteria, 89 nucleic acid regulatory elements were identified from 25 genes. 65 nucleic acid regulatory elements sequenced were identified for high expression in diaphragm and skeletal muscle (Table 3) and 31 nucleic acid regulatory elements sequenced were identified for high expression in diaphragm, skeletal muscle and heart (Table 4). 7 of these are common CREs and are therefore present in both tables.

TABLE 2
list of genes highly expressed in Diaphragm
and Skeletal Muscle or Diaphragm, Skeletal
Muscle and Heart (cf. also Table 3 & 4)

| High expressed genes | High expressed genes in |
|---|---|
| in Diaphragm and | Diaphragm and Skeletal |
| Skeletal Muscle | Muscle and Heart |
| 1) ACTA1 | 1) MYL2 |
| 2) CKM | 2) MB |
| 3) TPM2 | 3) DES |
| 4) MYL1 | 4) TNNC1 |
| 5) TNNC2 | 5) TCAP |
| 6) FHL1 | 6) MYH7 |
| 7) TNNT1 | 7) ALDOA |
| 8) TNNI2 | 8) TPM1 |
| 9) MYLPF | 9) ACTA1 |
| 10) TNNT3 | 10) CKM |
| 11) MYH2 | |
| 12) SLN | |
| 13) MYBPC1 | |
| 14) CA3 | |
| 15) ATP2A1 | |
| 16) MYH1 | |
| 17) ENO3 | |

### Example 2: In Vivo Comparison of Desmin Promoters

### Experimental Procedures

AAV vectors comprising the muscle-specific regulatory element Sk-SH4 were generated according to the protocol described in Example 3. Briefly, the muscle-specific regulatory element Sk-SH4 was synthesized by conventional oligonucleotide synthesis and cloned upstream of the human desmin 1.4kb promoter (SEQ ID NO: 92), the human desmin 1.0kb promoter (SEQ ID NO: 91) or the murine desmin promoter (SEQ ID NO: 90) in the context of the AAV vector backbone of AAVsc-hDes1.4kb-MVM-Luc, AAVsc-hDes1.0kb-MVM-Luc or AAVsc-mDes-MVM-Luc, respectively.

Adult CB17/IcrTac/PrkdcSCID (SCID, severe combined immunodeficient) mice were intravenously injected (n=5) at a dose of 1×10¹⁰ vg/mouse.

Mice were sacrified at two weeks and four weeks after injection of the vectors and the different muscle types (biceps, diaphragm, gastrocnemius, heart, quadriceps, tibialis and triceps) were isolated and quantified using bioluminescense imaging as described in Keyaerts M1, Caveliers V, Lahoutte T. Trends Mol Med. 2012 March; 18(3):164-72. doi: 10.1016/j.molmed.2012.01.005. Epub 2012 Feb. 8. Bioluminescence imaging: looking beyond the light.

### Results

Comparison of the luciferase expression induced by the different AAV vectors quantified as Photons signal shows that the expression cassette comprising Sk-SH4-hDES1.0kb or Sk-SH4-hDES1.4kb, especially Sk-SH4-hDES1.4kb, leads to a higher luciferase expression in the different muscle types of mice (Biceps, diaphragm, gastrocnemius, heart, quadriceps, tibialis and triceps) compared to the expression cassette comprising Sk-SH4-mDES (FIG. 4).

### Example 3: In Vivo Validation of Dph-CREs

The selected top diaphragm-specific nucleic acid regulatory elements (cf. Table 3 & 4) were cloned upstream of a promoter which is active in muscle, diaphragm and optionally heart tissue (e.g. mouse or human desmin promoter (DES, more preferably mDES, hDES1.0kb, or hDES1,4kb as depicted in SEQ ID NO: 90-92 respectively), or the SPc5-12 promoter (SEQ ID NO: 124), to drive expression of a reporter gene in an AAV vector backbone (e.g. pAAV_{SC} or pAAV_{SS}). Other promotors of genes highly expressed in diaphragm can also be used. In addition, other muscle specific promoters such as muscle creatine kinase promoter (MCK) (Wang B et al., 2008) alpha-myosin heavy chain (a-MHC), myosin light chain (MLC-2) or cardiac troponin C (cTnC), myogenin MYF4 promoters ((Pacak C. A. et al; 2008) or viral promoters such as murine stem cell virus (MSCV) promoter (Suga T et al., Plos One 2011) are all potential promoters that can be used to cloned downstream of the diaphragm nucleic acid regulatory elements.

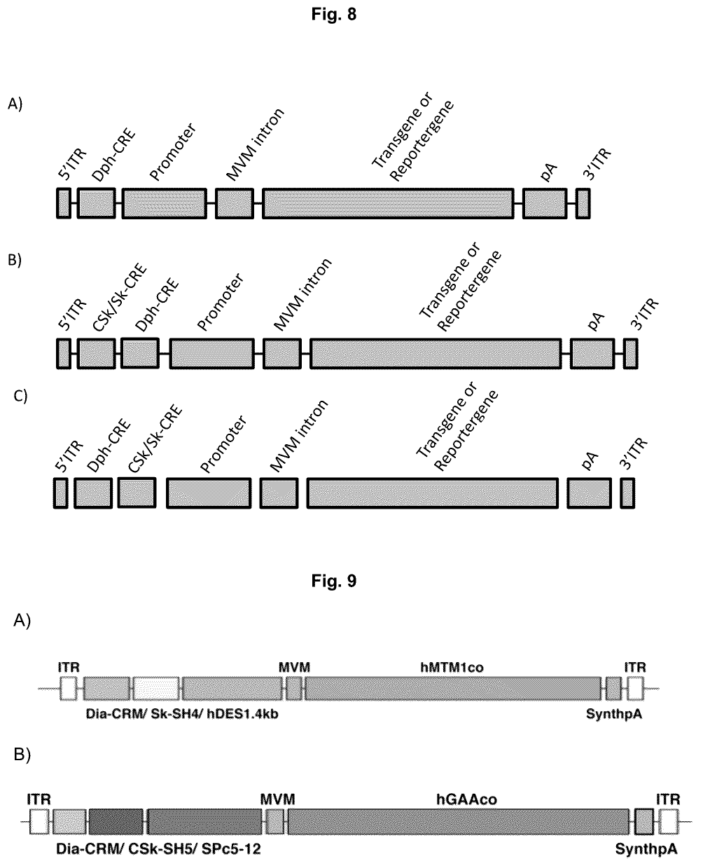

The plasmids also contain a Minute Virus of Mouse (MVM) intron (SEQ ID NO: 125) and a polyadenylation site (pA), e.g. the Simian Virus 40 polyadenylation site (SEQ ID NO: 126) or a synthetic poly-A site (SEQ ID NO: 127). This leads to the general AAV backbone pAAV-Dph-CRE01to89-Des/SPc-promoter-MVM-intron-Transgene/Reportergene-pA (FIG. 8 A)).

Some of these backbones were further complemented with a muscle/heart-CRE such as the ones disclosed in WO2015/110449. This leads to the AAV backbone pAAV-CSk/Sk-CRE-Dph-CRE01to89-Des/SPc-promoter-MVM-intron-Transgene/Reportergene-pA (FIG. 8 B)) or pAAV-Dph-CRE01to89-Sk/CSk-CRE-Des/SPc-promoter-MVM-intron-Transgene/Reportergene-pA (FIG. 8 C)), wherein the Sk-CRE/CSk-CRE is either one of the CRE's disclosed in WO2015/110449, preferably skeletal muscle CRE Sk-SH4 (SEQ ID NO: 121), or the skeletal muscle and cardiac CREs CSk-SH5 (SEQ ID NO: 122) and CSk-SH1 (SEQ ID NO: 123) regulatory elements.

The reporter gene is preferably a Luciferase reporter gene and the transgenes tested below are the human GAA and MTM1 genes or their codon-optimised variants (cf. example 4).

For studying the effect of the Dph-CREs on tissue specific expression, the following AAV vector backbones were used:
for the Dph-CREs from Table 3: pAAVss-Sk-SH4-hDES1.4kb-MVM-Luciferase-pA
for the Dph-CREs from Table 4: pAAVss-CSk-SH5-SPc5-12GTRM-MVM-Luciferase-pA, Wherein the different Dph-CREs are cloned before or after the Sk-SH4 or CSk-SH5 CRE.

Production of the AAV vector particles is achieved by transient co-transfection of AAV-reporter and AAV helper constructs, encoding AAV serotype 9 capsids into HEK293 cells, followed by a purification step based on cesium chloride (CsCl) density gradient ultracentrifugation, as described previously (Vanden Driessche et al., 2007 J Thromb Haemost 5:16-24), which is specifically incorporated by reference herein.

Briefly, two days post transfection, cells were harvested and vector particles were purified using isopycnic centrifugation methods. Harvested cells were lysed by successive freeze/thaw cycles and sonication, treated with benzonase (Novagen, Madison, WI) and deoxycholic acid (Sigma-Aldrich, St. Louis, MO) and subsequently subjected to 3 successive rounds of cesium chloride (Invitrogen Corp, Carlsbad, CA) density gradient ultracentrifugation. Fractions containing the AAV vector were collected, concentrated in 1 mM MgCl₂ in Dulbecco's phosphate buffered saline (PBS) (Gibco, BRL) and stored at −80° C.

Vector titers (in viral genomes (vg)/ml) were determined by quantitative real-time PCR using SYBR Green mix (which included SYBR Green dye, Taqman polymerase, ROX and dNTP's all in one) and luciferase specific primers on an ABI 7500 Real-Time PCR System (Applied Biosystem, Foster city, CA, USA). The forward and reverse primers used were 5′-CCCACCGTCGTATTCGTGAG-3′ (SEQ ID NO: 128) and 5′-TCAGGGCGATGGTTTTGTCCC-3′ (SEQ ID NO: 129), respectively.

Typically, for all vectors titers in the range of 1.5-6.1×10¹¹ vg/ml were achieved from a small production batch of 20 petri dishes of producer cells. If higher number of petri dishes such as 60 dishes of producer cells were used, a higher titer typically in the range of 10¹²-10¹³ gc/ml of AAV particles were achieved. Known copy numbers (10²-10⁷) of the respective vector plasmids used to generate the corresponding AAV vectors, carrying the appropriate cDNAs were used to generate the standard curves.

All animal procedures were approved by the institutional animal ethics committee of the Free University of Brussels (VUB) (Brussels, Belgium). All mice were housed under specific pathogen-free conditions; food and water were provided ad libitum.

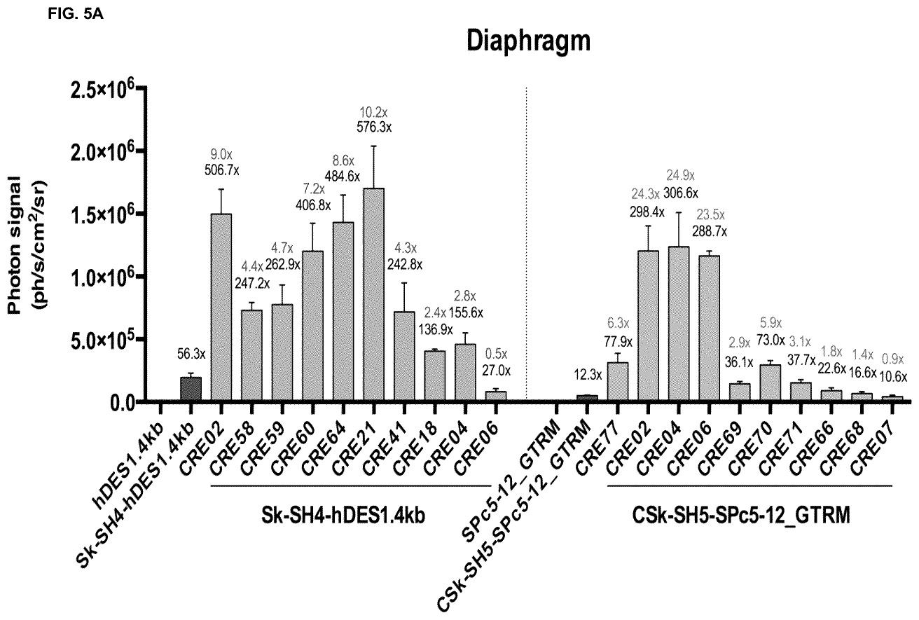

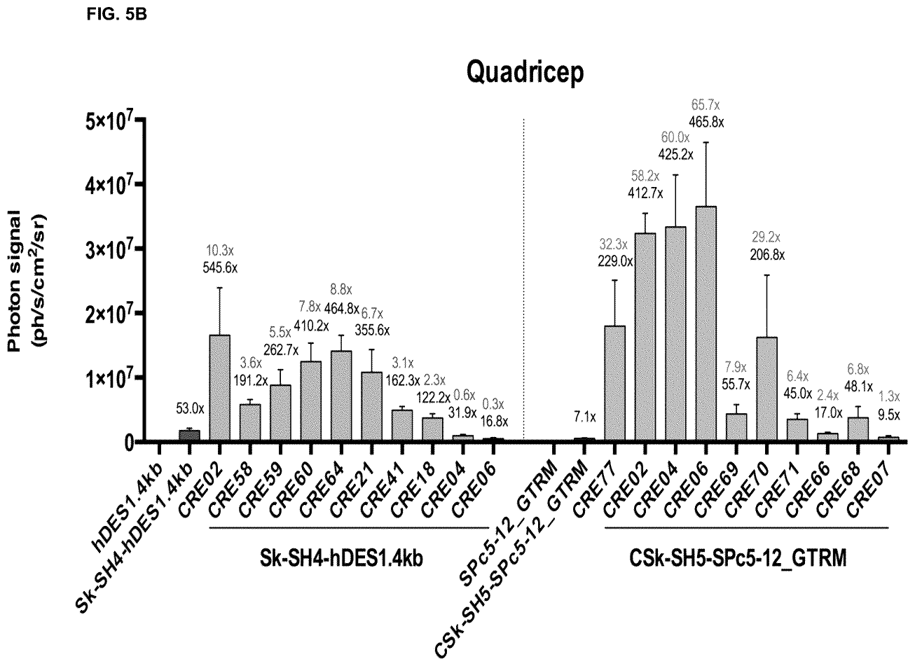

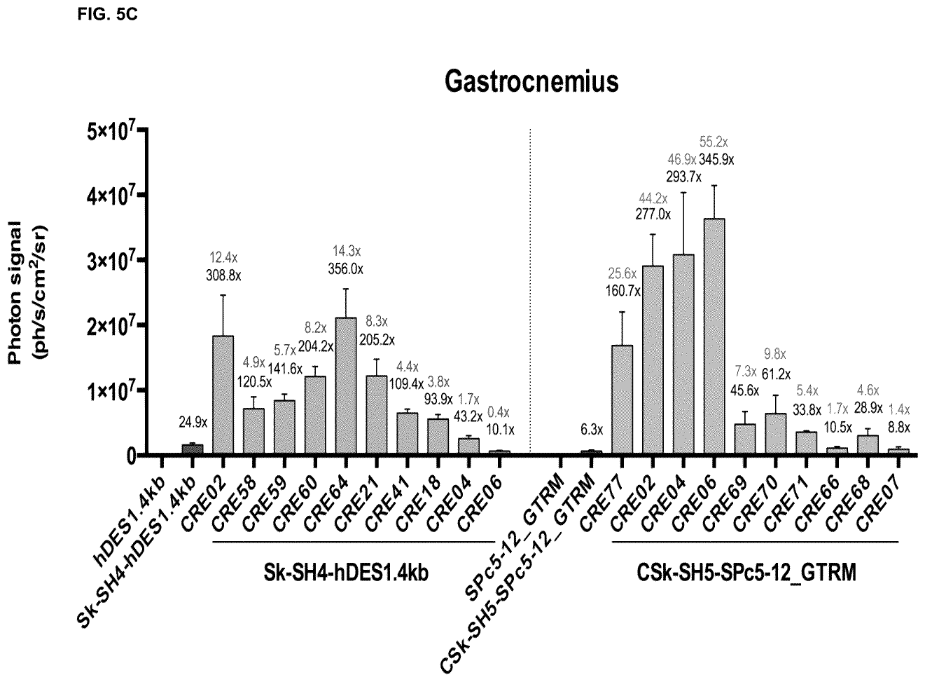

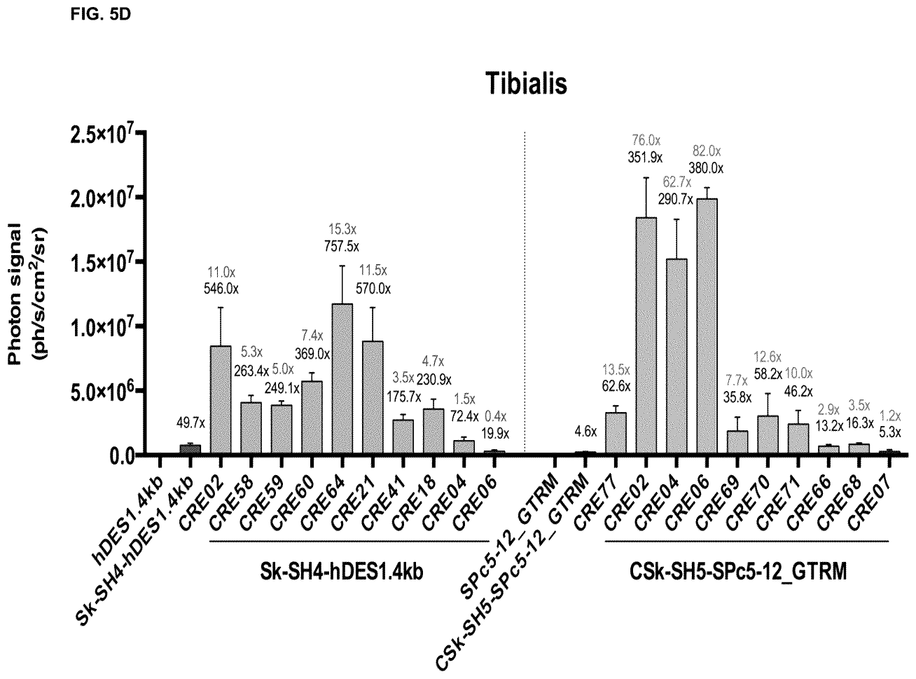

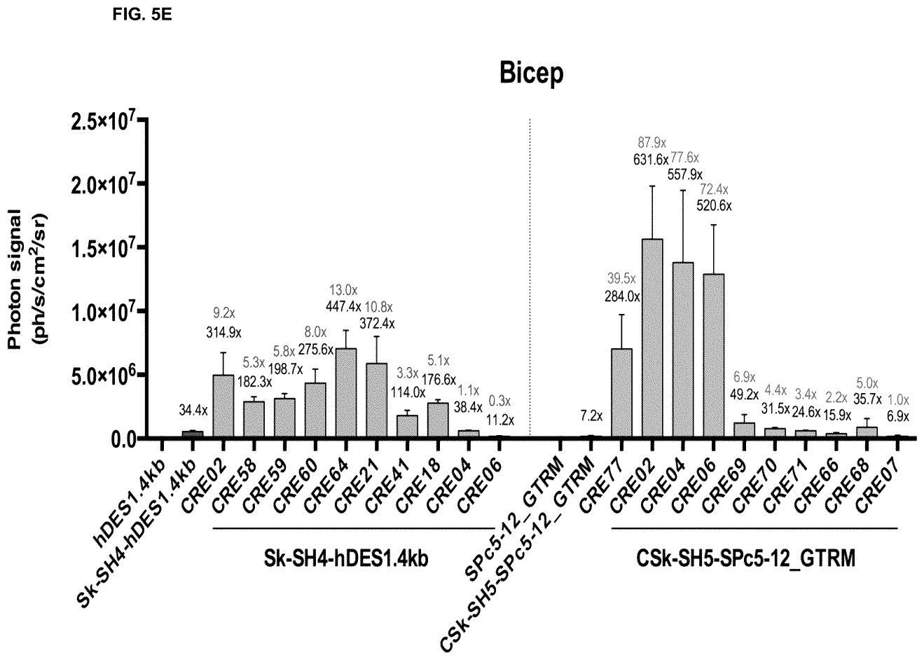

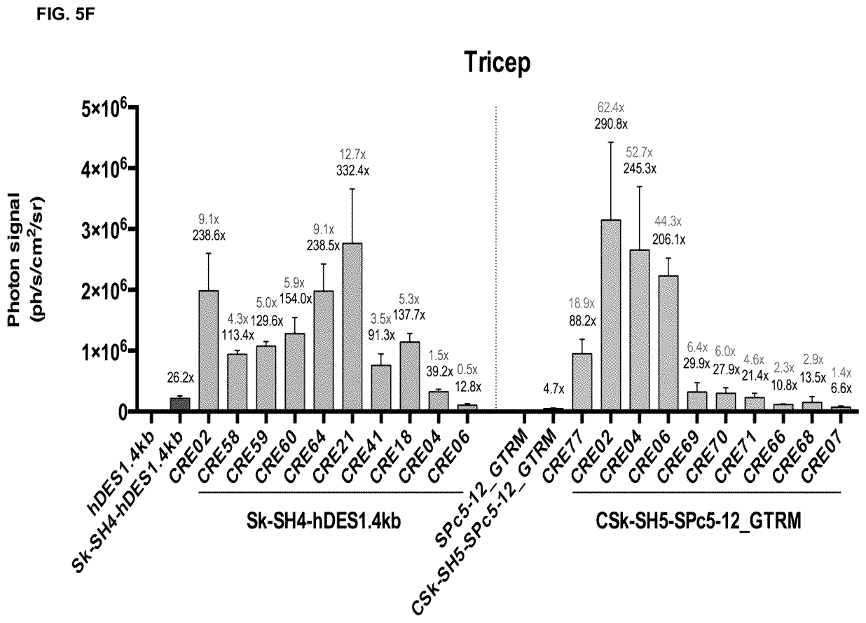

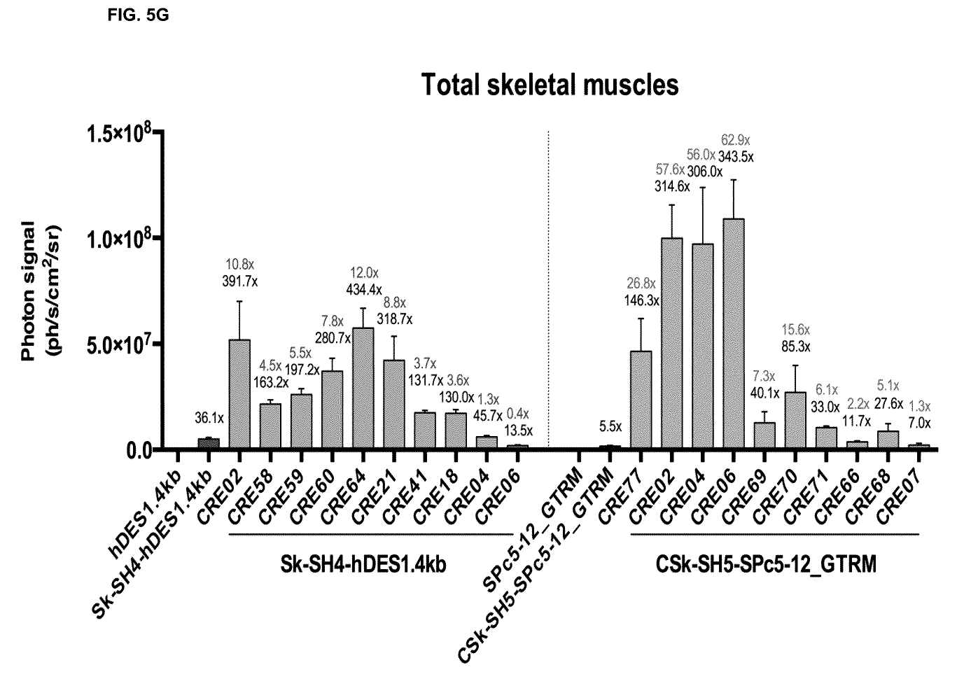

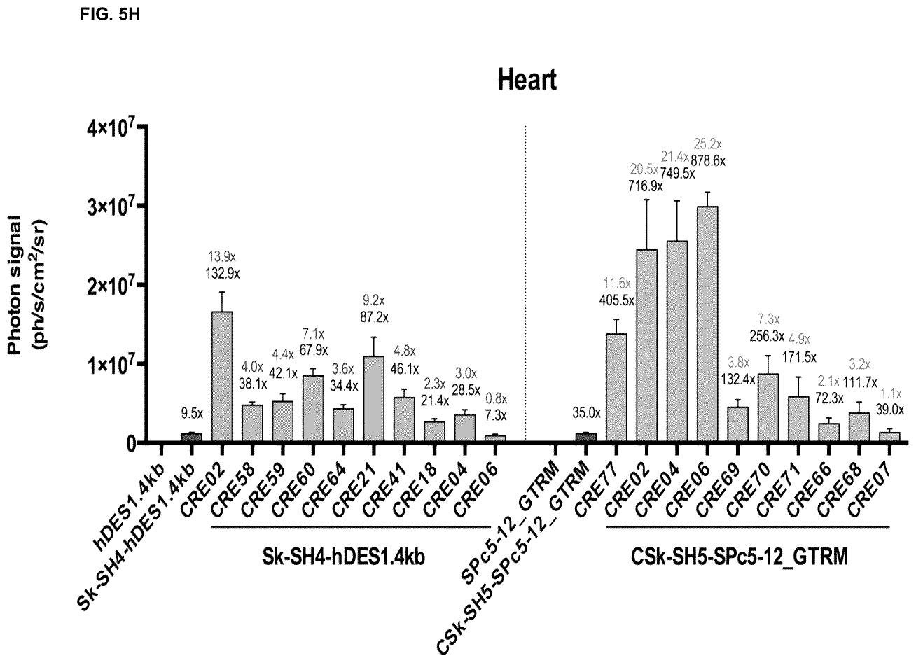

The purified AAV vectors are injected intravenously (i.v.) in 4 weeks old immunodeficient CB17-SCID mice (Janvier, France) at different vector doses, allowing the identification of the most robust diaphragm-specific nucleic acid regulatory elements by quantifying the reporter gene expression. In different muscle tissues (biceps, diaphragm, gastrocnemius, heart, quadriceps, tibialis and triceps) were quantified using bioluminescence imaging. Whole body bioluminescence was performed approx.1 week post-AAV injection and organs bioluminescence approx. 3 weeks post-AAV injection. Luciferase expression induced by the different AAV vectors quantified as Photons signal are shown in FIGS. 5A to 5I for Dph-CRE-02, -58, -59, -60, -64, -21, -41, -18, -04, -06 for the Sk-SH4 backbone) and Dph-CRE-77, -02, -04, -06, -69, -70, -71, -66, -68, and -07 for the CSk-SH5 backbone). All 20 tested Dph-CREs show an increased expression versus the base line expression using a Sk-SH4-hDES1,4kb or CSk-SH5-SPc5-12 backbone. 100% of the diaphragm CREs tested showed enhanced luciferase expression. In particular, Dph-CRE-02, 21 and 64 show a particularly higher expression levels compared to the Sk-SH4-hDES1,4kb backbone, while Dph-CRE-04, -02 and -06 show a particularly higher expression level compared to the CSk-SH5-SPc5-12 kb backbone.

The individual screening of the 20 diaphragm CREs clearly demonstrated the successful generation of synergistic CRE combinations (Diaphragm CRE+Skeletal muscle CREs) and (Diaphragm CRE+Skeletal muscle/Heart CREs) that led to robust gene expression in the diaphragm and skeletal muscle and/or heart, higher than with any conventional vector designs that are either currently being used in clinical trials or being considered for future trials.

The experiments show that the skeletal muscle specific-CRE (Sk-SH4) leads to a robust increase in reporter (or transgene) expression in vivo when compared to control without muscle specific CRE (hDES1.4kb). In particular, the diaphragm specific CRE (Dph-CRE64, CRE02, and CRE21) coupled to the muscle specific CRE (Sk-SH4) act synergistically as a combined module, leading to an extremely high level of enhancement of luciferase expression in vivo. The other Dph CREs (60, 58, 04, 18, 41, 69, 06) also showed moderate augmentation of luciferase gene expression.

Similarly, the combined cardiac/skeletal muscle specific-CRE (CSk-SH5) led to robust increased in luciferase expression in vivo when compared to control without CSk-SH5 CRE but just the SPc5-12-GTRM promotor driving the luciferase gene. In addition, we showed that the diaphragm specific CRE (Dph-CRE04, 02, 06) when coupled to the combined cardiac/skeletal muscle specific CRE (CSk-SH5) act synergistically, leading to an extremely high level of enhancement of luciferase expression in vivo. Other Dph CREs (77, 70, 66, 71, 69, 68, 07) showed moderate level of augmentation of luciferase gene expression.

Dph-CRE64 showed the highest increase in luciferase gene expressed when combined to the Sk-SH4 muscle CRE driven from the human Desmin 1.4 kb promoter. There is a clear synergistic effect of the Diaphragm CRE in combination with the muscle CRE.

Dph-CRE04 showed the highest increase in luciferase gene expressed when combined to the CSk-SH5 muscle CRE driven from the synthetic SPc5-12 promoter. There is a clear synergistic effect of the Diaphragm CRE in combination with the muscle CRE.

This demonstrates that the CREs can act synergistically in a modular fashion.

The individual organ data confirmed the data of the whole body. In the diaphragm tissues, for the group of diaphragm CREs combined with the Sk-SH4-hDes1.4kb expression cassette, the diaphragm CREs that are the most robust are: CRE02, CRE64 CRE60 and CRE21 leading to an augmentation of 400-600 fold luciferase expression when compared to luciferase expression driven from just the human Desmin 1.4kb promoter (normalized to hDes1.4kb, fold difference depicted in lower numbers above graphs in FIG. 5A left-hand). This 400-600 fold increased in luciferase expression is the contribution of the diaphragm CRE combined with the muscle CRE (Sk-SH4). When normalized to Sk-SH4-hDes1.4kb, up to 10 fold enhancement of luciferase expression can be detected from these 4 robust diaphragm CREs (fold difference upper numbers above graphs in FIG. 5A left-hand). This 10 fold difference is the contribution of the individual diaphragm CRE driven from this expression cassette.

Similarly, for the group of diaphragm CREs combined with the CSk-SH5-SPc5-12-GTRM expression cassette, the diaphragm CREs that are the most robust are: CRE02, CRE04, CRE06) leading to an augmentation of about 300 fold luciferase expression when compared to luciferase expression driven from just the SPc5-12-GTRM promoter (normalized to SPc5-12-GTRM, fold difference depicted in lower numbers above graphs in FIG. 5A right-hand). This 300 fold increased in luciferase expression is the contribution of the diaphragm CRE combined with the muscle/heart CRE (CSk-SH5). When normalized to CSk-SH5-SPc5-12-GTRM, up to 23-25 fold enhancement of luciferase expression can be detected from these 3 robust diaphragm CREs (fold difference upper numbers above graphs in FIG. 5A right-hand). This 23-25 fold enhancement is the sole contribution from the individual diaphragm CRE.

Similar comparisons are made for other muscle tissues: FIG. 5B Expression in Quadricep FIG. 5C Expression in Gastrocnemius FIG. 5D Expression in Tibialis FIG. 5E Expression in Bicep FIG. 5F Expression in Tricep FIG. 5G, Expression in heart FIG. 5H, Specificity of Diaphragm CREs (mainly expressed in diaphragm, skeletal muscle and heart but not in other tissues. The numbers of the Diaphragm CREs correspond to the numbering of Tables 3 & 4 FIG. 5I).

### Example 4: Codon-Optimisation of Therapeutic Genes GAA and MTM1

Experimental Procedures

### Generation of the AA V Plasmid Constructs

The human alpha-glucosidase (hGAA) gene and the human myotubularin 1 (hMTM1) gene were codon-optimized using the Gene optimizer (GeneArt, Life technologies, Germany).

The wild-type human alpha-glucosidase (hGAA; SEQ ID NO: 93) and codon-optimized hGAA genes (hGAAco; SEQ ID NO: 94) flanked by BsiWI and XmaJI restriction sites at the 5′ and 3′ ends, were cloned and driven from the SPc5-12 promoter, which was operably linked to the regulatory element CSk-SH5.

On the other hand, the wild-type human myotubularin 1 (hMTM1; SEQ ID NO: 95) and codon-optimized hMTM1 (hMTM1co; SEQ ID NO: 96) genes flanked by BsiWI and XmaJI restriction sites at the 5′ and 3′ends were cloned and driven from the hDES1.4kb (SEQ ID NO: 92), which was operably linked to the regulatory element Sk-SH4 (SEQ ID NO: 121).

The Sk-SH4 regulatory element (SEQ ID NO: 121) operably linked to the hDES1.4kb promoter (SEQ ID NO: 92), or the CSk-SH5 regulatory element (SEQ ID NO: 122) operably linked to the SPc5-12 promoter (SEQ ID NO: 124), were cloned upstream of the MVM intron (SEQ ID NO: 125) in the context of a single stranded adeno-associated viral vector (AAVss) backbone. The vector also contained a 49 bp synthetic polyadenylation site (Levitt N et al, 1989) (SEQ ID NO: 127). The generated constructs were designated as
pAAVss-Sk-SH4-hDES1.4kb-MVM-hMTM1
pAAVss-Sk-SH4-hDES1.4kb-MVM-hMTM1co
pAAVss-CSk-SH5-SPc5-12-MVM-hGAA
pAAVss-CSk-SH5-SPc5-12-MVM-hGAAco.

### AA V Vector Production and Purification

AAV vector production and purification were carried out as described in Example 2.

Animal Studies

All animal procedures were approved by the institutional animal ethics committee of the Free University of Brussels (VUB) (Brussels, Belgium). All mice were housed under specific pathogen-free conditions; food and water were provided ad libitum.

The concentrated vectors (5×10¹¹ vg/mouse) were injected into the tail-vein of 4 weeks old CB.17-SCID mice (Janvier, France). Four to five weeks post injection, the mice were euthanized and individual organs were analyzed to evaluate mRNA and protein expression.

hGAA(co) and hMTM1(co) ELISA

For all GAA and MTM1 ELISA, the protocol will follow as indicated by manufacturer's instruction. Both kits (MyBiosources) require PBS as the protein extraction cocktails. The protease inhibitor cocktail (Invitrogen, USA) is added to inhibit the protease activity and maximise the quality of the samples. For each ELISA, 50 mg of the tissue was taken from frozen storage. Then 500 uL of cold PBS with protease inhibitor were added to the tissue and then homoginized with Matrix D (MPBio) for 3 cycles of 20 s. Afterward, the lysates were centrifuged at 13,000 rpm for 5 mins at 4 C. The supernatants were collected and processed as mentioned in each ELISA kits or stored at −20 C until analysis.

mRNA Analysis

For qRT-PCR, the 30-50 mg of the samples were removed from the frozen storage. The samples were homogenised using Matrix D (MPBio) for 2 cycles of 20 s. Then the RNA were extracted using RNA Nucleospin (Macherey-Nagel). The cDNAs were synthesised using SuperScript III cDNA synthesis kit (Invitrogen, USA) according to the manufacture's protocol. Next, the cDNA was amplified by quantitative qPCR on an ABI 7700 (Applied Biosystems, USA). The primer sequences for each gene are listed table below.

| | | | Amplicon | SEQ |
|---|---|---|---|---|
| Target | Primer | Sequence | size (bp) | ID |
| hGAA | F | TGCCCTCGCA | 129 | 140 |
| | | GTATATCACA | | |
| | | G | | |
| | R | GAGACCCGTA | | 141 |
| | | GAGGTTCGC | | |
| hGAAco | F | ACCCCTTCAT | 148 | 142 |
| | | GCCTCCTTAT | | |
| | R | TCCATGTAGT | | 143 |
| | | CCAGGTCGTT | | |
| hMTM1 | F | GTTTGAGATC | 96 | 144 |
| | | CTCACGAGAT | | |
| | | ACG | | |
| | R | GTCCATCCAT | | 145 |
| | | CCACGTTAAA | | |
| | | CTT | | |
| hMTM1co | F | GGCAAGAGAA | 150 | 146 |
| | | ACAAGGACGA | | |
| | R | GGCATCGTCG | | 147 |
| | | GACTCATATC | | |

### Results

hGAA(co) Expression

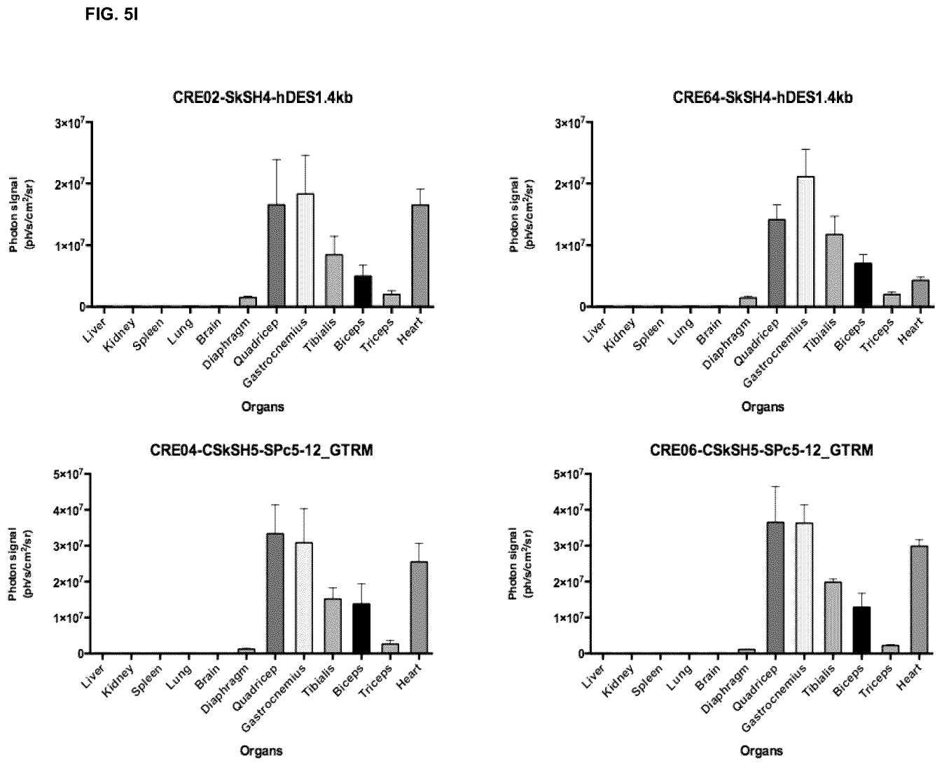

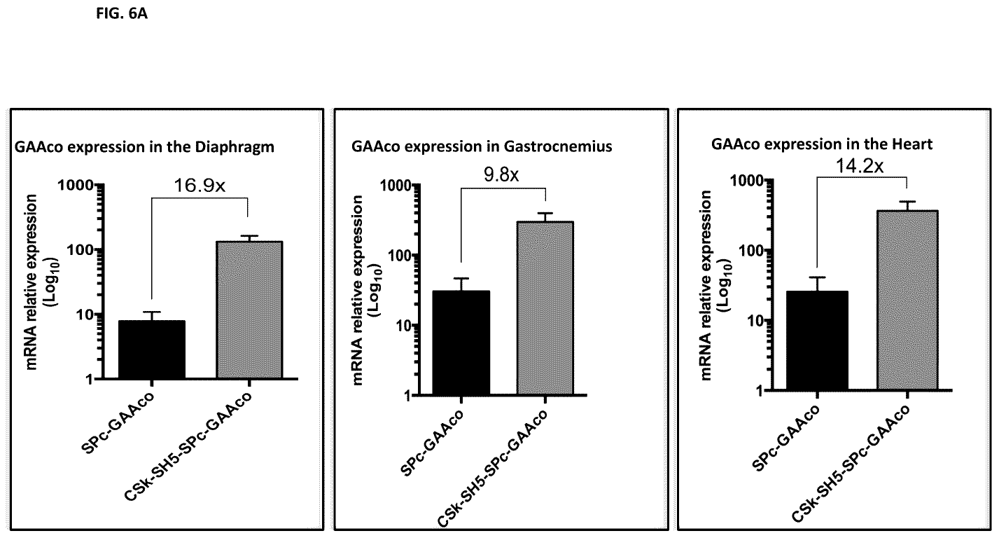

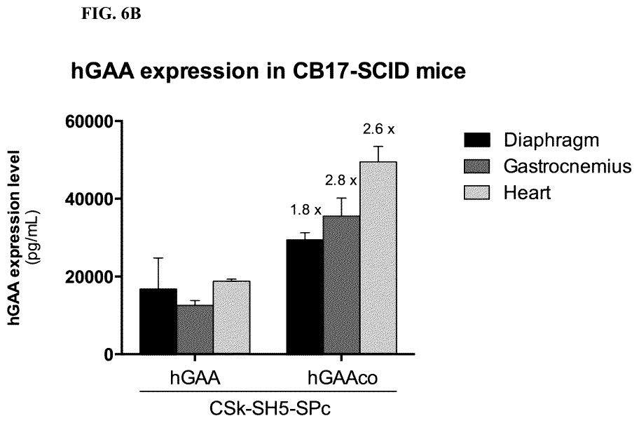

Using quantitative RT-PCR, the expression of the hGAA and hGAAco genes in diaphragm, gastrocnemius, and heart of CB.17-SCID mice injected with the SPc-hGAAco, CSk-SH5-SPc-hGAAco and CSk-SH5-SPc-hGAA constructs was assessed. The results show that CSk-SH5 can increase the hGAAco mRNA expression 16.9, 9.8, and 14.2 times respectively in diaphragm, gastrocnemius, and heart of CB.17-SCID mice (FIG. 6a) Furthermore, FIG. 6B shows that codon-optimized hGAA sequence itself improves the translation efficiency resulting in >2 folds higher hGAA protein expression in diaphragm, gastrocnemius, and heart of CB.17-SCID mice, compared to wild type hGAA.

hMTM1(co) Expression

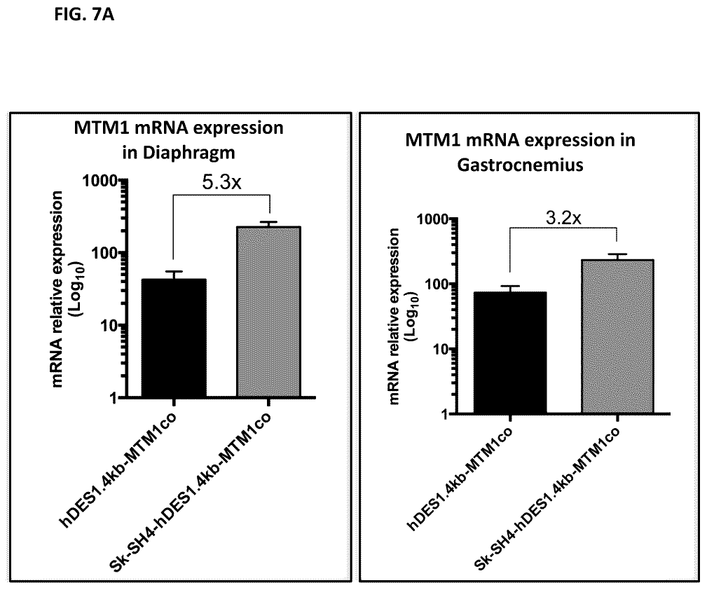

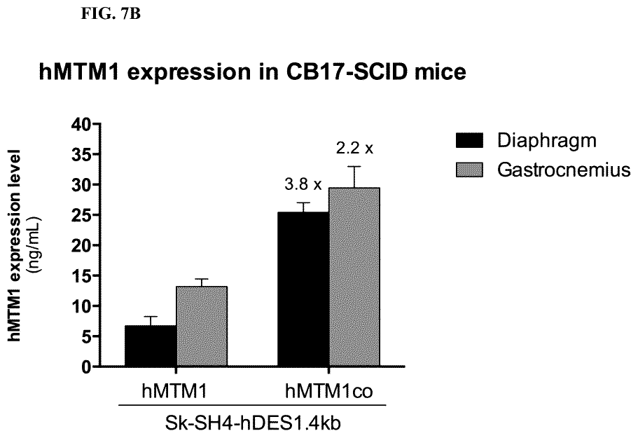

Using quantitative RT-PCR and ELISA, the expression of the hMTM1 and hMTM1co genes in diaphragm and gastrocnemius of CB.17-SCID mice injected with the hDES1.4kb-hMTM1co, Sk-SH4-hDES1.4kb-hMTM1co and Sk-SH4-hDES1.4kb-hMTM1 construct was assessed. The results show that Sk-SH4 can increase the hMTM1co mRNA expression 5.3 and 3.2 times respectively in diaphragm and gastrocnemius of CB.17-SCID mice (FIG. 7A). Furthermore, FIG. 7B shows that codon-optimized hMTM1 sequence alone can improve the translation efficiency resulting in 2-4 fold higher hMTM1 protein expression in diaphragm and gastrocnemius of CB.17-SCID mice, compared to wild type hMTM1.

### Example 5: Prototype AAV Vectors for MTM and GSD-II for Potential Clinical Trial for MTM and GSD II Related Diseases

The prototype AAV vector for MTM (FIG. 9 A)) was designed to maximize transcription and translation of MTM1. Transcription was enhanced by coupling the diaphragm-CRE (Dia-CRE) to the previously identified skeletal muscle CRE (Sk-SH4). These elements were cloned upstream of a 1.4 kb human desmin promoter to drive expression of a human bias codon-usage optimized human MTM1 gene (hMTM1co), in combination with the minute virus of mouse (MVM) intron and a synthetic polyadenylation site (Synth poly A).

The prototype AAV vector for GSD-II (FIG. 9 B)) was designed to maximize transcription and translation of GAA. Transcription was enhanced by coupling the diaphragm-CRE (Dia-CRE) to the previously identified cardiac/skeletal muscle CRE (CSk-SH5). These elements were cloned upstream of a synthetic SPc5-12-GTRM promoter to drive expression of a human bias codon-usage optimized human GAA gene (hGAAco), in combination with the minute virus of mouse (MVM) intron and the human acid α-glucosidase gene (hGAAco) and a synthetic polyadenylation site (Synth poly A).

The following 10 different AAV constructs have been evaluated for expressing therapeutic genes in vivo in mouse and the best performing ones will be the subject of a pre-clinical or phase I clinical trial:
1) pAAVss-hDes1.4kb-MVM-hMTM1-SynthpA (no diaphragm CRE, no muscle CRE Sk-SH4, only Desmin1.4kb promoter driving the MTM1 gene expression+MTM1) (SEQ ID NO; 135)
2) pAAVss-hDes1.4kb-MVM-hMTM1co-SynthpA (no diaphragm CRE, no muscle CRE Sk-SH4, +Des1.4kb+codon opt MTM1) (SEQ ID NO; 134)
3) pAAVss-Sk-SH4-hDes1.4kb-MVM-hMTM1-SynthpA (no diaphragm CRE, +muscle CRE Sk-SH4+Des1.4kb+MTM1) (SEQ ID NO; 137)
4) pAAVss-Sk-SH4-hDes1.4kb-MVM-hMTM1co-SynthpA (no diaphragm CRE, +muscle CRE Sk-SH4+Des1.4kb+codon opt MTM1) (SEQ ID NO; 136)
5) pAAVss-CRE64-Sk-SH4-hDes1.4kb-MVM-hMTM1co-SynthpA (contain best selected Diaphragm CRE64 combined with muscle CRE Sk-SH4) (SEQ ID NO; 131)
6) pAAVss-SPc5-12GTRM-MVM-hGAA-SynthpA (no diaphragm CRE, no muscle CRE, only SPc5-12-GTRM promoter driving the GAA gene expression)) (SEQ ID NO; 139)
7) pAAVss-SPc5-12GTRM-MVM-hGAAco-SynthpA (no diaphragm, no muscle CRE CSk-SH5, +SPc5-12-GTRM+codon opt GAA) (SEQ ID NO; 138)
8) pAAVss-CSk-SH5-SPc5-12GTRM-MVM-hGAA-SynthpA (no diaphragm CRE, +muscle CRE CSk-SH5+SPc5-12-GTRM+GAA) (SEQ ID NO; 133)
9) pAAVss-CSk-SH5-SPc-5-12GTRM-MVM-hGAAco-SynthpA ((no diaphragm CRE, +muscle CRE CSk-SH5+SPc5-12-GTRM+codon opt GAA) (SEQ ID NO; 132)
10) pAAVss-CRE04-CSK-SH5-SPc-5-12GTRM-MVM-hGAAco-SynthpA ((contain best selected Diaphragm CRE04 combined with muscle CRE CSk-SH5) (SEQ ID NO; 130)

Since Myotubular myopathy (MTM) patients do not have heart problems, it is not intended to target the heart for treating this disease. For this, the Sk-SH4 CRE with human Desmin 1,4kb promoter cassette was used in combination with Dia-CRE64 to express the MTM1 gene or codon optimised MTM1co in diaphragm and skeletal muscle tissue.

Since Pompe patients also suffer from heart problems, it is important to also target the heart. For this the CSk-SH5 CRE in combination with Dia-CRE04 is used with the synthetic SPc5-12 promoter cassette to express the therapeutic GAA gene or codon optimised GAAco gene because this combination leads to very robust expression in diaphragm, skeletal muscles and heart tissue.

### Example 6: In Vivo Validation of Prototype AAV Vectors pAAVss-CRE04-CSk-SH5-SPc-5-12GTRM-MVM-hGAAco-SynthpA and pAAVss-CRE64-Sk-SH4-hDes1.4kb-MVM-hMTM1co-SynthpA

AAV backbones comprising the diaphragm-specific regulatory element CRE04 (defined by SEQ ID NO:4) or CRE64 (defined by SEQ ID NO:64) combined respectively with a muscle-specific Sk-SH4 (SEQ ID NO:121) or a cardiac/muscle-specific CSk-SH5 (SEQ ID NO:122) regulatory element were injected into CB17-SCID pups at dose of 5×10¹⁰ vg/mouse. At 8-week post-injection time point, the organs were removed for total RNA extraction using AllPrep DNA/RNA mini kit (Qaigen) according to manufacturer's instruction. The RNAs were reversed-transcribed using Superscript VI cDNA synthesis kit (ThermoFisher). Afterwards, cDNA from each organ was subjected to perform qRT-PCR using hGAA, hGAAco, hMTM1 or hMTM1co therapeutic gene-specific primers. The mGapdh gene was used as internal control to normalize resulting in relative expression (2^{ΔΔCt}).

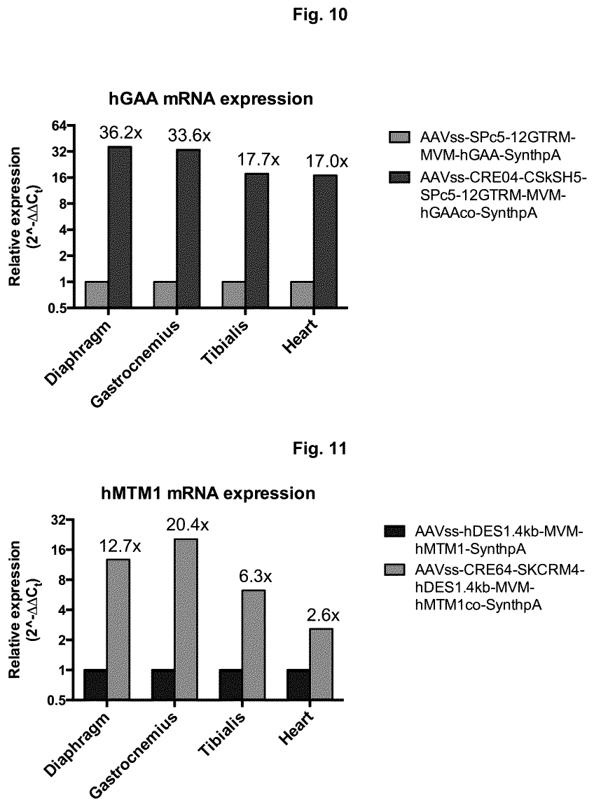

AAV containing a combination of diaphragm-specific CRE04 and skeletal muscle and heart specific CSk-SH5 (AAVss-CRE04-CskSH5-SPc5-12GTRM-MVM-hGAAco-SynthpA (SEQ ID NO:130)) increased the expression of hGAAco in the diaphragm, gastrocnemius, tibialis and heart at least 17-fold compared to control (FIG. 10).

AAV containing a combination of diaphragm-specific CRE64 and Sk-SH4 (called Sk-CRM4 in the figure—AAVss-CRE64-Sk-SH4-hDes1.4kb-MVM-hMTM1co-SynthpA (SEQ ID NO:131)) increased the expression of hMTM1co in the diaphragm, gastrocnemius, tibialis and heart at least 2.6-fold compared to control (FIG. 11).

1-30. (canceled)

31. A method for enhancing gene expression in diaphragm, skeletal muscle and heart tissue, comprising administering to a subject in need thereof, a nucleic acid regulatory element having a maximal length of 1000 nucleotides and comprising a sequence defined by SEQ ID NO: 2, a sequence having at least 95% identity to the sequence, a complement of the sequence, or a sequence hybridizing under stringent conditions to the nucleic acid regulatory element.

32. A nucleic acid expression cassette comprising at least one nucleic acid regulatory element having a maximal length of 1000 nucleotides, and comprising a sequence defined by SEQ ID NO: 2, a sequence having at least 95% identity to the sequence, a complement of the sequence, or a sequence hybridizing under stringent conditions to the nucleic acid regulatory element, operably linked to a promoter other than an alpha-actin-1 (ACTA-1), wherein the promoter is at least one of a diaphragm-specific promoter, a heart-specific promoter and a skeletal muscle-specific promoter.

33. The nucleic acid expression cassette according to claim 32, wherein the nucleic acid regulatory element is operably linked to the promoter and a transgene.

34. The nucleic acid expression cassette according to claim 32, wherein the promoter is selected from the group consisting of: an SPC5-12 promoter, an MYL2 promoter, an MB promoter, a DES promoter, a TNNC1 promoter, a TCAP promoter, an MYH7 promoter, an ALDA promoter, and a TPM1 promoter.

35. The nucleic acid expression cassette according to claim 33, wherein the transgene encodes a therapeutic protein or an immunogenic protein.

36. The nucleic acid expression cassette according to claim 33, wherein the transgene encodes a secretable protein or a structural protein selected from the group consisting of acid glucosidase (GAA), myotubularin (MTM1), follistatin, dystrophin, sarcoglycan, and dysferlin.

37. The nucleic acid expression cassette according to claim 33, wherein the transgene encodes acid glucosidase (GAA) as defined by SEQ ID NO: 93 or a codon-optimised human acid glucosidase gene (hGAAco) as defined by SEQ ID NO: 94.

38. The nucleic acid expression cassette according to claim 32, further comprising a Minute Virus of Mouse (MVM) intron.

39. The nucleic acid expression cassette according to claim 32, further comprising a polyadenylation signal selected from the group consisting of a synthetic polyadenylation signal (SEQ ID NO: 127) and a Simian Virus 40 (SV40) polyadenylation signal.

40. A vector comprising the nucleic acid expression cassette according to claim 32.

41. The vector according to claim 40, which is a viral vector.

42. The vector according to claim 40, which is a non-viral vector selected from the group consisting of a plasmid, a minicircle, an episomal vector, and a transposon-based vector.

43. The vector according to claim 40, further comprising a CRE CSk-SH1 regulatory element as defined in SEQ ID NO: 123, a CRE CSk-SH5 regulatory element as defined in SEQ ID NO: 122, or a CRE Sk-SH4 regulatory element as defined in SEQ ID NO: 121, positioned before or after the nucleic acid regulatory element.

44. The vector according to claim 40, further comprising (a) a CRE CSk-SH1 regulatory element as defined in SEQ ID NO: 123, a CRE-CSk-SH5 regulatory element as defined in SEQ ID NO: 122, or a CRE Sk-SH4 regulatory element as defined in SEQ ID NO: 121, (b) a nucleic acid regulatory element having a maximal length of 1000 nucleotides and comprising the sequence defined by SEQ ID NO: 2, a sequence having at least 95% identity to the sequence, a complement of the sequence, or a sequence hybridizing under stringent conditions to the nucleic acid regulatory element, (c) an MVM intron, (d) an SPc5-12 promoter, (e) a human GAA transgene (SEQ ID NO: 93) or a codon-optimised variant thereof (SEQ ID NO: 94), and (f) a synthetic poly A site (SEQ ID NO: 127).

45. A pharmaceutical composition comprising the vector according to claim 40, and a pharmaceutically acceptable carrier.

46. A method of treating a disease affecting a diaphragm comprising administering a therapeutically effective amount of the pharmaceutical composition according to claim 45 to a subject in need thereof, wherein the vector comprises (a) a CRE-CSk-SH1 regulatory element (SEQ ID NO: 123), a CRE-CSk-SH5 regulatory element (SEQ ID NO: 122), or a CRE Sk-SH4 regulatory element (SEQ ID NO: 121), (b) a nucleic acid regulatory element having a maximal length of 1000 nucleotides and comprising the sequence defined by SEQ ID NO: 2, a sequence having at least 95% identity to the sequence, a complement of the sequence, or a sequence hybridizing under stringent conditions to the nucleic acid regulatory element, (c) an SPC5-12 promoter, and (d) a human transgene that effects a function of the diaphragm when being defective or a codon-optimized variant thereof.

47. A method of treating Pompe disease comprising administering a therapeutically effective amount of the pharmaceutical composition according to claim 45 to a subject in need thereof, wherein the vector comprises (a) a CRE-CSk-SH1 regulatory element (SEQ ID NO: 123) or a CRE-CSk-SH5 regulatory element (SEQ ID NO: 122), (b) a nucleic acid regulatory element having a maximal length of 1000 nucleotides and comprising the sequence defined by SEQ ID NO: 2, a sequence having at least 95% identity to the sequence, a complement of the sequence, or a sequence hybridizing under stringent conditions to the nucleic acid regulatory element, (c) an SPC5-12 promoter, and (d) a human GAA transgene (SEQ ID NO: 93) or a codon-optimised variant thereof (SEQ ID NO: 94).

48. A vaccine comprising the pharmaceutical composition according to claim 45, and at least one adjuvant.

49. A method for expressing a transgene product in diaphragm, skeletal muscle, and heart cells, comprising:
introducing the vector according to claim 40, wherein the nucleic acid regulatory element is operably linked to the promoter and a transgene, into said cells; and
expressing the transgene product in the cells.

50. A method of treating Myotubular myopathy (MTM) comprising administering a therapeutically effective amount of the pharmaceutical composition according to claim 45 to a subject in need thereof.


SEQUENCE LISTING (147 sequences)

SEQ ID NO: 1 - DNA, 328 bp, synthetic construct
  misc_feature 1..328: note = Dph-CRE01
  source 1..328: mol_type = other DNA
ggagacactccatatacggcccggcccgcgttacctgggaccgggccaacccgctccttctttggtcaacgcaggggacccgggcgggggcccaggccgcgaaccggccgagggagggggctctagtgcccaacacccaaatatggctcgagaagggcagcgacattcctgcggggtggcgcggagggaatgcccgcgggctatataaaacctgagcagagggacaagcggccaccgcagcggacagcgccaagtgaagcctcgcttcccctccgcggcgaccagggcccgagccgagagtagcagttgtagctacccgcccaggtag

SEQ ID NO: 2 - DNA, 452 bp, synthetic construct
  misc_feature 1..452: note = Dph-CRE02
  source 1..452: mol_type = other DNA
gacaggtgcggttcccggagcgcaggcgcacacatgcacccaccggcgaacgcggtgaccctcgccccaccccatcccctccggcgggcaactgggtcgggtcaggaggggcaaacccgctagggagacactccatatacggcccggcccgcgttacctgggaccgggccaacccgctccttctttggtcaacgcaggggacccgggcgggggcccaggccgcgaaccggccgagggagggggctctagtgcccaacacccaaatatggctcgagaagggcagcgacattcctgcggggtggcgcggagggaatgcccgcgggctatataaaacctgagcagagggacaagcggccaccgcagcggacagcgccaagtgaagcctcgcttcccctccgcggcgaccagggcccgagccgagagtagcagttgtagctacccgcccaggtagg

SEQ ID NO: 3 - DNA, 239 bp, synthetic construct
  misc_feature 1..239: note = Dph-CRE03
  source 1..239: mol_type = other DNA
agagagagggacaggcaccaactgggtaacctctgctgacccccactctactttaccataagtagctccaaatccttctagaaaatctgaaaggcatagccccatatatcagtgatataaatagaacctgcagcaggctctggtaaatgatgactacaaggtggactgggaggcagcccggccttggcaggcatcatcctctaaatataaagatgagtttgttcagcctttgcagaagg

SEQ ID NO: 4 - DNA, 509 bp, synthetic construct
  misc_feature 1..509: note = Dph-CRE04
  source 1..509: mol_type = other DNA
ccttttagagaatccacacctgtcccagttgctgggttccactaccaaaagtgaattgcaactattttaggagcacttaagcacatccgaaaaatgagtgattctgttctggcccacaccacatcactgatgtacccccttaaagcatgtccctgagttcatcacagaagactgctcctcctgtgccctccacaaggttagaactgtccttgtcttagggaaaaaggagagagagagagagagagagagagagagagagagagagagagagagagagggacaggcaccaactgggtaacctctgctgacccccactctactttaccataagtagctccaaatccttctagaaaatctgaaaggcatagccccatatatcagtgatataaatagaacctgcagcaggctctggtaaatgatgactacaaggtggactgggaggcagcccggccttggcaggcatcatcctctaaatataaagatgagtttgttcagcctttgcagaagga

SEQ ID NO: 5 - DNA, 169 bp, synthetic construct
  misc_feature 1..169: note = Dph-CRE05
  source 1..169: mol_type = other DNA
ccttttagagaatccacacctgtcccagttgctgggttccactaccaaaagtgaattgcaactattttaggagcacttaagcacatccgaaaaatgagtgattctgttctggcccacaccacatcactgatgtacccccttaaagcatgtccctgagttcatcacagaa

SEQ ID NO: 6 - DNA, 400 bp, synthetic construct
  misc_feature 1..400: note = Dph-CRE06
  source 1..400: mol_type = other DNA
gggccaggggacggtggcttctacgtgcttgggacgttcccagccaccgtcccatgttcccggcggggggccagctgtccccaccgccagcccaactcagcacttggtcagggtatcagcttggtgggggggcgtgagcccagcccctggggcggctcagcccatacaaggccatggggctgggcgcaaagcatgcctgggttcagggtgggtatggtgcgggagcagggaggtgagaggctcagctgccctccagaactcctccctggggacaacccctcccagccaatagcacagcctaggtccccctatataaggccacggctgctggcccttcctttgggtcagtgtcacctccaggatacagacagccccccttcagcccagcccagccaggtac

SEQ ID NO: 7 - DNA, 255 bp, synthetic construct
  misc_feature 1..255: note = Dph-CRE07
  source 1..255: mol_type = other DNA
cccagcccccttccccgggaggtgggagcggccacccagggccccgtggctgcccttgtaaggaggcgaggcccgaggacacccgagacgcccggttataattaaccaggacacgtggcgaacccccctccaacacctgcccccgaacccccccatacccagcgcctcgggtctcggcctttgcggcagaggagacagcaaagcgccctctaaaaataactcctttcccggcgaccgagaccctccctgtccccc

SEQ ID NO: 8 - DNA, 354 bp, synthetic construct
  misc_feature 1..354: note = Dph-CRE08
  source 1..354: mol_type = other DNA
ctcctgggctcgagcaattctcctgccttgacctcccaaagtgctgggattacaggcatgagccactacaccctgccctagtgtgctttatatcaaaggggaaaccatttggggcttccaaacaggaaatgaacagtcgtaccttttgtcctcactaacattcagattgcaggaatgccgtgtcccatagggaaagagaaattcctgaggcaagtgtcatatctgaacttgagatagtcaccttccaggcagaaagctacacccgcctcttttcccagcctctggcatcagctgccggcggtgtgggtaagggatgcaaagaactcaaaacatgtagccaggattccccatttg

SEQ ID NO: 9 - DNA, 297 bp, synthetic construct
  misc_feature 1..297: note = Dph-CRE09
  source 1..297: mol_type = other DNA
tgacaagcacaagtgtccccggcccaagcaccgcagagagcgcgcagcatctctccccgtgaccatgacccagctactgcctctttaaccttgaatgcctttttgggggctcacgtgtcacccagtggcgagtgagccacccttacttcagaagaacggcatggggtgggggggccttaggtggtgcccgcctcacctatgactgccaaaagcggtcatggggttatttttaaacatggggaggaagtatttattgttcctgggctgcagagagctgggcggagtgtggaattcttc

SEQ ID NO: 10 - DNA, 608 bp, synthetic construct
  misc_feature 1..608: note = Dph-CRE10
  source 1..608: mol_type = other DNA
actcagggtaaactgaggcactcaaactgccgaggagctccgcctcccgagagacatttaatccggggggatttgcaggaaacttctaaattaagggtagcggctgctgcagctgagggggggcacgccggtccctgcgcccgggcagctgccgtgagctcacgccccgaaatagccccaggggccccagccgcagctgccactgggtccggctgtcactcagaggaagcacggagcccccagcccaagggtcccctccccttcgcatcgccggggtttttccagccgaccgtcggccactttttcctccgacggctggcagggaagagggggatgggggcgggaccccaagggaggcggtccccagtgggtgggcgaagggggcggccgcaccccccggccgggccgtgcttctgcccctacaaggtttgggccgaggtgggggagggtcctggttgccggccccgcccggtccctccccgccttttaggcgcccgcgtggccgggacgtcccagtcccgctccgtcctcctcgcctgccaccggtgcacccagtccgctcacccagcccagtccgtccggtcctcaccgcctgccggccggcccac

SEQ ID NO: 11 - DNA, 308 bp, synthetic construct
  misc_feature 1..308: note = Dph-CRE11
  source 1..308: mol_type = other DNA
tttttcctccgacggctggcagggaagagggggatgggggcgggaccccaagggaggcggtccccagtgggtgggcgaagggggcggccgcaccccccggccgggccgtgcttctgcccctacaaggtttgggccgaggtgggggagggtcctggttgccggccccgcccggtccctccccgccttttaggcgcccgcgtggccgggacgtcccagtcccgctccgtcctcctcgcctgccaccggtgcacccagtccgctcacccagcccagtccgtccggtcctcaccgcctgccggccggcccac

SEQ ID NO: 12 - DNA, 263 bp, synthetic construct
  misc_feature 1..263: note = Dph-CRE12
  source 1..263: mol_type = other DNA
gactcagggtaaactgaggcactcaaactgccgaggagctccgcctcccgagagacatttaatccggggggatttgcaggaaacttctaaattaagggtagcggctgctgcagctgagggggggcacgccggtccctgcgcccgggcagctgccgtgagctcacgccccgaaatagccccaggggccccagccgcagctgccactgggtccggctgtcactcagaggaagcacggagcccccagcccaagggtcccctcccct

SEQ ID NO: 13 - DNA, 324 bp, synthetic construct
  misc_feature 1..324: note = Dph-CRE13
  source 1..324: mol_type = other DNA
ctcagggaatgagctggatacaactaaaaatcagcacatttttgtttggttaaaacattctttgtggtcaatttctttctaacagatcgagttctctaaggaacagcaggatggtaagtttaaaagctatggttcttaaatgtgcacactcataaagatggcatgtgtgaaaaccaactcccttgggatgagtttagctcttcctaattatccccactggtgttgccattctgaatataaattgctctcgatcactctaaatataaattagctcaatctgaaacccttgctgatactcagtaatcaaaggtttaacaaacaaaa

SEQ ID NO: 14 - DNA, 326 bp, synthetic construct
  misc_feature 1..326: note = Dph-CRE14
  source 1..326: mol_type = other DNA
catttagttaaatcattaagatgcagaaagatattccctaattatcttttgcaatagctttttatgttcctagattcaaaagtatcctaaggcaaccttctaatgccaagacacctacatagcttcctaaaaatctcagagcaaccagcactcattcttctgtcaatgcatttgaaagagtactagttttttttctttctttctcttcttcgccactgcagtccttcagtgctgaccagattgctggtaagtgaattgagtttgtctgctacagatgtaggcacagcactgcctagtttctgcaatattacttatcttcaaggcat

SEQ ID NO: 15 - DNA, 596 bp, synthetic construct
  misc_feature 1..596: note = Dph-CRE15
  source 1..596: mol_type = other DNA
tttgtttgttaaacctttgattactgagtatcagcaagggtttcagattgagctaatttatatttagagtgatcgagagcaatttatattcagaatggcaacaccagtggggataattaggaagagctaaactcatcccaagggagttggttttcacacatgccatctttatgagtgtgcacatttaagaaccatagcttttaaacttaccatcctgctgttccttagagaactcgatctgttagaaagaaattgaccacaaagaatgttttaaccaaacaaaaatgtgctgatttttagttgtatccagctcattccctgagtaatcttcaaagtaagctccaaaagtcagcctacttttcagagttcaggcgtagcttggacactaacaggttaagccatagtgtaacatgccttgaagataagtaatattgcagaaactaggcagtgctgtgcctacatctgtagcagacaaactcaattcacttaccagcaatctggtcagcactgaaggactgcagtggcgaagaagagaaagaaagaaaaaaaactagtactctttcaaatgcattgacagaagaatgagtgctggttgc

SEQ ID NO: 16 - DNA, 290 bp, synthetic construct
  misc_feature 1..290: note = Dph-CRE16
  source 1..290: mol_type = other DNA
agaccataaaagtaccggcaggcttccatctcactatggctgtctactccaaggttctggtcatctaaaaatagctctcagggtacagatccatctttccctttgccctaagaaagctaaagaactctccaaggggtgtggcaacttatctctgaaacctgatgctagctgtgaggtcaaagcttgcccagaaataaaaggaagcctcagccagggatgaccccactcagggaccggagcagccctcaactcactcttcagcttccctgctgtgttgcagcccagccgct

SEQ ID NO: 17 - DNA, 632 bp, synthetic construct
  misc_feature 1..632: note = Dph-CRE17
  source 1..632: mol_type = other DNA
gtgctccctagaagggaagccactttaagacatgatgggctttccttatacgtctgcttttttatttttaaccaccaccacctctttgctgaccaacatttccttgcaaattatggcaaggggggaggagcatgagcaaaggagctgtgtgaaagttggtgatactccctgccaaaattccaagttgaaatggaatccttcagtaaagtctcttaggcaaggtgggaaaaaaagatttacaacttcagcagaactgtaattctaccaagaaaaagtacagtcaatctacctcactgtcaccaaaggatctgaagctggctttgtcatatatctttctcctttatatatggaggtgggggggggagaaaaaaaggaaagagagttaaaaaaaattgatgaagccctgaccctttagattccatttatagtctgagccggaatgccatcccccttgactagagaactgtccaatccagccgcatgtgtcaagattctattaggcactaagtgaaatatatatgcatgcccttatgccgtttaacactctgggtccatcttcaagacactgggctgtggatcaacccaaccaccactcctcttccaagaatcattttgacaggttcttttggagg

SEQ ID NO: 18 - DNA, 297 bp, synthetic construct
  misc_feature 1..297: note = Dph-CRE18
  source 1..297: mol_type = other DNA
cctttgacttttcttgaaaggggagggctgggatattccagagattgatccttaaggcttgctgactgcctactcacttctggaaacttccagcagtgtcattcatagacctgtgaagagctcttagcttgtttccttcacacagtggggactctgaggggtcagagtgagtcacccagcaggccagtggcaggggtgagccagaagcctggccaaaccctcctatcatcatggagagaagaaagcctcctccagaagacgggaggccggcagggcgtggggcctgctcagatgcag

SEQ ID NO: 19 - DNA, 309 bp, synthetic construct
  misc_feature 1..309: note = Dph-CRE19
  source 1..309: mol_type = other DNA
ggaacagggcaggccttcctcacaagaatctaggacgtcaaggcctgccacctgcttggaggcttaaatttctctgcaagggcccttggctaaattaggtaatgggttcagactgtgggaggggtgggactcgctgaccccaggatctgattgggcagggtctccagtgctggggagcagggaggtgggaggggagggtgcccctacaaatcccgggggctagagcaggccaggtcatctttgggtggtggagtgcaaaggaggcgacctgcaacagaggagtcccggtcaccagcaaccatggtaagg

SEQ ID NO: 20 - DNA, 267 bp, synthetic construct
  misc_feature 1..267: note = Dph-CRE20
  source 1..267: mol_type = other DNA
aattgtgacatttaagcagtgactttgcaaaattggacgtgtcagtaggaaaaagtactcttcccaccttaaagattgacaggaacatgagatggctgaagctgttctgagcttttttctggcaagagtacataggacaagaaatactctggagagctcaacattgaatcacatcatatcatagtgcatgactagttgccttaatcttgcagttggactcactcaggagaaattgttggcagagggattaagtagcctgacaaaatt

SEQ ID NO: 21 - DNA, 493 bp, synthetic construct
  misc_feature 1..493: note = Dph-CRE21
  source 1..493: mol_type = other DNA
cttttcttttctaattaggtcagttacttcatgtctgtttttcttttttgacatggtttcttctggctttggcaccacatattttccccatatgtcattgcctctggagcttcatgttgcaatagtttttcaaggggaacggagagcacattgctaagggtgggggatggcttttgcctcttttgcctgccctttgcttcagtgagtgttcgtatttctgttgggcctagttctgtttggttttgtagtcttcagagtcagttatgtttggactgaaagatacttaagtaaaaataatggcaagtcagagatatgtctggcaaaggtgcagcacatttgttaaggttactggtttatttatctcccttgtctctatggtgactaaatctgggtctgggatttaatggacttagttttgacctcttgtaacatctcctaacttttcccagcctctgatttagaaagaattcattttcacttgaggagagaaact

SEQ ID NO: 22 - DNA, 197 bp, synthetic construct
  misc_feature 1..197: note = Dph-CRE22
  source 1..197: mol_type = other DNA
tcatttgttttgattacccgggtgattgccaaatgttaatttaaaactgaaataggccatgagctcactcccctaacaatgctgagtgtttgagctaatacggcttgataatgagggagccagatccactgccagtgctcatgttatatgataaacagacatttggagaagtatgcagaacatcttggcagttttcc

SEQ ID NO: 23 - DNA, 637 bp, synthetic construct
  misc_feature 1..637: note = Dph-CRE23
  source 1..637: mol_type = other DNA
aaaaattggctttaaggtcatgagcctccttacagcagtccctctcaccttcagctgtattccctatttcagatatacttgtttctctgctgtaagtacacttttcttttctaattaggtcagttacttcatgtctgtttttcttttttgacatggtttcttctggctttggcaccacatattttccccatatgtcattgcctctggagcttcatgttgcaatagtttttcaaggggaacggagagcacattgctaagggtgggggatggcttttgcctcttttgcctgccctttgcttcagtgagtgttcgtatttctgttgggcctagttctgtttggttttgtagtcttcagagtcagttatgtttggactgaaagatacttaagtaaaaataatggcaagtcagagatatgtctggcaaaggtgcagcacatttgttaaggttactggtttatttatctcccttgtctctatggtgactaaatctgggtctgggatttaatggacttagttttgacctcttgtaacatctcctaacttttcccagcctctgatttagaaagaattcattttcacttgaggagagaaactgtctcacttagaaaaggggtcctaactggactctcgaaaagtgg

SEQ ID NO: 24 - DNA, 561 bp, synthetic construct
  misc_feature 1..561: note = Dph-CRE24
  source 1..561: mol_type = other DNA
gtctgcatcctaatcctgctgttgattggagataacttcagcggtcacagggcacctcgtctccatggcaacccctctaaatattcctctagtgacttgtgctctacattctggctggatcagagccctcctgtggcaaaatattctaaagatgttaaaagagagagagagaaaaaaaaaggctcaactaataaatctattctaaacgactgagtcatttgttttgattacccgggtgattgccaaatgttaatttaaaactgaaataggccatgagctcactcccctaacaatgctgagtgtttgagctaatacggcttgataatgagggagccagatccactgccagtgctcatgttatatgataaacagacatttggagaagtatgcagaacatcttggcagttttcctttaactctaagggttgtaaatacaaagtagaatgtgttaccatgagccatttcataggaagttgactttatttttttagaattcagaactttgcttctaaaaaggtaactttgcctcttgattctcatttttttccctaacttaggcac

SEQ ID NO: 25 - DNA, 511 bp, synthetic construct
  misc_feature 1..511: note = Dph-CRE25
  source 1..511: mol_type = other DNA
aaagacagggaggtgcaatctaagttaggaaattggtgacaaatagtcatagaacatgggtcacctggcttcaattagttatgtgacctttaagaaaacaagtgcccgtttcagaaaagactgacctttgaggctagagacatcaagctactcctctacttttaggaaaaaacaatgacagggttaatttaaaactctgatgcagacagagttgaaaaatccatgacagatttatataacgaacgtggatggtgttttgttttcccagaatgcagttttaaggtcagacctatgactcagagctgtgcacttccctggtggtatgcatcagtgacatgttgccagtctaatcgtccagaagggcttgtgcccagctcggcttgctccattataatccccagctgagaagccacctttcattttttttaaatagataaggaagacagcatgaacatttgaaactcaaatactatatgtagtttataacttttcagcctgaagacttgatgca

SEQ ID NO: 26 - DNA, 399 bp, synthetic construct
  misc_feature 1..399: note = Dph-CRE26
  source 1..399: mol_type = other DNA
aatgcagcataccagtgaagttgctaattacattgaatcattctgtacacgttctattttgaagggaaaaaacactttctgatttttgtatgaaattgtatgaaagactacttttcagccgtgactcatgagctccgtctcattgcagctacttaggtatttattcccaaatgtctgacgacacctccaggtgcattagcatgtctattacacaagtgtcctattcattggctaaggctttgctccatctgttgaatgttctgaggaaagcattataccctttctttagaagccagtgtttatatgtggagtcactaatgtactggtaggttggatttagtgaagaacagatctgaaataacctgatttttttccatttgggttgttgttcattgttta

SEQ ID NO: 27 - DNA, 360 bp, synthetic construct
  misc_feature 1..360: note = Dph-CRE27
  source 1..360: mol_type = other DNA
cccaacctttgaatgtatatgggggagaaaataacagacaaggttggaggcgaagataaggggggatggggtggtgcttctccgcaaacatccaacccttctgtaaagattcctccagactggggaagaaggcttgggtgtcggtccctttaagaaggaagggaaagggttaaagccactgcggggccatttcccttcccggccccgagagggcgcaggagctctcaggggcttaaaggaccgggcctgggggcgggttatgggggcggagggagggaagggtggtcttggaggttggggcccgaggatatcgggggtccccccgggcccccgacatcggtctcgggaagcgaagcagcc

SEQ ID NO: 28 - DNA, 471 bp, synthetic construct
  misc_feature 1..471: note = Dph-CRE28
  source 1..471: mol_type = other DNA
ctcaccttcctcctcctcctccgcagcctcctctggagatgggggcacagaagagaaggcgttaggagctgggggagggatgggggcggtggccagagaccagggttccagtctctgctggacgggggtccctctggcctcggctgcgatgggcacgttccccctggcggtgcagggatggcgcgaggagacgctccagacccggagagggcgggcgagggcccgagggctgagccgccccttccccgccaaagtgccacactcctcgcctcccctcccaagagctcctgggcgtgtccccatcccatagggccggcccactccctactcaccttccggctgctccctgcggacgggtgtggggagagaggagggaggggagagttagacctggggtgggagagcctctccaccactgcacgccccaacccctcccagtgcagcactcactcctcatattcctgctcctcg

SEQ ID NO: 29 - DNA, 393 bp, synthetic construct
  misc_feature 1..393: note = Dph-CRE29
  source 1..393: mol_type = other DNA
ggccgcctgggaagaccggggaggtgggggccagagagtcgggaccacatgggatggggcgggctagagccagccctgtagggacagagtgggcaatggtgtctgggggctggtgagcatggagggaggacccccaacacctcagagacctgtgctgcagggccttccctgcatgagcagcagggggcagcagatgccttcggaagccgctggggcctttaggggctcagtcctggccggatgcccctcccagtccccacacatctgggctgccttagcggctggggcctccacgctgctgtctcctctgatcctccccacggggctgcgaagccggcagggctggggccaagagcccccactccgagagggtgccaagtcacggtccagggg

SEQ ID NO: 30 - DNA, 331 bp, synthetic construct
  misc_feature 1..331: note = Dph-CRE30
  source 1..331: mol_type = other DNA
ctgtcctcaggctccttacgagaacgacagaggcatctccagcgcgtcaccgagccctaaatagagtagcccagccacggcaccccccaccaagacttcttggactgggcggcagcacgcggccaggccaggcggccggacaggtggggaggtctctgtggctctccacgcccccattggtctgaggaggactctatgccctttctgagcaggggcccagcctgggggaggccatttatacccctccccctgggcccaccagcccaactcgccgctgccggcctgacctcgctcccagccctgctgcccagattctaggtgaggcccagcc

SEQ ID NO: 31 - DNA, 306 bp, synthetic construct
  misc_feature 1..306: note = Dph-CRE31
  source 1..306: mol_type = other DNA
gctcccccaactcagcataggtcataggtcacagcctcaaggccctaaggcccagagaagatggcctggccctccccgctggcaaaagtgccccacccacccaccaagacgctgctgaaagagaggaagtggctgccccaagccttctggggcaggggaagtgtggctgtggcctggtcacaggggaatcacttcttctttctgattcctgcacttcccgcccccacctcaccggccgacctgcccccaactggccctcctctcccccagcctctggcctcccagcaccatgtgccacctggcaaa

SEQ ID NO: 32 - DNA, 268 bp, synthetic construct
  misc_feature 1..268: note = Dph-CRE32
  source 1..268: mol_type = other DNA
aactctggcacattccagcccctctttgggagaaagaacatctcttcttgcccaggccctagtgagacatgcatcgcaagacatgctccccccttcctccccgctgggctccagctgctgccagccctccaagaaaggagaggccctgagttgggctattttggtatctggggtgggcacccgcagggctaaggttaccttggtatgttaagggctccctggggcaggactatataaccccagagggactgccccatgctgactcctt

SEQ ID NO: 33 - DNA, 214 bp, synthetic construct
  misc_feature 1..214: note = Dph-CRE33
  source 1..214: mol_type = other DNA
tacctggtgcaggccctcgaggccgctggtgcctgctctgtgcagtccctgctacgcctcaggcccagcgcccgagggggaggtcgccgatacccaaaataaaacctgccctatccctctgacctcagctggctgggcggagagcgcctgccaaagctccaggagctgggcgggcagcaccttcccctgctggccacacggggccagtgcaggg

SEQ ID NO: 34 - DNA, 129 bp, synthetic construct
  misc_feature 1..129: note = Dph-CRE34
  source 1..129: mol_type = other DNA
tcttgggtttctgggcagaaacgtcttgcctatttctggacacctcagctgccactggctccttataaataacccaccccaggcgagggccacgctgcccccatcttgtggcagccgacaggtgtctgc

SEQ ID NO: 35 - DNA, 403 bp, synthetic construct
  misc_feature 1..403: note = Dph-CRE35
  source 1..403: mol_type = other DNA
ttacctggtgcaggccctcgaggccgctggtgcctgctctgtgcagtccctgctacgcctcaggcccagcgcccgagggggaggtcgccgatacccaaaataaaacctgccctatccctctgacctcagctggctgggcggagagcgcctgccaaagctccaggagctgggcgggcagcaccttcccctgctggccacacggggccagtgcagggttatggcacacccaccttcaggtgcgcccctcaggacccctggcctgggtgagggcttgggtgggcacaggcttcctaggcctgcagactgaaccacgtgtaaccagaatggtgggctcagggctccgttagagaagccggcagagcgagcctggaaaaggaggttggccccaccatgggagaggatg

SEQ ID NO: 36 - DNA, 415 bp, synthetic construct
  misc_feature 1..415: note = Dph-CRE36
  source 1..415: mol_type = other DNA
agagtgaatgcatgggaatgaatagatgggtggatgaatggacaactgtgtgaaatagcagacagcctggggacaccctccctcctgtccctcctctcccaggtcttgcactgggccctgacactcagccctgcagccttcagcgtctgggaagtcccttcgaggagcagcaccccagactctgatgttctggacttcactttgccattcccttgggtcctcacagccactattttctgaaggcttcatgtcaaaccctgcttgcctcatgaatacacacagttcagaaaagcaaaacctcgacagagccaccacccacacaagggaggtgacgtgcccccatcctttctcagatgccccagccactgagagaggatccaggccaccctccccagctggggaaccacccagtgac

SEQ ID NO: 37 - DNA, 352 bp, synthetic construct
  misc_feature 1..352: note = Dph-CRE37
  source 1..352: mol_type = other DNA
accaatctgtgcctgaaccgaagagttccgcagacaccaccgcccacgctcagcctgtggggtctgaggggagtgagggggtgggtcagggtgccagactgctctcccccacctggtgcctcagtttccctgatccaagaggcagggttatccaatctggtggcctcactggccagcatacaggccatcagaggccaggaaggatgtctgctagtcggacggtgccacctggagtcctgaggaaagaaaaccagtctccacacgccacaatgagcctgtgggagatgctggcacagatggtaacggccactgcgagtgcccagggtggggccccagggttagaaagggccta

SEQ ID NO: 38 - DNA, 349 bp, synthetic construct
  misc_feature 1..349: note = Dph-CRE38
  source 1..349: mol_type = other DNA
ctcctcagcccatccagaatgtacccgctgctgggagtaaaaatagcagctgacacctcctggaggcggagggaggaccttgcctccttctccaagcacgtcctctgcatcctggcctccttcagcctcctcctctggccattcctatacttggtaaggggcctgcacgggcatagccccccccagcaagactccgcacacaccccggccacccagtcactggccaatgggctcctaggaagatcaaatgtcactataacacgagggtgtgagccgggcgccagtgcctgcagccggtgctgtccacagggagctccagcccttctcacactcgacccgcaggtgggta

SEQ ID NO: 39 - DNA, 380 bp, synthetic construct
  misc_feature 1..380: note = Dph-CRE39
  source 1..380: mol_type = other DNA
taactatgtttaatgtgctgagagaaagccatttgtttacagaatcttattatagaacagaaatatacttgttccagagaagaggagtttaattggttccaagtcaaatgatgctaacaggcgtcacctcaccaatccaggacttaagatgggttattataaataggagtaacacatattttttttggctctttttcccacctgttatgcaagactgcataccactatgatgttcctcctgattatttccagcaactttgggggtggctcacgtgtgctcatgaacaagtactgtcaggtatcacatacggtctgtcattttcttgctcttgagctctggcacttctctgctgctgtctgctgctgaggcctgcagagat

SEQ ID NO: 40 - DNA, 575 bp, synthetic construct
  misc_feature 1..575: note = Dph-CRE40
  source 1..575: mol_type = other DNA
ttctctctcactgtaaaagtgtaaaaggttaggttactccccagcctgtcgaacatgtgcctgattgccttctgagatagcacatccctaatccaaaagaaaacctagcctggagcttgcatccagctaatcttcttccacattgctccaaacagggagttcagttttctcattactcatacagagagcagagatagattttcatgattcttcaggactctgttctctctgctttaatagccaaaagctaaagccaaatgaacttcttcacagagaacaacaagcaattttatctaatccttattattcttgctgtaactgcaattgtgttattctgactgacagattcccagaagagattcagtgaaaatacccttgatcagattcacaaaggaacagcatatttcaagtgggcgtgtctggctagaatctccctggacagttcagtaacagcacatcaacagactgctgaattcttatttccccgtccaaaaaataacaaagccaatctgaaatctggaagtgttctgagggcagatggtattagcccagtagtaaacatatttttggctgct

SEQ ID NO: 41 - DNA, 463 bp, synthetic construct
  misc_feature 1..463: note = Dph-CRE41
  source 1..463: mol_type = other DNA
ctacaataaatatataacttttttaatttgacaaaaaataattttgagtggttacaaaaagggacaggtgtggcttggtaactagaaaaacattcccagcctgggaaacaatctagtgagagcttggaggcagaagacagaactctgattacattcaaggagtttgccagatggcaacagataactgccaatgttcggttgcactataattattatacactctctgtcacttcagcaagcgctcttttcacaagacaagtggtgacagaatgttgtattaagattacccgttgctaagcttatgttaaaatgaggaaatgaaatggaaagtcttgttttggtaatgtctctggggtatgaggaatggagggaaaggtttgactatgagcataactgcatagagaattttgtttgtttatgccttttgggaatgcattattttgattgaccctaattgggaatt

SEQ ID NO: 42 - DNA, 379 bp, synthetic construct
  misc_feature 1..379: note = Dph-CRE42
  source 1..379: mol_type = other DNA
attcatgaagatgtaaaagacacatttaaaacccacatcaattgcactaaaagtcaactggaaacagaaggtttttaaatcctggtggtggcaaattggcagccaaaggaacaaagtcttagtcaacatatttccatgtcccccgaacacaaatagcctcgaaccttcggagggtgttcctttgagatgtttcatcagtgacatcacagtgattgccagcttccacggactactttaggcgctgcccagagtccaggagtccagacagcctgggaggggagaaggagttggagctcaagttggagacagcgaggagaaacctgccatagccagggtgtgtctttgatcctcttcaggtaactgcaggatttttatgctt

SEQ ID NO: 43 - DNA, 250 bp, synthetic construct
  misc_feature 1..250: note = Dph-CRE43
  source 1..250: mol_type = other DNA
aagaaggaataccctagggcaaatgcgtgctagaggggcttaatacttggcagaatccttctttttcagtagcctgcttttatgactgctccgactgcacctgttaggaagataaataaaggactggccaattctttcctctgaagcgccctaaatatatcactttaaaaagactttggaaaaacaagttgtatccttacttctcacccagtaaatctaaagaaattccctcttgttcccaggcctttca

SEQ ID NO: 44 - DNA, 194 bp, synthetic construct
  misc_feature 1..194: note = Dph-CRE44
  source 1..194: mol_type = other DNA
ccacataactaaagcatgatgctattgagttctaagtctagtttccacccttatatgaaaagtcagcttggcacagagtttggtagagctaagtctgaccagctgcactctcagccacaagggaaattagacgagacagtgtggctgcttcagtaccaatcaatcaaagtttttgaaaattatcttgaaaacac

SEQ ID NO: 45 - DNA, 264 bp, synthetic construct
  misc_feature 1..264: note = Dph-CRE45
  source 1..264: mol_type = other DNA
actttctctgtggattgcaatatttcctggcatatgttcacttcagtaagacgatcagtgtattaaaaacccaatggggtttctgcagaagccaatgatggcatggcacctcttttgtggcatgtgtgaaatctgcccttcgctgtaaacattcttatgcaacatgacactctgccctatgtccatttcatatgtggtgttcagtgtgctgggaatgctgaaaaaaacaattctctcttagagatttcccaagtagaatcacag

SEQ ID NO: 46 - DNA, 427 bp, synthetic construct
  misc_feature 1..427: note = Dph-CRE46
  source 1..427: mol_type = other DNA
agaaggaataccctagggcaaatgcgtgctagaggggcttaatacttggcagaatccttctttttcagtagcctgcttttatgactgctccgactgcacctgttaggaagataaataaaggactggccaattctttcctctgaagcgccctaaatatatcactttaaaaagactttggaaaaacaagttgtatccttacttctcacccagtaaatctaaagaaattccctcttgttcccaggcctttcaaacagtgaagtcaggagtaagggagactgtggactggtcaaaggtggaagttagactcactagctggtctaaggaatccaggagggtggaaaacatgaagaacttacaatcctttatgctctcaatgactcactttattttggccatgagacacagccttgaaacttttttttttttt

SEQ ID NO: 47 - DNA, 436 bp, synthetic construct
  misc_feature 1..436: note = Dph-CRE47
  source 1..436: mol_type = other DNA
ggtcataaaatactttctctgtggattgcaatatttcctggcatatgttcacttcagtaagacgatcagtgtattaaaaacccaatggggtttctgcagaagccaatgatggcatggcacctcttttgtggcatgtgtgaaatctgcccttcgctgtaaacattcttatgcaacatgacactctgccctatgtccatttcatatgtggtgttcagtgtgctgggaatgctgaaaaaaacaattctctcttagagatttcccaagtagaatcacagtgttaaacctatcagcagcctccaagatcattttttttaagcccctcattttacacaaaaagaaactgaggcccagaaagccccctgactttctcagagtaactaagctggttaatgatagatccagaaatagaacccaagggaccaacttcagattctag

SEQ ID NO: 48 - DNA, 359 bp, synthetic construct
  misc_feature 1..359: note = Dph-CRE48
  source 1..359: mol_type = other DNA
ctgagtcagcacatgcatctgtcagagagagaagagcaagacggagcacgaggctgcagctgaatttagaaagttagaggtgttctcatcagccacatgagatgggcaggagattccttccagtaggcgagagagcagagtgcagacaacagatgtgagaaaggtccccatagctgtcaagttcctgcatccttgtgctgaagaaagggaggtgccatcatgattgatgttcagaggcagtataaaagcactgggtttctccccaactgcttgtcacaccgacctgcaccatctctcgcctgcctgtggggtttctgtcaactagtcgtggagggaaggagactctttaaagaataaca

SEQ ID NO: 49 - DNA, 353 bp, synthetic construct
  misc_feature 1..353: note = Dph-CRE49
  source 1..353: mol_type = other DNA
gagatttcatcacattttaaagttttgccaagccaaaaagctacctgaatgtagagcagcattcatcccctcttgcctatttctaagcatttctcagggcttagtcatccatcagaaaaggacctcccttgatgggaaaactccttctcatatctcttctttgaccttcagtttctacctgcaagagagtgaatggcctatttcaaacaccgaaatcaatttgtctttggaaagtcatcttatatacaaatattatccttacctcaaattggggaaaggttaataatttatgcagaaaactaagagaaaggaataggggaggaggaaagatagtttatcaagggcaattgttt

SEQ ID NO: 50 - DNA, 354 bp, synthetic construct
  misc_feature 1..354: note = Dph-CRE50
  source 1..354: mol_type = other DNA
tcaaaagtcttttaaacatattttctttatataattaagtaaccagtgttgacaggctgtggtgatcagtttacaggttggccttacttagtggcagagggcattgatggatgaagtctttcacccaaaataaccacaccaaatgcatgtattaatatgggaacttaatcctgactgtcaggcagggagtccaacccctgctaaagtatcttatttgtaatgcaaatgatttacgtgtgtactaagtgtcctttttatctgctctgtcactttaacctctccgaagtgtacccagaagcaatacattctaccatgtttccaaactggaaacacaatattgttttaattattctg

SEQ ID NO: 51 - DNA, 351 bp, synthetic construct
  misc_feature 1..351: note = Dph-CRE51
  source 1..351: mol_type = other DNA
gctgtcccagcgttatcagtcgggcgccttgccagccgaaagggcctgtctaaattcgtttcctgtcccctaactcatcccggcgctggctggcctggagagggtaggatggggcggcgccgagaatggccgttatgaggaccctaagaggtgagaccctctcgccttctggggtggggggtcccgtcctttcccccactgaggacagaggcccgcccagcgatctgagcatgtgtggacgtcaatcttgcagcccctcttccaggccccctccccagccttgcagggctcaggttacccctggcctttcctaaaggtcactcattcctcttgacgtttgcaaaaggggaa

SEQ ID NO: 52 - DNA, 607 bp, synthetic construct
  misc_feature 1..607: note = Dph-CRE52
  source 1..607: mol_type = other DNA
tgtcccagcgttatcagtcgggcgccttgccagccgaaagggcctgtctaaattcgtttcctgtcccctaactcatcccggcgctggctggcctggagagggtaggatggggcggcgccgagaatggccgttatgaggaccctaagaggtgagaccctctcgccttctggggtggggggtcccgtcctttcccccactgaggacagaggcccgcccagcgatctgagcatgtgtggacgtcaatcttgcagcccctcttccaggccccctccccagccttgcagggctcaggttacccctggcctttcctaaaggtcactcattcctcttgacgtttgcaaaaggggaatgtaatcctggggtggggggagacccctcatctgtagcccctcccttgctcctcccaaagggtggaattagaacagggactgttattgggagacagaaagtgggggatagtagttgacctttggtaagggggcaggtgcccagggccagaggcttctgcttcaggctgtagtgggcacttggctgccagcccagtgtgaaggggggaggatggagagaaagagaggcggggctggctggggaccgagtggctcaggga

SEQ ID NO: 53 - DNA, 469 bp, synthetic construct
  misc_feature 1..469: note = Dph-CRE53
  source 1..469: mol_type = other DNA
agggctgggcggcttccggagcctgggctaggggcaaggggtcaccgagcgcggcccgggcggaaagggggttggttttctctctcctcccgagcgcggcgcgcatccctggcgtccacgccgaatcccacagtccccgacgcccctcgagtccgtgttcctcgacagccgcgcggctgagtcactggcgggctcgggcggggccgcacccgggcacgtggcggcgctccccgcccgccatctcctgacccctggcccaccgcacccctaccccagggtggaaaaatcccgggaggagcggcctgagatgagggggcggggcgagaggggagactggacgggtggcggggcaggtggcctggggtgggggctgggaggccgcgcgggccggcggggcggggagcaggggtggggagagggcggcgggggtgagtcaccgggcgcgcgctgccccggcgccgacgggaag

SEQ ID NO: 54 - DNA, 332 bp, synthetic construct
  misc_feature 1..332: note = Dph-CRE54
  source 1..332: mol_type = other DNA
tgggttcaccagggcccattctcagtctagaggattgtcccttctctggatcggctctagcccagggcctcaccccacatcccagccccgcccgccccaggcaggcaatgtctggatcaccggccgtgccccttggcctgtgtctgcagaggtcacagagcgcggacacccgcggggcttaacaggcggggctatttttcgaggcggaagaatactcaagttgcaagagtggcaactttttcaagaaggggaaagtcccacttcgggccctttgggtttcttcgcagtcagatgtgcgctcacagccacctggggagggcgggacgggcggg

SEQ ID NO: 55 - DNA, 206 bp, synthetic construct
  misc_feature 1..206: note = Dph-CRE55
  source 1..206: mol_type = other DNA
tacattccagggagggagaccagttgggacaaggcctaggctgctctcctacaaatttgccatgaacaggagccaaactctaagacctgattcaagtggcccagctgtcactggtgacacactcaggggctgggcctatcatgttcttatacaacctagtgagtcaagctttggaaaataacattgtcagggaagaacgtaaaagt

SEQ ID NO: 56 - DNA, 289 bp, synthetic construct
  misc_feature 1..289: note = Dph-CRE56
  source 1..289: mol_type = other DNA
ggagctgagagtttaagggcatttgtttactatgtttgcttggattttccgaactgccttagttcctgattccacactgctattgtgtggatcaaataatcccttgtcacaaaaataccctggggaatgactcccctggcgtccaggttatgagtgtggtcagtcattccacatgccttagggatgagctatctgtgccatgacgagcgatttccttttcgagttttgtaatccccaaatactaaatactattatcaataaaagttaattaaggaaatgtatatcgaaa

SEQ ID NO: 57 - DNA, 195 bp, synthetic construct
  misc_feature 1..195: note = Dph-CRE57
  source 1..195: mol_type = other DNA
gacagctgtcccgctcttggaattcattggcttcctctacccggcctcccaaacaccaccccaatctagtttagccccccgccccaccctcgctgacctaataaggccatgcagtgtgcgggggagctacataaaagcgcgggctcgcggcgactctgcaccacgcaggggaagagaaagcaggagccgtccagc

SEQ ID NO: 58 - DNA, 345 bp, synthetic construct
  misc_feature 1..345: note = Dph-CRE58
  source 1..345: mol_type = other DNA
cccctcagcttcagcccccacctccaggaggccctacccacgctcatgaccttgctattctgggccttgtgtcctgtagggagatggacaggagacagctgggcttccaggccacccaggcggggggctagccgagggaagcctgctggctctcctgcttgctctaatttctggggctccccaaaccttggcctcaggagactggggataggaccggccttgaaagtgggggaagctttggagagccgggtgctgggttcttagtgagatggccagtgaaggctgtggtgccccgaggtaagcagggcctgatcccctcctaatcttccagcagcaactggtgct

SEQ ID NO: 59 - DNA, 220 bp, synthetic construct
  misc_feature 1..220: note = Dph-CRE59
  source 1..220: mol_type = other DNA
ctcctccagcagctgccctggtggtaaccaagagacgcccccatcctggagcaggggtggggaggggcagctcagagcagctgcttctctgaggaagctgacaccaaggccagcattcagcaacaacttgtggctttgcacccagcgccggggtccccgcccacctggctccctgctgtccctcttccccactgctgctcggacttccctctgaccctgg

SEQ ID NO: 60 - DNA, 663 bp, synthetic construct
  misc_feature 1..663: note = Dph-CRE60
  source 1..663: mol_type = other DNA
ctttgctccctgggagggcctggccctgtgggcatttgagtttataacaccacccccattgtggcacacccctccaccccgtaaaacacaggctctgctcttggaatcagtcttcctgatctgtggctgtgccctccaacagagggcacccctgggcttcccagctctgggggtagtgggtgccaacaaggaggggcctggggctgaagaatcccacccgctgagctcggccttctcccttccccactgtccagctccgcctttcagcatcctgcctcactccccgcccaggcagcaaggagcccacaccctcatgcccctcagcttcagcccccacctccaggaggccctacccacgctcatgaccttgctattctgggccttgtgtcctgtagggagatggacaggagacagctgggcttccaggccacccaggcggggggctagccgagggaagcctgctggctctcctgcttgctctaatttctggggctccccaaaccttggcctcaggagactggggataggaccggccttgaaagtgggggaagctttggagagccgggtgctgggttcttagtgagatggccagtgaaggctgtggtgccccgaggtaagcagggcctgatcccctcctaatcttccagcagcaactggtgctct

SEQ ID NO: 61 - DNA, 594 bp, synthetic construct
  misc_feature 1..594: note = Dph-CRE61
  source 1..594: mol_type = other DNA
cagcagctgccctggtggtaaccaagagacgcccccatcctggagcaggggtggggaggggcagctcagagcagctgcttctctgaggaagctgacaccaaggccagcattcagcaacaacttgtggctttgcacccagcgccggggtccccgcccacctggctccctgctgtccctcttccccactgctgctcggacttccctctgaccctggtggctctgtgtctctgctccctttccccctaggtctagacatctgtccttatttcccccagacctgtccccagaagtccacccttccccattcctttggtctggagcccctgcttggtccagcttccccaggccccgacacctttctgtggggtctgcctagctcctgcacgcacacagcatgggcctgatcctgttcccctcgtggacagatgcagcagggcagagtgcagcgcagaccacaggcctctggggctggccacagaaaccccgttggttagagcacagtgtgggatgaggtgaccctcagtgcacgacttggggtgacccctgcccccatcctgagacagttacccctccccctctgccatcagcacattc

SEQ ID NO: 62 - DNA, 634 bp, synthetic construct
  misc_feature 1..634: note = Dph-CRE62
  source 1..634: mol_type = other DNA
ccttccccattcctttggtctggagcccctgcttggtccagcttccccaggccccgacacctttctgtggggtctgcctagctcctgcacgcacacagcatgggcctgatcctgttcccctcgtggacagatgcagcagggcagagtgcagcgcagaccacaggcctctggggctggccacagaaaccccgttggttagagcacagtgtgggatgaggtgaccctcagtgcacgacttggggtgacccctgcccccatcctgagacagttacccctccccctctgccatcagcacattctgtagcctcttgggttacttggctgccttggtgtcccattttcttgggggtggggtggggattccctatccaggatgggggggccctcagggctctgttcccagaggctgagttagagcgatggggaagggggggggcagttttggggagagacaggcagtgctggctttgctcaccagggcctggacactaaatcccttgttgatggctgtggcaacccctccctagggtagggttaccatcttcggccctgtccccttgactctctcccctcacttccccttgtccctctaggagccactcacttcctctagcccccaaaagatgttctccct

SEQ ID NO: 63 - DNA, 445 bp, synthetic construct
  misc_feature 1..445: note = Dph-CRE63
  source 1..445: mol_type = other DNA
cctcccctgcaccccagaggcaggttttattttaagctttaagggtgttctcagccaaaacaccgaagctaagccaccctcgcggcttcaagagcttggagagctcgggttacccacccgaactccgggctccgggtcccgccgcgatgccggctgcggcgcggggggccactgccactcccggcatgcgccgggcggacggccgctccaccaatccccgcgcccgtcggcgcccctgccccgccctccccagcctcctgacgctgattggtcgaggggaggactcgctcctagtggcgggaaagcgcggcggtgtgatgatgactccaaggagcccggcgcccggtcagggagggcactggcatccctcattacccgcccagcctggccttagcccttccccgcgctccctaggcacccccacccccgcagggcatctccaggg

SEQ ID NO: 64 - DNA, 408 bp, synthetic construct
  misc_feature 1..408: note = Dph-CRE64
  source 1..408: mol_type = other DNA
gtctccgaacgcaggccccgtcgcgttaagcacaagctggcagggcctctcctctcccttctcagatttgctccttgacatttgcctgctgcctggcggtggcaacagctggggcggggcgcgcgcaggaggccccgtaaccctatccccgctccggctccctcgtgaaaccggagcttccctgccttggccgagggggagggctgcgggggccagaccgcctgcgaagaccacagggtttttcctctcgggttttggctcccgtgggatggatgtggctgtgcggggggttggcctgagcttcgcttctaagccagcagcttggtcagggaaacctgaaagcattcccagctaatcccccaagtggtgcaagtctgtgcgcgcccatcccgctgagtaaggcggtgg

SEQ ID NO: 65 - DNA, 496 bp, synthetic construct
  misc_feature 1..496: note = Dph-CRE65
  source 1..496: mol_type = other DNA
ggcaacaaagtccagggattccagaacttagctgtctttgaaacaacaccttacattccaaaggccaacaactttaagattgccaacgactcaaattatttataggagattgattcatatacttaacccaatatgagaggaaatatttctaattatatccaaacatctttaatatgaggggaattaggcttggagaccttcaatgccaaaaacatgttttgccaaatatgttttaagaaagaggaggaggttacgtgttttccatatttgagcctataaaagtacccttggaatgagtatgacttgtctctcctcataaagcttcaaggtaagtgtgtgtgaggacagggctctgtctggaggacggtaagggatgtctggctgcccgcgtgcatgccacacatcctggctcccactgtccctggcagccctgccagtgaagcaccagcttactccgtagcaattttaattagatgttgtagtcttcccagtcatt

SEQ ID NO: 66 - DNA, 319 bp, synthetic construct
  misc_feature 1..319: note = Dph-CRE66
  source 1..319: mol_type = other DNA
ccaggtgacaggtggatggtggagctggaggagccactcggagcctccaaggctggggagggtggaggggggaggcaggtcggtgtcccaggccacacctgttgactaagggattaggatgttgtgtccctgccagccctcctccaataagcccctctgggcctgcagggagaggaggagccttgccatgtaaactgtatttttagttccctgtgcctctccccggctgctataagacacctctccccacccccagccctggccgcttggctggaggctctgcgaggacagctggggagaaggggagctgtggtcagta

SEQ ID NO: 67 - DNA, 372 bp, synthetic construct
  misc_feature 1..372: note = Dph-CRE67
  source 1..372: mol_type = other DNA
tcatcttctaataatttggcaagccatccatggtcttctaagcaaggcgccatgaatataacgaatatagagacagaaaaagacccctccccactggctgggttgagggactggagagcccagatggaagggcagaggtgcaggcttttttccttgttgccactagatggcagtagggcacccgttgtcagccctggggcaaggtcaccgactgtctttgcctttgcctccagcaagccaaaaccctgggcagactcaatccaaaaataaacaatcaaagagcatgttggcctggtcctttgctaggtactgtagagcaggtgagagagtgagggggaaggactccaaattagaccagttcttagccatgaa

SEQ ID NO: 68 - DNA, 293 bp, synthetic construct
  misc_feature 1..293: note = Dph-CRE68
  source 1..293: mol_type = other DNA
agaaaggagctgccctggggatggctgtgtatccctacaactgccaggcacatgccccttgaacacccgatgctacgtgtcccaaaggaaactggtctccacccacccccggcccgtcctcgtcctgggtaccccaccttagtaaatggcgccaccatctgcccggtcactcagcgagaaactcaactcctggcagcagatgacgggcactctggttaaatgactctctccagcctccaagttcaacctgcagggaagccctggaaatcctgtctccctctgccctgcctctc

SEQ ID NO: 69 - DNA, 482 bp, synthetic construct
  misc_feature 1..482: note = Dph-CRE69
  source 1..482: mol_type = other DNA
gaaacatggcttccacaggttccagttgaagaatcccagttccgtctataaattccagggaaggtctctgattggccctgctcattcccaggcccattccttgacccagtcactgaagtcagggagatgcagtaataagactggctggaatcagggtctttaggggtggagggatggggaggaggcacagcatgtcatcaaaataaggaaattgcaaaagaaagcttgcaggctactttgaatgacaatgagaaagacggtgctgcctgagtgtgttaaggatccacatggtctccaaaatcctccaggagcatacagtctagtctgggagatgagacacaaaaataaccagaacacaacagcttgcactgactcgagggctggataagaatatctggaactcccccatctatttcagaagcttgtctcttggatgaaaattagacacttaatgggaaagggctttgaaaagagtgcagtaa

SEQ ID NO: 70 - DNA, 398 bp, synthetic construct
  misc_feature 1..398: note = Dph-CRE70
  source 1..398: mol_type = other DNA
cctcaggtaccccctgccccccacagctcctctcctgtgccttgtttcccagccatgcgttctcctctataaatacccgctctggtatttggggttggcagctgttgctgccagggagatggttgggttgacatgcggctcctgacaaaacacaaacccctggtgtgtgtgggcgtgggtggtgtgagtagggggatgaatcagggagggggcgggggacccagggggcaggagccacacaaagtctgtgcgggggtgggagcgcacatagcaattggaaactgaaagcttatcagaccctttctggaaatcagcccactgtttataaacttgaggccccaccctcgacagtaccggggaggaagagggcctgcactagtccagagggaaactgaggc

SEQ ID NO: 71 - DNA, 241 bp, synthetic construct
  misc_feature 1..241: note = Dph-CRE71
  source 1..241: mol_type = other DNA
caggggcgcaggcctcttgcgggggagctggcctccccgcccccacggccacgggccgccctttcctggcaggacagcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgt

SEQ ID NO: 72 - DNA, 238 bp, synthetic construct
  misc_feature 1..238: note = Dph-CRE72
  source 1..238: mol_type = other DNA
cccccaggcagcggcacggtggactttgatgagttcctggtcatgatggttcggtgcatgaaggacgacagcaaagggaaatctgaggaggagctgtctgacctcttccgcatgtttgacaagtgagcacgtgacccttgacctctgaccctgacccacactcaagccgagctgtacaggagggcagtctcagattccaggcctagggaccctgtggcctctgcctgataggggagag

SEQ ID NO: 73 - DNA, 465 bp, synthetic construct
  misc_feature 1..465: note = Dph-CRE73
  source 1..465: mol_type = other DNA
agccagctctgggggcacgcctgcttatcctgtgggagtccatggagccggggttgggacagccctccacccagtgcccatacaaggcctggcggagttggggactaattttggcttctgaggcggcactagcaggccagggggccagataacgctgccccaccccctgcatgccaaagtccccagaacaatcaccaggtttaactttgttcctcgttaaaaatagcccagtggccaccctggtcaggttaccgtgggtggcttgcctgcctccacactggttttattatcccaacttgagggacagctgtccttcgggccacccagcttgagtttcatcaggggccgaaagggcattgagtggtcactgactattgttactgagggtcaccttggtcctgaagggggtgcccacctgtcaccctggccctgagcccagtcgcagtgaggccagctgggtcacgt

SEQ ID NO: 74 - DNA, 285 bp, synthetic construct
  misc_feature 1..285: note = Dph-CRE74
  source 1..285: mol_type = other DNA
ttttcattcattccagaaaccttttcagagagtccctttggggagtgtgggggacaggagggaaagaaacctggtccttgtagccgttcgtctgctccctgccctgggcagaggacgtggggactcaggccagcctgagatcactgggaccagaggaggggctggaggatactacacgcaggggtgggctgggctgggctgggctgggccaggaatgcagcggggcagggctatttaagtcaagggccggctggcaaccccagcaagctgtcctgtgagccgcca

SEQ ID NO: 75 - DNA, 332 bp, synthetic construct
  misc_feature 1..332: note = Dph-CRE75
  source 1..332: mol_type = other DNA
tgctctgtttacagacaagctgctgtcctccctgcaaaggggagtgggtggggcagagggcaagtgccaggggggcacaaggctgggcatgtggctggcatgagacggtgtctgagtaatgtcaggcacctggaggcattgaccccaggaccttggaccccagacctctgaccggggggcagccagcgtccaggtaccccaacccctgccctgggtccggcgtccccccattagtgagtcttggctctacttatagcatctgacaccagaggggccgaaaatagcccctggagaagggggaggagggggctatttaaagggcctgggagggg

SEQ ID NO: 76 - DNA, 381 bp, synthetic construct
  misc_feature 1..381: note = Dph-CRE76
  source 1..381: mol_type = other DNA
gcctgtggtcttggtggtcgtggtcagttccctctcctgccagctgtggaatgtgaggcctggcctgggagatatttttgctgcactttgagccaccccgccccctggaactcagaccctgcacagtccatgccataacaatgacgaccacttccaattgtttcctagctggagaggcggggaggggagcactgtttgggaagggggggagcctgggggaaatgcttctagtgacaacagccctttctaaatccggctagggactgggtgccgttgggggtgggggtgccctgctgccccatatatacagcccctgagaccaggtctggctccacagctctgtcctgctctgtgtctttccctgctgctctcaggtaggag

SEQ ID NO: 77 - DNA, 316 bp, synthetic construct
  misc_feature 1..316: note = Dph-CRE77
  source 1..316: mol_type = other DNA
ggaataccctaccttaaaaacaaaacaaaccgtgctacttggccctcttcccttgagttgttgtctaatctcattcatttcactgccaaactcctcaaatgcatggtgtacacccactgcttccacttcctgtccactcatctttttcctcattaactccctgtggcctggctttgcctgtgacactacacaagctgcttgctccaagataatccaagttcttttctctgtcttcctgatacacaggctttctttggcatctgacactgctggccacctgctcttcttgaaatgcttcttttggcttctaggaaac

SEQ ID NO: 78 - DNA, 401 bp, synthetic construct
  misc_feature 1..401: note = Dph-CRE78
  source 1..401: mol_type = other DNA
ccaagtggtgggtgtagcctgggggttcaatcctcctgtggcgccccagaacccggtgcctcctccaacgtccggcatctgatgaggatccgacccaggcgggcggcggcgggattcgctcttccccttcgctccccgcgagaaagccccgagggccgcggcggcgcagagccggtgacagttgaagcttaggcgggaagagggaggcgcgaggcgggaagagggagtttgggcctcggcagccgccgtacaaacaccgctctggtcaccatggcaacagcgggatgccgcgaacggcttctgggcggggccggtccctcggacgattggacctagcttggcgcggaatccgtgaattgcccgcggcccgagggtgcaggtgatgggtgctgaccgactgg

SEQ ID NO: 79 - DNA, 281 bp, synthetic construct
  misc_feature 1..281: note = Dph-CRE79
  source 1..281: mol_type = other DNA
gactacaacctgccagctcaggacgagagctgtcaggaagagtccaggaatggacttcccacgggagggcacatttctggtattcctggcaagataaggagttgactaagtaatccacgagaaaaggcatttccggcagaggaaacagtctgggggtgagagggaggctgcagcatttggggaactgctagggctatcgtgtgtttggaagagggggagggagagaggtaggcagggctaaattgggaattttgtcactgacataaattttaagtgccagg

SEQ ID NO: 80 - DNA, 393 bp, synthetic construct
  misc_feature 1..393: note = Dph-CRE80
  source 1..393: mol_type = other DNA
cgttttgtggtaccagggggtccctcctctcctgtccccagccaaaccttttcctttcccctcgggaaagctgccttggctgtcactacctgctgcctattccacatcctgaaccctgtgacctaggcccagggctgctgcgcggacggtagctccccctgcaggaagcaaggttcctccgggcccccagactgctgctggacctgtgcagaagcctgcaactttcctctgcctagcccggcccacttcctggatgcttgctgcccccagcccaccagagctgtgagttccattcctaccccctgccccactgagccctgatctaggtatgatcggtgcattcatttttttgctcaacaacatttattactgagcaccttctcaaggccaggc

SEQ ID NO: 81 - DNA, 169 bp, synthetic construct
  misc_feature 1..169: note = Dph-CRE81
  source 1..169: mol_type = other DNA
cctaggcccagggctgctgcgcggacggtagctccccctgcaggaagcaaggttcctccgggcccccagactgctgctggacctgtgcagaagcctgcaactttcctctgcctagcccggcccacttcctggatgcttgctgcccccagcccaccagagctgtgagttc

SEQ ID NO: 82 - DNA, 390 bp, synthetic construct
  misc_feature 1..390: note = Dph-CRE82
  source 1..390: mol_type = other DNA
gaagccaggtatcctggctgcccagccactgcctttatccaccagcttgactctttcagatcatcttctcaaattatccataggagatttatccacatactcaataagaaaaatatttctaattatatccaaccattcttaatatgaatgagaattatgcggggacgctagattgccaagaggtattttgccaaacaattccttttgacttaagaaagaagaggcagctgcattgtttccatagctatccatataaaagagcccttggaatgaggctgactcgtcctgctttaaaaagctccaaggtaagtgggagcaggacgggcctttcaagagggacactggtcacaccgcccagtgtcagcagcagctgctagttctggtactgct

SEQ ID NO: 83 - DNA, 294 bp, synthetic construct
  misc_feature 1..294: note = Dph-CRE83
  source 1..294: mol_type = other DNA
cagctgctagttctggtactgctccttctggaactgcacgttttacctctatgaattttgttacttctgtttacaatagcaagccatgccataaatgggattttttccccctctttttggtcaactaatcaagtgcttttcccttttcttttatagaactgtctcactcccaggctacatcttctcacttgctaacaaggtaagatttggactaaccagttcctggaggagaatgcaagaggctctgggatggatttctgtcacttagcaacctttcagaaagtgcttgtctca

SEQ ID NO: 84 - DNA, 460 bp, synthetic construct
  misc_feature 1..460: note = Dph-CRE84
  source 1..460: mol_type = other DNA
taaagacctttctcagagatccaaaccagccccaccccccgcccccaggaagcatagcatattttctgtgaggccttgatggtagcatcacaaacccttgggaaacacaactcccagtgtttgaggagagcatgccgtatcttgttccagggacactgagtcgtgagcataaaacgctgcattccaaaagagacgaagaagcagtcgtctctccatttaattatagattcttcactttcccttaattgcttcagtcagcactgttgactctgggggagtcacagtacaccggcagggctattgctgttaaacaaggggtgactatcaggtaatgaggttttcattttgttttttcaaacaaacaaaccctgatgtacatattcaagtgggcattcctgttaaaggtgtcacattgggaaatgatgctcatgttgactctcctttgtaaccaaatattgat

SEQ ID NO: 85 - DNA, 292 bp, synthetic construct
  misc_feature 1..292: note = Dph-CRE85
  source 1..292: mol_type = other DNA
taggaaggaacaagaagaggtagaaatgaaatggctcccaaaagaaaaacctcccttgggtggtattttaggacattagctcaggctgccttgtcctcagtcatctcagtggcatttaagtagccccttgggttacagatcacggcaggtgctgggaagtgaagaaggccatgctaaaaatactggcctcttctggaactctgccggcagccctgatggagctccctcctcaagcagcagtacctcagcaaaagaacagtttcctccgccttaagcagtaagaaaagtctgg

SEQ ID NO: 86 - DNA, 472 bp, synthetic construct
  misc_feature 1..472: note = Dph-CRE86
  source 1..472: mol_type = other DNA
aagaaagggtctttacaaaaccaaaagaaagcccattgcttacttttaataacctaggcccacccataggcctctaaatcaaataacctccactgagaattaagctgttagcactaggtctggcataggtgcagaaacaaaagtttagaaatcctaatcttgaatttacctaacacggctgcctcctagagcctgagtgggttgtagcgagacctcaagtagaggagaggcacctgggccatccgcctccatgggcttttttttttttgcatcgaatttcatggtctgcaaagtaagggtggggctttacttgccattcaggaggttgcaaaggtcacctccacatatgttcctgttaaggacatcagcagaaacttgagaagcaagtataaaaatataaaaatgagcaggtggcattcaataggaaaagaatgcagggtttgctgggaagtcatgattgaaaccagtccaa

SEQ ID NO: 87 - DNA, 476 bp, synthetic construct
  misc_feature 1..476: note = Dph-CRE87
  source 1..476: mol_type = other DNA
aaggaagtgaccaggccaggtctggatccctggaactgactcctaactccctattcttgcctcctctgctgagctccaacctggggagtcagctccctggagtccacgcatctggatatcgtccacatgcctggaactttgtcactgttccggtggctgggttgacacaccgtgattaaagggctctggtcaatttctgtgccacgcagcccctatgctaatggtcagatctaatgttctgaccattaggtcagtgtatcttttccctcctgggcagttaggttagtggaagaaacccgataaaatcttggagaggaatttgatcattctctgaaggacttacaagttgtttggcctgcctaatctgctcagataccgtccggggaatatttggtaaccatgcagtacagactgtgaattattctgtggggactattaacaagaccctcaccaaccctgcctcagctgatctcagg

SEQ ID NO: 88 - DNA, 459 bp, synthetic construct
  misc_feature 1..459: note = Dph-CRE88
  source 1..459: mol_type = other DNA
acagaccaaattgcctgagtctaaactcaatgttctttccccttagactgccacccgcacacacacgcacacatgcacacctttttggaggcactgcacagcaagcacagaagtacgccactgagtcctgtgtccctaaagtatttgactgtccctccgctgggggaaagggcaggcagaaaacaagactccgtgtgctgtcgctgtgccgccccctgcctctctgacccgcgcccgcagagaaagtctcaagagccgccccaggctttctcccacgttctccctttctctgctgcagttgagtttccagagcgtgagcgcgcaggatgacacctggctggctgagagctgccggggaggcgctggcgggtgccgagagcgcactgaccctgacgcggggtgcagcacggctgggaagcccccgggcctttggctaagcgcgccgggggacggcacagg

SEQ ID NO: 89 - DNA, 378 bp, synthetic construct
  misc_feature 1..378: note = Dph-CRE89
  source 1..378: mol_type = other DNA
gccccaacgccctctccctggcgcgcaggtttagaaacagggcggcctctccggccgccgcctcggcggctcgggtccccatatatagtcatatccaccgtcaactgggaggccggcggccggcagcgaatgggcgagcggcccccgcgggaggagcggggagggggcacggggcggagggaggagaggaggaaggggggcaggagaaaaaagcttttccaaaaaagtattggctgtcttgaggaatgcggtcgcccccttgggaaagtacatatctgggagaagcaggcggctccgcgctcgcactcccgctcctccgcccgaccgcgcgctcgccccgccgctcctgctgcagccccagggcccctcgccgccgcc

SEQ ID NO: 90 - DNA, 983 bp, synthetic construct
  misc_feature 1..983: note = mouse desmin promotor
  source 1..983: mol_type = other DNA
accttgcttcctagctgggcctttccttctcctctataaataccagctctggtatttcgccttggcagctgttgctgctagggagacggctggcttgacatgcatctcctgacaaaacacaaacccgtggtgtgagtgggtgtgggcggtgtgagtagggggatgaatcagagagggggcgagggagacaggggcgcaggagtcaggcaaaggcgatgcgggggtgcgactacacgcagttggaaacagtcgtcagaagattctggaaactatcttgctggctataaacttgagggaagcagaaggccaacattcctcccaagggaaactgaggctcagagttaaaacccaggtatcagtgatatgcatgtgccccggccagggtcactctctgactaaccggtacctaccctacaggcctacctagagactcttttgaaaggatggtagagacctgtccgggctttgcccacagtcgttggaaacctcagcattttctaggcaacttgtgcgaataaaacacttcgggggtccttcttgttcattccaataacctaaaacctctcctcggagaaaatagggggcctcaaacaaacgaaattctctagcccgctttccccaggataaggcaggcatccaaatggaaaaaaaggggccggccgggggtctcctgtcagctccttgccctgtgaaacccagcaggcctgcctgtcttctgtcctcttggggctgtccaggggcgcaggcctcttgcgggggagctggcctccccgccccctcgcctgtggccgcccttttcctggcaggacagagggatcctgcagctgtcaggggaggggcgccggggggtgatgtcaggagggctacaaatagtgcagacagctaaggggctccgtcacccatcttcacatccactccagccggctgcccgcccgctgcctcctctgtgcgtccgcccagccagcctcgtccacgccgccacc

SEQ ID NO: 91 - DNA, 1099 bp, synthetic construct
  misc_feature 1..1099: note = human 1.0kb desmin promotor
  source 1..1099: mol_type = other DNA
tgtacaacgcgttacgcctcaggtaccccctgccccccacagctcctctcctgtgccttgtttcccagccatgcgttctcctctataaatacccgctctggtatttggggttggcagctgttgctgccagggagatggttgggttgacatgcggctcctgacaaaacacaaacccctggtgtgtgtgggcgtgggtggtgtgagtagggggatgaatcagggagggggcgggggacccagggggcaggagccacacaaagtctgtgcgggggtgggagcgcacatagcaattggaaactgaaagcttatcagaccctttctggaaatcagcccactgtttataaacttgaggccccaccctcgacagtaccggggaggaagagggcctgcactagtccagagggaaactgaggctcagggctagctcgcccatagacatacatggcaggcaggctttggccaggatccctccgcctgccaggcgtctccctgccctcccttcctgcctagagacccccaccctcaagcctggctggtctttgcctgagacccaaacctcttcgacttcaagagaatatttaggaacaaggtggtttagggcctttcctgggaacaggccttgaccctttaagaaatgacccaaagtctctccttgaccaaaaaggggaccctcaaactaaagggaagcctctcttctgctgtctcccctgaccccactcccccccaccccaggacgaggagataaccagggctgaaagaggcccgcctgggggctgcagacatgcttgctgcctgccctggcgaaggattggcaggcttgcccgtcacaggacccccgctggctgactcaggggcgcaggcctcttgcgggggagctggcctccccgcccccacggccacgggccgccctttcctggcaggacagcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcacctctaga

SEQ ID NO: 92 - DNA, 1426 bp, synthetic construct
  misc_feature 1..1426: note = human 1.4kb desmin promotor
  source 1..1426: mol_type = other DNA
acacacctactagtaacccctccagctggtgatggcaggtctagggtaggaccagtgactggctcctaatcgagcactctattttcagggtttgcattccaaaagggtcaggtccaagagggacctggagtgccaagtggaggtgtagaggcacggccagtacccatggagaatggtggatgtccttaggggttagcaagtgccgtgtgctaaggagggggctttggaggttgggcaggccctctgtggggctccatttttgtgggggtgggggctggagcattatagggggtgggaagtgattggggctgtcaccctagccttccttatctgacgcccacccatgcctcctcaggtaccccctgccccccacagctcctctcctgtgccttgtttcccagccatgcgttctcctctataaatacccgctctggtatttggggttggcagctgttgctgccagggagatggttgggttgacatgcggctcctgacaaaacacaaacccctggtgtgtgtgggcgtgggtggtgtgagtagggggatgaatcagggagggggcgggggacccagggggcaggagccacacaaagtctgtgcgggggtgggagcgcacatagcaattggaaactgaaagcttatcagaccctttctggaaatcagcccactgtttataaacttgaggccccaccctcgacagtaccggggaggaagagggcctgcactagtccagagggaaactgaggctcagggctagctcgcccatagacatacatggcaggcaggctttggccaggatccctccgcctgccaggcgtctccctgccctcccttcctgcctagagacccccaccctcaagcctggctggtctttgcctgagacccaaacctcttcgacttcaagagaatatttaggaacaaggtggtttagggcctttcctgggaacaggccttgaccctttaagaaatgacccaaagtctctccttgaccaaaaaggggaccctcaaactaaagggaagcctctcttctgctgtctcccctgaccccactcccccccaccccaggacgaggagataaccagggctgaaagaggcccgcctgggggctgcagacatgcttgctgcctgccctggcgaaggattggcaggcttgcccgtcacaggacccccgctggctgactcaggggcgcaggcctcttgcgggggagctggcctccccgcccccacggccacgggccgccctttcctggcaggacagcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcacc

SEQ ID NO: 93 - DNA, 2859 bp, synthetic construct
  misc_feature 1..2859: note = human GAA sequence
  source 1..2859: mol_type = other DNA
atgggagtgaggcacccgccctgctcccaccggctcctggccgtctgcgccctcgtgtccttggcaaccgctgcactcctggggcacatcctactccatgatttcctgctggttccccgagagctgagtggctcctccccagtcctggaggagactcacccagctcaccagcagggagccagcagaccagggccccgggatgcccaggcacaccccggccgtcccagagcagtgcccacacagtgcgacgtcccccccaacagccgcttcgattgcgcccctgacaaggccatcacccaggaacagtgcgaggcccgcggctgttgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcatgtccacagccgggcaccgtccccactctacagcgtggagttctccgaggagcccttcggggtgatcgtgcgccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccagtggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcaggggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccagtagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggggaggcccgaggggagctgttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgttag

SEQ ID NO: 94 - DNA, 2859 bp, synthetic construct
  misc_feature 1..2859: note = human GAAco sequence
  source 1..2859: mol_type = other DNA
atgggcgtcagacatcctccatgttctcacagactgctggccgtgtgtgctctggtgtctcttgctacagctgccctgctgggacatatcctgctgcacgattttctgctggtgcccagagagctgtctggcagctctcctgtgctggaagaaacacaccctgcacatcagcagggcgcctctagacctggacctagagatgctcaagcccatcctggcagacctagagccgtgcctacacagtgtgacgtgccacctaacagcagattcgactgcgcccctgacaaggccatcacacaagagcagtgtgaagccagaggctgctgctacattcctgccaaacaaggactgcagggcgctcagatgggacagccttggtgcttcttcccaccatcttaccccagctacaagctggaaaacctgagcagcagcgagatgggctacaccgccacactgaccagaaccacacctacattcttcccaaaggacatcctgacactgcggctggacgtgatgatggaaaccgagaaccggctgcacttcaccatcaaggaccccgccaatagaagatacgaggtgcccctggaaacccctcacgtgcactctagagccccatctccactgtacagcgtggaattcagcgaggaaccctttggcgtgatcgtgcggagacagctggatggcagagtgctgctgaataccacagtggcccctctgttcttcgccgaccagtttctgcagctgagcacaagcctgcctagccagtatatcacaggcctggccgaacacctgtctccactgatgctgagcaccagctggaccagaatcaccctgtggaacagagatctggcccctacacctggcgccaatctgtacggctctcaccctttttatctggccctggaagatggcggaagcgcccacggtgtctttctgctgaacagcaacgccatggacgtggtgctgcaaccatctcctgctctgtcttggagaagcaccggcggcatcctggacgtgtacatctttctgggacccgagcctaagagcgtggtgcagcagtatctggatgtcgtgggctaccccttcatgcctccttattggggcctgggcttccacctgtgtagatggggatacagctccaccgccatcaccagacaggtggtggaaaacatgacccgggctcacttcccactggatgtgcagtggaacgacctggactacatggactccagacgggacttcacctttaacaaggacggcttcagagacttccccgccatggtgcaagaactgcatcaaggcggcagacggtacatgatgatcgtggatcctgccatctcttctagcggccctgccggaagctacagaccttatgatgagggcctgagaagaggcgtgttcatcaccaatgagacaggccagcctctgatcggcaaagtgtggcctggaagcaccgcctttccagacttcaccaatccaaccgctctggcttggtgggaagatatggtggccgagttccacgatcaggtgcccttcgatggcatgtggatcgacatgaacgagcccagcaacttcatcaggggcagcgaggatggctgccccaacaacgaactggaaaatcctccttacgtgccaggcgttgtcggaggaacactgcaggccgccacaatttgtgccagcagccatcagtttctgagcacccactacaacctgcacaacctgtacggcctgaccgaggccattgcctctcatagagccctggttaaggccagaggcacccggccttttgtgatcagcagaagcacatttgccggccacggcagatatgccggacattggacaggggacgtttggtctagttgggagcagctggcctctagcgtgcccgagatcctgcagtttaatctgctgggagtgcccctcgtgggagccgatgtttgtggatttctgggcaacacctccgaggaactgtgcgtcagatggacacagctgggcgccttctatcccttcatgagaaaccacaacagcctgctgagcctgcctcaagagccttacagctttagcgaacccgcacagcaggccatgagaaaggccctgactctgagatacgctctgctgccccacctgtacaccctgtttcatcaagctcatgtggccggcgagacagtggccagaccactgtttctggaattccccaaggacagcagcacctggacagtggatcatcagctgctctggggagaagccctgctcattacacctgtgctgcaggctggcaaggccgaagtgacaggatactttcccctcggcacttggtacgacctgcagacagttcctgtggaagctctgggatctctgcctccacctcctgctgctcctagagagcctgccattcactctgaaggccagtgggttacactgcccgctccactggacaccatcaatgtgcacctgagagccggctacatcatccctctgcaaggccctggactgaccacaaccgaaagcagacagcagccaatggctctggccgtggctctgacaaaaggcggagaagctagaggcgaactgttctgggatgacggcgagagcctggaagtgctggaacggggagcctacacacaagtgatctttctcgcccggaacaacaccatcgtgaacgaactcgtcagagtgaccagtgaaggtgccggactgcagctccagaaagtgacagtgcttggagtggccacagcaccccagcaggttttgtctaatggcgtgcccgtgtccaacttcacatacagccctgacaccaaggtgctggacatctgtgtgtctctgctgatgggcgagcagttcctggtgtcctggtgttga

SEQ ID NO: 95 - DNA, 1812 bp, synthetic construct
  misc_feature 1..1812: note = human MTM1 sequence
  source 1..1812: mol_type = other DNA
atggcttctgcatcaacttctaaatataattcacactccttggagaatgagtctattaagaggacgtctcgagatggagtcaatcgagatctcactgaggctgttcctcgacttccaggagaaacactaatcactgacaaagaagttatttacatatgtcctttcaatggccccattaagggaagagtttacatcacaaattatcgtctttatttaagaagtttggaaacggattcttctctaatacttgatgttcctctgggtgtgatctcgagaattgaaaaaatgggaggcgcgacaagtagaggagaaaattcctatggtctagatattacttgtaaagacatgagaaacctgaggttcgctttgaaacaggaaggccacagcagaagagatatgtttgagatcctcacgagatacgcgtttcccctggctcacagtctgccattatttgcatttttaaatgaagaaaagtttaacgtggatggatggacagtttacaatccagtggaagaatacaggaggcagggcttgcccaatcaccattggagaataacttttattaataagtgctatgagctctgtgacacttaccctgctcttttggtggttccgtatcgtgcctcagatgatgacctccggagagttgcaacttttaggtcccgaaatcgaattccagtgctgtcatggattcatccagaaaataagacggtcattgtgcgttgcagtcagcctcttgtcggtatgagtgggaaacgaaataaagatgatgagaaatatctcgatgttatcagggagactaataaacaaatttctaaactcaccatttatgatgcaagacccagcgtaaatgcagtggccaacaaggcaacaggaggaggatatgaaagtgatgatgcatatcataacgccgaacttttcttcttagacattcataatattcatgttatgcgggaatctttaaaaaaagtgaaggacattgtttatcctaatgtagaagaatctcattggttgtccagtttggagtctactcattggttagaacatatcaagctcgttttgacaggagccattcaagtagcagacaaagtttcttcagggaagagttcagtgcttgtgcattgcagtgacggatgggacaggactgctcagctgacatccttggccatgctgatgttggatagcttctataggagcattgaagggttcgaaatactggtacaaaaaaaatggataagttttggacataaatttgcatctcgaataggtcatggtgataaaaaccacaccgatgctgaccgttctcctatttttctccagtttattgattgtgtgtggcaaatgtcaaaacagttccctacagcttttgaattcaatgaacaatttttgattataattttggatcatctgtatagttgccgatttggtactttcttattcaactgtgaatctgctcgagaaagacagaaggttacagaaaggactgtttctttatggtcactgataaacagtaataaagaaaaattcaaaaaccccttctatactaaagaaatcaatcgagttttatatccagttgccagtatgcgtcacttggaactctgggtgaattactacattagatggaaccccaggatcaagcaacaacagccgaatccagtggagcagcgttacatggagctcttagccttacgcgacgaatacataaagcggcttgaggaactgcagctcgccaactctgccaagctttctgatcccccaacttcaccttccagtccttcgcaaatgatgccccatgtgcaaactcacttctga

SEQ ID NO: 96 - DNA, 1812 bp, synthetic construct
  misc_feature 1..1812: note = human MTM1co sequence
  source 1..1812: mol_type = other DNA
atggccagcgccagcacaagcaagtacaacagccacagcctggaaaacgagagcatcaagcggaccagcagagatggcgtgaacagagatctgaccgaggccgttcctagactgcctggcgagacactgatcaccgacaaagaagtgatctacatctgccccttcaacggccccatcaagggaagagtgtacatcaccaactaccggctgtacctgcggtccctggaaaccgatagcagcctgattctggatgtgcccctgggcgtgatcagccggattgaaaaaatgggcggagccacctccagaggcgagaatagctatggcctggatatcacatgcaaggacatgcggaacctgagattcgccctgaagcaagagggccacagcagacgggacatgttcgagatcctgaccagatacgcctttcctctggctcactctctgcccctgttcgccttcctgaacgaagagaagttcaacgtggacggctggaccgtgtacaaccccgtggaagagtatagacggcagggactgcccaatcaccactggcggatcaccttcatcaacaagtgctacgagctgtgcgacacataccccgcactgctggtggtgccttacagagcctctgacgacgatctgagaagagtggccacctttcggagccggaacagaatccctgtgctgagctggattcaccccgagaacaagaccgtgatcgtgcggtgttctcagcctctcgtgggcatgagcggcaagagaaacaaggacgacgagaagtacctggacgtgatccgcgagacaaacaagcagatcagcaagctgaccatctacgacgccagaccttctgtgaacgccgtggccaacaaagccacaggcggcggatatgagtccgacgatgcctatcacaacgccgagctgttcttcctggacattcacaacatccatgtgatgcgcgagagcctgaagaaagtgaaggacatcgtgtaccccaatgtggaagagagccactggctgtctagcctggaatccacacactggctggaacacatcaagctggtgctgacaggcgccatccaggtggcagacaaagtgtctagcggcaagtctagcgtgctggtgcactgtagcgacggatgggatagaacagcccagctgacatccctggccatgctgatgctggacagcttctacagatccatcgagggctttgagatcctggtgcagaagaagtggatcagcttcggccacaagttcgcctctagaatcggacacggcgacaagaaccacaccgacgccgatagaagccccatcttcctgcagttcatcgactgcgtgtggcagatgtccaagcagttccctaccgccttcgagttcaacgagcagttcctgatcatcatcctggaccacctgtactcttgcagattcggcaccttcctgttcaactgcgagagcgccagagaacggcagaaagtgaccgagagaaccgtgtctctgtggtccctgatcaacagcaacaaagagaaattcaagaaccccttctacaccaaagaaatcaaccgggtgctgtaccccgtggccagcatgagacatctggaactgtgggtcaactactacatccggtggaaccccagaatcaagcagcagcagcccaatcctgtggaacagcggtatatggaactgctggccctgcgggacgagtacatcaagagactggaagaactgcagctggccaacagcgccaagctgagcgatcctcctacaagccctagcagcccctctcagatgatgccccatgtgcagacccacttttga

SEQ ID NO: 97 - DNA, 6024 bp, synthetic construct
  misc_feature 1..6024: note = plasmid pAAVss-CRE02-CSkSH5-SPc5-12_GTRM-Luc2
  source 1..6024: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgacaggtgcggttcccggagcgcaggcgcacacatgcacccaccggcgaacgcggtgaccctcgccccaccccatcccctccggcgggcaactgggtcgggtcaggaggggcaaacccgctagggagacactccatatacggcccggcccgcgttacctgggaccgggccaacccgctccttctttggtcaacgcaggggacccgggcgggggcccaggccgcgaaccggccgagggagggggctctagtgcccaacacccaaatatggctcgagaagggcagcgacattcctgcggggtggcgcggagggaatgcccgcgggctatataaaacctgagcagagggacaagcggccaccgcagcggacagcgccaagtgaagcctcgcttcccctccgcggcgaccagggcccgagccgagagtagcagttgtagctacccgcccaggtaggggcgcgccacgcgtcagtttactcaccagggattcagaggcagcactgctgaaccctgagcccttggcacatcaggttggctgtcagaagtcggcctttgtacatacacagttcccttgtgaggcccagctgcgtgtcctaggagcggggcctctctccacagcagagctcagcctctcaagtgtatggacagcacgggtgcctgatgggtggatttagccatgagttgaaggtggcttggggagaatgagagttctagagatagggagaaggggttgccaataggagagtggaattcctgagcacctcgtcacaggcagccgacagaacatgagccgcagggcccaggctatttatacctcgcctgtcactatcagggtccccacagctccccccacctccagccacacacagcaggtccttttgctctttctggtcccttctctactcctccccctccctacctaaggtacccaacgcgttacgtggccaccgccttcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagatctagaaagaggtaagggtttaagggatggttggttggtggggtattaatgtttaattacctggagcacctgcctgaaatcactttttttcaggttggaagcttatggaagatgccaaaaacattaagaagggcccagcgccattctacccactcgaggacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaattcgaaacgtcaagtccatcttgagcatctgacttctggctaaataaaagatctttattttcattagatctgtgtgttggttttttgtgtgcgtcgagatccacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 98 - DNA, 6081 bp, synthetic construct
  misc_feature 1..6081: note = plasmid pAAVss-CRE04-CSkSH5-SPc5-12_GTRM-Luc2
  source 1..6081: mol_type = other DNA
acatgtcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccccttttagagaatccacacctgtcccagttgctgggttccactaccaaaagtgaattgcaactattttaggagcacttaagcacatccgaaaaatgagtgattctgttctggcccacaccacatcactgatgtacccccttaaagcatgtccctgagttcatcacagaagactgctcctcctgtgccctccacaaggttagaactgtccttgtcttagggaaaaaggagagagagagagagagagagagagagagagagagagagagagagagagagggacaggcaccaactgggtaacctctgctgacccccactctactttaccataagtagctccaaatccttctagaaaatctgaaaggcatagccccatatatcagtgatataaatagaacctgcagcaggctctggtaaatgatgactacaaggtggactgggaggcagcccggccttggcaggcatcatcctctaaatataaagatgagtttgttcagcctttgcagaaggaggcgcgccacgcgtcagtttactcaccagggattcagaggcagcactgctgaaccctgagcccttggcacatcaggttggctgtcagaagtcggcctttgtacatacacagttcccttgtgaggcccagctgcgtgtcctaggagcggggcctctctccacagcagagctcagcctctcaagtgtatggacagcacgggtgcctgatgggtggatttagccatgagttgaaggtggcttggggagaatgagagttctagagatagggagaaggggttgccaataggagagtggaattcctgagcacctcgtcacaggcagccgacagaacatgagccgcagggcccaggctatttatacctcgcctgtcactatcagggtccccacagctccccccacctccagccacacacagcaggtccttttgctctttctggtcccttctctactcctccccctccctacctaaggtacccaacgcgttacgtggccaccgccttcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagatctagaaagaggtaagggtttaagggatggttggttggtggggtattaatgtttaattacctggagcacctgcctgaaatcactttttttcaggttggaagcttatggaagatgccaaaaacattaagaagggcccagcgccattctacccactcgaggacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaattcgaaacgtcacgtccatcttgagcatctgacttctggctaaataaaagatctttattttcattagatctgtgtgttggttttttgtgtgcgtcgagatccacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctc

SEQ ID NO: 99 - DNA, 5972 bp, synthetic construct
  misc_feature 1..5972: note = plasmid pAAVss-CRE06-CSkSH5-SPc5-12_GTRM-Luc2
  source 1..5972: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgggccaggggacggtggcttctacgtgcttgggacgttcccagccaccgtcccatgttcccggcggggggccagctgtccccaccgccagcccaactcagcacttggtcagggtatcagcttggtgggggggcgtgagcccagcccctggggcggctcagcccatacaaggccatggggctgggcgcaaagcatgcctgggttcagggtgggtatggtgcgggagcagggaggtgagaggctcagctgccctccagaactcctccctggggacaacccctcccagccaatagcacagcctaggtccccctatataaggccacggctgctggcccttcctttgggtcagtgtcacctccaggatacagacagccccccttcagcccagcccagccaggtacggcgcgccacgcgtcagtttactcaccagggattcagaggcagcactgctgaaccctgagcccttggcacatcaggttggctgtcagaagtcggcctttgtacatacacagttcccttgtgaggcccagctgcgtgtcctaggagcggggcctctctccacagcagagctcagcctctcaagtgtatggacagcacgggtgcctgatgggtggatttagccatgagttgaaggtggcttggggagaatgagagttctagagatagggagaaggggttgccaataggagagtggaattcctgagcacctcgtcacaggcagccgacagaacatgagccgcagggcccaggctatttatacctcgcctgtcactatcagggtccccacagctccccccacctccagccacacacagcaggtccttttgctctttctggtcccttctctactcctccccctccctacctaaggtacccaacgcgttacgtggccaccgccttcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagatctagaaagaggtaagggtttaagggatggttggttggtggggtattaatgtttaattacctggagcacctgcctgaaatcactttttttcaggttggaagcttatggaagatgccaaaaacattaagaagggcccagcgccattctacccactcgaggacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaattcgaaacgtctagtccatcttgagcatctgacttctggctaaataaaagatctttattttcattagatctgtgtgttggttttttgtgtgcgtcgagatccacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 100 - DNA, 5827 bp, synthetic construct
  misc_feature 1..5827: note = plasmid pAAVss-CRE07-CSkSH5-SPc5-12_GTRM-Luc2
  source 1..5827: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcccccagcccccttccccgggaggtgggagcggccacccagggccccgtggctgcccttgtaaggaggcgaggcccgaggacacccgagacgcccggttataattaaccaggacacgtggcgaacccccctccaacacctgcccccgaacccccccatacccagcgcctcgggtctcggcctttgcggcagaggagacagcaaagcgccctctaaaaataactcctttcccggcgaccgagaccctccctgtcccccggcgcgccacgcgtcagtttactcaccagggattcagaggcagcactgctgaaccctgagcccttggcacatcaggttggctgtcagaagtcggcctttgtacatacacagttcccttgtgaggcccagctgcgtgtcctaggagcggggcctctctccacagcagagctcagcctctcaagtgtatggacagcacgggtgcctgatgggtggatttagccatgagttgaaggtggcttggggagaatgagagttctagagatagggagaaggggttgccaataggagagtggaattcctgagcacctcgtcacaggcagccgacagaacatgagccgcagggcccaggctatttatacctcgcctgtcactatcagggtccccacagctccccccacctccagccacacacagcaggtccttttgctctttctggtcccttctctactcctccccctccctacctaaggtacccaacgcgttacgtggccaccgccttcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagatctagaaagaggtaagggtttaagggatggttggttggtggggtattaatgtttaattacctggagcacctgcctgaaatcactttttttcaggttggaagcttatggaagatgccaaaaacattaagaagggcccagcgccattctacccactcgaggacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaattcgaaacgtcttgtccatcttgagcatctgacttctggctaaataaaagatctttattttcattagatctgtgtgttggttttttgtgtgcgtcgagatccacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 101 - DNA, 5891 bp, synthetic construct
  misc_feature 1..5891: note = plasmid pAAVss-CRE66-CSkSH5-SPc5-12_GTRM-Luc2
  source 1..5891: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccccaggtgacaggtggatggtggagctggaggagccactcggagcctccaaggctggggagggtggaggggggaggcaggtcggtgtcccaggccacacctgttgactaagggattaggatgttgtgtccctgccagccctcctccaataagcccctctgggcctgcagggagaggaggagccttgccatgtaaactgtatttttagttccctgtgcctctccccggctgctataagacacctctccccacccccagccctggccgcttggctggaggctctgcgaggacagctggggagaaggggagctgtggtcagtaggcgcgccacgcgtcagtttactcaccagggattcagaggcagcactgctgaaccctgagcccttggcacatcaggttggctgtcagaagtcggcctttgtacatacacagttcccttgtgaggcccagctgcgtgtcctaggagcggggcctctctccacagcagagctcagcctctcaagtgtatggacagcacgggtgcctgatgggtggatttagccatgagttgaaggtggcttggggagaatgagagttctagagatagggagaaggggttgccaataggagagtggaattcctgagcacctcgtcacaggcagccgacagaacatgagccgcagggcccaggctatttatacctcgcctgtcactatcagggtccccacagctccccccacctccagccacacacagcaggtccttttgctctttctggtcccttctctactcctccccctccctacctaaggtacccaacgcgttacgtggccaccgccttcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagatctagaaagaggtaagggtttaagggatggttggttggtggggtattaatgtttaattacctggagcacctgcctgaaatcactttttttcaggttggaagcttatggaagatgccaaaaacattaagaagggcccagcgccattctacccactcgaggacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaattcgaaacgtactgtccatcttgagcatctgacttctggctaaataaaagatctttattttcattagatctgtgtgttggttttttgtgtgcgtcgagatccacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 102 - DNA, 5865 bp, synthetic construct
  misc_feature 1..5865: note = plasmid pAAVss-CRE68-CSkSH5-SPc5-12_GTRM-Luc2
  source 1..5865: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccagaaaggagctgccctggggatggctgtgtatccctacaactgccaggcacatgccccttgaacacccgatgctacgtgtcccaaaggaaactggtctccacccacccccggcccgtcctcgtcctgggtaccccaccttagtaaatggcgccaccatctgcccggtcactcagcgagaaactcaactcctggcagcagatgacgggcactctggttaaatgactctctccagcctccaagttcaacctgcagggaagccctggaaatcctgtctccctctgccctgcctctcggcgcgccacgcgtcagtttactcaccagggattcagaggcagcactgctgaaccctgagcccttggcacatcaggttggctgtcagaagtcggcctttgtacatacacagttcccttgtgaggcccagctgcgtgtcctaggagcggggcctctctccacagcagagctcagcctctcaagtgtatggacagcacgggtgcctgatgggtggatttagccatgagttgaaggtggcttggggagaatgagagttctagagatagggagaaggggttgccaataggagagtggaattcctgagcacctcgtcacaggcagccgacagaacatgagccgcagggcccaggctatttatacctcgcctgtcactatcagggtccccacagctccccccacctccagccacacacagcaggtccttttgctctttctggtcccttctctactcctccccctccctacctaaggtacccaacgcgttacgtggccaccgccttcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagatctagaaagaggtaagggtttaagggatggttggttggtggggtattaatgtttaattacctggagcacctgcctgaaatcactttttttcaggttggaagcttatggaagatgccaaaaacattaagaagggcccagcgccattctacccactcgaggacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaattcgaaacgtacggtccatcttgagcatctgacttctggctaaataaaagatctttattttcattagatctgtgtgttggttttttgtgtgcgtcgagatccacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 103 - DNA, 6054 bp, synthetic construct
  misc_feature 1..6054: note = plasmid pAAVss-CRE69-CSkSH5-SPc5-12_GTRM-Luc2
  source 1..6054: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgaaacatggcttccacaggttccagttgaagaatcccagttccgtctataaattccagggaaggtctctgattggccctgctcattcccaggcccattccttgacccagtcactgaagtcagggagatgcagtaataagactggctggaatcagggtctttaggggtggagggatggggaggaggcacagcatgtcatcaaaataaggaaattgcaaaagaaagcttgcaggctactttgaatgacaatgagaaagacggtgctgcctgagtgtgttaaggatccacatggtctccaaaatcctccaggagcatacagtctagtctgggagatgagacacaaaaataaccagaacacaacagcttgcactgactcgagggctggataagaatatctggaactcccccatctatttcagaagcttgtctcttggatgaaaattagacacttaatgggaaagggctttgaaaagagtgcagtaaggcgcgccacgcgtcagtttactcaccagggattcagaggcagcactgctgaaccctgagcccttggcacatcaggttggctgtcagaagtcggcctttgtacatacacagttcccttgtgaggcccagctgcgtgtcctaggagcggggcctctctccacagcagagctcagcctctcaagtgtatggacagcacgggtgcctgatgggtggatttagccatgagttgaaggtggcttggggagaatgagagttctagagatagggagaaggggttgccaataggagagtggaattcctgagcacctcgtcacaggcagccgacagaacatgagccgcagggcccaggctatttatacctcgcctgtcactatcagggtccccacagctccccccacctccagccacacacagcaggtccttttgctctttctggtcccttctctactcctccccctccctacctaaggtacccaacgcgttacgtggccaccgccttcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagatctagaaagaggtaagggtttaagggatggttggttggtggggtattaatgtttaattacctggagcacctgcctgaaatcactttttttcaggttggaagcttatggaagatgccaaaaacattaagaagggcccagcgccattctacccactcgaggacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaattcgaaacgtatcgtccatcttgagcatctgacttctggctaaataaaagatctttattttcattagatctgtgtgttggttttttgtgtgcgtcgagatccacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 104 - DNA, 5970 bp, synthetic construct
  misc_feature 1..5970: note = plasmid pAAVss-CRE70-CSkSH5-SPc5-12_GTRM-Luc2
  source 1..5970: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcccctcaggtaccccctgccccccacagctcctctcctgtgccttgtttcccagccatgcgttctcctctataaatacccgctctggtatttggggttggcagctgttgctgccagggagatggttgggttgacatgcggctcctgacaaaacacaaacccctggtgtgtgtgggcgtgggtggtgtgagtagggggatgaatcagggagggggcgggggacccagggggcaggagccacacaaagtctgtgcgggggtgggagcgcacatagcaattggaaactgaaagcttatcagaccctttctggaaatcagcccactgtttataaacttgaggccccaccctcgacagtaccggggaggaagagggcctgcactagtccagagggaaactgaggcggcgcgccacgcgtcagtttactcaccagggattcagaggcagcactgctgaaccctgagcccttggcacatcaggttggctgtcagaagtcggcctttgtacatacacagttcccttgtgaggcccagctgcgtgtcctaggagcggggcctctctccacagcagagctcagcctctcaagtgtatggacagcacgggtgcctgatgggtggatttagccatgagttgaaggtggcttggggagaatgagagttctagagatagggagaaggggttgccaataggagagtggaattcctgagcacctcgtcacaggcagccgacagaacatgagccgcagggcccaggctatttatacctcgcctgtcactatcagggtccccacagctccccccacctccagccacacacagcaggtccttttgctctttctggtcccttctctactcctccccctccctacctaaggtacccaacgcgttacgtggccaccgccttcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagatctagaaagaggtaagggtttaagggatggttggttggtggggtattaatgtttaattacctggagcacctgcctgaaatcactttttttcaggttggaagcttatggaagatgccaaaaacattaagaagggcccagcgccattctacccactcgaggacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaattcgaaacgtatggtccatcttgagcatctgacttctggctaaataaaagatctttattttcattagatctgtgtgttggttttttgtgtgcgtcgagatccacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 105 - DNA, 5813 bp, synthetic construct
  misc_feature 1..5813: note = plasmid pAAVss-CRE71-CSkSH5-SPc5-12_GTRM-Luc2
  source 1..5813: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcccaggggcgcaggcctcttgcgggggagctggcctccccgcccccacggccacgggccgccctttcctggcaggacagcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtggcgcgccacgcgtcagtttactcaccagggattcagaggcagcactgctgaaccctgagcccttggcacatcaggttggctgtcagaagtcggcctttgtacatacacagttcccttgtgaggcccagctgcgtgtcctaggagcggggcctctctccacagcagagctcagcctctcaagtgtatggacagcacgggtgcctgatgggtggatttagccatgagttgaaggtggcttggggagaatgagagttctagagatagggagaaggggttgccaataggagagtggaattcctgagcacctcgtcacaggcagccgacagaacatgagccgcagggcccaggctatttatacctcgcctgtcactatcagggtccccacagctccccccacctccagccacacacagcaggtccttttgctctttctggtcccttctctactcctccccctccctacctaaggtacccaacgcgttacgtggccaccgccttcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagatctagaaagaggtaagggtttaagggatggttggttggtggggtattaatgtttaattacctggagcacctgcctgaaatcactttttttcaggttggaagcttatggaagatgccaaaaacattaagaagggcccagcgccattctacccactcgaggacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaattcgaaacgtacagtccatcttgagcatctgacttctggctaaataaaagatctttattttcattagatctgtgtgttggttttttgtgtgcgtcgagatccacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 106 - DNA, 5888 bp, synthetic construct
  misc_feature 1..5888: note = plasmid pAAVss-CRE77-CSkSH5-SPc5-12_GTRM-Luc2
  source 1..5888: mol_type = other DNA
acatgtcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccggaataccctaccttaaaaacaaaacaaaccgtgctacttggccctcttcccttgagttgttgtctaatctcattcatttcactgccaaactcctcaaatgcatggtgtacacccactgcttccacttcctgtccactcatctttttcctcattaactccctgtggcctggctttgcctgtgacactacacaagctgcttgctccaagataatccaagttcttttctctgtcttcctgatacacaggctttctttggcatctgacactgctggccacctgctcttcttgaaatgcttcttttggcttctaggaaacggcgcgccacgcgtcagtttactcaccagggattcagaggcagcactgctgaaccctgagcccttggcacatcaggttggctgtcagaagtcggcctttgtacatacacagttcccttgtgaggcccagctgcgtgtcctaggagcggggcctctctccacagcagagctcagcctctcaagtgtatggacagcacgggtgcctgatgggtggatttagccatgagttgaaggtggcttggggagaatgagagttctagagatagggagaaggggttgccaataggagagtggaattcctgagcacctcgtcacaggcagccgacagaacatgagccgcagggcccaggctatttatacctcgcctgtcactatcagggtccccacagctccccccacctccagccacacacagcaggtccttttgctctttctggtcccttctctactcctccccctccctacctaaggtacccaacgcgttacgtggccaccgccttcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagatctagaaagaggtaagggtttaagggatggttggttggtggggtattaatgtttaattacctggagcacctgcctgaaatcactttttttcaggttggaagcttatggaagatgccaaaaacattaagaagggcccagcgccattctacccactcgaggacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaattcgaaacgtagggtccatcttgagcatctgacttctggctaaataaaagatctttattttcattagatctgtgtgttggttttttgtgtgcgtcgagatccacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctc

SEQ ID NO: 107 - DNA, 5572 bp, synthetic construct
  misc_feature 1..5572: note = plasmid pAAVss-CSkSH5-SPc5-12_GTRM-Luc2
  source 1..5572: mol_type = other DNA
acatgtcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccggcgcgccacgcgtcagtttactcaccagggattcagaggcagcactgctgaaccctgagcccttggcacatcaggttggctgtcagaagtcggcctttgtacatacacagttcccttgtgaggcccagctgcgtgtcctaggagcggggcctctctccacagcagagctcagcctctcaagtgtatggacagcacgggtgcctgatgggtggatttagccatgagttgaaggtggcttggggagaatgagagttctagagatagggagaaggggttgccaataggagagtggaattcctgagcacctcgtcacaggcagccgacagaacatgagccgcagggcccaggctatttatacctcgcctgtcactatcagggtccccacagctccccccacctccagccacacacagcaggtccttttgctctttctggtcccttctctactcctccccctccctacctaaggtacccaacgcgttacgtggccaccgccttcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagatctagaaagaggtaagggtttaagggatggttggttggtggggtattaatgtttaattacctggagcacctgcctgaaatcactttttttcaggttggaagcttatggaagatgccaaaaacattaagaagggcccagcgccattctacccactcgaggacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaattcgaaacgttcggtccatcttgagcatctgacttctggctaaataaaagatctttattttcattagatctgtgtgttggttttttgtgtgcgtcgagatccacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctc

SEQ ID NO: 108 - DNA, 5118 bp, synthetic construct
  misc_feature 1..5118: note = plasmid pAAVss-SPc5-12_GTRM-Luc2
  source 1..5118: mol_type = other DNA
acatgtcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccggcgcgccacgcgtggtacccaacgcgttacgtggccaccgccttcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagatctagaaagaggtaagggtttaagggatggttggttggtggggtattaatgtttaattacctggagcacctgcctgaaatcactttttttcaggttggaagcttatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaattcgaaacgttgtgtccatcttgagcatctgacttctggctaaataaaagatctttattttcattagatctgtgtgttggttttttgtgtgcgtcgagatccacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctc

SEQ ID NO: 109 - DNA, 7097 bp, synthetic construct
  misc_feature 1..7097: note = plasmid pAAVss-CRE02-SkSH4-hDES1.4kb-Luc2
  source 1..7097: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgacaggtgcggttcccggagcgcaggcgcacacatgcacccaccggcgaacgcggtgaccctcgccccaccccatcccctccggcgggcaactgggtcgggtcaggaggggcaaacccgctagggagacactccatatacggcccggcccgcgttacctgggaccgggccaacccgctccttctttggtcaacgcaggggacccgggcgggggcccaggccgcgaaccggccgagggagggggctctagtgcccaacacccaaatatggctcgagaagggcagcgacattcctgcggggtggcgcggagggaatgcccgcgggctatataaaacctgagcagagggacaagcggccaccgcagcggacagcgccaagtgaagcctcgcttcccctccgcggcgaccagggcccgagccgagagtagcagttgtagctacccgcccaggtaggggcgcgccacgcgtttctgagtcctctaaggtccctcactcccaactcagccccatgtcctgtcaattcccactcagtgtctgatctccttctcctcacctttcccatctcccgtttgacccaagcttcctgagctctcctcccattcccctttttggagtcctcctcctctcccagaacccagtaataagtgggctcctccctggcctggacccccgtggtaaccctataaggcgaggcagctgctgtctgaggcagggaggggctggtgtgggaggctaagggcagctgctaagtttagggtggctccttctctcttcttagagacaacaggtggctggggcctcagtgcccagaaaagaaaatgtcttagaggtatcggcatgggcctggaggaggggggacagggcagggggaggcatcttcctcaggacatcgggtcctagaggggtacccaacgggttacgacacacctactagtaacccctccagctggtgatggcaggtctagggtaggaccagtgactggctcctaatcgagcactctattttcagggtttgcattccaaaagggtcaggtccaagagggacctggagtgccaagtggaggtgtagaggcacggccagtacccatggagaatggtggatgtccttaggggttagcaagtgccgtgtgctaaggagggggctttggaggttgggcaggccctctgtggggctccatttttgtgggggtgggggctggagcattatagggggtgggaagtgattggggctgtcaccctagccttccttatctgacgcccacccatgcctcctcaggtaccccctgccccccacagctcctctcctgtgccttgtttcccagccatgcgttctcctctataaatacccgctctggtatttggggttggcagctgttgctgccagggagatggttgggttgacatgcggctcctgacaaaacacaaacccctggtgtgtgtgggcgtgggtggtgtgagtagggggatgaatcagggagggggcgggggacccagggggcaggagccacacaaagtctgtgcgggggtgggagcgcacatagcaattggaaactgaaagcttatcagaccctttctggaaatcagcccactgtttataaacttgaggccccaccctcgacagtaccggggaggaagagggcctgcactagtccagagggaaactgaggctcagggctagctcgcccatagacatacatggcaggcaggctttggccaggatccctccgcctgccaggcgtctccctgccctcccttcctgcctagagacccccaccctcaagcctggctggtctttgcctgagacccaaacctcttcgacttcaagagaatatttaggaacaaggtggtttagggcctttcctgggaacaggccttgaccctttaagaaatgacccaaagtctctccttgaccaaaaaggggaccctcaaactaaagggaagcctctcttctgctgtctcccctgaccccactcccccccaccccaggacgaggagataaccagggctgaaagaggcccgcctgggggctgcagacatgcttgctgcctgccctggcgaaggattggcaggcttgcccgtcacaggacccccgctggctgactcaggggcgcaggcctcttgcgggggagctggcctccccgcccccacggccacgggccgccctttcctggcaggacagcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcacctctagaaagaggtaagggtttaagggatggttggttggtggggtattaatgtttaattacctggagcacctgcctgaaatcactttttttcaggttggaagcttatggaagatgccaaaaacattaagaagggcccagcgccattctacccactcgaggacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaattcgaaacgaaatgtccatcttgagcatctgacttctggctaaataaaagatctttattttcattagatctgtgtgttggttttttgtgtgcgtcgagatccacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 110 - DNA, 7154 bp, synthetic construct
  misc_feature 1..7154: note = plasmid pAAVss-CRE04-SkSH4-hDES1.4kb-Luc2
  source 1..7154: mol_type = other DNA
acatgtcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccccttttagagaatccacacctgtcccagttgctgggttccactaccaaaagtgaattgcaactattttaggagcacttaagcacatccgaaaaatgagtgattctgttctggcccacaccacatcactgatgtacccccttaaagcatgtccctgagttcatcacagaagactgctcctcctgtgccctccacaaggttagaactgtccttgtcttagggaaaaaggagagagagagagagagagagagagagagagagagagagagagagagagagggacaggcaccaactgggtaacctctgctgacccccactctactttaccataagtagctccaaatccttctagaaaatctgaaaggcatagccccatatatcagtgatataaatagaacctgcagcaggctctggtaaatgatgactacaaggtggactgggaggcagcccggccttggcaggcatcatcctctaaatataaagatgagtttgttcagcctttgcagaaggaggcgcgccacgcgtttctgagtcctctaaggtccctcactcccaactcagccccatgtcctgtcaattcccactcagtgtctgatctccttctcctcacctttcccatctcccgtttgacccaagcttcctgagctctcctcccattcccctttttggagtcctcctcctctcccagaacccagtaataagtgggctcctccctggcctggacccccgtggtaaccctataaggcgaggcagctgctgtctgaggcagggaggggctggtgtgggaggctaagggcagctgctaagtttagggtggctccttctctcttcttagagacaacaggtggctggggcctcagtgcccagaaaagaaaatgtcttagaggtatcggcatgggcctggaggaggggggacagggcagggggaggcatcttcctcaggacatcgggtcctagaggggtacccaacgggttacgacacacctactagtaacccctccagctggtgatggcaggtctagggtaggaccagtgactggctcctaatcgagcactctattttcagggtttgcattccaaaagggtcaggtccaagagggacctggagtgccaagtggaggtgtagaggcacggccagtacccatggagaatggtggatgtccttaggggttagcaagtgccgtgtgctaaggagggggctttggaggttgggcaggccctctgtggggctccatttttgtgggggtgggggctggagcattatagggggtgggaagtgattggggctgtcaccctagccttccttatctgacgcccacccatgcctcctcaggtaccccctgccccccacagctcctctcctgtgccttgtttcccagccatgcgttctcctctataaatacccgctctggtatttggggttggcagctgttgctgccagggagatggttgggttgacatgcggctcctgacaaaacacaaacccctggtgtgtgtgggcgtgggtggtgtgagtagggggatgaatcagggagggggcgggggacccagggggcaggagccacacaaagtctgtgcgggggtgggagcgcacatagcaattggaaactgaaagcttatcagaccctttctggaaatcagcccactgtttataaacttgaggccccaccctcgacagtaccggggaggaagagggcctgcactagtccagagggaaactgaggctcagggctagctcgcccatagacatacatggcaggcaggctttggccaggatccctccgcctgccaggcgtctccctgccctcccttcctgcctagagacccccaccctcaagcctggctggtctttgcctgagacccaaacctcttcgacttcaagagaatatttaggaacaaggtggtttagggcctttcctgggaacaggccttgaccctttaagaaatgacccaaagtctctccttgaccaaaaaggggaccctcaaactaaagggaagcctctcttctgctgtctcccctgaccccactcccccccaccccaggacgaggagataaccagggctgaaagaggcccgcctgggggctgcagacatgcttgctgcctgccctggcgaaggattggcaggcttgcccgtcacaggacccccgctggctgactcaggggcgcaggcctcttgcgggggagctggcctccccgcccccacggccacgggccgccctttcctggcaggacagcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcacctctagaaagaggtaagggtttaagggatggttggttggtggggtattaatgtttaattacctggagcacctgcctgaaatcactttttttcaggttggaagcttatggaagatgccaaaaacattaagaagggcccagcgccattctacccactcgaggacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaattcgaaacgaaaggtccatcttgagcatctgacttctggctaaataaaagatctttattttcattagatctgtgtgttggttttttgtgtgcgtcgagatccacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctc

SEQ ID NO: 111 - DNA, 7045 bp, synthetic construct
  misc_feature 1..7045: note = plasmid pAAVss-CRE06-SkSH4-hDES1.4kb-Luc2
  source 1..7045: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgggccaggggacggtggcttctacgtgcttgggacgttcccagccaccgtcccatgttcccggcggggggccagctgtccccaccgccagcccaactcagcacttggtcagggtatcagcttggtgggggggcgtgagcccagcccctggggcggctcagcccatacaaggccatggggctgggcgcaaagcatgcctgggttcagggtgggtatggtgcgggagcagggaggtgagaggctcagctgccctccagaactcctccctggggacaacccctcccagccaatagcacagcctaggtccccctatataaggccacggctgctggcccttcctttgggtcagtgtcacctccaggatacagacagccccccttcagcccagcccagccaggtacggcgcgccacgcgtttctgagtcctctaaggtccctcactcccaactcagccccatgtcctgtcaattcccactcagtgtctgatctccttctcctcacctttcccatctcccgtttgacccaagcttcctgagctctcctcccattcccctttttggagtcctcctcctctcccagaacccagtaataagtgggctcctccctggcctggacccccgtggtaaccctataaggcgaggcagctgctgtctgaggcagggaggggctggtgtgggaggctaagggcagctgctaagtttagggtggctccttctctcttcttagagacaacaggtggctggggcctcagtgcccagaaaagaaaatgtcttagaggtatcggcatgggcctggaggaggggggacagggcagggggaggcatcttcctcaggacatcgggtcctagaggggtacccaacgggttacgacacacctactagtaacccctccagctggtgatggcaggtctagggtaggaccagtgactggctcctaatcgagcactctattttcagggtttgcattccaaaagggtcaggtccaagagggacctggagtgccaagtggaggtgtagaggcacggccagtacccatggagaatggtggatgtccttaggggttagcaagtgccgtgtgctaaggagggggctttggaggttgggcaggccctctgtggggctccatttttgtgggggtgggggctggagcattatagggggtgggaagtgattggggctgtcaccctagccttccttatctgacgcccacccatgcctcctcaggtaccccctgccccccacagctcctctcctgtgccttgtttcccagccatgcgttctcctctataaatacccgctctggtatttggggttggcagctgttgctgccagggagatggttgggttgacatgcggctcctgacaaaacacaaacccctggtgtgtgtgggcgtgggtggtgtgagtagggggatgaatcagggagggggcgggggacccagggggcaggagccacacaaagtctgtgcgggggtgggagcgcacatagcaattggaaactgaaagcttatcagaccctttctggaaatcagcccactgtttataaacttgaggccccaccctcgacagtaccggggaggaagagggcctgcactagtccagagggaaactgaggctcagggctagctcgcccatagacatacatggcaggcaggctttggccaggatccctccgcctgccaggcgtctccctgccctcccttcctgcctagagacccccaccctcaagcctggctggtctttgcctgagacccaaacctcttcgacttcaagagaatatttaggaacaaggtggtttagggcctttcctgggaacaggccttgaccctttaagaaatgacccaaagtctctccttgaccaaaaaggggaccctcaaactaaagggaagcctctcttctgctgtctcccctgaccccactcccccccaccccaggacgaggagataaccagggctgaaagaggcccgcctgggggctgcagacatgcttgctgcctgccctggcgaaggattggcaggcttgcccgtcacaggacccccgctggctgactcaggggcgcaggcctcttgcgggggagctggcctccccgcccccacggccacgggccgccctttcctggcaggacagcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcacctctagaaagaggtaagggtttaagggatggttggttggtggggtattaatgtttaattacctggagcacctgcctgaaatcactttttttcaggttggaagcttatggaagatgccaaaaacattaagaagggcccagcgccattctacccactcgaggacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaattcgaaacgaagggtccatcttgagcatctgacttctggctaaataaaagatctttattttcattagatctgtgtgttggttttttgtgtgcgtcgagatccacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 112 - DNA, 6942 bp, synthetic construct
  misc_feature 1..6942: note = plasmid pAAVss-CRE18-SkSH4-hDES1.4kb-Luc2
  source 1..6942: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcccctttgacttttcttgaaaggggagggctgggatattccagagattgatccttaaggcttgctgactgcctactcacttctggaaacttccagcagtgtcattcatagacctgtgaagagctcttagcttgtttccttcacacagtggggactctgaggggtcagagtgagtcacccagcaggccagtggcaggggtgagccagaagcctggccaaaccctcctatcatcatggagagaagaaagcctcctccagaagacgggaggccggcagggcgtggggcctgctcagatgcagggcgcgccacgcgtttctgagtcctctaaggtccctcactcccaactcagccccatgtcctgtcaattcccactcagtgtctgatctccttctcctcacctttcccatctcccgtttgacccaagcttcctgagctctcctcccattcccctttttggagtcctcctcctctcccagaacccagtaataagtgggctcctccctggcctggacccccgtggtaaccctataaggcgaggcagctgctgtctgaggcagggaggggctggtgtgggaggctaagggcagctgctaagtttagggtggctccttctctcttcttagagacaacaggtggctggggcctcagtgcccagaaaagaaaatgtcttagaggtatcggcatgggcctggaggaggggggacagggcagggggaggcatcttcctcaggacatcgggtcctagaggggtacccaacgggttacgacacacctactagtaacccctccagctggtgatggcaggtctagggtaggaccagtgactggctcctaatcgagcactctattttcagggtttgcattccaaaagggtcaggtccaagagggacctggagtgccaagtggaggtgtagaggcacggccagtacccatggagaatggtggatgtccttaggggttagcaagtgccgtgtgctaaggagggggctttggaggttgggcaggccctctgtggggctccatttttgtgggggtgggggctggagcattatagggggtgggaagtgattggggctgtcaccctagccttccttatctgacgcccacccatgcctcctcaggtaccccctgccccccacagctcctctcctgtgccttgtttcccagccatgcgttctcctctataaatacccgctctggtatttggggttggcagctgttgctgccagggagatggttgggttgacatgcggctcctgacaaaacacaaacccctggtgtgtgtgggcgtgggtggtgtgagtagggggatgaatcagggagggggcgggggacccagggggcaggagccacacaaagtctgtgcgggggtgggagcgcacatagcaattggaaactgaaagcttatcagaccctttctggaaatcagcccactgtttataaacttgaggccccaccctcgacagtaccggggaggaagagggcctgcactagtccagagggaaactgaggctcagggctagctcgcccatagacatacatggcaggcaggctttggccaggatccctccgcctgccaggcgtctccctgccctcccttcctgcctagagacccccaccctcaagcctggctggtctttgcctgagacccaaacctcttcgacttcaagagaatatttaggaacaaggtggtttagggcctttcctgggaacaggccttgaccctttaagaaatgacccaaagtctctccttgaccaaaaaggggaccctcaaactaaagggaagcctctcttctgctgtctcccctgaccccactcccccccaccccaggacgaggagataaccagggctgaaagaggcccgcctgggggctgcagacatgcttgctgcctgccctggcgaaggattggcaggcttgcccgtcacaggacccccgctggctgactcaggggcgcaggcctcttgcgggggagctggcctccccgcccccacggccacgggccgccctttcctggcaggacagcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcacctctagaaagaggtaagggtttaagggatggttggttggtggggtattaatgtttaattacctggagcacctgcctgaaatcactttttttcaggttggaagcttatggaagatgccaaaaacattaagaagggcccagcgccattctacccactcgaggacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaattcgaaacgagacgtccatcttgagcatctgacttctggctaaataaaagatctttattttcattagatctgtgtgttggttttttgtgtgcgtcgagatccacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 113 - DNA, 7138 bp, synthetic construct
  misc_feature 1..7138: note = plasmid pAAVss-CRE21-SkSH4-hDES1.4kb-Luc2
  source 1..7138: mol_type = other DNA
acatgtcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcccttttcttttctaattaggtcagttacttcatgtctgtttttcttttttgacatggtttcttctggctttggcaccacatattttccccatatgtcattgcctctggagcttcatgttgcaatagtttttcaaggggaacggagagcacattgctaagggtgggggatggcttttgcctcttttgcctgccctttgcttcagtgagtgttcgtatttctgttgggcctagttctgtttggttttgtagtcttcagagtcagttatgtttggactgaaagatacttaagtaaaaataatggcaagtcagagatatgtctggcaaaggtgcagcacatttgttaaggttactggtttatttatctcccttgtctctatggtgactaaatctgggtctgggatttaatggacttagttttgacctcttgtaacatctcctaacttttcccagcctctgatttagaaagaattcattttcacttgaggagagaaactggcgcgccacgcgtttctgagtcctctaaggtccctcactcccaactcagccccatgtcctgtcaattcccactcagtgtctgatctccttctcctcacctttcccatctcccgtttgacccaagcttcctgagctctcctcccattcccctttttggagtcctcctcctctcccagaacccagtaataagtgggctcctccctggcctggacccccgtggtaaccctataaggcgaggcagctgctgtctgaggcagggaggggctggtgtgggaggctaagggcagctgctaagtttagggtggctccttctctcttcttagagacaacaggtggctggggcctcagtgcccagaaaagaaaatgtcttagaggtatcggcatgggcctggaggaggggggacagggcagggggaggcatcttcctcaggacatcgggtcctagaggggtacccaacgggttacgacacacctactagtaacccctccagctggtgatggcaggtctagggtaggaccagtgactggctcctaatcgagcactctattttcagggtttgcattccaaaagggtcaggtccaagagggacctggagtgccaagtggaggtgtagaggcacggccagtacccatggagaatggtggatgtccttaggggttagcaagtgccgtgtgctaaggagggggctttggaggttgggcaggccctctgtggggctccatttttgtgggggtgggggctggagcattatagggggtgggaagtgattggggctgtcaccctagccttccttatctgacgcccacccatgcctcctcaggtaccccctgccccccacagctcctctcctgtgccttgtttcccagccatgcgttctcctctataaatacccgctctggtatttggggttggcagctgttgctgccagggagatggttgggttgacatgcggctcctgacaaaacacaaacccctggtgtgtgtgggcgtgggtggtgtgagtagggggatgaatcagggagggggcgggggacccagggggcaggagccacacaaagtctgtgcgggggtgggagcgcacatagcaattggaaactgaaagcttatcagaccctttctggaaatcagcccactgtttataaacttgaggccccaccctcgacagtaccggggaggaagagggcctgcactagtccagagggaaactgaggctcagggctagctcgcccatagacatacatggcaggcaggctttggccaggatccctccgcctgccaggcgtctccctgccctcccttcctgcctagagacccccaccctcaagcctggctggtctttgcctgagacccaaacctcttcgacttcaagagaatatttaggaacaaggtggtttagggcctttcctgggaacaggccttgaccctttaagaaatgacccaaagtctctccttgaccaaaaaggggaccctcaaactaaagggaagcctctcttctgctgtctcccctgaccccactcccccccaccccaggacgaggagataaccagggctgaaagaggcccgcctgggggctgcagacatgcttgctgcctgccctggcgaaggattggcaggcttgcccgtcacaggacccccgctggctgactcaggggcgcaggcctcttgcgggggagctggcctccccgcccccacggccacgggccgccctttcctggcaggacagcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcacctctagaaagaggtaagggtttaagggatggttggttggtggggtattaatgtttaattacctggagcacctgcctgaaatcactttttttcaggttggaagcttatggaagatgccaaaaacattaagaagggcccagcgccattctacccactcgaggacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaattcgaaacgattcgtccatcttgagcatctgacttctggctaaataaaagatctttattttcattagatctgtgtgttggttttttgtgtgcgtcgagatccacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctc

SEQ ID NO: 114 - DNA, 7108 bp, synthetic construct
  misc_feature 1..7108: note = plasmid pAAVss-CRE41-SkSH4-hDES1.4kb-Luc2
  source 1..7108: mol_type = other DNA
acatgtcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccctacaataaatatataacttttttaatttgacaaaaaataattttgagtggttacaaaaagggacaggtgtggcttggtaactagaaaaacattcccagcctgggaaacaatctagtgagagcttggaggcagaagacagaactctgattacattcaaggagtttgccagatggcaacagataactgccaatgttcggttgcactataattattatacactctctgtcacttcagcaagcgctcttttcacaagacaagtggtgacagaatgttgtattaagattacccgttgctaagcttatgttaaaatgaggaaatgaaatggaaagtcttgttttggtaatgtctctggggtatgaggaatggagggaaaggtttgactatgagcataactgcatagagaattttgtttgtttatgccttttgggaatgcattattttgattgaccctaattgggaattggcgcgccacgcgtttctgagtcctctaaggtccctcactcccaactcagccccatgtcctgtcaattcccactcagtgtctgatctccttctcctcacctttcccatctcccgtttgacccaagcttcctgagctctcctcccattcccctttttggagtcctcctcctctcccagaacccagtaataagtgggctcctccctggcctggacccccgtggtaaccctataaggcgaggcagctgctgtctgaggcagggaggggctggtgtgggaggctaagggcagctgctaagtttagggtggctccttctctcttcttagagacaacaggtggctggggcctcagtgcccagaaaagaaaatgtcttagaggtatcggcatgggcctggaggaggggggacagggcagggggaggcatcttcctcaggacatcgggtcctagaggggtacccaacgggttacgacacacctactagtaacccctccagctggtgatggcaggtctagggtaggaccagtgactggctcctaatcgagcactctattttcagggtttgcattccaaaagggtcaggtccaagagggacctggagtgccaagtggaggtgtagaggcacggccagtacccatggagaatggtggatgtccttaggggttagcaagtgccgtgtgctaaggagggggctttggaggttgggcaggccctctgtggggctccatttttgtgggggtgggggctggagcattatagggggtgggaagtgattggggctgtcaccctagccttccttatctgacgcccacccatgcctcctcaggtaccccctgccccccacagctcctctcctgtgccttgtttcccagccatgcgttctcctctataaatacccgctctggtatttggggttggcagctgttgctgccagggagatggttgggttgacatgcggctcctgacaaaacacaaacccctggtgtgtgtgggcgtgggtggtgtgagtagggggatgaatcagggagggggcgggggacccagggggcaggagccacacaaagtctgtgcgggggtgggagcgcacatagcaattggaaactgaaagcttatcagaccctttctggaaatcagcccactgtttataaacttgaggccccaccctcgacagtaccggggaggaagagggcctgcactagtccagagggaaactgaggctcagggctagctcgcccatagacatacatggcaggcaggctttggccaggatccctccgcctgccaggcgtctccctgccctcccttcctgcctagagacccccaccctcaagcctggctggtctttgcctgagacccaaacctcttcgacttcaagagaatatttaggaacaaggtggtttagggcctttcctgggaacaggccttgaccctttaagaaatgacccaaagtctctccttgaccaaaaaggggaccctcaaactaaagggaagcctctcttctgctgtctcccctgaccccactcccccccaccccaggacgaggagataaccagggctgaaagaggcccgcctgggggctgcagacatgcttgctgcctgccctggcgaaggattggcaggcttgcccgtcacaggacccccgctggctgactcaggggcgcaggcctcttgcgggggagctggcctccccgcccccacggccacgggccgccctttcctggcaggacagcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcacctctagaaagaggtaagggtttaagggatggttggttggtggggtattaatgtttaattacctggagcacctgcctgaaatcactttttttcaggttggaagcttatggaagatgccaaaaacattaagaagggcccagcgccattctacccactcgaggacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaattcgaaacgagaagtccatcttgagcatctgacttctggctaaataaaagatctttattttcattagatctgtgtgttggttttttgtgtgcgtcgagatccacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctc

SEQ ID NO: 115 - DNA, 6990 bp, synthetic construct
  misc_feature 1..6990: note = plasmid pAAVss-CRE58-SkSH4-hDES1.4kb-Luc2
  source 1..6990: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcccccctcagcttcagcccccacctccaggaggccctacccacgctcatgaccttgctattctgggccttgtgtcctgtagggagatggacaggagacagctgggcttccaggccacccaggcggggggctagccgagggaagcctgctggctctcctgcttgctctaatttctggggctccccaaaccttggcctcaggagactggggataggaccggccttgaaagtgggggaagctttggagagccgggtgctgggttcttagtgagatggccagtgaaggctgtggtgccccgaggtaagcagggcctgatcccctcctaatcttccagcagcaactggtgctggcgcgccacgcgtttctgagtcctctaaggtccctcactcccaactcagccccatgtcctgtcaattcccactcagtgtctgatctccttctcctcacctttcccatctcccgtttgacccaagcttcctgagctctcctcccattcccctttttggagtcctcctcctctcccagaacccagtaataagtgggctcctccctggcctggacccccgtggtaaccctataaggcgaggcagctgctgtctgaggcagggaggggctggtgtgggaggctaagggcagctgctaagtttagggtggctccttctctcttcttagagacaacaggtggctggggcctcagtgcccagaaaagaaaatgtcttagaggtatcggcatgggcctggaggaggggggacagggcagggggaggcatcttcctcaggacatcgggtcctagaggggtacccaacgggttacgacacacctactagtaacccctccagctggtgatggcaggtctagggtaggaccagtgactggctcctaatcgagcactctattttcagggtttgcattccaaaagggtcaggtccaagagggacctggagtgccaagtggaggtgtagaggcacggccagtacccatggagaatggtggatgtccttaggggttagcaagtgccgtgtgctaaggagggggctttggaggttgggcaggccctctgtggggctccatttttgtgggggtgggggctggagcattatagggggtgggaagtgattggggctgtcaccctagccttccttatctgacgcccacccatgcctcctcaggtaccccctgccccccacagctcctctcctgtgccttgtttcccagccatgcgttctcctctataaatacccgctctggtatttggggttggcagctgttgctgccagggagatggttgggttgacatgcggctcctgacaaaacacaaacccctggtgtgtgtgggcgtgggtggtgtgagtagggggatgaatcagggagggggcgggggacccagggggcaggagccacacaaagtctgtgcgggggtgggagcgcacatagcaattggaaactgaaagcttatcagaccctttctggaaatcagcccactgtttataaacttgaggccccaccctcgacagtaccggggaggaagagggcctgcactagtccagagggaaactgaggctcagggctagctcgcccatagacatacatggcaggcaggctttggccaggatccctccgcctgccaggcgtctccctgccctcccttcctgcctagagacccccaccctcaagcctggctggtctttgcctgagacccaaacctcttcgacttcaagagaatatttaggaacaaggtggtttagggcctttcctgggaacaggccttgaccctttaagaaatgacccaaagtctctccttgaccaaaaaggggaccctcaaactaaagggaagcctctcttctgctgtctcccctgaccccactcccccccaccccaggacgaggagataaccagggctgaaagaggcccgcctgggggctgcagacatgcttgctgcctgccctggcgaaggattggcaggcttgcccgtcacaggacccccgctggctgactcaggggcgcaggcctcttgcgggggagctggcctccccgcccccacggccacgggccgccctttcctggcaggacagcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcacctctagaaagaggtaagggtttaagggatggttggttggtggggtattaatgtttaattacctggagcacctgcctgaaatcactttttttcaggttggaagcttatggaagatgccaaaaacattaagaagggcccagcgccattctacccactcgaggacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaattcgaaacgaattgtccatcttgagcatctgacttctggctaaataaaagatctttattttcattagatctgtgtgttggttttttgtgtgcgtcgagatccacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 116 - DNA, 6865 bp, synthetic construct
  misc_feature 1..6865: note = plasmid pAAVss-CRE59-SkSH4-hDES1.4kb-Luc2
  source 1..6865: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccctcctccagcagctgccctggtggtaaccaagagacgcccccatcctggagcaggggtggggaggggcagctcagagcagctgcttctctgaggaagctgacaccaaggccagcattcagcaacaacttgtggctttgcacccagcgccggggtccccgcccacctggctccctgctgtccctcttccccactgctgctcggacttccctctgaccctggggcgcgccacgcgtttctgagtcctctaaggtccctcactcccaactcagccccatgtcctgtcaattcccactcagtgtctgatctccttctcctcacctttcccatctcccgtttgacccaagcttcctgagctctcctcccattcccctttttggagtcctcctcctctcccagaacccagtaataagtgggctcctccctggcctggacccccgtggtaaccctataaggcgaggcagctgctgtctgaggcagggaggggctggtgtgggaggctaagggcagctgctaagtttagggtggctccttctctcttcttagagacaacaggtggctggggcctcagtgcccagaaaagaaaatgtcttagaggtatcggcatgggcctggaggaggggggacagggcagggggaggcatcttcctcaggacatcgggtcctagaggggtacccaacgggttacgacacacctactagtaacccctccagctggtgatggcaggtctagggtaggaccagtgactggctcctaatcgagcactctattttcagggtttgcattccaaaagggtcaggtccaagagggacctggagtgccaagtggaggtgtagaggcacggccagtacccatggagaatggtggatgtccttaggggttagcaagtgccgtgtgctaaggagggggctttggaggttgggcaggccctctgtggggctccatttttgtgggggtgggggctggagcattatagggggtgggaagtgattggggctgtcaccctagccttccttatctgacgcccacccatgcctcctcaggtaccccctgccccccacagctcctctcctgtgccttgtttcccagccatgcgttctcctctataaatacccgctctggtatttggggttggcagctgttgctgccagggagatggttgggttgacatgcggctcctgacaaaacacaaacccctggtgtgtgtgggcgtgggtggtgtgagtagggggatgaatcagggagggggcgggggacccagggggcaggagccacacaaagtctgtgcgggggtgggagcgcacatagcaattggaaactgaaagcttatcagaccctttctggaaatcagcccactgtttataaacttgaggccccaccctcgacagtaccggggaggaagagggcctgcactagtccagagggaaactgaggctcagggctagctcgcccatagacatacatggcaggcaggctttggccaggatccctccgcctgccaggcgtctccctgccctcccttcctgcctagagacccccaccctcaagcctggctggtctttgcctgagacccaaacctcttcgacttcaagagaatatttaggaacaaggtggtttagggcctttcctgggaacaggccttgaccctttaagaaatgacccaaagtctctccttgaccaaaaaggggaccctcaaactaaagggaagcctctcttctgctgtctcccctgaccccactcccccccaccccaggacgaggagataaccagggctgaaagaggcccgcctgggggctgcagacatgcttgctgcctgccctggcgaaggattggcaggcttgcccgtcacaggacccccgctggctgactcaggggcgcaggcctcttgcgggggagctggcctccccgcccccacggccacgggccgccctttcctggcaggacagcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcacctctagaaagaggtaagggtttaagggatggttggttggtggggtattaatgtttaattacctggagcacctgcctgaaatcactttttttcaggttggaagcttatggaagatgccaaaaacattaagaagggcccagcgccattctacccactcgaggacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaattcgaaacgaatcgtccatcttgagcatctgacttctggctaaataaaagatctttattttcattagatctgtgtgttggttttttgtgtgcgtcgagatccacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 117 - DNA, 7308 bp, synthetic construct
  misc_feature 1..7308: note = plasmid pAAVss-CRE60-SkSH4-hDES1.4kb-Luc2
  source 1..7308: mol_type = other DNA
acatgtcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccctttgctccctgggagggcctggccctgtgggcatttgagtttataacaccacccccattgtggcacacccctccaccccgtaaaacacaggctctgctcttggaatcagtcttcctgatctgtggctgtgccctccaacagagggcacccctgggcttcccagctctgggggtagtgggtgccaacaaggaggggcctggggctgaagaatcccacccgctgagctcggccttctcccttccccactgtccagctccgcctttcagcatcctgcctcactccccgcccaggcagcaaggagcccacaccctcatgcccctcagcttcagcccccacctccaggaggccctacccacgctcatgaccttgctattctgggccttgtgtcctgtagggagatggacaggagacagctgggcttccaggccacccaggcggggggctagccgagggaagcctgctggctctcctgcttgctctaatttctggggctccccaaaccttggcctcaggagactggggataggaccggccttgaaagtgggggaagctttggagagccgggtgctgggttcttagtgagatggccagtgaaggctgtggtgccccgaggtaagcagggcctgatcccctcctaatcttccagcagcaactggtgctctggcgcgccacgcgtttctgagtcctctaaggtccctcactcccaactcagccccatgtcctgtcaattcccactcagtgtctgatctccttctcctcacctttcccatctcccgtttgacccaagcttcctgagctctcctcccattcccctttttggagtcctcctcctctcccagaacccagtaataagtgggctcctccctggcctggacccccgtggtaaccctataaggcgaggcagctgctgtctgaggcagggaggggctggtgtgggaggctaagggcagctgctaagtttagggtggctccttctctcttcttagagacaacaggtggctggggcctcagtgcccagaaaagaaaatgtcttagaggtatcggcatgggcctggaggaggggggacagggcagggggaggcatcttcctcaggacatcgggtcctagaggggtacccaacgggttacgacacacctactagtaacccctccagctggtgatggcaggtctagggtaggaccagtgactggctcctaatcgagcactctattttcagggtttgcattccaaaagggtcaggtccaagagggacctggagtgccaagtggaggtgtagaggcacggccagtacccatggagaatggtggatgtccttaggggttagcaagtgccgtgtgctaaggagggggctttggaggttgggcaggccctctgtggggctccatttttgtgggggtgggggctggagcattatagggggtgggaagtgattggggctgtcaccctagccttccttatctgacgcccacccatgcctcctcaggtaccccctgccccccacagctcctctcctgtgccttgtttcccagccatgcgttctcctctataaatacccgctctggtatttggggttggcagctgttgctgccagggagatggttgggttgacatgcggctcctgacaaaacacaaacccctggtgtgtgtgggcgtgggtggtgtgagtagggggatgaatcagggagggggcgggggacccagggggcaggagccacacaaagtctgtgcgggggtgggagcgcacatagcaattggaaactgaaagcttatcagaccctttctggaaatcagcccactgtttataaacttgaggccccaccctcgacagtaccggggaggaagagggcctgcactagtccagagggaaactgaggctcagggctagctcgcccatagacatacatggcaggcaggctttggccaggatccctccgcctgccaggcgtctccctgccctcccttcctgcctagagacccccaccctcaagcctggctggtctttgcctgagacccaaacctcttcgacttcaagagaatatttaggaacaaggtggtttagggcctttcctgggaacaggccttgaccctttaagaaatgacccaaagtctctccttgaccaaaaaggggaccctcaaactaaagggaagcctctcttctgctgtctcccctgaccccactcccccccaccccaggacgaggagataaccagggctgaaagaggcccgcctgggggctgcagacatgcttgctgcctgccctggcgaaggattggcaggcttgcccgtcacaggacccccgctggctgactcaggggcgcaggcctcttgcgggggagctggcctccccgcccccacggccacgggccgccctttcctggcaggacagcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcacctctagaaagaggtaagggtttaagggatggttggttggtggggtattaatgtttaattacctggagcacctgcctgaaatcactttttttcaggttggaagcttatggaagatgccaaaaacattaagaagggcccagcgccattctacccactcgaggacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaattcgaaacgaatggtccatcttgagcatctgacttctggctaaataaaagatctttattttcattagatctgtgtgttggttttttgtgtgcgtcgagatccacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctc

SEQ ID NO: 118 - DNA, 7053 bp, synthetic construct
  misc_feature 1..7053: note = plasmid pAAVss-CRE64-SkSH4-hDES1.4kb-Luc2
  source 1..7053: mol_type = other DNA
acatgtcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgtctccgaacgcaggccccgtcgcgttaagcacaagctggcagggcctctcctctcccttctcagatttgctccttgacatttgcctgctgcctggcggtggcaacagctggggcggggcgcgcgcaggaggccccgtaaccctatccccgctccggctccctcgtgaaaccggagcttccctgccttggccgagggggagggctgcgggggccagaccgcctgcgaagaccacagggtttttcctctcgggttttggctcccgtgggatggatgtggctgtgcggggggttggcctgagcttcgcttctaagccagcagcttggtcagggaaacctgaaagcattcccagctaatcccccaagtggtgcaagtctgtgcgcgcccatcccgctgagtaaggcggtggggcgcgccacgcgtttctgagtcctctaaggtccctcactcccaactcagccccatgtcctgtcaattcccactcagtgtctgatctccttctcctcacctttcccatctcccgtttgacccaagcttcctgagctctcctcccattcccctttttggagtcctcctcctctcccagaacccagtaataagtgggctcctccctggcctggacccccgtggtaaccctataaggcgaggcagctgctgtctgaggcagggaggggctggtgtgggaggctaagggcagctgctaagtttagggtggctccttctctcttcttagagacaacaggtggctggggcctcagtgcccagaaaagaaaatgtcttagaggtatcggcatgggcctggaggaggggggacagggcagggggaggcatcttcctcaggacatcgggtcctagaggggtacccaacgggttacgacacacctactagtaacccctccagctggtgatggcaggtctagggtaggaccagtgactggctcctaatcgagcactctattttcagggtttgcattccaaaagggtcaggtccaagagggacctggagtgccaagtggaggtgtagaggcacggccagtacccatggagaatggtggatgtccttaggggttagcaagtgccgtgtgctaaggagggggctttggaggttgggcaggccctctgtggggctccatttttgtgggggtgggggctggagcattatagggggtgggaagtgattggggctgtcaccctagccttccttatctgacgcccacccatgcctcctcaggtaccccctgccccccacagctcctctcctgtgccttgtttcccagccatgcgttctcctctataaatacccgctctggtatttggggttggcagctgttgctgccagggagatggttgggttgacatgcggctcctgacaaaacacaaacccctggtgtgtgtgggcgtgggtggtgtgagtagggggatgaatcagggagggggcgggggacccagggggcaggagccacacaaagtctgtgcgggggtgggagcgcacatagcaattggaaactgaaagcttatcagaccctttctggaaatcagcccactgtttataaacttgaggccccaccctcgacagtaccggggaggaagagggcctgcactagtccagagggaaactgaggctcagggctagctcgcccatagacatacatggcaggcaggctttggccaggatccctccgcctgccaggcgtctccctgccctcccttcctgcctagagacccccaccctcaagcctggctggtctttgcctgagacccaaacctcttcgacttcaagagaatatttaggaacaaggtggtttagggcctttcctgggaacaggccttgaccctttaagaaatgacccaaagtctctccttgaccaaaaaggggaccctcaaactaaagggaagcctctcttctgctgtctcccctgaccccactcccccccaccccaggacgaggagataaccagggctgaaagaggcccgcctgggggctgcagacatgcttgctgcctgccctggcgaaggattggcaggcttgcccgtcacaggacccccgctggctgactcaggggcgcaggcctcttgcgggggagctggcctccccgcccccacggccacgggccgccctttcctggcaggacagcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcacctctagaaagaggtaagggtttaagggatggttggttggtggggtattaatgtttaattacctggagcacctgcctgaaatcactttttttcaggttggaagcttatggaagatgccaaaaacattaagaagggcccagcgccattctacccactcgaggacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaattcgaaacgaacggtccatcttgagcatctgacttctggctaaataaaagatctttattttcattagatctgtgtgttggttttttgtgtgcgtcgagatccacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctc

SEQ ID NO: 119 - DNA, 6210 bp, synthetic construct
  misc_feature 1..6210: note = plasmid pAAVss-hDES1.4kb-Luc2
  source 1..6210: mol_type = other DNA
acatgtcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccggcgcgccacgcgtggtacccaacgggttacgacacacctactagtaacccctccagctggtgatggcaggtctagggtaggaccagtgactggctcctaatcgagcactctattttcagggtttgcattccaaaagggtcaggtccaagagggacctggagtgccaagtggaggtgtagaggcacggccagtacccatggagaatggtggatgtccttaggggttagcaagtgccgtgtgctaaggagggggctttggaggttgggcaggccctctgtggggctccatttttgtgggggtgggggctggagcattatagggggtgggaagtgattggggctgtcaccctagccttccttatctgacgcccacccatgcctcctcaggtaccccctgccccccacagctcctctcctgtgccttgtttcccagccatgcgttctcctctataaatacccgctctggtatttggggttggcagctgttgctgccagggagatggttgggttgacatgcggctcctgacaaaacacaaacccctggtgtgtgtgggcgtgggtggtgtgagtagggggatgaatcagggagggggcgggggacccagggggcaggagccacacaaagtctgtgcgggggtgggagcgcacatagcaattggaaactgaaagcttatcagaccctttctggaaatcagcccactgtttataaacttgaggccccaccctcgacagtaccggggaggaagagggcctgcactagtccagagggaaactgaggctcagggctagctcgcccatagacatacatggcaggcaggctttggccaggatccctccgcctgccaggcgtctccctgccctcccttcctgcctagagacccccaccctcaagcctggctggtctttgcctgagacccaaacctcttcgacttcaagagaatatttaggaacaaggtggtttagggcctttcctgggaacaggccttgaccctttaagaaatgacccaaagtctctccttgaccaaaaaggggaccctcaaactaaagggaagcctctcttctgctgtctcccctgaccccactcccccccaccccaggacgaggagataaccagggctgaaagaggcccgcctgggggctgcagacatgcttgctgcctgccctggcgaaggattggcaggcttgcccgtcacaggacccccgctggctgactcaggggcgcaggcctcttgcgggggagctggcctccccgcccccacggccacgggccgccctttcctggcaggacagcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcacctctagaaagaggtaagggtttaagggatggttggttggtggggtattaatgtttaattacctggagcacctgcctgaaatcactttttttcaggttggaagcttatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaattcgaaacgttgagtccatcttgagcatctgacttctggctaaataaaagatctttattttcattagatctgtgtgttggttttttgtgtgcgtcgagatccacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctc

SEQ ID NO: 120 - DNA, 6645 bp, synthetic construct
  misc_feature 1..6645: note = plasmid pAAVss-SkSH4-hDES1.4kb-Luc2
  source 1..6645: mol_type = other DNA
acatgtcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccggcgcgccacgcgtttctgagtcctctaaggtccctcactcccaactcagccccatgtcctgtcaattcccactcagtgtctgatctccttctcctcacctttcccatctcccgtttgacccaagcttcctgagctctcctcccattcccctttttggagtcctcctcctctcccagaacccagtaataagtgggctcctccctggcctggacccccgtggtaaccctataaggcgaggcagctgctgtctgaggcagggaggggctggtgtgggaggctaagggcagctgctaagtttagggtggctccttctctcttcttagagacaacaggtggctggggcctcagtgcccagaaaagaaaatgtcttagaggtatcggcatgggcctggaggaggggggacagggcagggggaggcatcttcctcaggacatcgggtcctagaggggtacccaacgggttacgacacacctactagtaacccctccagctggtgatggcaggtctagggtaggaccagtgactggctcctaatcgagcactctattttcagggtttgcattccaaaagggtcaggtccaagagggacctggagtgccaagtggaggtgtagaggcacggccagtacccatggagaatggtggatgtccttaggggttagcaagtgccgtgtgctaaggagggggctttggaggttgggcaggccctctgtggggctccatttttgtgggggtgggggctggagcattatagggggtgggaagtgattggggctgtcaccctagccttccttatctgacgcccacccatgcctcctcaggtaccccctgccccccacagctcctctcctgtgccttgtttcccagccatgcgttctcctctataaatacccgctctggtatttggggttggcagctgttgctgccagggagatggttgggttgacatgcggctcctgacaaaacacaaacccctggtgtgtgtgggcgtgggtggtgtgagtagggggatgaatcagggagggggcgggggacccagggggcaggagccacacaaagtctgtgcgggggtgggagcgcacatagcaattggaaactgaaagcttatcagaccctttctggaaatcagcccactgtttataaacttgaggccccaccctcgacagtaccggggaggaagagggcctgcactagtccagagggaaactgaggctcagggctagctcgcccatagacatacatggcaggcaggctttggccaggatccctccgcctgccaggcgtctccctgccctcccttcctgcctagagacccccaccctcaagcctggctggtctttgcctgagacccaaacctcttcgacttcaagagaatatttaggaacaaggtggtttagggcctttcctgggaacaggccttgaccctttaagaaatgacccaaagtctctccttgaccaaaaaggggaccctcaaactaaagggaagcctctcttctgctgtctcccctgaccccactcccccccaccccaggacgaggagataaccagggctgaaagaggcccgcctgggggctgcagacatgcttgctgcctgccctggcgaaggattggcaggcttgcccgtcacaggacccccgctggctgactcaggggcgcaggcctcttgcgggggagctggcctccccgcccccacggccacgggccgccctttcctggcaggacagcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcacctctagaaagaggtaagggtttaagggatggttggttggtggggtattaatgtttaattacctggagcacctgcctgaaatcactttttttcaggttggaagcttatggaagatgccaaaaacattaagaagggcccagcgccattctacccactcgaggacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaattcgaaacgttccgtccatcttgagcatctgacttctggctaaataaaagatctttattttcattagatctgtgtgttggttttttgtgtgcgtcgagatccacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctc

SEQ ID NO: 121 - DNA, 435 bp, synthetic construct
  misc_feature 1..435: note = Muscle CRE Sk-SH4
  source 1..435: mol_type = other DNA
ttctgagtcctctaaggtccctcactcccaactcagccccatgtcctgtcaattcccactcagtgtctgatctccttctcctcacctttcccatctcccgtttgacccaagcttcctgagctctcctcccattcccctttttggagtcctcctcctctcccagaacccagtaataagtgggctcctccctggcctggacccccgtggtaaccctataaggcgaggcagctgctgtctgaggcagggaggggctggtgtgggaggctaagggcagctgctaagtttagggtggctccttctctcttcttagagacaacaggtggctggggcctcagtgcccagaaaagaaaatgtcttagaggtatcggcatgggcctggaggaggggggacagggcagggggaggcatcttcctcaggacatcgggtcctagagg

SEQ ID NO: 122 - DNA, 454 bp, synthetic construct
  misc_feature 1..454: note = Heart-Muscle CRE CSk-SH5
  source 1..454: mol_type = other DNA
cagtttactcaccagggattcagaggcagcactgctgaaccctgagcccttggcacatcaggttggctgtcagaagtcggcctttgtacatacacagttcccttgtgaggcccagctgcgtgtcctaggagcggggcctctctccacagcagagctcagcctctcaagtgtatggacagcacgggtgcctgatgggtggatttagccatgagttgaaggtggcttggggagaatgagagttctagagatagggagaaggggttgccaataggagagtggaattcctgagcacctcgtcacaggcagccgacagaacatgagccgcagggcccaggctatttatacctcgcctgtcactatcagggtccccacagctccccccacctccagccacacacagcaggtccttttgctctttctggtcccttctctactcctccccctccctacctaa

SEQ ID NO: 123 - DNA, 495 bp, synthetic construct
  misc_feature 1..495: note = Heart-Muscle CRE CSk-SH1
  source 1..495: mol_type = other DNA
ctcactccccgcccaggcagcaaggagcccacaccctcatgcccctcagcttcagcccccacctccaggaggccctacccacgctcatgaccttgctattctgggccttgtgtcctgtagggagatggacaggagacagctgggcttccaggccacccaggcggggggctagccgagggaagcctgctggctctcctgcttgctctaatttctggggctccccaaaccttggcctcaggagactggggataggaccggccttgaaagtgggggaagctttggagagccgggtgctgggttcttagtgagatggccagtgaaggctgtggtgccccgaggtaagcagggcctgatcccctcctaatcttccagcagcaactggtgctctgaggctccccctcccccagccctgccagccttcagggacctgccttccaaagatgggcaggggagggggacgaggacacccacccactcctcagaccagcatgtctt

SEQ ID NO: 124 - DNA, 334 bp, synthetic construct
  misc_feature 1..334: note = SPc-5-12 promotor
  source 1..334: mol_type = other DNA
tggccaccgccttcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctaga

SEQ ID NO: 125 - DNA, 92 bp, synthetic construct
  misc_feature 1..92: note = MVM intron
  source 1..92: mol_type = other DNA
aagaggtaagggtttaagggatggttggttggtggggtattaatgtttaattacctggagcacctgcctgaaatcactttttttcaggttgg

SEQ ID NO: 126 - DNA, 134 bp, synthetic construct
  misc_feature 1..134: note = SV40 Polyadenylation signal
  source 1..134: mol_type = other DNA
atgctttatttgtgaaatttgtgatgctattgctttatttgtaaccattataagctgcaataaacaagttaacaacaacaattgcattcattttatgtttcaggttcagggggaggtgtgggaggttttttaaa

SEQ ID NO: 127 - DNA, 49 bp, synthetic construct
  misc_feature 1..49: note = synthetic Polyadenylation signal
  source 1..49: mol_type = other DNA
aataaaagatctttattttcattagatctgtgtgttggttttttgtgtg

SEQ ID NO: 128 - DNA, 20 bp, synthetic construct
  misc_feature 1..20: note = forward primer luciferase
  source 1..20: mol_type = other DNA
cccaccgtcgtattcgtgag

SEQ ID NO: 129 - DNA, 21 bp, synthetic construct
  misc_feature 1..21: note = reverse primer luciferase
  source 1..21: mol_type = other DNA
tcagggcgatggttttgtccc

SEQ ID NO: 130 - DNA, 7303 bp, synthetic construct
  misc_feature 1..7303: note = plasmid pAAVss-CRE04-CSkSH5-SPc5-12GTRM-MVM-hGAAco-SynthpA
  source 1..7303: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtaccggtccttttagagaatccacacctgtcccagttgctgggttccactaccaaaagtgaattgcaactattttaggagcacttaagcacatccgaaaaatgagtgattctgttctggcccacaccacatcactgatgtacccccttaaagcatgtccctgagttcatcacagaagactgctcctcctgtgccctccacaaggttagaactgtccttgtcttagggaaaaaggagagagagagagagagagagagagagagagagagagagagagagagagagggacaggcaccaactgggtaacctctgctgacccccactctactttaccataagtagctccaaatccttctagaaaatctgaaaggcatagccccatatatcagtgatataaatagaacctgcagcaggctctggtaaatgatgactacaaggtggactgggaggcagcccggccttggcaggcatcatcctctaaatataaagatgagtttgttcagcctttgcagaaggaggcgcgccacgcgtcagtttactcaccagggattcagaggcagcactgctgaaccctgagcccttggcacatcaggttggctgtcagaagtcggcctttgtacatacacagttcccttgtgaggcccagctgcgtgtcctaggagcggggcctctctccacagcagagctcagcctctcaagtgtatggacagcacgggtgcctgatgggtggatttagccatgagttgaaggtggcttggggagaatgagagttctagagatagggagaaggggttgccaataggagagtggaattcctgagcacctcgtcacaggcagccgacagaacatgagccgcagggcccaggctatttatacctcgcctgtcactatcagggtccccacagctccccccacctccagccacacacagcaggtccttttgctctttctggtcccttctctactcctccccctccctacctaaggtacccaacccgttacgtggccaccgccttcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagatctagaactagtaagaggtaagggtttaagggatggttggttggtggggtattaatgtttaattacctggagcacctgcctgaaatcactttttttcaggttggcgtacggccaccatgggcgtcagacatcctccatgttctcacagactgctggccgtgtgtgctctggtgtctcttgctacagctgccctgctgggacatatcctgctgcacgattttctgctggtgcccagagagctgtctggcagctctcctgtgctggaagaaacacaccctgcacatcagcagggcgcctctagacctggacctagagatgctcaagcccatcctggcagacctagagccgtgcctacacagtgtgacgtgccacctaacagcagattcgactgcgcccctgacaaggccatcacacaagagcagtgtgaagccagaggctgctgctacattcctgccaaacaaggactgcagggcgctcagatgggacagccttggtgcttcttcccaccatcttaccccagctacaagctggaaaacctgagcagcagcgagatgggctacaccgccacactgaccagaaccacacctacattcttcccaaaggacatcctgacactgcggctggacgtgatgatggaaaccgagaaccggctgcacttcaccatcaaggaccccgccaatagaagatacgaggtgcccctggaaacccctcacgtgcactctagagccccatctccactgtacagcgtggaattcagcgaggaaccctttggcgtgatcgtgcggagacagctggatggcagagtgctgctgaataccacagtggcccctctgttcttcgccgaccagtttctgcagctgagcacaagcctgcctagccagtatatcacaggcctggccgaacacctgtctccactgatgctgagcaccagctggaccagaatcaccctgtggaacagagatctggcccctacacctggcgccaatctgtacggctctcaccctttttatctggccctggaagatggcggaagcgcccacggtgtctttctgctgaacagcaacgccatggacgtggtgctgcaaccatctcctgctctgtcttggagaagcaccggcggcatcctggacgtgtacatctttctgggacccgagcctaagagcgtggtgcagcagtatctggatgtcgtgggctaccccttcatgcctccttattggggcctgggcttccacctgtgtagatggggatacagctccaccgccatcaccagacaggtggtggaaaacatgacccgggctcacttcccactggatgtgcagtggaacgacctggactacatggactccagacgggacttcacctttaacaaggacggcttcagagacttccccgccatggtgcaagaactgcatcaaggcggcagacggtacatgatgatcgtggatcctgccatctcttctagcggccctgccggaagctacagaccttatgatgagggcctgagaagaggcgtgttcatcaccaatgagacaggccagcctctgatcggcaaagtgtggcctggaagcaccgcctttccagacttcaccaatccaaccgctctggcttggtgggaagatatggtggccgagttccacgatcaggtgcccttcgatggcatgtggatcgacatgaacgagcccagcaacttcatcaggggcagcgaggatggctgccccaacaacgaactggaaaatcctccttacgtgccaggcgttgtcggaggaacactgcaggccgccacaatttgtgccagcagccatcagtttctgagcacccactacaacctgcacaacctgtacggcctgaccgaggccattgcctctcatagagccctggttaaggccagaggcacccggccttttgtgatcagcagaagcacatttgccggccacggcagatatgccggacattggacaggggacgtttggtctagttgggagcagctggcctctagcgtgcccgagatcctgcagtttaatctgctgggagtgcccctcgtgggagccgatgtttgtggatttctgggcaacacctccgaggaactgtgcgtcagatggacacagctgggcgccttctatcccttcatgagaaaccacaacagcctgctgagcctgcctcaagagccttacagctttagcgaacccgcacagcaggccatgagaaaggccctgactctgagatacgctctgctgccccacctgtacaccctgtttcatcaagctcatgtggccggcgagacagtggccagaccactgtttctggaattccccaaggacagcagcacctggacagtggatcatcagctgctctggggagaagccctgctcattacacctgtgctgcaggctggcaaggccgaagtgacaggatactttcccctcggcacttggtacgacctgcagacagttcctgtggaagctctgggatctctgcctccacctcctgctgctcctagagagcctgccattcactctgaaggccagtgggttacactgcccgctccactggacaccatcaatgtgcacctgagagccggctacatcatccctctgcaaggccctggactgaccacaaccgaaagcagacagcagccaatggctctggccgtggctctgacaaaaggcggagaagctagaggcgaactgttctgggatgacggcgagagcctggaagtgctggaacggggagcctacacacaagtgatctttctcgcccggaacaacaccatcgtgaacgaactcgtcagagtgaccagtgaaggtgccggactgcagctccagaaagtgacagtgcttggagtggccacagcaccccagcaggttttgtctaatggcgtgcccgtgtccaacttcacatacagccctgacaccaaggtgctggacatctgtgtgtctctgctgatgggcgagcagttcctggtgtcctggtgttgacctaggaataaaagatctttattttcattagatctgtgtgttggttttttgtgtgacccgtctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 131 - DNA, 7224 bp, synthetic construct
  misc_feature 1..7224: note = plasmid pAAVss-CRE64-SKCRM4-hDES1.4kb-MVM-hMTM1co-SynthpA
  source 1..7224: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcgcgccaccggtgtctccgaacgcaggccccgtcgcgttaagcacaagctggcagggcctctcctctcccttctcagatttgctccttgacatttgcctgctgcctggcggtggcaacagctggggcggggcgcgcgcaggaggccccgtaaccctatccccgctccggctccctcgtgaaaccggagcttccctgccttggccgagggggagggctgcgggggccagaccgcctgcgaagaccacagggtttttcctctcgggttttggctcccgtgggatggatgtggctgtgcggggggttggcctgagcttcgcttctaagccagcagcttggtcagggaaacctgaaagcattcccagctaatcccccaagtggtgcaagtctgtgcgcgcccatcccgctgagtaaggcggtggggcgcgccacgcgtttctgagtcctctaaggtccctcactcccaactcagccccatgtcctgtcaattcccactcagtgtctgatctccttctcctcacctttcccatctcccgtttgacccaagcttcctgagctctcctcccattcccctttttggagtcctcctcctctcccagaacccagtaataagtgggctcctccctggcctggacccccgtggtaaccctataaggcgaggcagctgctgtctgaggcagggaggggctggtgtgggaggctaagggcagctgctaagtttagggtggctccttctctcttcttagagacaacaggtggctggggcctcagtgcccagaaaagaaaatgtcttagaggtatcggcatgggcctggaggaggggggacagggcagggggaggcatcttcctcaggacatcgggtcctagaggggtacccaacgggttacgacacacctactagtaacccctccagctggtgatggcaggtctagggtaggaccagtgactggctcctaatcgagcactctattttcagggtttgcattccaaaagggtcaggtccaagagggacctggagtgccaagtggaggtgtagaggcacggccagtacccatggagaatggtggatgtccttaggggttagcaagtgccgtgtgctaaggagggggctttggaggttgggcaggccctctgtggggctccatttttgtgggggtgggggctggagcattatagggggtgggaagtgattggggctgtcaccctagccttccttatctgacgcccacccatgcctcctcaggtaccccctgccccccacagctcctctcctgtgccttgtttcccagccatgcgttctcctctataaatacccgctctggtatttggggttggcagctgttgctgccagggagatggttgggttgacatgcggctcctgacaaaacacaaacccctggtgtgtgtgggcgtgggtggtgtgagtagggggatgaatcagggagggggcgggggacccagggggcaggagccacacaaagtctgtgcgggggtgggagcgcacatagcaattggaaactgaaagcttatcagaccctttctggaaatcagcccactgtttataaacttgaggccccaccctcgacagtaccggggaggaagagggcctgcactagtccagagggaaactgaggctcagggctagctcgcccatagacatacatggcaggcaggctttggccaggatccctccgcctgccaggcgtctccctgccctcccttcctgcctagagacccccaccctcaagcctggctggtctttgcctgagacccaaacctcttcgacttcaagagaatatttaggaacaaggtggtttagggcctttcctgggaacaggccttgaccctttaagaaatgacccaaagtctctccttgaccaaaaaggggaccctcaaactaaagggaagcctctcttctgctgtctcccctgaccccactcccccccaccccaggacgaggagataaccagggctgaaagaggcccgcctgggggctgcagacatgcttgctgcctgccctggcgaaggattggcaggcttgcccgtcacaggacccccgctggctgactcaggggcgcaggcctcttgcgggggagctggcctccccgcccccacggccacgggccgccctttcctggcaggacagcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcacctctagaactagtaagaggtaagggtttaagggatggttggttggtggggtattaatgtttaattacctggagcacctgcctgaaatcactttttttcaggttggcgtacggccaccatggccagcgccagcacaagcaagtacaacagccacagcctggaaaacgagagcatcaagcggaccagcagagatggcgtgaacagagatctgaccgaggccgttcctagactgcctggcgagacactgatcaccgacaaagaagtgatctacatctgccccttcaacggccccatcaagggaagagtgtacatcaccaactaccggctgtacctgcggtccctggaaaccgatagcagcctgattctggatgtgcccctgggcgtgatcagccggattgaaaaaatgggcggagccacctccagaggcgagaatagctatggcctggatatcacatgcaaggacatgcggaacctgagattcgccctgaagcaagagggccacagcagacgggacatgttcgagatcctgaccagatacgcctttcctctggctcactctctgcccctgttcgccttcctgaacgaagagaagttcaacgtggacggctggaccgtgtacaaccccgtggaagagtatagacggcagggactgcccaatcaccactggcggatcaccttcatcaacaagtgctacgagctgtgcgacacataccccgcactgctggtggtgccttacagagcctctgacgacgatctgagaagagtggccacctttcggagccggaacagaatccctgtgctgagctggattcaccccgagaacaagaccgtgatcgtgcggtgttctcagcctctcgtgggcatgagcggcaagagaaacaaggacgacgagaagtacctggacgtgatccgcgagacaaacaagcagatcagcaagctgaccatctacgacgccagaccttctgtgaacgccgtggccaacaaagccacaggcggcggatatgagtccgacgatgcctatcacaacgccgagctgttcttcctggacattcacaacatccatgtgatgcgcgagagcctgaagaaagtgaaggacatcgtgtaccccaatgtggaagagagccactggctgtctagcctggaatccacacactggctggaacacatcaagctggtgctgacaggcgccatccaggtggcagacaaagtgtctagcggcaagtctagcgtgctggtgcactgtagcgacggatgggatagaacagcccagctgacatccctggccatgctgatgctggacagcttctacagatccatcgagggctttgagatcctggtgcagaagaagtggatcagcttcggccacaagttcgcctctagaatcggacacggcgacaagaaccacaccgacgccgatagaagccccatcttcctgcagttcatcgactgcgtgtggcagatgtccaagcagttccctaccgccttcgagttcaacgagcagttcctgatcatcatcctggaccacctgtactcttgcagattcggcaccttcctgttcaactgcgagagcgccagagaacggcagaaagtgaccgagagaaccgtgtctctgtggtccctgatcaacagcaacaaagagaaattcaagaaccccttctacaccaaagaaatcaaccgggtgctgtaccccgtggccagcatgagacatctggaactgtgggtcaactactacatccggtggaaccccagaatcaagcagcagcagcccaatcctgtggaacagcggtatatggaactgctggccctgcgggacgagtacatcaagagactggaagaactgcagctggccaacagcgccaagctgagcgatcctcctacaagccctagcagcccctctcagatgatgccccatgtgcagacccacttttgacctaggaataaaagatctttattttcattagatctgtgtgttggttttttgtgtgacccgtctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 132 - DNA, 6780 bp, synthetic construct
  misc_feature 1..6780: note = plasmid pAAVss-CSkSH5-SPc5-12GTRM-MVM-hGAAco-SynthpA
  source 1..6780: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtaccggtcagtttactcaccagggattcagaggcagcactgctgaaccctgagcccttggcacatcaggttggctgtcagaagtcggcctttgtacatacacagttcccttgtgaggcccagctgcgtgtcctaggagcggggcctctctccacagcagagctcagcctctcaagtgtatggacagcacgggtgcctgatgggtggatttagccatgagttgaaggtggcttggggagaatgagagttctagagatagggagaaggggttgccaataggagagtggaattcctgagcacctcgtcacaggcagccgacagaacatgagccgcagggcccaggctatttatacctcgcctgtcactatcagggtccccacagctccccccacctccagccacacacagcaggtccttttgctctttctggtcccttctctactcctccccctccctacctaaggtacccaacccgttacgtggccaccgccttcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagatctagaactagtaagaggtaagggtttaagggatggttggttggtggggtattaatgtttaattacctggagcacctgcctgaaatcactttttttcaggttggcgtacggccaccatgggcgtcagacatcctccatgttctcacagactgctggccgtgtgtgctctggtgtctcttgctacagctgccctgctgggacatatcctgctgcacgattttctgctggtgcccagagagctgtctggcagctctcctgtgctggaagaaacacaccctgcacatcagcagggcgcctctagacctggacctagagatgctcaagcccatcctggcagacctagagccgtgcctacacagtgtgacgtgccacctaacagcagattcgactgcgcccctgacaaggccatcacacaagagcagtgtgaagccagaggctgctgctacattcctgccaaacaaggactgcagggcgctcagatgggacagccttggtgcttcttcccaccatcttaccccagctacaagctggaaaacctgagcagcagcgagatgggctacaccgccacactgaccagaaccacacctacattcttcccaaaggacatcctgacactgcggctggacgtgatgatggaaaccgagaaccggctgcacttcaccatcaaggaccccgccaatagaagatacgaggtgcccctggaaacccctcacgtgcactctagagccccatctccactgtacagcgtggaattcagcgaggaaccctttggcgtgatcgtgcggagacagctggatggcagagtgctgctgaataccacagtggcccctctgttcttcgccgaccagtttctgcagctgagcacaagcctgcctagccagtatatcacaggcctggccgaacacctgtctccactgatgctgagcaccagctggaccagaatcaccctgtggaacagagatctggcccctacacctggcgccaatctgtacggctctcaccctttttatctggccctggaagatggcggaagcgcccacggtgtctttctgctgaacagcaacgccatggacgtggtgctgcaaccatctcctgctctgtcttggagaagcaccggcggcatcctggacgtgtacatctttctgggacccgagcctaagagcgtggtgcagcagtatctggatgtcgtgggctaccccttcatgcctccttattggggcctgggcttccacctgtgtagatggggatacagctccaccgccatcaccagacaggtggtggaaaacatgacccgggctcacttcccactggatgtgcagtggaacgacctggactacatggactccagacgggacttcacctttaacaaggacggcttcagagacttccccgccatggtgcaagaactgcatcaaggcggcagacggtacatgatgatcgtggatcctgccatctcttctagcggccctgccggaagctacagaccttatgatgagggcctgagaagaggcgtgttcatcaccaatgagacaggccagcctctgatcggcaaagtgtggcctggaagcaccgcctttccagacttcaccaatccaaccgctctggcttggtgggaagatatggtggccgagttccacgatcaggtgcccttcgatggcatgtggatcgacatgaacgagcccagcaacttcatcaggggcagcgaggatggctgccccaacaacgaactggaaaatcctccttacgtgccaggcgttgtcggaggaacactgcaggccgccacaatttgtgccagcagccatcagtttctgagcacccactacaacctgcacaacctgtacggcctgaccgaggccattgcctctcatagagccctggttaaggccagaggcacccggccttttgtgatcagcagaagcacatttgccggccacggcagatatgccggacattggacaggggacgtttggtctagttgggagcagctggcctctagcgtgcccgagatcctgcagtttaatctgctgggagtgcccctcgtgggagccgatgtttgtggatttctgggcaacacctccgaggaactgtgcgtcagatggacacagctgggcgccttctatcccttcatgagaaaccacaacagcctgctgagcctgcctcaagagccttacagctttagcgaacccgcacagcaggccatgagaaaggccctgactctgagatacgctctgctgccccacctgtacaccctgtttcatcaagctcatgtggccggcgagacagtggccagaccactgtttctggaattccccaaggacagcagcacctggacagtggatcatcagctgctctggggagaagccctgctcattacacctgtgctgcaggctggcaaggccgaagtgacaggatactttcccctcggcacttggtacgacctgcagacagttcctgtggaagctctgggatctctgcctccacctcctgctgctcctagagagcctgccattcactctgaaggccagtgggttacactgcccgctccactggacaccatcaatgtgcacctgagagccggctacatcatccctctgcaaggccctggactgaccacaaccgaaagcagacagcagccaatggctctggccgtggctctgacaaaaggcggagaagctagaggcgaactgttctgggatgacggcgagagcctggaagtgctggaacggggagcctacacacaagtgatctttctcgcccggaacaacaccatcgtgaacgaactcgtcagagtgaccagtgaaggtgccggactgcagctccagaaagtgacagtgcttggagtggccacagcaccccagcaggttttgtctaatggcgtgcccgtgtccaacttcacatacagccctgacaccaaggtgctggacatctgtgtgtctctgctgatgggcgagcagttcctggtgtcctggtgttgacctaggaataaaagatctttattttcattagatctgtgtgttggttttttgtgtgacccgtctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 133 - DNA, 6780 bp, synthetic construct
  misc_feature 1..6780: note = plasmid pAAVss-CSkSH5-SPc5-12GTRM-MVM-hGAA-SynthpA
  source 1..6780: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtaccggtcagtttactcaccagggattcagaggcagcactgctgaaccctgagcccttggcacatcaggttggctgtcagaagtcggcctttgtacatacacagttcccttgtgaggcccagctgcgtgtcctaggagcggggcctctctccacagcagagctcagcctctcaagtgtatggacagcacgggtgcctgatgggtggatttagccatgagttgaaggtggcttggggagaatgagagttctagagatagggagaaggggttgccaataggagagtggaattcctgagcacctcgtcacaggcagccgacagaacatgagccgcagggcccaggctatttatacctcgcctgtcactatcagggtccccacagctccccccacctccagccacacacagcaggtccttttgctctttctggtcccttctctactcctccccctccctacctaaggtacccaacccgttacgtggccaccgccttcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagatctagaactagtaagaggtaagggtttaagggatggttggttggtggggtattaatgtttaattacctggagcacctgcctgaaatcactttttttcaggttggcgtacggccaccatgggagtgaggcacccgccctgctcccaccggctcctggccgtctgcgccctcgtgtccttggcaaccgctgcactcctggggcacatcctactccatgatttcctgctggttccccgagagctgagtggctcctccccagtcctggaggagactcacccagctcaccagcagggagccagcagaccagggccccgggatgcccaggcacaccccggccgtcccagagcagtgcccacacagtgcgacgtcccccccaacagccgcttcgattgcgcccctgacaaggccatcacccaggaacagtgcgaggcccgcggctgttgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcatgtccacagccgggcaccgtccccactctacagcgtggagttctccgaggagcccttcggggtgatcgtgcgccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccagtggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcaggggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccagtagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggggaggcccgaggggagctgttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgttagcctaggaataaaagatctttattttcattagatctgtgtgttggttttttgtgtgacccgtctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 134 - DNA, 6355 bp, synthetic construct
  misc_feature 1..6355: note = plasmid pAAVss-hDES1.4kb-MVM-hMTM1co-SynthpA
  source 1..6355: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcggcgcgccaccggtacacacctactagtaacccctccagctggtgatggcaggtctagggtaggaccagtgactggctcctaatcgagcactctattttcagggtttgcattccaaaagggtcaggtccaagagggacctggagtgccaagtggaggtgtagaggcacggccagtacccatggagaatggtggatgtccttaggggttagcaagtgccgtgtgctaaggagggggctttggaggttgggcaggccctctgtggggctccatttttgtgggggtgggggctggagcattatagggggtgggaagtgattggggctgtcaccctagccttccttatctgacgcccacccatgcctcctcaggtaccccctgccccccacagctcctctcctgtgccttgtttcccagccatgcgttctcctctataaatacccgctctggtatttggggttggcagctgttgctgccagggagatggttgggttgacatgcggctcctgacaaaacacaaacccctggtgtgtgtgggcgtgggtggtgtgagtagggggatgaatcagggagggggcgggggacccagggggcaggagccacacaaagtctgtgcgggggtgggagcgcacatagcaattggaaactgaaagcttatcagaccctttctggaaatcagcccactgtttataaacttgaggccccaccctcgacagtaccggggaggaagagggcctgcactagtccagagggaaactgaggctcagggctagctcgcccatagacatacatggcaggcaggctttggccaggatccctccgcctgccaggcgtctccctgccctcccttcctgcctagagacccccaccctcaagcctggctggtctttgcctgagacccaaacctcttcgacttcaagagaatatttaggaacaaggtggtttagggcctttcctgggaacaggccttgaccctttaagaaatgacccaaagtctctccttgaccaaaaaggggaccctcaaactaaagggaagcctctcttctgctgtctcccctgaccccactcccccccaccccaggacgaggagataaccagggctgaaagaggcccgcctgggggctgcagacatgcttgctgcctgccctggcgaaggattggcaggcttgcccgtcacaggacccccgctggctgactcaggggcgcaggcctcttgcgggggagctggcctccccgcccccacggccacgggccgccctttcctggcaggacagcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcacctctagaactagtaagaggtaagggtttaagggatggttggttggtggggtattaatgtttaattacctggagcacctgcctgaaatcactttttttcaggttggcgtacggccaccatggccagcgccagcacaagcaagtacaacagccacagcctggaaaacgagagcatcaagcggaccagcagagatggcgtgaacagagatctgaccgaggccgttcctagactgcctggcgagacactgatcaccgacaaagaagtgatctacatctgccccttcaacggccccatcaagggaagagtgtacatcaccaactaccggctgtacctgcggtccctggaaaccgatagcagcctgattctggatgtgcccctgggcgtgatcagccggattgaaaaaatgggcggagccacctccagaggcgagaatagctatggcctggatatcacatgcaaggacatgcggaacctgagattcgccctgaagcaagagggccacagcagacgggacatgttcgagatcctgaccagatacgcctttcctctggctcactctctgcccctgttcgccttcctgaacgaagagaagttcaacgtggacggctggaccgtgtacaaccccgtggaagagtatagacggcagggactgcccaatcaccactggcggatcaccttcatcaacaagtgctacgagctgtgcgacacataccccgcactgctggtggtgccttacagagcctctgacgacgatctgagaagagtggccacctttcggagccggaacagaatccctgtgctgagctggattcaccccgagaacaagaccgtgatcgtgcggtgttctcagcctctcgtgggcatgagcggcaagagaaacaaggacgacgagaagtacctggacgtgatccgcgagacaaacaagcagatcagcaagctgaccatctacgacgccagaccttctgtgaacgccgtggccaacaaagccacaggcggcggatatgagtccgacgatgcctatcacaacgccgagctgttcttcctggacattcacaacatccatgtgatgcgcgagagcctgaagaaagtgaaggacatcgtgtaccccaatgtggaagagagccactggctgtctagcctggaatccacacactggctggaacacatcaagctggtgctgacaggcgccatccaggtggcagacaaagtgtctagcggcaagtctagcgtgctggtgcactgtagcgacggatgggatagaacagcccagctgacatccctggccatgctgatgctggacagcttctacagatccatcgagggctttgagatcctggtgcagaagaagtggatcagcttcggccacaagttcgcctctagaatcggacacggcgacaagaaccacaccgacgccgatagaagccccatcttcctgcagttcatcgactgcgtgtggcagatgtccaagcagttccctaccgccttcgagttcaacgagcagttcctgatcatcatcctggaccacctgtactcttgcagattcggcaccttcctgttcaactgcgagagcgccagagaacggcagaaagtgaccgagagaaccgtgtctctgtggtccctgatcaacagcaacaaagagaaattcaagaaccccttctacaccaaagaaatcaaccgggtgctgtaccccgtggccagcatgagacatctggaactgtgggtcaactactacatccggtggaaccccagaatcaagcagcagcagcccaatcctgtggaacagcggtatatggaactgctggccctgcgggacgagtacatcaagagactggaagaactgcagctggccaacagcgccaagctgagcgatcctcctacaagccctagcagcccctctcagatgatgccccatgtgcagacccacttttgacctaggaataaaagatctttattttcattagatctgtgtgttggttttttgtgtgacccgtctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 135 - DNA, 6355 bp, synthetic construct
  misc_feature 1..6355: note = plasmid pAAVss-hDES1.4kb-MVM-hMTM1-SynthpA
  source 1..6355: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcggcgcgccaccggtacacacctactagtaacccctccagctggtgatggcaggtctagggtaggaccagtgactggctcctaatcgagcactctattttcagggtttgcattccaaaagggtcaggtccaagagggacctggagtgccaagtggaggtgtagaggcacggccagtacccatggagaatggtggatgtccttaggggttagcaagtgccgtgtgctaaggagggggctttggaggttgggcaggccctctgtggggctccatttttgtgggggtgggggctggagcattatagggggtgggaagtgattggggctgtcaccctagccttccttatctgacgcccacccatgcctcctcaggtaccccctgccccccacagctcctctcctgtgccttgtttcccagccatgcgttctcctctataaatacccgctctggtatttggggttggcagctgttgctgccagggagatggttgggttgacatgcggctcctgacaaaacacaaacccctggtgtgtgtgggcgtgggtggtgtgagtagggggatgaatcagggagggggcgggggacccagggggcaggagccacacaaagtctgtgcgggggtgggagcgcacatagcaattggaaactgaaagcttatcagaccctttctggaaatcagcccactgtttataaacttgaggccccaccctcgacagtaccggggaggaagagggcctgcactagtccagagggaaactgaggctcagggctagctcgcccatagacatacatggcaggcaggctttggccaggatccctccgcctgccaggcgtctccctgccctcccttcctgcctagagacccccaccctcaagcctggctggtctttgcctgagacccaaacctcttcgacttcaagagaatatttaggaacaaggtggtttagggcctttcctgggaacaggccttgaccctttaagaaatgacccaaagtctctccttgaccaaaaaggggaccctcaaactaaagggaagcctctcttctgctgtctcccctgaccccactcccccccaccccaggacgaggagataaccagggctgaaagaggcccgcctgggggctgcagacatgcttgctgcctgccctggcgaaggattggcaggcttgcccgtcacaggacccccgctggctgactcaggggcgcaggcctcttgcgggggagctggcctccccgcccccacggccacgggccgccctttcctggcaggacagcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcacctctagaactagtaagaggtaagggtttaagggatggttggttggtggggtattaatgtttaattacctggagcacctgcctgaaatcactttttttcaggttggcgtacggccaccatggcttctgcatcaacttctaaatataattcacactccttggagaatgagtctattaagaggacgtctcgagatggagtcaatcgagatctcactgaggctgttcctcgacttccaggagaaacactaatcactgacaaagaagttatttacatatgtcctttcaatggccccattaagggaagagtttacatcacaaattatcgtctttatttaagaagtttggaaacggattcttctctaatacttgatgttcctctgggtgtgatctcgagaattgaaaaaatgggaggcgcgacaagtagaggagaaaattcctatggtctagatattacttgtaaagacatgagaaacctgaggttcgctttgaaacaggaaggccacagcagaagagatatgtttgagatcctcacgagatacgcgtttcccctggctcacagtctgccattatttgcatttttaaatgaagaaaagtttaacgtggatggatggacagtttacaatccagtggaagaatacaggaggcagggcttgcccaatcaccattggagaataacttttattaataagtgctatgagctctgtgacacttaccctgctcttttggtggttccgtatcgtgcctcagatgatgacctccggagagttgcaacttttaggtcccgaaatcgaattccagtgctgtcatggattcatccagaaaataagacggtcattgtgcgttgcagtcagcctcttgtcggtatgagtgggaaacgaaataaagatgatgagaaatatctcgatgttatcagggagactaataaacaaatttctaaactcaccatttatgatgcaagacccagcgtaaatgcagtggccaacaaggcaacaggaggaggatatgaaagtgatgatgcatatcataacgccgaacttttcttcttagacattcataatattcatgttatgcgggaatctttaaaaaaagtgaaggacattgtttatcctaatgtagaagaatctcattggttgtccagtttggagtctactcattggttagaacatatcaagctcgttttgacaggagccattcaagtagcagacaaagtttcttcagggaagagttcagtgcttgtgcattgcagtgacggatgggacaggactgctcagctgacatccttggccatgctgatgttggatagcttctataggagcattgaagggttcgaaatactggtacaaaaaaaatggataagttttggacataaatttgcatctcgaataggtcatggtgataaaaaccacaccgatgctgaccgttctcctatttttctccagtttattgattgtgtgtggcaaatgtcaaaacagttccctacagcttttgaattcaatgaacaatttttgattataattttggatcatctgtatagttgccgatttggtactttcttattcaactgtgaatctgctcgagaaagacagaaggttacagaaaggactgtttctttatggtcactgataaacagtaataaagaaaaattcaaaaaccccttctatactaaagaaatcaatcgagttttatatccagttgccagtatgcgtcacttggaactctgggtgaattactacattagatggaaccccaggatcaagcaacaacagccgaatccagtggagcagcgttacatggagctcttagccttacgcgacgaatacataaagcggcttgaggaactgcagctcgccaactctgccaagctttctgatcccccaacttcaccttccagtccttcgcaaatgatgccccatgtgcaaactcacttctgacctaggaataaaagatctttattttcattagatctgtgtgttggttttttgtgtgacccgtctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 136 - DNA, 6808 bp, synthetic construct
  misc_feature 1..6808: note = plasmid pAAVss-SKCRM4-hDES1.4kb-MVM-hMTM1co-SynthpA
  source 1..6808: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcggcgcgccaccggtttctgagtcctctaaggtccctcactcccaactcagccccatgtcctgtcaattcccactcagtgtctgatctccttctcctcacctttcccatctcccgtttgacccaagcttcctgagctctcctcccattcccctttttggagtcctcctcctctcccagaacccagtaataagtgggctcctccctggcctggacccccgtggtaaccctataaggcgaggcagctgctgtctgaggcagggaggggctggtgtgggaggctaagggcagctgctaagtttagggtggctccttctctcttcttagagacaacaggtggctggggcctcagtgcccagaaaagaaaatgtcttagaggtatcggcatgggcctggaggaggggggacagggcagggggaggcatcttcctcaggacatcgggtcctagaggggtacccaacgggttacgacacacctactagtaacccctccagctggtgatggcaggtctagggtaggaccagtgactggctcctaatcgagcactctattttcagggtttgcattccaaaagggtcaggtccaagagggacctggagtgccaagtggaggtgtagaggcacggccagtacccatggagaatggtggatgtccttaggggttagcaagtgccgtgtgctaaggagggggctttggaggttgggcaggccctctgtggggctccatttttgtgggggtgggggctggagcattatagggggtgggaagtgattggggctgtcaccctagccttccttatctgacgcccacccatgcctcctcaggtaccccctgccccccacagctcctctcctgtgccttgtttcccagccatgcgttctcctctataaatacccgctctggtatttggggttggcagctgttgctgccagggagatggttgggttgacatgcggctcctgacaaaacacaaacccctggtgtgtgtgggcgtgggtggtgtgagtagggggatgaatcagggagggggcgggggacccagggggcaggagccacacaaagtctgtgcgggggtgggagcgcacatagcaattggaaactgaaagcttatcagaccctttctggaaatcagcccactgtttataaacttgaggccccaccctcgacagtaccggggaggaagagggcctgcactagtccagagggaaactgaggctcagggctagctcgcccatagacatacatggcaggcaggctttggccaggatccctccgcctgccaggcgtctccctgccctcccttcctgcctagagacccccaccctcaagcctggctggtctttgcctgagacccaaacctcttcgacttcaagagaatatttaggaacaaggtggtttagggcctttcctgggaacaggccttgaccctttaagaaatgacccaaagtctctccttgaccaaaaaggggaccctcaaactaaagggaagcctctcttctgctgtctcccctgaccccactcccccccaccccaggacgaggagataaccagggctgaaagaggcccgcctgggggctgcagacatgcttgctgcctgccctggcgaaggattggcaggcttgcccgtcacaggacccccgctggctgactcaggggcgcaggcctcttgcgggggagctggcctccccgcccccacggccacgggccgccctttcctggcaggacagcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcacctctagaactagtaagaggtaagggtttaagggatggttggttggtggggtattaatgtttaattacctggagcacctgcctgaaatcactttttttcaggttggcgtacggccaccatggccagcgccagcacaagcaagtacaacagccacagcctggaaaacgagagcatcaagcggaccagcagagatggcgtgaacagagatctgaccgaggccgttcctagactgcctggcgagacactgatcaccgacaaagaagtgatctacatctgccccttcaacggccccatcaagggaagagtgtacatcaccaactaccggctgtacctgcggtccctggaaaccgatagcagcctgattctggatgtgcccctgggcgtgatcagccggattgaaaaaatgggcggagccacctccagaggcgagaatagctatggcctggatatcacatgcaaggacatgcggaacctgagattcgccctgaagcaagagggccacagcagacgggacatgttcgagatcctgaccagatacgcctttcctctggctcactctctgcccctgttcgccttcctgaacgaagagaagttcaacgtggacggctggaccgtgtacaaccccgtggaagagtatagacggcagggactgcccaatcaccactggcggatcaccttcatcaacaagtgctacgagctgtgcgacacataccccgcactgctggtggtgccttacagagcctctgacgacgatctgagaagagtggccacctttcggagccggaacagaatccctgtgctgagctggattcaccccgagaacaagaccgtgatcgtgcggtgttctcagcctctcgtgggcatgagcggcaagagaaacaaggacgacgagaagtacctggacgtgatccgcgagacaaacaagcagatcagcaagctgaccatctacgacgccagaccttctgtgaacgccgtggccaacaaagccacaggcggcggatatgagtccgacgatgcctatcacaacgccgagctgttcttcctggacattcacaacatccatgtgatgcgcgagagcctgaagaaagtgaaggacatcgtgtaccccaatgtggaagagagccactggctgtctagcctggaatccacacactggctggaacacatcaagctggtgctgacaggcgccatccaggtggcagacaaagtgtctagcggcaagtctagcgtgctggtgcactgtagcgacggatgggatagaacagcccagctgacatccctggccatgctgatgctggacagcttctacagatccatcgagggctttgagatcctggtgcagaagaagtggatcagcttcggccacaagttcgcctctagaatcggacacggcgacaagaaccacaccgacgccgatagaagccccatcttcctgcagttcatcgactgcgtgtggcagatgtccaagcagttccctaccgccttcgagttcaacgagcagttcctgatcatcatcctggaccacctgtactcttgcagattcggcaccttcctgttcaactgcgagagcgccagagaacggcagaaagtgaccgagagaaccgtgtctctgtggtccctgatcaacagcaacaaagagaaattcaagaaccccttctacaccaaagaaatcaaccgggtgctgtaccccgtggccagcatgagacatctggaactgtgggtcaactactacatccggtggaaccccagaatcaagcagcagcagcccaatcctgtggaacagcggtatatggaactgctggccctgcgggacgagtacatcaagagactggaagaactgcagctggccaacagcgccaagctgagcgatcctcctacaagccctagcagcccctctcagatgatgccccatgtgcagacccacttttgacctaggaataaaagatctttattttcattagatctgtgtgttggttttttgtgtgacccgtctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 137 - DNA, 6808 bp, synthetic construct
  misc_feature 1..6808: note = plasmid pAAVss-SKCRM4-hDES1.4kb-MVM-hMTM1-SynthpA
  source 1..6808: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcggcgcgccaccggtttctgagtcctctaaggtccctcactcccaactcagccccatgtcctgtcaattcccactcagtgtctgatctccttctcctcacctttcccatctcccgtttgacccaagcttcctgagctctcctcccattcccctttttggagtcctcctcctctcccagaacccagtaataagtgggctcctccctggcctggacccccgtggtaaccctataaggcgaggcagctgctgtctgaggcagggaggggctggtgtgggaggctaagggcagctgctaagtttagggtggctccttctctcttcttagagacaacaggtggctggggcctcagtgcccagaaaagaaaatgtcttagaggtatcggcatgggcctggaggaggggggacagggcagggggaggcatcttcctcaggacatcgggtcctagaggggtacccaacgggttacgacacacctactagtaacccctccagctggtgatggcaggtctagggtaggaccagtgactggctcctaatcgagcactctattttcagggtttgcattccaaaagggtcaggtccaagagggacctggagtgccaagtggaggtgtagaggcacggccagtacccatggagaatggtggatgtccttaggggttagcaagtgccgtgtgctaaggagggggctttggaggttgggcaggccctctgtggggctccatttttgtgggggtgggggctggagcattatagggggtgggaagtgattggggctgtcaccctagccttccttatctgacgcccacccatgcctcctcaggtaccccctgccccccacagctcctctcctgtgccttgtttcccagccatgcgttctcctctataaatacccgctctggtatttggggttggcagctgttgctgccagggagatggttgggttgacatgcggctcctgacaaaacacaaacccctggtgtgtgtgggcgtgggtggtgtgagtagggggatgaatcagggagggggcgggggacccagggggcaggagccacacaaagtctgtgcgggggtgggagcgcacatagcaattggaaactgaaagcttatcagaccctttctggaaatcagcccactgtttataaacttgaggccccaccctcgacagtaccggggaggaagagggcctgcactagtccagagggaaactgaggctcagggctagctcgcccatagacatacatggcaggcaggctttggccaggatccctccgcctgccaggcgtctccctgccctcccttcctgcctagagacccccaccctcaagcctggctggtctttgcctgagacccaaacctcttcgacttcaagagaatatttaggaacaaggtggtttagggcctttcctgggaacaggccttgaccctttaagaaatgacccaaagtctctccttgaccaaaaaggggaccctcaaactaaagggaagcctctcttctgctgtctcccctgaccccactcccccccaccccaggacgaggagataaccagggctgaaagaggcccgcctgggggctgcagacatgcttgctgcctgccctggcgaaggattggcaggcttgcccgtcacaggacccccgctggctgactcaggggcgcaggcctcttgcgggggagctggcctccccgcccccacggccacgggccgccctttcctggcaggacagcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcacctctagaactagtaagaggtaagggtttaagggatggttggttggtggggtattaatgtttaattacctggagcacctgcctgaaatcactttttttcaggttggcgtacggccaccatggcttctgcatcaacttctaaatataattcacactccttggagaatgagtctattaagaggacgtctcgagatggagtcaatcgagatctcactgaggctgttcctcgacttccaggagaaacactaatcactgacaaagaagttatttacatatgtcctttcaatggccccattaagggaagagtttacatcacaaattatcgtctttatttaagaagtttggaaacggattcttctctaatacttgatgttcctctgggtgtgatctcgagaattgaaaaaatgggaggcgcgacaagtagaggagaaaattcctatggtctagatattacttgtaaagacatgagaaacctgaggttcgctttgaaacaggaaggccacagcagaagagatatgtttgagatcctcacgagatacgcgtttcccctggctcacagtctgccattatttgcatttttaaatgaagaaaagtttaacgtggatggatggacagtttacaatccagtggaagaatacaggaggcagggcttgcccaatcaccattggagaataacttttattaataagtgctatgagctctgtgacacttaccctgctcttttggtggttccgtatcgtgcctcagatgatgacctccggagagttgcaacttttaggtcccgaaatcgaattccagtgctgtcatggattcatccagaaaataagacggtcattgtgcgttgcagtcagcctcttgtcggtatgagtgggaaacgaaataaagatgatgagaaatatctcgatgttatcagggagactaataaacaaatttctaaactcaccatttatgatgcaagacccagcgtaaatgcagtggccaacaaggcaacaggaggaggatatgaaagtgatgatgcatatcataacgccgaacttttcttcttagacattcataatattcatgttatgcgggaatctttaaaaaaagtgaaggacattgtttatcctaatgtagaagaatctcattggttgtccagtttggagtctactcattggttagaacatatcaagctcgttttgacaggagccattcaagtagcagacaaagtttcttcagggaagagttcagtgcttgtgcattgcagtgacggatgggacaggactgctcagctgacatccttggccatgctgatgttggatagcttctataggagcattgaagggttcgaaatactggtacaaaaaaaatggataagttttggacataaatttgcatctcgaataggtcatggtgataaaaaccacaccgatgctgaccgttctcctatttttctccagtttattgattgtgtgtggcaaatgtcaaaacagttccctacagcttttgaattcaatgaacaatttttgattataattttggatcatctgtatagttgccgatttggtactttcttattcaactgtgaatctgctcgagaaagacagaaggttacagaaaggactgtttctttatggtcactgataaacagtaataaagaaaaattcaaaaaccccttctatactaaagaaatcaatcgagttttatatccagttgccagtatgcgtcacttggaactctgggtgaattactacattagatggaaccccaggatcaagcaacaacagccgaatccagtggagcagcgttacatggagctcttagccttacgcgacgaatacataaagcggcttgaggaactgcagctcgccaactctgccaagctttctgatcccccaacttcaccttccagtccttcgcaaatgatgccccatgtgcaaactcacttctgacctaggaataaaagatctttattttcattagatctgtgtgttggttttttgtgtgacccgtctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 138 - DNA, 6308 bp, synthetic construct
  misc_feature 1..6308: note = plasmid pAAVss-SPc5-12GTRM-MVM-hGAAco-SynthpA
  source 1..6308: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtaccggttggccaccgccttcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagatctagaactagtaagaggtaagggtttaagggatggttggttggtggggtattaatgtttaattacctggagcacctgcctgaaatcactttttttcaggttggcgtacggccaccatgggcgtcagacatcctccatgttctcacagactgctggccgtgtgtgctctggtgtctcttgctacagctgccctgctgggacatatcctgctgcacgattttctgctggtgcccagagagctgtctggcagctctcctgtgctggaagaaacacaccctgcacatcagcagggcgcctctagacctggacctagagatgctcaagcccatcctggcagacctagagccgtgcctacacagtgtgacgtgccacctaacagcagattcgactgcgcccctgacaaggccatcacacaagagcagtgtgaagccagaggctgctgctacattcctgccaaacaaggactgcagggcgctcagatgggacagccttggtgcttcttcccaccatcttaccccagctacaagctggaaaacctgagcagcagcgagatgggctacaccgccacactgaccagaaccacacctacattcttcccaaaggacatcctgacactgcggctggacgtgatgatggaaaccgagaaccggctgcacttcaccatcaaggaccccgccaatagaagatacgaggtgcccctggaaacccctcacgtgcactctagagccccatctccactgtacagcgtggaattcagcgaggaaccctttggcgtgatcgtgcggagacagctggatggcagagtgctgctgaataccacagtggcccctctgttcttcgccgaccagtttctgcagctgagcacaagcctgcctagccagtatatcacaggcctggccgaacacctgtctccactgatgctgagcaccagctggaccagaatcaccctgtggaacagagatctggcccctacacctggcgccaatctgtacggctctcaccctttttatctggccctggaagatggcggaagcgcccacggtgtctttctgctgaacagcaacgccatggacgtggtgctgcaaccatctcctgctctgtcttggagaagcaccggcggcatcctggacgtgtacatctttctgggacccgagcctaagagcgtggtgcagcagtatctggatgtcgtgggctaccccttcatgcctccttattggggcctgggcttccacctgtgtagatggggatacagctccaccgccatcaccagacaggtggtggaaaacatgacccgggctcacttcccactggatgtgcagtggaacgacctggactacatggactccagacgggacttcacctttaacaaggacggcttcagagacttccccgccatggtgcaagaactgcatcaaggcggcagacggtacatgatgatcgtggatcctgccatctcttctagcggccctgccggaagctacagaccttatgatgagggcctgagaagaggcgtgttcatcaccaatgagacaggccagcctctgatcggcaaagtgtggcctggaagcaccgcctttccagacttcaccaatccaaccgctctggcttggtgggaagatatggtggccgagttccacgatcaggtgcccttcgatggcatgtggatcgacatgaacgagcccagcaacttcatcaggggcagcgaggatggctgccccaacaacgaactggaaaatcctccttacgtgccaggcgttgtcggaggaacactgcaggccgccacaatttgtgccagcagccatcagtttctgagcacccactacaacctgcacaacctgtacggcctgaccgaggccattgcctctcatagagccctggttaaggccagaggcacccggccttttgtgatcagcagaagcacatttgccggccacggcagatatgccggacattggacaggggacgtttggtctagttgggagcagctggcctctagcgtgcccgagatcctgcagtttaatctgctgggagtgcccctcgtgggagccgatgtttgtggatttctgggcaacacctccgaggaactgtgcgtcagatggacacagctgggcgccttctatcccttcatgagaaaccacaacagcctgctgagcctgcctcaagagccttacagctttagcgaacccgcacagcaggccatgagaaaggccctgactctgagatacgctctgctgccccacctgtacaccctgtttcatcaagctcatgtggccggcgagacagtggccagaccactgtttctggaattccccaaggacagcagcacctggacagtggatcatcagctgctctggggagaagccctgctcattacacctgtgctgcaggctggcaaggccgaagtgacaggatactttcccctcggcacttggtacgacctgcagacagttcctgtggaagctctgggatctctgcctccacctcctgctgctcctagagagcctgccattcactctgaaggccagtgggttacactgcccgctccactggacaccatcaatgtgcacctgagagccggctacatcatccctctgcaaggccctggactgaccacaaccgaaagcagacagcagccaatggctctggccgtggctctgacaaaaggcggagaagctagaggcgaactgttctgggatgacggcgagagcctggaagtgctggaacggggagcctacacacaagtgatctttctcgcccggaacaacaccatcgtgaacgaactcgtcagagtgaccagtgaaggtgccggactgcagctccagaaagtgacagtgcttggagtggccacagcaccccagcaggttttgtctaatggcgtgcccgtgtccaacttcacatacagccctgacaccaaggtgctggacatctgtgtgtctctgctgatgggcgagcagttcctggtgtcctggtgttgacctaggaataaaagatctttattttcattagatctgtgtgttggttttttgtgtgacccgtctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 139 - DNA, 6308 bp, synthetic construct
  misc_feature 1..6308: note = plasmid pAAVss-SPc5-12GTRM-MVM-hGAA-SynthpA
  source 1..6308: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtaccggttggccaccgccttcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagatctagaactagtaagaggtaagggtttaagggatggttggttggtggggtattaatgtttaattacctggagcacctgcctgaaatcactttttttcaggttggcgtacggccaccatgggagtgaggcacccgccctgctcccaccggctcctggccgtctgcgccctcgtgtccttggcaaccgctgcactcctggggcacatcctactccatgatttcctgctggttccccgagagctgagtggctcctccccagtcctggaggagactcacccagctcaccagcagggagccagcagaccagggccccgggatgcccaggcacaccccggccgtcccagagcagtgcccacacagtgcgacgtcccccccaacagccgcttcgattgcgcccctgacaaggccatcacccaggaacagtgcgaggcccgcggctgttgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcatgtccacagccgggcaccgtccccactctacagcgtggagttctccgaggagcccttcggggtgatcgtgcgccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccagtggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcaggggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccagtagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggggaggcccgaggggagctgttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgttagcctaggaataaaagatctttattttcattagatctgtgtgttggttttttgtgtgacccgtctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 140 - DNA, 21 bp, synthetic construct
  misc_feature 1..21: note = Forward Primer hGAA
  source 1..21: mol_type = other DNA
tgccctcgcagtatatcacag

SEQ ID NO: 141 - DNA, 19 bp, synthetic construct
  misc_feature 1..19: note = Reverse Primer hGAA
  source 1..19: mol_type = other DNA
gagacccgtagaggttcgc

SEQ ID NO: 142 - DNA, 20 bp, synthetic construct
  misc_feature 1..20: note = Forward Primer hGAAco
  source 1..20: mol_type = other DNA
accccttcatgcctccttat

SEQ ID NO: 143 - DNA, 20 bp, synthetic construct
  misc_feature 1..20: note = Reverse Primer hGAAco
  source 1..20: mol_type = other DNA
tccatgtagtccaggtcgtt

SEQ ID NO: 144 - DNA, 23 bp, synthetic construct
  misc_feature 1..23: note = Forward Primer hMTM1
  source 1..23: mol_type = other DNA
gtttgagatcctcacgagatacg

SEQ ID NO: 145 - DNA, 23 bp, synthetic construct
  misc_feature 1..23: note = Reverse Primer hMTM1
  source 1..23: mol_type = other DNA
gtccatccatccacgttaaactt

SEQ ID NO: 146 - DNA, 20 bp, synthetic construct
  misc_feature 1..20: note = Forward Primer hMTM1co
  source 1..20: mol_type = other DNA
ggcaagagaaacaaggacga

SEQ ID NO: 147 - DNA, 20 bp, synthetic construct
  misc_feature 1..20: note = Reverse Primer hMTM1co
  source 1..20: mol_type = other DNA
ggcatcgtcggactcatatc